(19) United States
(12) Patent Application Publication
(10) Pub. No.: US 2024/0382609 A1
(43) Pub. Date: Nov. 21, 2024

(54) MUSCLE TARGETING COMPLEXES AND USES THEREOF FOR TREATING DYSTROPHINOPATHIES

(71) Applicant: Dyne Therapeutics, Inc., Waltham, MA (US)

(72) Inventors: Cody A. Desjardins, Waltham, MA (US); Kim Tang, Waltham, MA (US); James McSwiggen, Arvada, CO (US); Romesh R. Subramanian, Framingham, MA (US); Timothy Weeden, Waltham, MA (US); Mohammed T. Qatanani, Waltham, MA (US); Brendan Quinn, Waltham, MA (US); John Najim, Waltham, MA (US)

(73) Assignee: Dyne Therapeutics, Inc., Waltham, MA (US)

(21) Appl. No.: 18/577,472

(22) PCT Filed: Jul. 8, 2022
(86) PCT No.: PCT/US2022/073534

§ 371 Date: Jan. 8, 2024

Related U.S. Application Data
(60) Provisional application No. 63/220,030, filed on Jul. 9, 2021.

(51) Int. Cl. A61K 47/68; A61P 21/00; C07K 16/28; C12N 15/113

(52) U.S. Cl. CPC A61K 47/6807; A61K 47/6849; A61P 21/00; C07K 16/2881; C12N 15/113; C07K 2317/55; C12N 2310/14; C12N 2310/314; C12N 2310/3233; C12N 2310/3513; C12N 2320/33

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload promotes the expression or activity of a functional dystrophin protein. In some embodiments, the molecular payload is an oligonucleotide, such as an antisense oligonucleotide, e.g., an oligonucleotide that causes exon skipping in a mRNA expressed from a mutant DMD allele.

### RELATED APPLICATIONS

This application claims priority under 35 U.S.C. § 119(e) to U.S. Provisional Application Ser. No. 63/220,030, entitled “MUSCLE TARGETING COMPLEXES AND USES THEREOF FOR TREATING DYSTROPHINOPATHIES”, filed on Jul. 9, 2021, the contents of which are incorporated herein by reference in their entirety.

### FIELD OF THE INVENTION

The present application relates to targeting complexes for delivering molecular payloads (e.g., oligonucleotides) to cells and uses thereof, particularly uses relating to treatment of disease.

### REFERENCE TO AN ELECTRONIC SEQUENCE LISTING

The contents of the electronic sequence listing (D082470066W000-SEQ-COB.xml; Size: 1,203,807 bytes; and Date of Creation: Jul. 7, 2022) are herein incorporated by reference in their entirety.

### BACKGROUND OF INVENTION

Dystrophinopathies are a group of distinct neuromuscular diseases that result from mutations in the gene encoding dystrophin. Dystrophinopathies include Duchenne muscular dystrophy, Becker muscular dystrophy, and X-linked dilated cardiomyopathy. The DMD gene (“DMD”), which encodes dystrophin, is a large gene, containing 79 exons and about 2.6 million total base pairs. Numerous mutations in DMD, including exonic frameshift, deletion, substitution, and duplicative mutations, are able to diminish the expression of functional dystrophin, leading to dystrophinopathies. Several agents that target exons of human DMD have been approved by the U.S. Food and Drug Administration (FDA), including casimersen, viltolarsen, golodirsen, and eteplirsen. Of these, eteplirsen targets exon 51.

### SUMMARY OF INVENTION

According to some aspects, the disclosure provides complexes that target muscle cells for purposes of delivering molecular payloads to those cells, as well as molecular payloads that can be used therein. In some embodiments, complexes provided herein are particularly useful for delivering molecular payloads that increase or restore expression or activity of functional dystrophin protein. In some embodiments, complexes comprise oligonucleotide based molecular payloads that promote expression of functional dystrophin protein through an in-frame exon skipping mechanism or suppression of stop codons, such as by facilitating skipping of DMD exon 51. In some embodiments, molecular payloads provided herein are useful for facilitating exon skipping in a DMD sequence, such as skipping of DMD exon 51. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can promote expression of functional dystrophin protein (e.g., through an exon skipping mechanism, such as by facilitating skipping of DMD exon 51) in the muscle cells. In some embodiments, the oligonucleotides are released by endosomal cleavage of covalent linkers connecting oligonucleotides and muscle-targeting agents of the complexes. Complexes and molecular payloads provided herein can be used for treating subjects having a mutated DMD gene, such as a mutated DMD gene that is amenable to exon 51 skipping.

According to some aspects, complexes comprising an anti-transferrin receptor 1 (TfR1) antibody covalently linked to an oligonucleotide configured for inducing skipping of exon 51 in a DMD pre-mRNA are provided herein, wherein the oligonucleotide comprises a region of complementarity that is complementary with at least 8 consecutive nucleotides of any one of SEQ ID NOs: 160-383.

In some embodiments, the anti-TfR1 antibody comprises:
(i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
(ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
(iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
(iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
(v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
(vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
(vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.

In some embodiments, the anti-TfR1 antibody comprises:
(i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
(ii) a VH comprising an amino acid sequence at least 85% identical to SEQ ID NO: 69; and/or a VL comprising an amino acid sequence at least 85% identical to SEQ ID NO: 70;
(iii) a VH comprising an amino acid sequence at least 85% identical to SEQ ID NO: 71; and/or a VL comprising an amino acid sequence at least 85% identical to SEQ ID NO: 70;
(iv) a VH comprising an amino acid sequence at least 85% identical to SEQ ID NO: 72; and/or a VL comprising an amino acid sequence at least 85% identical to SEQ ID NO: 70;
(v) a VH comprising an amino acid sequence at least 85% identical to SEQ ID NO: 73; and/or a VL comprising an amino acid sequence at least 85% identical to SEQ ID NO: 74;
(vi) a VH comprising an amino acid sequence at least 85% identical to SEQ ID NO: 73; and/or a VL comprising an amino acid sequence at least 85% identical to SEQ ID NO: 75;
(vii) a VH comprising an amino acid sequence at least 85% identical to SEQ ID NO: 76; and/or a VL comprising an amino acid sequence at least 85% identical to SEQ ID NO: 74;
(viii) a VH comprising an amino acid sequence at least 85% identical to SEQ ID NO: 77; and/or a VL comprising an amino acid sequence at least 85% identical to SEQ ID NO: 78;
(ix) a VH comprising an amino acid sequence at least 85% identical to SEQ ID NO: 79; and/or a VL comprising an amino acid sequence at least 85% identical to SEQ ID NO: 80; or
(x) a VH comprising an amino acid sequence at least 85% identical to SEQ ID NO: 77; and/or a VL comprising an amino acid sequence at least 85% identical to SEQ ID NO: 80.

In some embodiments, the anti-TfR1 antibody comprises:
(i) a VH comprising the amino acid sequence of SEQ ID NO: 76 and a VL comprising the amino acid sequence of SEQ ID NO: 75;
(ii) a VH comprising the amino acid sequence of SEQ ID NO: 69 and a VL comprising the amino acid sequence of SEQ ID NO: 70;
(iii) a VH comprising the amino acid sequence of SEQ ID NO: 71 and a VL comprising the amino acid sequence of SEQ ID NO: 70;
(iv) a VH comprising the amino acid sequence of SEQ ID NO: 72 and a VL comprising the amino acid sequence of SEQ ID NO: 70;
(v) a VH comprising the amino acid sequence of SEQ ID NO: 73 and a VL comprising the amino acid sequence of SEQ ID NO: 74;
(vi) a VH comprising the amino acid sequence of SEQ ID NO: 73 and a VL comprising the amino acid sequence of SEQ ID NO: 75;
(vii) a VH comprising the amino acid sequence of SEQ ID NO: 76 and a VL comprising the amino acid sequence of SEQ ID NO: 74;
(viii) a VH comprising the amino acid sequence of SEQ ID NO: 77 and a VL comprising the amino acid sequence of SEQ ID NO: 78;
(ix) a VH comprising the amino acid sequence of SEQ ID NO: 79 and a VL comprising the amino acid sequence of SEQ ID NO: 80; or
(x) a VH comprising the amino acid sequence of SEQ ID NO: 77 and a VL comprising the amino acid sequence of SEQ ID NO: 80.

In some embodiments, the anti-TfR1 antibody is a Fab fragment, a Fab′ fragment, a F(ab′)₂ fragment, an scFv, an Fv, or a full-length IgG.

In some embodiments, the anti-TfR1 antibody is a Fab fragment.

In some embodiments, the anti-TfR1 antibody comprises:
(i) a heavy chain comprising an amino acid sequence at least 85% identical to SEQ ID NO: 101; and/or a light chain comprising an amino acid sequence at least 85% identical to SEQ ID NO: 90;
(ii) a heavy chain comprising an amino acid sequence at least 85% identical to SEQ ID NO: 97; and/or a light chain comprising an amino acid sequence at least 85% identical to SEQ ID NO: 85;
(iii) a heavy chain comprising an amino acid sequence at least 85% identical to SEQ ID NO: 98; and/or a light chain comprising an amino acid sequence at least 85% identical to SEQ ID NO: 85;
(iv) a heavy chain comprising an amino acid sequence at least 85% identical to SEQ ID NO: 99; and/or a light chain comprising an amino acid sequence at least 85% identical to SEQ ID NO: 85;
(v) a heavy chain comprising an amino acid sequence at least 85% identical to SEQ ID NO: 100; and/or a light chain comprising an amino acid sequence at least 85% identical to SEQ ID NO: 89;
(vi) a heavy chain comprising an amino acid sequence at least 85% identical to SEQ ID NO: 100; and/or a light chain comprising an amino acid sequence at least 85% identical to SEQ ID NO: 90;
(vii) a heavy chain comprising an amino acid sequence at least 85% identical to SEQ ID NO: 101; and/or a light chain comprising an amino acid sequence at least 85% identical to SEQ ID NO: 89;
(viii) a heavy chain comprising an amino acid sequence at least 85% identical to SEQ ID NO: 102; and/or a light chain comprising an amino acid sequence at least 85% identical to SEQ ID NO: 93;
(ix) a heavy chain comprising an amino acid sequence at least 85% identical to SEQ ID NO: 103; and/or a light chain comprising an amino acid sequence at least 85% identical to SEQ ID NO: 95; or
(x) a heavy chain comprising an amino acid sequence at least 85% identical to SEQ ID NO: 102; and/or a light chain comprising an amino acid sequence at least 85% identical to SEQ ID NO: 95.

In some embodiments, the anti-TfR1 antibody comprises:
(i) a heavy chain comprising the amino acid sequence of SEQ ID NO: 101; and a light chain comprising the amino acid sequence of SEQ ID NO: 90;
(ii) a heavy chain comprising the amino acid sequence of SEQ ID NO: 97; and a light chain comprising the amino acid sequence of SEQ ID NO: 85;
(iii) a heavy chain comprising the amino acid sequence of SEQ ID NO: 98; and a light chain comprising the amino acid sequence of SEQ ID NO: 85;
(iv) a heavy chain comprising the amino acid sequence of SEQ ID NO: 99; and a light chain comprising the amino acid sequence of SEQ ID NO: 85;
(v) a heavy chain comprising the amino acid sequence of SEQ ID NO: 100; and a light chain comprising the amino acid sequence of SEQ ID NO: 89;
(vi) a heavy chain comprising the amino acid sequence of SEQ ID NO: 100; and a light chain comprising the amino acid sequence of SEQ ID NO: 90;
(vii) a heavy chain comprising the amino acid sequence of SEQ ID NO: 101; and a light chain comprising the amino acid sequence of SEQ ID NO: 89;
(viii) a heavy chain comprising the amino acid sequence of SEQ ID NO: 102; and a light chain comprising the amino acid sequence of SEQ ID NO: 93;
(ix) a heavy chain comprising the amino acid sequence of SEQ ID NO: 103; and a light chain comprising the amino acid sequence of SEQ ID NO: 95; or
(x) a heavy chain comprising the amino acid sequence of SEQ ID NO: 102; and a light chain comprising the amino acid sequence of SEQ ID NO: 95.

In some embodiments, the anti-TfR1 antibody does not specifically bind to the transferrin binding site of the transferrin receptor 1 and/or the anti-TfR1 antibody does not inhibit binding of transferrin to the transferrin receptor 1.

In some embodiments, the oligonucleotide comprises a region of complementarity to at least 4 consecutive nucleotides of a splicing feature of the DMD pre-mRNA.

In some embodiments, the splicing feature is an exonic splicing enhancer (ESE) in exon 51 of the DMD pre-mRNA, optionally wherein the ESE comprises a sequence of any one of SEQ ID NOs: 860-894.

In some embodiments, the splicing feature is a branch point, a splice donor site, or a splice acceptor site, optionally wherein the splicing feature is across the junction of exon 50 and intron 50, in intron 50, across the junction of intron 50 and exon 51, across the junction of exon 51 and intron 51, in intron 51, or across the junction of intron 51 and exon 52 of the DMD pre-mRNA, and further optionally wherein the splicing feature comprises a sequence of any one of SEQ ID NOs: 855-859 and 895-898.

In some embodiments, the oligonucleotide comprises a sequence complementary to any one of SEQ ID NOs: 160-383 or comprises a sequence of any one of SEQ ID NOs: 384-831, wherein each thymine base (T) may independently and optionally be replaced with a uracil base (U), and each U may independently and optionally be replaced with a T.

In some embodiments, the oligonucleotide comprises one or more phosphorodiamidate morpholinos, optionally wherein the oligonucleotide is a phosphorodiamidate morpholino oligomer (PMO).

In some embodiments, the anti-TfR1 antibody is covalently linked to the oligonucleotide via a cleavable linker, optionally wherein the cleavable linker comprises a valine-citrulline sequence.

In some embodiments, the anti-TfR1 antibody is covalently linked to the oligonucleotide via conjugation to a lysine residue or a cysteine residue of the antibody.

According to some aspects, oligonucleotides that target DMD are provided herein, wherein the oligonucleotide comprises a region of complementarity to any one of SEQ ID NOs: 160-383, optionally wherein the region of complementarity comprises at least 15 consecutive nucleosides complementary to any one of SEQ ID NOs: 160-383.

In some embodiments, the oligonucleotide comprises at least 15 consecutive nucleosides of any one of SEQ ID NOs: 384-831, optionally wherein the oligonucleotide comprises a sequence of any one of SEQ ID NOs: 384-831, wherein each thymine base (T) may independently and optionally be replaced with a uracil base (U), and each U may independently and optionally be replaced with a T.

According to some aspects, methods of delivering an oligonucleotide to a cell are provided herein, the method comprising contacting the cell with a complex disclosed herein or with an oligonucleotide disclosed herein.

According to some aspects, methods of promoting the expression or activity of a dystrophin protein in a cell are provided herein, the method comprising contacting the cell with a complex disclosed herein or with an oligonucleotide disclosed herein in an amount effective for promoting internalization of the oligonucleotide to the cell, optionally wherein the cell is a muscle cell.

In some embodiments, the subject has a DMD gene that is amenable to skipping of exon 51.

In some embodiments, the DMD protein is a truncated DMD protein.

### BRIEF DESCRIPTION OF THE DRAWINGS

FIG. 1 shows data illustrating that conjugates containing anti-TfR1 Fab (3M12 VH4/Vκ3) conjugated to a DMD exon-skipping oligonucleotide resulted in enhanced exon skipping compared to the naked DMD exon skipping oligo in Duchenne muscular dystrophy patient myotubes.

### DETAILED DESCRIPTION OF INVENTION

Aspects of the disclosure relate to a recognition that while certain molecular payloads (e.g., oligonucleotides, peptides, small molecules) can have beneficial effects in muscle cells, it has proven challenging to effectively target such cells. Accordingly, as described herein, the present disclosure provides complexes comprising muscle-targeting agents covalently linked to molecular payloads in order to overcome such challenges. In some embodiments, the complexes are particularly useful for delivering molecular payloads that modulate (e.g., promote) the expression or activity of dystrophin protein (e.g., a truncated dystrophin protein) or DMD (e.g., a mutated DMD allele). In some embodiments, complexes provided herein may comprise oligonucleotides that promote expression and activity of dystrophin protein or DMD, such as by facilitating in-frame exon skipping and/or suppression of premature stop codons. For example, complexes may comprise oligonucleotides that induce skipping of exon(s) of DMD RNA (e.g., pre-mRNA), such as oligonucleotides that induce skipping of exon 51. In some embodiments, synthetic nucleic acid payloads (e.g., DNA or RNA payloads) may be used that express one or more proteins that promote normal expression and activity of dystrophin protein or DMD.

Duchenne muscular dystrophy is an X-linked muscular disorder caused by one or more mutations in the DMD gene located on Xp21. Dystrophin protein typically forms the dystrophin-associated glycoprotein complex (DGC) at the sarcolemma, which links the muscle sarcomeric structure to the extracellular matrix and protects the sarcolemma from contraction-induced injury. In patients with Duchenne muscular dystrophy, the dystrophin protein is generally absent and muscle fibers typically become damaged due to mechanical overextension. Mutations in the DMD gene are associated with two types of muscular dystrophy, Duchenne muscular dystrophy and Becker muscular dystrophy, depending on whether the translational reading frame is lost or maintained. Becker muscular dystrophy is a clinically milder form of Duchenne muscular dystrophy, and is characterized by features similar to Duchenne muscular dystrophy. In some embodiments, exon skipping induced by oligonucleotides (e.g., delivered using complexes provided herein) can be used to restore the reading frame of a mutated DMD allele resulting in production of a truncated dystrophin protein that is sufficiently functional to improve muscle function. In some embodiments, such exon skipping converts a Duchenne muscular dystrophy phenotype into a milder Becker muscular dystrophy phenotype.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).
Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).
Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444⁻⁶⁴⁴⁸; Poljak, R. J., et al. (1994) Structure 2:1121⁻¹¹²³). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93⁻¹⁰¹) and use of a cysteine residue, a marker peptide and a C-terminal polyhistidine tag to make bivalent and biotinylated scFv molecules (Kipriyanov, S. M., et al. (1994) Mol. Immunol. 31:1047⁻¹⁰⁵⁸).
Branch point: As used herein, the term “branch point” or “branch site” refers to a nucleic acid sequence motif within an intron of a gene or pre-mRNA that is involved in splicing of pre-mRNA into mRNA (i.e., removing introns from the pre-mRNA), and can be referred to as a splicing feature. A branch point is typically located 18 to 40 nucleotides from the 3′ end of an intron, and contains an adenine but is otherwise relatively unrestricted in sequence. Common sequence motifs for branch points are YNYYRAY, YTRAC, and YNYTRAY, where Y is a pyrimidine, N is any nucleotide, R is any purine, and A is adenine. During splicing, the pre-mRNA is cleaved at the 5′ end of the intron, which then attaches to the branch point region downstream through transesterification bonding between guanines and adenines from the 5′ end and the branch point, respectively, to form a looped lariat structure.
CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593⁻⁵⁹⁷ (2005); Lefranc, M.-P. et al., Nucleic Acids Res., 37:D1006⁻¹⁰¹² (2009); Lefranc, M.-P. et al., Nucleic Acids Res., 43:D413-422 (2015); Chothia et al., (1989) Nature 342:877; Chothia, C. et al. (1987) J. Mol. Biol. 196:901⁻⁹¹⁷, Al-lazikani et al (1997) J. Molec. Biol. 273:927⁻⁹⁴⁸; and Almagro, J. Mol. Recognit. 17:132-143 (2004). See also bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133⁻¹³⁹ (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.
Chimeric antibody: The term “chimeric antibody” refers to antibodies which comprise heavy and light chain variable region sequences from one species and constant region sequences from another species, such as antibodies having murine heavy and light chain variable regions linked to human constant regions.
Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.
Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.
Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.
Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.
DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene (DMD or DMD gene) may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2, NM_004009.3, NM_004010.3 and NM_004011.3) have been characterized that encode different protein isoforms.
DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 45, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657⁻⁶⁶, the contents of which are incorporated herein by reference in its entirety.
Dystrophinopathy: As used herein, the term “dystrophinopathy” refers to a muscle disease results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.
Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106⁻¹⁰. (2000), incorporated herein by reference. ESEs can be referred to as splicing features. ESEs may direct or enhance splicing, for example, to remove one or more introns and/or one or more exons from a gene transcript. ESE motifs are typically 6⁻⁸ nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.
Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.
Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.
Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.
Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.
Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.
Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382⁻³⁹¹ and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.
Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.
Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.
Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.
Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.
Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62⁻⁷⁰; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287⁻⁶²⁹⁵; Kellermann S-A., and Green L. L. (2002) Current Opinion in Biotechnology 13:593⁻⁵⁹⁷; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.
Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.
Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10-4 M, 10-5 M, 10-6 M, 10-7 M, 10-8 M, 10-9 M, 10-10 M, 10-11 M, 10-12 M, 10-13 M, or less. In some embodiments, an antibody specifically binds to the transferrin receptor, e.g., an epitope of the apical domain of transferrin receptor.
Splice acceptor site: As used herein, the term “splice acceptor site” or “splice acceptor” refers to a nucleic acid sequence motif at the 3′ end of an intron or across an intron/exon junction of a gene or pre-mRNA that is involved in splicing of pre-mRNA into mRNA (i.e., removing introns from the pre-mRNA), and can be referred to as a splicing feature. A splice acceptor site includes a terminal AG sequence at the 3′ end of an intron, which is typically preceded (5′-ward) by a region high in pyrimidines (C/U). Upstream from the splice acceptor site is the branch point. Formation of a lariat loop intermediate structure by a transesterification reaction between the branch point and the splice donor site releases a 3′-OH of the 5′ exon, which subsequently reacts with the first nucleotide of the 3′ exon, thereby joining the exons and releasing the intron lariat. The AG sequence at the 3′ end of the intron in the splice acceptor site is known to be critical for proper splicing, as changing one of these nucleotides results in inhibition of splicing. Rarely, alternative splice acceptor sites have an AC at the 3′ end of the intron, instead of the more common AG. A common splice acceptor site motif has a sequence of or similar to [Y-rich region]-NCAGG or Y_{X}NYAGG, in which Y represents a pyrimidine, N represents any nucleotide, and x is a number from 4 to 20. The cut site follows the AG, which represent the 3′-terminal nucleotides of the excised intron.
Splice donor site: As used herein, the term “splice donor site” or “splice donor” refers to a nucleic acid sequence motif at the 5′ end of an intron or across an exon/intron junction of a gene or pre-mRNA that is involved in splicing of pre-mRNA into mRNA (i.e., removing introns from the pre-mRNA), and can be referred to as a splicing feature. A splice donor site includes a terminal GU sequence at the 5′ end of the intron, within a larger and fairly unconstrained sequence. During splicing, the 2′-OH of a nucleotide within the branch point initiates a transesterification reaction via a nucleophilic attack on the 5′ G of the intron within the splice donor site. The G is thereby cleaved from the pre-mRNA and bonds instead to the branch point nucleotide, forming a loop lariat structure. The 3′ nucleotide of the upstream exon subsequently binds the splice acceptor site, joining the exons and excising the intron. A typical splice donor site has a sequence of or similar to GGGURAGU or AGGURNG, in which R represents a purine and N represents any nucleotide. The cut site precedes the first GU (i.e., GG/GURAGU or AG/GURNG), which represent the 5′-terminal nucleotides of the excised intron.
Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy. In some embodiments, a subject is a patient that has a mutation of the DMD gene that is amenable to exon 51 skipping.
Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).
2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O-N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

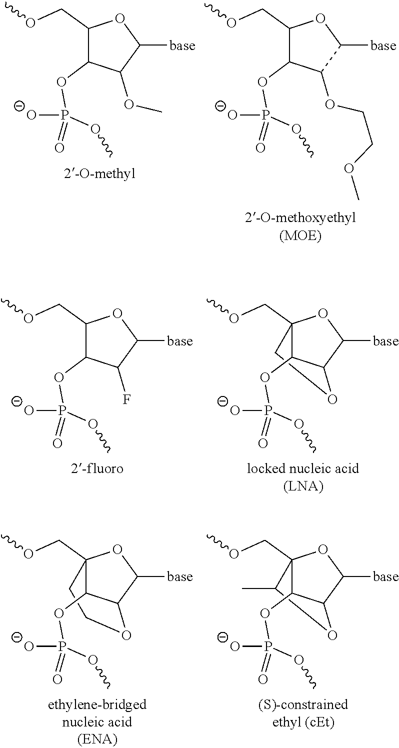

These examples are shown with phosphate groups, but any internucleoside linkages are contemplated between 2′-modified nucleosides.

### II. Complexes

Provided herein are complexes that comprise a targeting agent, e.g. an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present within a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell.

In some embodiments, a complex comprises a muscle-targeting agent, e.g., an anti-transferrin receptor antibody, covalently linked to a molecular payload, e.g., an antisense oligonucleotide that targets DMD to promote exon skipping, e.g., in a transcript encoded from a mutated DMD allele. In some embodiments, the complex targets a DMD pre-mRNA to promote skipping of exon 51 in the DMD pre-mRNA.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure. It should also be appreciated that any muscle targets (e.g., muscle surface proteins) can be targeted by any type of muscle-targeting agent described herein. For example, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). The muscle-targeting agent may comprise, or consist of, a small molecule. Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861⁻³; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 Mar. 39(13):78309; the entire contents of each of which are incorporated herein by reference.

a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34⁻⁴¹.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEEENTDN
NTKPNGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEENTDN
NTKANGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLOWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDVPTSSRLYWADLKTLLSEKLNSIEFADTIKQLS
QNTYTPREAGSQKDESLAYYIENQFHEFKFSKVWRDEHYVKIQVKSSIGQ
NMVTIVQSNGNLDPVESPEGYVAFSKPTEVSGKLVHANFGTKKDFEELSY
SVNGSLVIVRAGEITFAEKVANAQSFNAIGVLIYMDKNKFPVVEADLALF
GHAHLGTGDPYTPGFPSFNHTQFPPSQSSGLPNIPVQTISRAAAEKLFGK
MEGSCPARWNIDSSCKLELSQNQNVKLIVKNVLKERRILNIFGVIKGYEE
PDRYVVVGAQRDALGAGVAAKSSVGTGLLLKLAQVFSDMISKDGFRPSRS
IIFASWTAGDFGAVGATEWLEGYLSSLHLKAFTYINLDKVVLGTSNFKVS
ASPLLYTLMGKIMQDVKHPVDGKSLYRDSNWISKVEKLSFDNAAYPFLAY
SGIPAVSFCFCEDADYPYLGTRLDTYEALTQKVPQLNQMVRTAAEVAGQL
IIKLTHDVELNLDYEMYNSKLLSFMKDLNQFKTDIRDMGLSLQWLYSARG
DYFRATSRLTTDFHNAEKTNRFVMREINDRIMKVEYHFLSPYVSPRESPF
RHIFWGSGSHTLSALVENLKLRQKNITAFNETLFRNQLALATWTIQGVAN
ALSGDIWNIDNEF

In some embodiments, an anti-TfR1 antibody binds to an amino acid segment of the receptor as follows: FVKIQVKDSAQNSVIIVDKNGRLVYLVENPGGYVAYSKAATVTGKLVHANFGTKKDFE DLYTPVNGSIVIVRAGKITFAEKVANAESLNAIGVLIYMDQTKFPIVNAELSFFGHAHLG TGDPYTPGFPSFNHTQFPPSRSSGLPNIPVQTISRAAAEKLFGNMEGDCPSDWKTDSTCR MVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O- glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a K_{D} of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY (SEQ ID NO: | DDYMY (SEQ ID NO: 7) | GFNIKDD (SEQ ID NO: 12) |
| | H1 | 1) | | |
| | CDR- | IDPENGDT (SEQ ID NO: | WIDPENGDTEYASKFQD | ENG (SEQ ID NO: 13) |
| | H2 | 2) | (SEQ ID NO: 8) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPENGDT | | |
| | | EYASKFQDKATVTADTSSNTAYLQLSSLTSEDTAVYYCTLWLRRGLDYWGQGTSVTVS | | |
| | | S (SEQ ID NO: 17) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-A4 | CDR- | GFNIKDDY (SEQ ID NO: | DDYMY (SEQ ID NO: 7) | GFNIKDD (SEQ ID NO: 12) |
| N54T* | H1 | 1) | | |
| | CDR- | IDPETGDT (SEQ ID NO: | WIDPETGDTEYASKFQD | ETG (SEQ ID NO: 21) |
| | H2 | 19) | (SEQ ID NO: 20) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS(SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6 | | |
| | VH | EVQLQQSGAELVRPGASVKLSCTASGFNIKDDYMYWVKQRPEQGLEWIGWIDPETGDT | | |
| | | EYASKFQDKATVTADTSSNTAYLQLSSLTSEDTAVYYCTLWLRRGLDYWGQGTSVTVS | | |
| | | S (SEQ ID NO: 22) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-A4 | CDR- | GFNIKDDY (SEQ ID NO: | DDYMY (SEQ ID NO: 7) | GFNIKDD (SEQ ID NO: 12) |
| N54S* | H1 | 1) | | |
| | CDR- | IDPESGDT (SEQ ID NO: | WIDPESGDTEYASKFQD | ESG (SEQ ID NO: 25) |
| | H2 | 23) | (SEQ ID NO: 24) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPESGDT | | |
| | | EYASKFQDKATVTADTSSNTAYLQLSSLTSEDTAVYYCTLWLRRGLDYWGQGTSVTVS | | |
| | | S (SEQ ID NO: 26) | | |
| | VL | DIVMTQAAPSVPVTPGESVSISCRSSKSLLHSNGYTYLFWFLQRPGQSPQLLIYRMSN | | |
| | | LASGVPDRFSGSGSGTAFTLRISRVEAEDVGVYYCMQHLEYPFTFGGGTKLEIK | | |
| | | (SEQ ID NO: 18) | | |
| 3-M12 | CDR- | GYSITSGYY (SEQ ID | SGYYWN (SEQ ID NO: 33) | GYSITSGY (SEQ ID NO: |
| | H1 | NO: 27) | | 38) |
| | CDR- | ITFDGAN (SEQ ID NO: | YITFDGANNYNPSLKN (SEQ | FDG (SEQ ID NO: 39) |
| | H2 | 28) | ID NO: 34) | |
| | CDR- | TRSSYDYDVLDY (SEQ | SSYDYDVLDY (SEQ ID NO: | SYDYDVLD (SEQ ID NO: |
| | H3 | ID NO: 29) | 35) | 40) |
| | CDR- | QDISNF (SEQ ID NO: 30) | RASQDISNFLN (SEQ ID NO: | SQDISNF (SEQ ID NO: 41) |
| | L1 | | 36) | |
| | CDR- | YTS (SEQ ID NO: 31) | YTSRLHS (SEQ ID NO: 37) | YTS (SEQ ID NO: 31) |
| | L2 | | | |
| | CDR- | QQGHTLPYT (SEQ ID | QQGHTLPYT (SEQ ID NO: 32) | GHTLPY (SEQ ID NO: 42) |
| | L3 | NO: 32) | | |
| | VH | DVQLQESGPGLVKPSQSLSLTCSVTGYSITSGYYWNWIRQFPGNKLEWMGYITFDGAN | | |
| | | NYNPSLKNRISITRDTSKNQFFLKLTSVTTEDTATYYCTRSSYDYDVLDYWGQGTTLT | | |
| | | VSS (SEQ ID NO: 43) | | |
| | VL | DIQMTQTTSSLSASLGDRVTISCRASQDISNFLNWYQQRPDGTVKLLIYYTSRLHSGV | | |
| | | PSRFSGSGSGTDFSLTVSNLEQEDIATYFCQQGHTLPYTFGGGTKLEIK (SEQ ID | | |
| | | NO: 44) | | |
| 5-H12 | CDR- | GYSFTDYC (SEQ ID NO: | DYCIN (SEQ ID NO: 51) | GYSFTDY (SEQ ID NO: 56) |
| | H1 | 45) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYCINWVNQRPGQGLEWIGWIYPGSGNT | | |
| | | RYSERFKGKATLTVDTSSNTAYMQLSSLTSEDSAVYFCAREDYYPYHGMDYWGQGTSV | | |
| | | TVSS (SEQ ID NO: 61) | | |
| | VL | DIVLTQSPTSLAVSLGQRATISCRASESVDGYDNSFMHWYQQKPGQPPKLLIFRASNL | | |
| | | ESGIPARFSGSGSRTDFTLTINPVEAADVATYYCQQSSEDPWTFGGGTKLEIK (SEQ | | |
| | | ID NO: 62) | | |
| 5-H12 | CDR | GYSFTDYY (SEQ ID | DYYIN (SEQ ID NO: 64) | GYSFTDY (SEQ ID NO: 56) |
| C33Y* | H1 | NO: 63) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYYINWVNQRPGQGLEWIGWIYPGSGNT | | |
| | | RYSERFKGKATLTVDTSSNTAYMQLSSLTSEDSAVYFCAREDYYPYHGMDYWGQGTSV | | |
| | | TVSS (SEQ ID NO: 65) | | |
| | VL | DIVLTQSPTSLAVSLGQRATISCRASESVDGYDNSFMHWYQQKPGQPPKLLIFRASNL | | |
| | | ESGIPARFSGSGSRTDFTLTINPVEAADVATYYCQQSSEDPWTFGGGTKLEIK (SEQ | | |
| | | ID NO: 62) | | |
| 5-H12 | CDR- | GYSFTDYD (SEQ ID | DYDIN (SEQ ID NO: 67) | GYSFTDY (SEQ ID NO: 56) |
| C33D* | H1 | NO: 66) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYDINWVNQRPGQGLEWIGWIYPGSGNT | | |
| | | RYSERFKGKATLTVDTSSNTAYMQLSSLTSEDSAVYFCAREDYYPYHGMDYWGQGTSV | | |
| | | TVSS (SEQ ID NO: 68) | | |
| | VL | DIVLTQSPTSLAVSLGQRATISCRASESVDGYDNSFMHWYQQKPGQPPKLLIFRASNL | | |
| | | ESGIPARFSGSGSRTDFTLTINPVEAADVATYYCQQSSEDPWTFGGGTKLEIK (SEQ | | |
| | | ID NO: 62) | | |
| Anti- | CDR- | GYSFTSYW (SEQ ID | SYWIG (SEQ ID NO: 144) | GYSFTSY (SEQ ID NO: |
| TfR | H1 | NO: 138) | | 149) |
| clone 8 | | | | |
| | CDR- | IYPGDSDT (SEQ ID NO: | IIYPGDSDTRYSPSFQGQ | GDS (SEQ ID NO: 150) |
| | H2 | 139) | (SEQ ID NO: 145) | |
| | CDR- | ARFPYDSSGYYSFDY | FPYDSSGYYSFDY (SEQ ID | PYDSSGYYSFD (SEQ ID |
| | H3 | (SEQ ID NO: 140) | NO: 146) | NO: 151) |
| | CDR- | QSISSY (SEQ ID NO: | RASQSISSYLN (SEQ ID NO: | SQSISSY (SEQ ID NO: 152) |
| | L1 | 141) | 147) | |
| | CDR- | AAS (SEQ ID NO: 142) | AASSLQS (SEQ ID NO: 148) | AAS (SEQ ID NO: 142) |
| | L2 | | | |
| | CDR- | QQSYSTPLT (SEQ ID | QQSYSTPLT (SEQ ID NO: | SYSTPL (SEQ ID NO: 153) |
| | L3 | NO: 143) | 143) | |

*mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of anti-TfR1 antibodies described herein are provided in Table 3.

TABLE 3
Variable Regions of Anti-TfR1 Antibodies

| Antibody | Variable Region Amino Acid Sequence** |
|---|---|
| 3A4 | V_{H}: |
| VH3 | EVQLVQSGSELKKPGASVKVSCTASGFNIKDDYMYWVRQPPGKGLEWIGWIDP |
| (N54T*)/ | ETGDTEYASKFQDRVTVTADTSTNTAYMELSSLRSEDTAVYYCTLWLRRGLDY |
| Vκ4 | WGQGTLVTVSS (SEQ ID NO: 69) |
| | V_{L}: |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTFGGG |
| | TKVEIK (SEQ ID NO: 70) |
| 3A4 | V_{H}: |
| VH3 | EVQLVQSGSELKKPGASVKVSCTASGFNIKDDYMYWVRQPPGKGLEWIGWIDP |
| (N54S*)/ | ESGDTEYASKFQDRVTVTADTSTNTAYMELSSLRSEDTAVYYCTLWLRRGLDY |
| Vκ4 | WGQGTLVTVSS (SEQ ID NO: 71) |
| | V_{L}: |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTFGGG |
| | TKVEIK (SEQ ID NO: 70) |
| 3A4 | V_{H}: |
| VH3/Vκ4 | EVQLVQSGSELKKPGASVKVSCTASGFNIKDDYMYWVRQPPGKGLEWIGWIDP |
| | ENGDTEYASKFQDRVTVTADTSTNTAYMELSSLRSEDTAVYYCTLWLRRGLDY |
| | WGQGTLVTVSS (SEQ ID NO: 72) |
| | V_{L}: |
| | DIVMTQSPLSLPVTPGEPASISCRSSKSLLHSNGYTYLFWFQQRPGQSPRLLI |
| | YRMSNLASGVPDRFSGSGSGTDFTLKISRVEAEDVGVYYCMQHLEYPFTFGGG |
| | TKVEIK (SEQ ID NO: 70) |
| 3M12 | V_{H}: |
| VH3/Vκ2 | QVQLQESGPGLVKPSQTLSLTCSVTGYSITSGYYWNWIRQPPGKGLEWMGYIT |
| | FDGANNYNPSLKNRVSISRDTSKNQFSLKLSSVTAEDTATYYCTRSSYDYDVL |
| | DYWGQGTTVTVSS (SEQ ID NO: 73) |
| | V_{L}: |
| | DIQMTQSPSSLSASVGDRVTITCRASQDISNFLNWYQQKPGQPVKLLIYYTSR |
| | LHSGVPSRFSGSGSGTDFTLTISSLQPEDFATYFCQQGHTLPYTFGQGTKLEI |
| | K (SEQ ID NO: 74) |
| 3M12 | V_{H}: |
| VH3/Vκ3 | QVQLQESGPGLVKPSQTLSLTCSVTGYSITSGYYWNWIRQPPGKGLEWMGYIT |
| | FDGANNYNPSLKNRVSISRDTSKNQFSLKLSSVTAEDTATYYCTRSSYDYDVL |
| | DYWGQGTTVTVSS (SEQ ID NO: 73) |
| | V_{L}: |
| | DIQMTQSPSSLSASVGDRVTITCRASQDISNFLNWYQQKPGQPVKLLIYYTSR |
| | LHSGVPSRFSGSGSGTDFTLTISSLQPEDFATYYCQQGHTLPYTFGQGTKLEI |
| | K (SEQ ID NO: 75) |
| 3M12 | V_{H} |
| VH4/Vκ2 | QVQLQESGPGLVKPSQTLSLTCTVTGYSITSGYYWNWIRQPPGKGLEWIGYIT |
| | FDGANNYNPSLKNRVSISRDTSKNQFSLKLSSVTAEDTATYYCTRSSYDYDVL |
| | DYWGQGTTVTVSS (SEQ ID NO: 76) |
| | V_{L}: |
| | DIQMTQSPSSLSASVGDRVTITCRASQDISNFLNWYQQKPGQPVKLLIYYTSR |
| | LHSGVPSRFSGSGSGTDFTLTISSLQPEDFATYFCQQGHTLPYTFGQGTKLEI |
| | K (SEQ ID NO: 74) |
| 3M12 | V_{H}: |
| VH4/Vκ3 | QVQLQESGPGLVKPSQTLSLTCTVTGYSITSGYYWNWIRQPPGKGLEWIGYIT |
| | FDGANNYNPSLKNRVSISRDTSKNQFSLKLSSVTAEDTATYYCTRSSYDYDVL |
| | DYWGQGTTVTVSS (SEQ ID NO: 76) |
| | V_{L}: |
| | DIQMTQSPSSLSASVGDRVTITCRASQDISNFLNWYQQKPGQPVKLLIYYTSR |
| | LHSGVPSRFSGSGSGTDFTLTISSLQPEDFATYYCQQGHTLPYTFGQGTKLEI |
| | K (SEQ ID NO: 75) |
| 5H12 | V_{H}: |
| VH5 | QVQLVQSGAEVKKPGASVKVSCKASGYSFTDYYINWVRQAPGQGLEWMGWIYP |
| (C33Y*)/ | GSGNTRYSERFKGRVTITRDTSASTAYMELSSLRSEDTAVYYCAREDYYPYHG |
| Vκ3 | MDYWGQGTLVTVSS (SEQ ID NO: 77) |
| | V_{L}: |
| | DIVLTQSPDSLAVSLGERATINCRASESVDGYDNSFMHWYQQKPGQPPKLLIF |
| | RASNLESGVPDRFSGSGSRTDFTLTISSLQAEDVAVYYCQQSSEDPWTFGQGT |
| | KLEIK (SEQ ID NO: 78) |
| 5H12 | V_{H}: |
| VH5 | QVQLVQSGAEVKKPGASVKVSCKASGYSFTDYDINWVRQAPGQGLEWMGWIYP |
| (C33D*)/ | GSGNTRYSERFKGRVTITRDTSASTAYMELSSLRSEDTAVYYCAREDYYPYHG |
| Vκ4 | MDYWGQGTLVTVSS (SEQ ID NO: 79) |
| | V_{L}: |
| | DIVMTQSPDSLAVSLGERATINCRASESVDGYDNSFMHWYQQKPGQPPKLLIF |
| | RASNLESGVPDRFSGSGSGTDFTLTISSLQAEDVAVYYCQQSSEDPWTFGQGT |
| | KLEIK (SEQ ID NO: 80) |
| 5H12 | V_{H}: |
| VH5 | QVQLVQSGAEVKKPGASVKVSCKASGYSFTDYYINWVRQAPGQGLEWMGWIYP |
| (C33Y*)/ | GSGNTRYSERFKGRVTITRDTSASTAYMELSSLRSEDTAVYYCAREDYYPYHG |
| Vκ4 | MDYWGQGTLVTVSS (SEQ ID NO: 77) |
| | V_{L}: |
| | DIVMTQSPDSLAVSLGERATINCRASESVDGYDNSFMHWYQQKPGQPPKLLIF |
| | RASNLESGVPDRFSGSGSGTDFTLTISSLQAEDVAVYYCQQSSEDPWTFGQGT |
| | KLEIK (SEQ ID NO: 80) |
| Anti-TfR | V_{H}: |
| clone 8 | QVQLVQSGAEVKKPGESLKISCKGSGYSFTSYWIGWVRQMPGKGLEWMGIIYP |
| | GDSDTRYSPSFQGQVTISADKSISTAYLQWSSLKASDTAMYYCARFPYDSSGY |
| | YSFDYWGQGTLVTVSS (SEQ ID NO: 154) |
| | V_{L}: |
| | DIQMTQSPSSLSASVGDRVTITCRASQSISSYLNWYQQKPGKAPKLLIYAASS |
| | LQSGVPSRFSGSGSGTDFTLTISSLQPEDFATYYCQQSYSTPLTFGGGTKVEI |
| | K (SEQ ID NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php, both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of
examples of anti-TfR1 IgGs

| Antibody | IgG Heavy Chain/Light Chain Sequences** |
|---|---|
| 3A4 | Heavy Chain (with wild type human IgG1 constant |
| VH3 | region) |
| (N54T*)/ | EVQLVQSGSELKKPGASVKVSCTASGFNIKDDYMYWVRQPPGKGLEWIGWIDPE |
| Vκ4 | TGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEAL |
| | HNHYTQKSLSLSPGK (SEQ ID NO: 84) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3A4 | Heavy Chain (with wild type human IgG1 constant |
| VH3 | region) |
| (N54S*)/ | EVQLVQSGSELKKPGASVKVSCTASGFNIKDDYMYWVRQPPGKGLEWIGWIDPE |
| Vκ4 | SGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEAL |
| | HNHYTQKSLSLSPGK (SEQ ID NO: 86) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3A4 | Heavy Chain (with wild type human IgG1 constant |
| VH3/Vκ4 | region) |
| | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHED |
| | PEVKENWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEAL |
| | HNHYTQKSLSLSPGK (SEQ ID NO: 87) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3M12 | Heavy Chain (with wild type human IgG1 constant |
| VH3/Vκ2 | region) |
| | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK (SEQ ID NO: 88) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 89) |
| 3M12 | Heavy Chain (with wild type human IgG1 constant |
| VH3/Vκ3 | region) |
| | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK (SEQ ID NO: 88) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 90) |
| 3M12 | Heavy Chain (with wild type human IgG1 constant |
| VH4/Vκ2 | region) |
| | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK (SEQ ID NO: 91) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 89) |
| 3M12 | Heavy Chain (with wild type human IgG1 constant |
| VH4/Vκ3 | region) |
| | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHE |
| | ALHNHYTQKSLSLSPGK (SEQ ID NO: 91) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 90) |
| 5H12 | Heavy Chain (with wild type human IgG1 constant |
| VH5 | region) |
| (C33Y*)/ | QVQLVQSGAEVKKPGASVKVSCKASGYSFTDYYINWVRQAPGQGLEWMGWIYPG |
| Vκ3 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 92) |
| | Light Chain (with kappa light chain constant |
| | region) |
| | DIVLTQSPDSLAVSLGERATINCRASESVDGYDNSFMHWYQQKPGQPPKLLIFR |
| | ASNLESGVPDRESGSGSRTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 93) |
| 5H12 | Heavy Chain (with wild type human IgG1 constant |
| VH5 | region) |
| (C33D*)/ | QVQLVQSGAEVKKPGASVKVSCKASGYSFTDYDINWVRQAPGQGLEWMGWIYPG |
| Vκ4 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 94) |
| | Light Chain (with kappa light chain constant |
| | region) |
| | DIVMTQSPDSLAVSLGERATINCRASESVDGYDNSFMHWYQQKPGQPPKLLIFR |
| | ASNLESGVPDRESGSGSGTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 95) |
| 5H12 | Heavy Chain (with wild type human IgG1 constant |
| VH5 | region) |
| (C33Y*)/ | QVQLVQSGAEVKKPGASVKVSCKASGYSFTDYYINWVRQAPGQGLEWMGWIYPG |
| Vκ4 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDVS |
| | HEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMH |
| | EALHNHYTQKSLSLSPGK (SEQ ID NO: 92) |
| | Light Chain (with kappa light chain constant |
| | region) |
| | DIVMTQSPDSLAVSLGERATINCRASESVDGYDNSFMHWYQQKPGQPPKLLIFR |
| | ASNLESGVPDRESGSGSGTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 95) |
| Anti-TfR | VH: |
| clone 8 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYS |
| | FDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSV |
| | MHEALHNHYTQKSLSLSPGK (SEQ ID NO: 156) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPLTFGGGTKVEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 157) |

*mutation positions are according to Kabat numbering of the respective VH sequences containing the mutations
**CDRs according to the Kabat numbering system are bolded;
VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHT

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of
examples of anti-TfR1 Fabs

| Antibody | Fab Heavy Chain/Light Chain Sequences** |
|---|---|
| 3A4 | Heavy Chain (with partial human IgG1 constant |
| VH3 | region) |
| (N54T*)/ | EVQLVQSGSELKKPGASVKVSCTASGFNIKDDYMYWVRQPPGKGLEWIGWIDPE |
| Vκ4 | TGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHT (SEQ ID NO: 97) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3A4 | Heavy Chain (with partial human IgG1 constant |
| VH3 | region) |
| (N54S*)/ | EVQLVQSGSELKKPGASVKVSCTASGFNIKDDYMYWVRQPPGKGLEWIGWIDPE |
| Vκ4 | SGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHT (SEQ ID NO: 98) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3A4 | Heavy Chain (with partial human IgG1 constant |
| VH3/Vκ4 | region) |
| | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVE |
| | PKSCDKTHT (SEQ ID NO: 99) |
| | Light Chain (with kappa light chain constant |
| | region) |
| | DIVMTQSPLSLPVTPGEPASISCRSSKSLLHSNGYTYLFWFQQRPGQSPRLLIY |
| | RMSNLASGVPDRESGSGSGTDFTLKISRVEAEDVGVYYCMQHLEYPFTFGGGTK |
| | VEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC (SEQ ID NO: 85) |
| 3M12 | Heavy Chain (with partial human IgG1 constant |
| VH3/Vκ2 | region) |
| | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHT (SEQ ID NO: 100) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 89) |
| 3M12 | Heavy Chain (with partial human IgG1 constant |
| VH3/Vκ3 | region) |
| | QVQLQESGPGLVKPSQTLSLTCSVTGYSITSGYYWNWIRQPPGKGLEWMGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHT (SEQ ID NO: 100) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 90) |
| 3M12 | Heavy Chain (with partial human IgG1 constant |
| VH4/Vκ2 | region) |
| | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHT (SEQ ID NO: 101) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYFCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 89) |
| 3M12 | Heavy Chain (with partial human IgG1 constant |
| VH4/Vκ3 | region) |
| | QVQLQESGPGLVKPSQTLSLTCTVTGYSITSGYYWNWIRQPPGKGLEWIGYITF |
| | DGANNYNPSLKNRVSISRDTSKNQFSLKLSSVTAEDTATYYCTRSSYDYDVLDY |
| | WGQGTTVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKK |
| | VEPKSCDKTHT (SEQ ID NO: 101) |
| | Light Chain (with kappa light chain constant |
| | region) |
| | DIQMTQSPSSLSASVGDRVTITCRASQDISNFLNWYQQKPGQPVKLLIYYTSRL |
| | HSGVPSRFSGSGSGTDFTLTISSLQPEDFATYYCQQGHTLPYTFGQGTKLEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 90) |
| 5H12 | Heavy Chain (with partial human IgG1 constant |
| VH5 | region) |
| (C33Y*)/ | QVQLVQSGAEVKKPGASVKVSCKASGYSFTDYYINWVRQAPGQGLEWMGWIYPG |
| Vκ3 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHT (SEQ ID NO: 102) |
| | Light Chain (with kappa light chain constant |
| | region) |
| | DIVLTQSPDSLAVSLGERATINCRASESVDGYDNSFMHWYQQKPGQPPKLLIFR |
| | ASNLESGVPDRESGSGSRTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 93) |
| 5H12 | Heavy Chain (with partial human IgG1 constant |
| VH5 | region) |
| (C33D*)/ | QVQLVQSGAEVKKPGASVKVSCKASGYSFTDYDINWVRQAPGQGLEWMGWIYPG |
| Vκ4 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHT (SEQ ID NO: 103) |
| | Light Chain (with kappa light chain constant |
| | region) |
| | DIVMTQSPDSLAVSLGERATINCRASESVDGYDNSFMHWYQQKPGQPPKLLIFR |
| | ASNLESGVPDRESGSGSGTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 95) |
| 5H12 | Heavy Chain (with partial human IgG1 constant |
| VH5 | region) |
| (C33Y*)/ | QVQLVQSGAEVKKPGASVKVSCKASGYSFTDYYINWVRQAPGQGLEWMGWIYPG |
| Vκ4 | SGNTRYSERFKGRVTITRDTSASTAYMELSSLRSEDTAVYYCAREDYYPYHGMD |
| | YWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDK |
| | KVEPKSCDKTHT (SEQ ID NO: 102) |
| | Light Chain (with kappa light chain constant |
| | region) |
| | DIVMTQSPDSLAVSLGERATINCRASESVDGYDNSFMHWYQQKPGQPPKLLIFR |
| | ASNLESGVPDRESGSGSGTDFTLTISSLQAEDVAVYYCQQSSEDPWTFGQGTKL |
| | EIKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRG |
| | EC (SEQ ID NO: 95) |
| Anti-TfR | VH: |
| clone 8 | QVQLVQSGAEVKKPGESLKISCKGSGYSFTSYWIGWVRQMPGKGLEWMGIIYPG |
| Version 1 | DSDTRYSPSFQGQVTISADKSISTAYLQWSSLKASDTAMYYCARFPYDSSGYYS |
| | FDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDKTHTCP (SEQ ID NO: 158) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPLTFGGGTKVEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 157) |
| Anti-TfR | VH: |
| clone 8 | QVQLVQSGAEVKKPGESLKISCKGSGYSFTSYWIGWVRQMPGKGLEWMGIIYPG |
| Version 2 | DSDTRYSPSFQGQVTISADKSISTAYLQWSSLKASDTAMYYCARFPYDSSGYYS |
| | FDYWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKKVEPKSCDKTHT (SEQ ID NO: 159) |
| | VL: |
| | DIQMTQSPSSLSASVGDRVTITCRASQSISSYLNWYQQKPGKAPKLLIYAASSL |
| | QSGVPSRFSGSGSGTDFTLTISSLQPEDFATYYCQQSYSTPLTFGGGTKVEIKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 157) |

*mutation positions are according to Kabat numbering of the respective VH sequences containing the mutations
**CDRs according to the Kabat numbering system are bolded;
VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the an-TfR1 antibody of the present disclosure comprises a heavy chain comprising the amino acid sequence of SEQ ID NO: 159 and a light chain comprising the amino acid sequence of SEQ ID NO: 157.

Other known anti-TfR1 antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the and-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated
references and binding epitope information.

| Antibody Clone | | |
|---|---|---|
| Name | Reference(s) | Epitope/Notes |
| OKT9 | U.S. Pat. No. 4,364,934, filed Dec. 4, 1979, | Apical domain of TfR1 |
| | entitled “MONOCLONAL ANTIBODY TO | (residues 305-366 of |
| | A HUMAN EARLY THYMOCYTE | human TfR1 sequence |
| | ANTIGEN AND METHODS FOR | XM_052730.3, available |
| | PREPARING SAME” | in GenBank) |
| | Schneider C. et al. “Structural features of the | |
| | cell surface receptor for transferrin that is | |
| | recognized by the monoclonal antibody | |
| | OKT9.” J Biol Chem. 1982, 257: 14, 8516-8522. | |
| (From JCR) | WO 2015/098989, filed Dec. 24, 2014, | Apical domain (residues |
| Clone M11 | “Novel anti-Transferrin receptor antibody | 230-244 and 326-347 of |
| Clone M23 | that passes through blood-brain barrier” | TfR1) and protease-like |
| Clone M27 | U.S. Pat. No. 9,994,641, filed | domain (residues 461- |
| Clone B84 | Dec. 24, 2014, “Novel anti-Transferrin | 473) |
| | receptor antibody that passes through | |
| | blood-brain barrier” | |
| (From | WO 2016/081643, filed May 26, 2016, | Apical domain and non- |
| Genentech) | entitled “ANTI-TRANSFERRIN | apical regions |
| 7A4, 8A2, 15D2, | RECEPTOR ANTIBODIES AND | |
| 10D11, 7B10, | METHODS OF USE” | |
| 15G11, 16G5, | U.S. Pat. No. 9,708,406, filed | |
| 13C3, 16G4, | May 20, 2014, “Anti-transferrin receptor | |
| 16F6, 7G7, 4C2, | antibodies and methods of use” | |
| 1B12, and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” | |
| | 2000, J Pharmacol. Exp. Ther., 292: 1048-1052. | |
| | US Patent App. 2010/077498, filed | |
| | Sep. 11, 2008, entitled “COMPOSITIONS AND | |
| | METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of | |
| | the human transferrin receptor in plant cells: | |
| | effects of tyrphostin A23 and brefeldin A. | |
| | Plant J 48: 757-70 (2006). | |
| 1A1B2, 66IG10, | Commercially available anti- | Novus Biologicals |
| MEM-189, | transferrin receptor antibodies. | 8100 Southpark Way, |
| JF0956, 29806, | | A-8 Littleton CO |
| 1A1B2, | | 80120 |
| TFRC/1818, | | |
| 1E6, 66Ig10, | | |
| TFRC/1059, | | |
| Q1/71, 23D10, | | |
| 13E4, | | |
| TFRC/1149, ER- | | |
| MP21, | | |
| YTA74.4, BU54, | | |
| 2B6, RI7 217 | | |
| (From INSERM) | US Patent App. 2011/0311544A1, | Does not compete with |
| BA120g | filed Jun. 15, 2005, entitled “ANTI-CD71 | OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti- | BioXcell |
| 5E9C11, | transferrin receptor antibodies. | 10 Technology Dr., |
| OKT9 (BE0023 | | Suite 2B |
| clone) | | West Lebanon, NH |
| | | 03784-1671 USA |
| BK19.9, B3/25, | Gatter, K. C. et al. “Transferrin receptors | |
| T56/14 and | in human tissues: their distribution and | |
| T58/1 | possible clinical relevance.” J Clin | |
| | Pathol. 1983 May; 36(5): 539-45. | |

Anti-TfR1 antibody
CDRH1 (SEQ ID NO: 952)
CDRH2 (SEQ ID NO: 953)
CDRH3 (SEQ ID NO: 954)
CDRL1 (SEQ ID NO: 955)
CDRL2 (SEQ ID NO: 956)
CDRL3 (SEQ ID NO: 957)
VH (SEQ ID NO: 958)
VL (SEQ ID NO: 959)

Additional Anti-TfR1 antibody SEQ ID NOs

| | VH/VL | CDR1 | CDR2 | CDR3 |
|---|---|---|---|---|
| VH1 | 967 | 960 | 961 | 954 |
| VH2 | 968 | 960 | 962 | 954 |
| VH3 | 969 | 960 | 963 | 954 |
| VH4 | 970 | 960 | 962 | 954 |
| VL1 | 971 | 955 | 956 | 115 |
| VL2 | 972 | 955 | 956 | 115 |
| VL3 | 973 | 955 | 964 | 957 |
| VL4 | 974 | 965 | 966 | 957 |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any ofthe CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of
an example of a known anti-TfR1 antibody

| Sequence Type | Kabat | Chothia | Contact |
|---|---|---|---|
| CDR-H1 | SYWMH (SEQ ID | GYTFTSY (SEQ ID NO: | TSYWMH (SEQ ID NO: |
| | NO: 110) | 116) | 118) |
| CDR-H2 | EINPTNGRTNYIE | NPTNGR (SEQ ID NO: | WIGEINPTNGRTN (SEQ ID |
| | KFKS (SEQ ID | 117) | NO: 119) |
| | NO: 111) | | |
| CDR-H3 | GTRAYHY (SEQ | GTRAYHY (SEQ ID NO: | ARGTRA (SEQ ID NO: |
| | ID NO: 112) | 112) | 120) |
| CDR-L1 | RASDNLYSNLA | RASDNLYSNLA (SEQ ID | YSNLAWY (SEQ ID NO: |
| | (SEQ ID NO: | NO: 113) | 121) |
| | 113) | | |
| CDR-L2 | DATNLAD (SEQ | DATNLAD (SEQ ID NO: | LLVYDATNLA (SEQ ID NO: |
| | ID NO: 114) | 114) | 122) |
| CDR-L3 | QHFWGTPLT | QHFWGTPLT (SEQ ID | QHFWGTPL (SEQ ID NO: |
| | (SEQ ID NO: | NO: 115) | 123) |
| | 115) | | |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATN |
| | LADGVPSRFSGSGSGTQYSLKINSLQSEDFGTYYCQHFWGTPLTFGAGTKLEL |
| | K (SEQ ID NO: 125) |
| Humanized VH | EVQLVQSGAEVKKPGASVKVSCKASGYTFTSYWMHWVRQAPGQRLEWIGEINP |
| | TNGRTNYIEKFKSRATLTVDKSASTAYMELSSLRSEDTAVYYCARGTRAYHYW |
| | GQGTMVTVSS (SEQ ID NO: 128) |
| Humanized VL | DIQMTQSPSSLSASVGDRVTITCRASDNLYSNLAWYQQKPGKSPKLLVYDATN |
| | LADGVPSRFSGSGSGTDYTLTISSLQPEDFATYYCQHFWGTPLTFGQGTKVEI |
| | K (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDV |
| | SHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSC |
| | SVMHEALHNHYTQKSLSLSPGK (SEQ ID NO: 132) |
| LC of chimeric | DIQMTQSPASLSVSVGETVTITCRASDNLYSNLAWYQQKQGKSPQLLVYDATN |
| full-length IgG1 | LADGVPSRFSGSGSGTQYSLKINSLQSEDFGTYYCQHFWGTPLTFGAGTKLEL |
| | KRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRG |
| | EC (SEQ ID NO: 133) |
| HC of fully human | EVQLVQSGAEVKKPGASVKVSCKASGYTFTSYWMHWVRQAPGQRLEWIGEINP |
| full-length IgG1 | TNGRTNYIEKFKSRATLTVDKSASTAYMELSSLRSEDTAVYYCARGTRAYHYW |
| | GQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCVVVDV |
| | SHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSC |
| | SVMHEALHNHYTQKSLSLSPGK (SEQ ID NO: 134) |
| LC of fully human | DIQMTQSPSSLSASVGDRVTITCRASDNLYSNLA WYQQKPGKSPKLLVYDAT |
| full-length IgG1 | NLADGVPSRFSGSGSGTDYTLTISSLQPEDFATYYCQHFWGTPLTFGQGTKVE |
| | IKRTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNR |
| | GEC (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDK |
| | KVEPKSCDKTHTCP (SEQ ID NO: 136) |
| HC of fully human | EVQLVQSGAEVKKPGASVKVSCKASGYTFTSYWMHWVRQAPGQRLEWIGEINP |
| Fab | TNGRTNYIEKFKSRATLTVDKSASTAYMELSSLRSEDTAVYYCARGTRAYHYW |
| | GQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDK |
| | KVEPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the anti-TfR1 antibody described herein is an scFv. In some embodiments, the anti-TfR1 antibody described herein is an scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fc receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O- glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, myosin IIb or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxKl, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A.M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 943) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 943). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for Duchenne muscular dystrophy. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 944) was identified and this muscle-targeting peptide showed improved binding to C2C12 cells relative to the ASSLNIA (SEQ ID NO: 943) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 945) appeared most frequently. Accordingly, in some embodiments, the muscle-targeting agent comprises the amino acid sequence TARGEHKEEELI (SEQ ID NO: 945).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 946), CSERSMNFC (SEQ ID NO: 947), CPKTRRVPC (SEQ ID NO: 948), WLSEAGPVVTVRALRGTGSW (SEQ ID NO: 949), ASSLNIA (SEQ ID NO: 943), CMQHSMRVC (SEQ ID NO: 950), and DDTRHWG (SEQ ID NO: 951). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10-15 kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., for modulating a biological outcome, e.g., the transcription of a DNA sequence, the splicing and processing of a RNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of molecular payloads may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a mutated DMD allele. Exemplary molecular payloads are described in further detail herein, however, it should be appreciated that the exemplary molecular payloads provided herein are not meant to be limiting.

i. Oligonucleotides

Aspects of the disclosure relate to oligonucleotides configured to modulate (e.g., increase) expression of dystrophin, e.g., from a DMD allele. In some embodiments, oligonucleotides provided herein are configured to alter splicing of DMD pre-mRNA to promote expression of dystrophin protein (e.g., a functional truncated dystrophin protein). In some embodiments, oligonucleotides provided herein are configured to promote skipping of one or more exons in DMD, e.g., in a mutated DMD allele, in order to restore the reading frame. In some embodiments, the oligonucleotides allow for functional dystrophin protein expression (e.g., as described in Kinali M, Arechevala-Gomeza V, Feng L, et al. Local restoration of dystrophin expression with the morpholino oligomer AVI-4658 in Duchenne muscular dystrophy: a single-blind, placebo-controlled, dose-escalation, proof-of-concept study. Lancet Neurol. 2009; 8(10):918⁻⁹²⁸ and Watanabe N, Nagata T, Satou Y, et al. NS-065/NCNP-01: an antisense oligonucleotide for potential treatment of exon 53 skipping in Duchenne muscular dystrophy. Mol Ther Nucleic Acids. 2018; 13:442-449). In some embodiments, oligonucleotides provided are configured to promote skipping of exon 51 to produce a shorter but functional version of dystrophin (e.g., containing an in-frame deletion). In some embodiments, oligonucleotides are provided that promote exon 51 skipping (e.g., which may be relevant in a substantial number of patients, including, for example, patients amenable to exon 51 skipping, such as those having deletions in DMD exons 3-50, 4-50, 5-50, 6-50, 9-50, 10-50, 11-50, 13-50, 14-50, 15-50, 16-50, 17-50, 19-50, 21-50, 23-50, 24-50, 25-50, 26-50, 27-50, 28-50, 29-50, 30-50, 31-50, 32-50, 33-50, 34-50, 35-50, 36-50, 37-50, 38-50, 39-50, 40-50, 41-50, 42-50, 43-50, 45-50, 47-50, 48-50, 49-50, 50, 52, 52-58, 52-61, 52-63, 52-64, 52-66, 52-76, or 52-77).

Table 8 provides non-limiting examples of sequences of oligonucleotides that are useful for targeting DMD, e.g., for exon skipping, and for target sequences within DMD. In some embodiments, an oligonucleotide may comprise any antisense sequence provided in Table 8 or a sequence complementary to a target sequence provided in Table 8.

TABLE 8
Oligonucleotide sequences for targeting DMD.

| SEQ | Target | SEQ | Antisense | SEQ | Antisense | |
|---|---|---|---|---|---|---|
| ID | sequence^{†} | ID | Sequence^{†} | ID | Sequence^{†} | Target |
| NO | (5′ to 3′) | NO | (5′ to 3′) | NO | (5′ to 3′) | Site |
| 160 | GUAAGUAUACUGG | 384 | GAAUGGGAUCCAG | 608 | GAATGGGATCCAG | Intron 50 |
| | AUCCCAUUC | | UAUACUUAC | | TATACTTAC | |
| 161 | GUAAGUAUACUGG | 385 | AGAAUGGGAUCCA | 609 | AGAATGGGATCCA | Intron 50 |
| | AUCCCAUUCU | | GUAUACUUAC | | GTATACTTAC | |
| 162 | GUAAGUAUACUGG | 386 | GAGAAUGGGAUCC | 610 | GAGAATGGGATCC | Intron 50 |
| | AUCCCAUUCUC | | AGUAUACUUAC | | AGTATACTTAC | |
| 163 | GUAAGUAUACUGG | 387 | AGAGAAUGGGAUC | 611 | AGAGAATGGGATC | Intron 50 |
| | AUCCCAUUCUCU | | CAGUAUACUUAC | | CAGTATACTTAC | |
| 164 | UAAGUAUACUGGA | 388 | GAGAAUGGGAUCC | 612 | GAGAATGGGATCC | Intron 50 |
| | UCCCAUUCUC | | AGUAUACUUA | | AGTATACTTA | |
| 165 | AAGUAUACUGGAU | 389 | GAGAAUGGGAUCC | 613 | GAGAATGGGATCC | Intron 50 |
| | CCCAUUCUC | | AGUAUACUU | | AGTATACTT | |
| 166 | AGUAUACUGGAUC | 390 | GAGAAUGGGAUCC | 614 | GAGAATGGGATCC | Intron 50 |
| | CCAUUCUC | | AGUAUACU | | AGTATACT | |
| 167 | GUAUACUGGAUCC | 391 | CCAAAGAGAAUGG | 615 | CCAAAGAGAATGG | Intron 50 |
| | CAUUCUCUUUGG | | GAUCCAGUAUAC | | GATCCAGTATAC | |
| 168 | UACUGGAUCCCAU | 392 | GAGCCAAAGAGAA | 616 | GAGCCAAAGAGAA | Intron 50 |
| | UCUCUUUGGCUC | | UGGGAUCCAGUA | | TGGGATCCAGTA | |
| 169 | ACUGGAUCCCAUU | 393 | GAGCCAAAGAGAA | 617 | GAGCCAAAGAGAA | Intron 50 |
| | CUCUUUGGCUC | | UGGGAUCCAGU | | TGGGATCCAGT | |
| 170 | UGUGGUUACUAAG | 394 | AUGGCAGUUUCCU | 618 | ATGGCAGTTTCCT | Exon 51 |
| | GAAACUGCCAU | | UAGUAACCACA | | TAGTAACCACA | |
| 171 | UGUGGUUACUAAG | 395 | GAUGGCAGUUUCC | 619 | GATGGCAGTTTCC | Exon 51 |
| | GAAACUGCCAUC | | UUAGUAACCACA | | TTAGTAACCACA | |
| 172 | GUGGUUACUAAGG | 396 | AUGGCAGUUUCCU | 620 | ATGGCAGTTTCCT | Exon 51 |
| | AAACUGCCAU | | UAGUAACCAC | | TAGTAACCAC | |
| 173 | GUGGUUACUAAGG | 397 | GAUGGCAGUUUCC | 621 | GATGGCAGTTTCC | Exon 51 |
| | AAACUGCCAUC | | UUAGUAACCAC | | TTAGTAACCAC | |
| 174 | GUGGUUACUAAGG | 398 | AGAUGGCAGUUUC | 622 | AGATGGCAGTTTC | Exon 51 |
| | AAACUGCCAUCU | | CUUAGUAACCAC | | CTTAGTAACCAC | |
| 175 | UGGUUACUAAGGA | 399 | GAUGGCAGUUUCC | 623 | GATGGCAGTTTCC | Exon 51 |
| | AACUGCCAUC | | UUAGUAACCA | | TTAGTAACCA | |
| 176 | GGUUACUAAGGAA | 400 | GAUGGCAGUUUCC | 624 | GATGGCAGTTTCC | Exon 51 |
| | ACUGCCAUC | | UUAGUAACC | | TTAGTAACC | |
| 177 | GAAACUGCCAUCU | 401 | UUCUAGUUUGGAG | 625 | TTCTAGTTTGGAG | Exon 51 |
| | CCAAACUAGAA | | AUGGCAGUUUC | | ATGGCAGTTTC | |
| 178 | AAACUGCCAUCUC | 402 | CUAGUUUGGAGAU | 626 | CTAGTTTGGAGAT | Exon 51 |
| | CAAACUAG | | GGCAGUUU | | GGCAGTTT | |
| 179 | AAACUGCCAUCUC | 403 | UCUAGUUUGGAGA | 627 | TCTAGTTTGGAGA | Exon 51 |
| | CAAACUAGA | | UGGCAGUUU | | TGGCAGTTT | |
| 180 | AAACUGCCAUCUC | 404 | UUCUAGUUUGGAG | 628 | TTCTAGTTTGGAG | Exon 51 |
| | CAAACUAGAA | | AUGGCAGUUU | | ATGGCAGTTT | |
| 181 | AACUGCCAUCUCC | 405 | CUAGUUUGGAGAU | 629 | CTAGTTTGGAGAT | Exon 51 |
| | AAACUAG | | GGCAGUU | | GGCAGTT | |
| 182 | AACUGCCAUCUCC | 406 | UCUAGUUUGGAGA | 630 | TCTAGTTTGGAGA | Exon 51 |
| | AAACUAGA | | UGGCAGUU | | TGGCAGTT | |
| 183 | AACUGCCAUCUCC | 407 | UUCUAGUUUGGAG | 631 | TTCTAGTTTGGAG | Exon 51 |
| | AAACUAGAA | | AUGGCAGUU | | ATGGCAGTT | |
| 184 | ACUGCCAUCUCCA | 408 | UCUAGUUUGGAGA | 632 | TCTAGTTTGGAGA | Exon 51 |
| | AACUAGA | | UGGCAGU | | TGGCAGT | |
| 185 | ACUGCCAUCUCCA | 409 | UUCUAGUUUGGAG | 633 | TTCTAGTTTGGAG | Exon 51 |
| | AACUAGAA | | AUGGCAGU | | ATGGCAGT | |
| 186 | UCUCCAAACUAGA | 410 | GAUGGCAUUUCUA | 634 | GATGGCATTTCTA | Exon 51 |
| | AAUGCCAUC | | GUUUGGAGA | | GTTTGGAGA | |
| 187 | CUCCAAACUAGAA | 411 | GAUGGCAUUUCUA | 635 | GATGGCATTTCTA | Exon 51 |
| | AUGCCAUC | | GUUUGGAG | | GTTTGGAG | |
| 188 | UCCAAACUAGAAA | 412 | GAUGGCAUUUCUA | 636 | GATGGCATTTCTA | Exon 51 |
| | UGCCAUC | | GUUUGGA | | GTTTGGA | |
| 189 | GAUUUCAACCGGG | 413 | UCUGUCCAAGCCC | 637 | TCTGTCCAAGCCC | Exon 51 |
| | CUUGGACAGA | | GGUUGAAAUC | | GGTTGAAATC | |
| 190 | GAUUUCAACCGGG | 414 | UUCUGUCCAAGCC | 638 | TTCTGTCCAAGCC | Exon 51 |
| | CUUGGACAGAA | | CGGUUGAAAUC | | CGGTTGAAATC | |
| 191 | AUUUCAACCGGGC | 415 | UCUGUCCAAGCCC | 639 | TCTGTCCAAGCCC | Exon 51 |
| | UUGGACAGA | | GGUUGAAAU | | GGTTGAAAT | |
| 192 | AUUUCAACCGGGC | 416 | AGUUCUGUCCAAG | 640 | AGTTCTGTCCAAG | Exon 51 |
| | UUGGACAGAACU | | CCCGGUUGAAAU | | CCCGGTTGAAAT | |
| 193 | UUCAACCGGGCUU | 417 | AGUUCUGUCCAAG | 641 | AGTTCTGTCCAAG | Exon 51 |
| | GGACAGAACU | | CCCGGUUGAA | | CCCGGTTGAA | |
| 194 | UCAACCGGGCUUG | 418 | UUCUGUCCAAGCC | 642 | TTCTGTCCAAGCC | Exon 51 |
| | GACAGAA | | CGGUUGA | | CGGTTGA | |
| 195 | UCAACCGGGCUUG | 419 | AGUUCUGUCCAAG | 643 | AGTTCTGTCCAAG | Exon 51 |
| | GACAGAACU | | CCCGGUUGA | | CCCGGTTGA | |
| 196 | UCAACCGGGCUUG | 420 | GUAAGUUCUGUCC | 644 | GTAAGTTCTGTCC | Exon 51 |
| | GACAGAACUUAC | | AAGCCCGGUUGA | | AAGCCCGGTTGA | |
| 197 | CAACCGGGCUUGG | 421 | AGUUCUGUCCAAG | 645 | AGTTCTGTCCAAG | Exon 51 |
| | ACAGAACU | | CCCGGUUG | | CCCGGTTG | |
| 198 | CAACCGGGCUUGG | 422 | GUAAGUUCUGUCC | 646 | GTAAGTTCTGTCC | Exon 51 |
| | ACAGAACUUAC | | AAGCCCGGUUG | | AAGCCCGGTTG | |
| 199 | CAACCGGGCUUGG | 423 | GGUAAGUUCUGUC | 647 | GGTAAGTTCTGTC | Exon 51 |
| | ACAGAACUUACC | | CAAGCCCGGUUG | | CAAGCCCGGTTG | |
| 200 | AUGAUCAUCAAGC | 424 | UACCUUCUGCUUG | 648 | TACCTTCTGCTTG | Exon 51/intron 51 |
| | AGAAGGUA | | AUGAUCAU | | ATGATCAT | junction |
| 201 | AUGAUCAUCAAGC | 425 | CAUACCUUCUGCU | 649 | CATACCTTCTGCT | Exon 51/intron 51 |
| | AGAAGGUAUG | | UGAUGAUCAU | | TGATGATCAT | junction |
| 202 | AUGAUCAUCAAGC | 426 | UCAUACCUUCUGC | 650 | TCATACCTTCTGC | Exon 51/intron 51 |
| | AGAAGGUAUGA | | UUGAUGAUCAU | | TTGATGATCAT | junction |
| 203 | AUGAUCAUCAAGC | 427 | CUCAUACCUUCUG | 651 | CTCATACCTTCTG | Exon 51/intron 51 |
| | AGAAGGUAUGAG | | CUUGAUGAUCAU | | CTTGATGATCAT | junction |
| 204 | UGAUCAUCAAGCA | 428 | UACCUUCUGCUUG | 652 | TACCTTCTGCTTG | Exon 51/intron 51 |
| | GAAGGUA | | AUGAUCA | | ATGATCA | junction |
| 205 | UGAUCAUCAAGCA | 429 | CAUACCUUCUGCU | 653 | CATACCTTCTGCT | Exon 51/intron 51 |
| | GAAGGUAUG | | UGAUGAUCA | | TGATGATCA | junction |
| 206 | UGAUCAUCAAGCA | 430 | UCAUACCUUCUGC | 654 | TCATACCTTCTGC | Exon 51/intron 51 |
| | GAAGGUAUGA | | UUGAUGAUCA | | TTGATGATCA | junction |
| 207 | UGAUCAUCAAGCA | 431 | CUCAUACCUUCUG | 655 | CTCATACCTTCTG | Exon 51/intron 51 |
| | GAAGGUAUGAG | | CUUGAUGAUCA | | CTTGATGATCA | junction |
| 208 | UGAUCAUCAAGCA | 432 | UCUCAUACCUUCU | 656 | TCTCATACCTTCT | Exon 51/intron 51 |
| | GAAGGUAUGAGA | | GCUUGAUGAUCA | | GCTTGATGATCA | junction |
| 209 | GAUCAUCAAGCAG | 433 | CAUACCUUCUGCU | 657 | CATACCTTCTGCT | Exon 51/intron 51 |
| | AAGGUAUG | | UGAUGAUC | | TGATGATC | junction |
| 210 | GAUCAUCAAGCAG | 434 | UCAUACCUUCUGC | 658 | TCATACCTTCTGC | Exon 51/intron 51 |
| | AAGGUAUGA | | UUGAUGAUC | | TTGATGATC | junction |
| 211 | GAUCAUCAAGCAG | 435 | CUCAUACCUUCUG | 659 | CTCATACCTTCTG | Exon 51/intron 51 |
| | AAGGUAUGAG | | CUUGAUGAUC | | CTTGATGATC | junction |
| 212 | GAUCAUCAAGCAG | 436 | UCUCAUACCUUCU | 660 | TCTCATACCTTCT | Exon 51/intron 51 |
| | AAGGUAUGAGA | | GCUUGAUGAUC | | GCTTGATGATC | junction |
| 213 | GAUCAUCAAGCAG | 437 | UUCUCAUACCUUC | 661 | TTCTCATACCTTC | Exon 51/intron 51 |
| | AAGGUAUGAGAA | | UGCUUGAUGAUC | | TGCTTGATGATC | junction |
| 214 | AUCAUCAAGCAGA | 438 | CAUACCUUCUGCU | 662 | CATACCTTCTGCT | Exon 51/intron 51 |
| | AGGUAUG | | UGAUGAU | | TGATGAT | junction |
| 215 | AUCAUCAAGCAGA | 439 | UCAUACCUUCUGC | 663 | TCATACCTTCTGC | Exon 51/intron 51 |
| | AGGUAUGA | | UUGAUGAU | | TTGATGAT | junction |
| 216 | AUCAUCAAGCAGA | 440 | CUCAUACCUUCUG | 664 | CTCATACCTTCTG | Exon 51/intron 51 |
| | AGGUAUGAG | | CUUGAUGAU | | CTTGATGAT | junction |
| 217 | AUCAUCAAGCAGA | 441 | UCUCAUACCUUCU | 665 | TCTCATACCTTCT | Exon 51/intron 51 |
| | AGGUAUGAGA | | GCUUGAUGAU | | GCTTGATGAT | junction |
| 218 | AUCAUCAAGCAGA | 442 | UUCUCAUACCUUC | 666 | TTCTCATACCTTC | Exon 51/intron 51 |
| | AGGUAUGAGAA | | UGCUUGAUGAU | | TGCTTGATGAT | junction |
| 219 | AUCAUCAAGCAGA | 443 | UUUCUCAUACCUU | 667 | TTTCTCATACCTT | Exon 51/intron 51 |
| | AGGUAUGAGAAA | | CUGCUUGAUGAU | | CTGCTTGATGAT | junction |
| 220 | UCAUCAAGCAGAA | 444 | UCAUACCUUCUGC | 668 | TCATACCTTCTGC | Exon 51/intron 51 |
| | GGUAUGA | | UUGAUGA | | TTGATGA | junction |
| 221 | UCAUCAAGCAGAA | 445 | CUCAUACCUUCUG | 669 | CTCATACCTTCTG | Exon 51/intron 51 |
| | GGUAUGAG | | CUUGAUGA | | CTTGATGA | junction |
| 222 | UCAUCAAGCAGAA | 446 | UCUCAUACCUUCU | 670 | TCTCATACCTTCT | Exon 51/intron 51 |
| | GGUAUGAGA | | GCUUGAUGA | | GCTTGATGA | junction |
| 223 | UCAUCAAGCAGAA | 447 | UUCUCAUACCUUC | 671 | TTCTCATACCTTC | Exon 51/intron 51 |
| | GGUAUGAGAA | | UGCUUGAUGA | | TGCTTGATGA | junction |
| 224 | UCAUCAAGCAGAA | 448 | UUUCUCAUACCUU | 672 | TTTCTCATACCTT | Exon 51/intron 51 |
| | GGUAUGAGAAA | | CUGCUUGAUGA | | CTGCTTGATGA | junction |
| 225 | UCAUCAAGCAGAA | 449 | UUUUCUCAUACCU | 673 | TTTTCTCATACCT | Exon 51/intron 51 |
| | GGUAUGAGAAAA | | UCUGCUUGAUGA | | TCTGCTTGATGA | junction |
| 226 | CAUCAAGCAGAAG | 450 | CUCAUACCUUCUG | 674 | CTCATACCTTCTG | Exon 51/intron 51 |
| | GUAUGAG | | CUUGAUG | | CTTGATG | junction |
| 227 | CAUCAAGCAGAAG | 451 | UCUCAUACCUUCU | 675 | TCTCATACCTTCT | Exon 51/intron 51 |
| | GUAUGAGA | | GCUUGAUG | | GCTTGATG | junction |
| 228 | CAUCAAGCAGAAG | 452 | UUCUCAUACCUUC | 676 | TTCTCATACCTTC | Exon 51/intron 51 |
| | GUAUGAGAA | | UGCUUGAUG | | TGCTTGATG | junction |
| 229 | CAUCAAGCAGAAG | 453 | UUUCUCAUACCUU | 677 | TTTCTCATACCTT | Exon 51/intron 51 |
| | GUAUGAGAAA | | CUGCUUGAUG | | CTGCTTGATG | junction |
| 230 | CAUCAAGCAGAAG | 454 | UUUUCUCAUACCU | 678 | TTTTCTCATACCT | Exon 51/intron 51 |
| | GUAUGAGAAAA | | UCUGCUUGAUG | | TCTGCTTGATG | junction |
| 231 | CAUCAAGCAGAAG | 455 | UUUUUCUCAUACC | 679 | TTTTTCTCATACC | Exon 51/intron 51 |
| | GUAUGAGAAAAA | | UUCUGCUUGAUG | | TTCTGCTTGATG | junction |
| 232 | AUCAAGCAGAAGG | 456 | UCUCAUACCUUCU | 680 | TCTCATACCTTCT | Exon 51/intron 51 |
| | UAUGAGA | | GCUUGAU | | GCTTGAT | junction |
| 233 | AUCAAGCAGAAGG | 457 | UUCUCAUACCUUC | 681 | TTCTCATACCTTC | Exon 51/intron 51 |
| | UAUGAGAA | | UGCUUGAU | | TGCTTGAT | junction |
| 234 | AUCAAGCAGAAGG | 458 | UUUCUCAUACCUU | 682 | TTTCTCATACCTT | Exon 51/intron 51 |
| | UAUGAGAAA | | CUGCUUGAU | | CTGCTTGAT | junction |
| 235 | AUCAAGCAGAAGG | 459 | UUUUCUCAUACCU | 683 | TTTTCTCATACCT | Exon 51/intron 51 |
| | UAUGAGAAAA | | UCUGCUUGAU | | TCTGCTTGAT | junction |
| 236 | AUCAAGCAGAAGG | 460 | UUUUUCUCAUACC | 684 | TTTTTCTCATACC | Exon 51/intron 51 |
| | UAUGAGAAAAA | | UUCUGCUUGAU | | TTCTGCTTGAT | junction |
| 237 | AUCAAGCAGAAGG | 461 | UUUUUUCUCAUAC | 685 | TTTTTTCTCATAC | Exon 51/intron 51 |
| | UAUGAGAAAAAA | | CUUCUGCUUGAU | | CTTCTGCTTGAT | junction |
| 238 | UCAAGCAGAAGGU | 462 | UUCUCAUACCUUC | 686 | TTCTCATACCTTC | Exon 51/intron 51 |
| | AUGAGAA | | UGCUUGA | | TGCTTGA | junction |
| 239 | UCAAGCAGAAGGU | 463 | UUUCUCAUACCUU | 687 | TTTCTCATACCTT | Exon 51/intron 51 |
| | AUGAGAAA | | CUGCUUGA | | CTGCTTGA | junction |
| 240 | UCAAGCAGAAGGU | 464 | UUUUCUCAUACCU | 688 | TTTTCTCATACCT | Exon 51/intron 51 |
| | AUGAGAAAA | | UCUGCUUGA | | TCTGCTTGA | junction |
| 241 | UCAAGCAGAAGGU | 465 | UUUUUCUCAUACC | 689 | TTTTTCTCATACC | Exon 51/intron 51 |
| | AUGAGAAAAA | | UUCUGCUUGA | | TTCTGCTTGA | junction |
| 242 | UCAAGCAGAAGGU | 466 | UUUUUUCUCAUAC | 690 | TTTTTTCTCATAC | Exon 51/intron 51 |
| | AUGAGAAAAAA | | CUUCUGCUUGA | | CTTCTGCTTGA | junction |
| 243 | UCAAGCAGAAGGU | 467 | AUUUUUUCUCAUA | 691 | ATTTTTTCTCATA | Exon 51/intron 51 |
| | AUGAGAAAAAAU | | CCUUCUGCUUGA | | CCTTCTGCTTGA | junction |
| 244 | CAAGCAGAAGGUA | 468 | UUUCUCAUACCUU | 692 | TTTCTCATACCTT | Exon 51/intron 51 |
| | UGAGAAA | | CUGCUUG | | CTGCTTG | junction |
| 245 | CAAGCAGAAGGUA | 469 | UUUUCUCAUACCU | 693 | TTTTCTCATACCT | Exon 51/intron 51 |
| | UGAGAAAA | | UCUGCUUG | | TCTGCTTG | junction |
| 246 | CAAGCAGAAGGUA | 470 | UUUUUCUCAUACC | 694 | TTTTTCTCATACC | Exon 51/intron 51 |
| | UGAGAAAAA | | UUCUGCUUG | | TTCTGCTTG | junction |
| 247 | CAAGCAGAAGGUA | 471 | UUUUUUCUCAUAC | 695 | TTTTTTCTCATAC | Exon 51/intron 51 |
| | UGAGAAAAAA | | CUUCUGCUUG | | CTTCTGCTTG | junction |
| 248 | CAAGCAGAAGGUA | 472 | AUUUUUUCUCAUA | 696 | ATTTTTTCTCATA | Exon 51/intron 51 |
| | UGAGAAAAAAU | | CCUUCUGCUUG | | CCTTCTGCTTG | junction |
| 249 | AAGCAGAAGGUAU | 473 | UUUUCUCAUACCU | 697 | TTTTCTCATACCT | Exon 51/intron 51 |
| | GAGAAAA | | UCUGCUU | | TCTGCTT | junction |
| 250 | AAGCAGAAGGUAU | 474 | UUUUUCUCAUACC | 698 | TTTTTCTCATACC | Exon 51/intron 51 |
| | GAGAAAAA | | UUCUGCUU | | TTCTGCTT | junction |
| 251 | AAGCAGAAGGUAU | 475 | UUUUUUCUCAUAC | 699 | TTTTTTCTCATAC | Exon 51/intron 51 |
| | GAGAAAAAA | | CUUCUGCUU | | CTTCTGCTT | junction |
| 252 | AAGCAGAAGGUAU | 476 | AUUUUUUCUCAUA | 700 | ATTTTTTCTCATA | Exon 51/intron 51 |
| | GAGAAAAAAU | | CCUUCUGCUU | | CCTTCTGCTT | junction |
| 253 | AGCAGAAGGUAUG | 477 | UUUUUCUCAUACC | 701 | TTTTTCTCATACC | Exon 51/intron 51 |
| | AGAAAAA | | UUCUGCU | | TTCTGCT | junction |
| 254 | AGCAGAAGGUAUG | 478 | UUUUUUCUCAUAC | 702 | TTTTTTCTCATAC | Exon 51/intron 51 |
| | AGAAAAAA | | CUUCUGCU | | CTTCTGCT | junction |
| 255 | AGCAGAAGGUAUG | 479 | AUUUUUUCUCAUA | 703 | ATTTTTTCTCATA | Exon 51/intron 51 |
| | AGAAAAAAU | | CCUUCUGCU | | CCTTCTGCT | junction |
| 256 | GCAGAAGGUAUGA | 480 | UUUUUUCUCAUAC | 704 | TTTTTTCTCATAC | Exon 51/intron 51 |
| | GAAAAAA | | CUUCUGC | | CTTCTGC | junction |
| 257 | GCAGAAGGUAUGA | 481 | AUUUUUUCUCAUA | 705 | ATTTTTTCTCATA | Exon 51/intron 51 |
| | GAAAAAAU | | CCUUCUGC | | CCTTCTGC | junction |
| 258 | CAGAAGGUAUGAG | 482 | AUUUUUUCUCAUA | 706 | ATTTTTTCTCATA | Exon 51/intron 51 |
| | AAAAAAU | | CCUUCUG | | CCTTCTG | junction |
| 259 | AAAUGAUAAAAGU | 483 | ACUUCUGCCAACU | 707 | ACTTCTGCCAACT | Intron 51 |
| | UGGCAGAAGU | | UUUAUCAUUU | | TTTATCATTT | |
| 260 | UCACUUUACUCUC | 484 | AUGGUCUAGGAGA | 708 | ATGGTCTAGGAGA | Intron 51 |
| | CUAGACCAU | | GUAAAGUGA | | GTAAAGTGA | |
| 261 | UCACUUUACUCUC | 485 | AAUGGUCUAGGAG | 709 | AATGGTCTAGGAG | Intron 51 |
| | CUAGACCAUU | | AGUAAAGUGA | | AGTAAAGTGA | |
| 262 | UCACUUUACUCUC | 486 | AAAUGGUCUAGGA | 710 | AAATGGTCTAGGA | Intron 51 |
| | CUAGACCAUUU | | GAGUAAAGUGA | | GAGTAAAGTGA | |
| 263 | CACUUUACUCUCC | 487 | GGAAAUGGUCUAG | 711 | GGAAATGGTCTAG | Intron 51 |
| | UAGACCAUUUCC | | GAGAGUAAAGUG | | GAGAGTAAAGTG | |
| 264 | ACUUUACUCUCCU | 488 | GGAAAUGGUCUAG | 712 | GGAAATGGTCTAG | Intron 51 |
| | AGACCAUUUCC | | GAGAGUAAAGU | | GAGAGTAAAGT | |
| 265 | CUUUACUCUCCUA | 489 | UGGGAAAUGGUCU | 713 | TGGGAAATGGTCT | Intron 51 |
| | GACCAUUUCCCA | | AGGAGAGUAAAG | | AGGAGAGTAAAG | |
| 266 | UUACUCUCCUAGA | 490 | UGGGAAAUGGUCU | 714 | TGGGAAATGGTCT | Intron 51 |
| | CCAUUUCCCA | | AGGAGAGUAA | | AGGAGAGTAA | |
| 267 | UUACUCUCCUAGA | 491 | GUGGGAAAUGGUC | 715 | GTGGGAAATGGTC | Intron 51 |
| | CCAUUUCCCAC | | UAGGAGAGUAA | | TAGGAGAGTAA | |
| 268 | UUACUCUCCUAGA | 492 | GGUGGGAAAUGGU | 716 | GGTGGGAAATGGT | Intron 51 |
| | CCAUUUCCCACC | | CUAGGAGAGUAA | | CTAGGAGAGTAA | |
| 269 | UACUCUCCUAGAC | 493 | UGGGAAAUGGUCU | 717 | TGGGAAATGGTCT | Intron 51 |
| | CAUUUCCCA | | AGGAGAGUA | | AGGAGAGTA | |
| 270 | UACUCUCCUAGAC | 494 | GUGGGAAAUGGUC | 718 | GTGGGAAATGGTC | Intron 51 |
| | CAUUUCCCAC | | UAGGAGAGUA | | TAGGAGAGTA | |
| 271 | UACUCUCCUAGAC | 495 | GGUGGGAAAUGGU | 719 | GGTGGGAAATGGT | Intron 51 |
| | CAUUUCCCACC | | CUAGGAGAGUA | | CTAGGAGAGTA | |
| 272 | UACUCUCCUAGAC | 496 | UGGUGGGAAAUGG | 720 | TGGTGGGAAATGG | Intron 51 |
| | CAUUUCCCACCA | | UCUAGGAGAGUA | | TCTAGGAGAGTA | |
| 273 | ACUCUCCUAGACC | 497 | UGGGAAAUGGUCU | 721 | TGGGAAATGGTCT | Intron 51 |
| | AUUUCCCA | | AGGAGAGU | | AGGAGAGT | |
| 274 | ACUCUCCUAGACC | 498 | GUGGGAAAUGGUC | 722 | GTGGGAAATGGTC | Intron 51 |
| | AUUUCCCAC | | UAGGAGAGU | | TAGGAGAGT | |
| 275 | ACUCUCCUAGACC | 499 | GGUGGGAAAUGGU | 723 | GGTGGGAAATGGT | Intron 51 |
| | AUUUCCCACC | | CUAGGAGAGU | | CTAGGAGAGT | |
| 276 | ACUCUCCUAGACC | 500 | UGGUGGGAAAUGG | 724 | TGGTGGGAAATGG | Intron 51 |
| | AUUUCCCACCA | | UCUAGGAGAGU | | TCTAGGAGAGT | |
| 277 | ACUCUCCUAGACC | 501 | CUGGUGGGAAAUG | 725 | CTGGTGGGAAATG | Intron 51 |
| | AUUUCCCACCAG | | GUCUAGGAGAGU | | GTCTAGGAGAGT | |
| 278 | CUCUCCUAGACCA | 502 | UGGGAAAUGGUCU | 726 | TGGGAAATGGTCT | Intron 51 |
| | UUUCCCA | | AGGAGAG | | AGGAGAG | |
| 279 | CUCUCCUAGACCA | 503 | GUGGGAAAUGGUC | 727 | GTGGGAAATGGTC | Intron 51 |
| | UUUCCCAC | | UAGGAGAG | | TAGGAGAG | |
| 280 | CUCUCCUAGACCA | 504 | GGUGGGAAAUGGU | 728 | GGTGGGAAATGGT | Intron 51 |
| | UUUCCCACC | | CUAGGAGAG | | CTAGGAGAG | |
| 281 | CUCUCCUAGACCA | 505 | UGGUGGGAAAUGG | 729 | TGGTGGGAAATGG | Intron 51 |
| | UUUCCCACCA | | UCUAGGAGAG | | TCTAGGAGAG | |
| 282 | CUCUCCUAGACCA | 506 | CUGGUGGGAAAUG | 730 | CTGGTGGGAAATG | Intron 51 |
| | UUUCCCACCAG | | GUCUAGGAGAG | | GTCTAGGAGAG | |
| 283 | UCUCCUAGACCAU | 507 | GUGGGAAAUGGUC | 731 | GTGGGAAATGGTC | Intron 51 |
| | UUCCCAC | | UAGGAGA | | TAGGAGA | |
| 284 | UCUCCUAGACCAU | 508 | GGUGGGAAAUGGU | 732 | GGTGGGAAATGGT | Intron 51 |
| | UUCCCACC | | CUAGGAGA | | CTAGGAGA | |
| 285 | UCUCCUAGACCAU | 509 | UGGUGGGAAAUGG | 733 | TGGTGGGAAATGG | Intron 51 |
| | UUCCCACCA | | UCUAGGAGA | | TCTAGGAGA | |
| 286 | UCUCCUAGACCAU | 510 | CUGGUGGGAAAUG | 734 | CTGGTGGGAAATG | Intron 51 |
| | UUCCCACCAG | | GUCUAGGAGA | | GTCTAGGAGA | |
| 287 | UCUCCUAGACCAU | 511 | AACUGGUGGGAAA | 735 | AACTGGTGGGAAA | Intron 51 |
| | UUCCCACCAGUU | | UGGUCUAGGAGA | | TGGTCTAGGAGA | |
| 288 | CUCCUAGACCAUU | 512 | GGUGGGAAAUGGU | 736 | GGTGGGAAATGGT | Intron 51 |
| | UCCCACC | | CUAGGAG | | CTAGGAG | |
| 289 | CUCCUAGACCAUU | 513 | UGGUGGGAAAUGG | 737 | TGGTGGGAAATGG | Intron 51 |
| | UCCCACCA | | UCUAGGAG | | TCTAGGAG | |
| 290 | CUCCUAGACCAUU | 514 | CUGGUGGGAAAUG | 738 | CTGGTGGGAAATG | Intron 51 |
| | UCCCACCAG | | GUCUAGGAG | | GTCTAGGAG | |
| 291 | CUCCUAGACCAUU | 515 | AACUGGUGGGAAA | 739 | AACTGGTGGGAAA | Intron 51 |
| | UCCCACCAGUU | | UGGUCUAGGAG | | TGGTCTAGGAG | |
| 292 | UCCUAGACCAUUU | 516 | UGGUGGGAAAUGG | 740 | TGGTGGGAAATGG | Intron 51 |
| | CCCACCA | | UCUAGGA | | TCTAGGA | |
| 293 | UCCUAGACCAUUU | 517 | CUGGUGGGAAAUG | 741 | CTGGTGGGAAATG | Intron 51 |
| | CCCACCAG | | GUCUAGGA | | GTCTAGGA | |
| 294 | UCCUAGACCAUUU | 518 | AACUGGUGGGAAA | 742 | AACTGGTGGGAAA | Intron 51 |
| | CCCACCAGUU | | UGGUCUAGGA | | TGGTCTAGGA | |
| 295 | UCCUAGACCAUUU | 519 | AGAACUGGUGGGA | 743 | AGAACTGGTGGGA | Intron 51 |
| | CCCACCAGUUCU | | AAUGGUCUAGGA | | AATGGTCTAGGA | |
| 296 | CCUAGACCAUUUC | 520 | CUGGUGGGAAAUG | 744 | CTGGTGGGAAATG | Intron 51 |
| | CCACCAG | | GUCUAGG | | GTCTAGG | |
| 297 | CCUAGACCAUUUC | 521 | AACUGGUGGGAAA | 745 | AACTGGTGGGAAA | Intron 51 |
| | CCACCAGUU | | UGGUCUAGG | | TGGTCTAGG | |
| 298 | CCUAGACCAUUUC | 522 | AGAACUGGUGGGA | 746 | AGAACTGGTGGGA | Intron 51 |
| | CCACCAGUUCU | | AAUGGUCUAGG | | AATGGTCTAGG | |
| 299 | CCUAGACCAUUUC | 523 | AAGAACUGGUGGG | 747 | AAGAACTGGTGGG | Intron 51 |
| | CCACCAGUUCUU | | AAAUGGUCUAGG | | AAATGGTCTAGG | |
| 300 | CUAGACCAUUUCC | 524 | AACUGGUGGGAAA | 748 | AACTGGTGGGAAA | Intron 51 |
| | CACCAGUU | | UGGUCUAG | | TGGTCTAG | |
| 301 | CUAGACCAUUUCC | 525 | AGAACUGGUGGGA | 749 | AGAACTGGTGGGA | Intron 51 |
| | CACCAGUUCU | | AAUGGUCUAG | | AATGGTCTAG | |
| 302 | CUAGACCAUUUCC | 526 | AAGAACUGGUGGG | 750 | AAGAACTGGTGGG | Intron 51 |
| | CACCAGUUCUU | | AAAUGGUCUAG | | AAATGGTCTAG | |
| 303 | UAGACCAUUUCCC | 527 | AACUGGUGGGAAA | 751 | AACTGGTGGGAAA | Intron 51 |
| | ACCAGUU | | UGGUCUA | | TGGTCTA | |
| 304 | UAGACCAUUUCCC | 528 | AGAACUGGUGGGA | 752 | AGAACTGGTGGGA | Intron 51 |
| | ACCAGUUCU | | AAUGGUCUA | | AATGGTCTA | |
| 305 | UAGACCAUUUCCC | 529 | AAGAACUGGUGGG | 753 | AAGAACTGGTGGG | Intron 51 |
| | ACCAGUUCUU | | AAAUGGUCUA | | AAATGGTCTA | |
| 306 | UAGACCAUUUCCC | 530 | CUAAGAACUGGUG | 754 | CTAAGAACTGGTG | Intron 51 |
| | ACCAGUUCUUAG | | GGAAAUGGUCUA | | GGAAATGGTCTA | |
| 307 | AGACCAUUUCCCA | 531 | AGAACUGGUGGGA | 755 | AGAACTGGTGGGA | Intron 51 |
| | CCAGUUCU | | AAUGGUCU | | AATGGTCT | |
| 308 | AGACCAUUUCCCA | 532 | AAGAACUGGUGGG | 756 | AAGAACTGGTGGG | Intron 51 |
| | CCAGUUCUU | | AAAUGGUCU | | AAATGGTCT | |
| 309 | AGACCAUUUCCCA | 533 | CUAAGAACUGGUG | 757 | CTAAGAACTGGTG | Intron 51 |
| | CCAGUUCUUAG | | GGAAAUGGUCU | | GGAAATGGTCT | |
| 310 | AGACCAUUUCCCA | 534 | CCUAAGAACUGGU | 758 | CCTAAGAACTGGT | Intron 51 |
| | CCAGUUCUUAGG | | GGGAAAUGGUCU | | GGGAAATGGTCT | |
| 311 | GACCAUUUCCCAC | 535 | AGAACUGGUGGGA | 759 | AGAACTGGTGGGA | Intron 51 |
| | CAGUUCU | | AAUGGUC | | AATGGTC | |
| 312 | GACCAUUUCCCAC | 536 | AAGAACUGGUGGG | 760 | AAGAACTGGTGGG | Intron 51 |
| | CAGUUCUU | | AAAUGGUC | | AAATGGTC | |
| 313 | GACCAUUUCCCAC | 537 | CUAAGAACUGGUG | 761 | CTAAGAACTGGTG | Intron 51 |
| | CAGUUCUUAG | | GGAAAUGGUC | | GGAAATGGTC | |
| 314 | GACCAUUUCCCAC | 538 | CCUAAGAACUGGU | 762 | CCTAAGAACTGGT | Intron 51 |
| | CAGUUCUUAGG | | GGGAAAUGGUC | | GGGAAATGGTC | |
| 315 | GACCAUUUCCCAC | 539 | GCCUAAGAACUGG | 763 | GCCTAAGAACTGG | Intron 51 |
| | CAGUUCUUAGGC | | UGGGAAAUGGUC | | TGGGAAATGGTC | |
| 316 | ACCAUUUCCCACC | 540 | GCCUAAGAACUGG | 764 | GCCTAAGAACTGG | Intron 51 |
| | AGUUCUUAGGC | | UGGGAAAUGGU | | TGGGAAATGGT | |
| 317 | ACCAUUUCCCACC | 541 | UGCCUAAGAACUG | 765 | TGCCTAAGAACTG | Intron 51 |
| | AGUUCUUAGGCA | | GUGGGAAAUGGU | | GTGGGAAATGGT | |
| 318 | CCAUUUCCCACCA | 542 | GCCUAAGAACUGG | 766 | GCCTAAGAACTGG | Intron 51 |
| | GUUCUUAGGC | | UGGGAAAUGG | | TGGGAAATGG | |
| 319 | CCAUUUCCCACCA | 543 | UGCCUAAGAACUG | 767 | TGCCTAAGAACTG | Intron 51 |
| | GUUCUUAGGCA | | GUGGGAAAUGG | | GTGGGAAATGG | |
| 320 | CCAUUUCCCACCA | 544 | UUGCCUAAGAACU | 768 | TTGCCTAAGAACT | Intron 51 |
| | GUUCUUAGGCAA | | GGUGGGAAAUGG | | GGTGGGAAATGG | |
| 321 | CAUUUCCCACCAG | 545 | UGCCUAAGAACUG | 769 | TGCCTAAGAACTG | Intron 51 |
| | UUCUUAGGCA | | GUGGGAAAUG | | GTGGGAAATG | |
| 322 | CAUUUCCCACCAG | 546 | UUGCCUAAGAACU | 770 | TTGCCTAAGAACT | Intron 51 |
| | UUCUUAGGCAA | | GGUGGGAAAUG | | GGTGGGAAATG | |
| 323 | AGUGUUUUGGCUG | 547 | GUGAGACCAGCCA | 771 | GTGAGACCAGCCA | Intron 51 |
| | GUCUCAC | | AAACACU | | AAACACT | |
| 324 | AGUGUUUUGGCUG | 548 | UGUGAGACCAGCC | 772 | TGTGAGACCAGCC | Intron 51 |
| | GUCUCACA | | AAAACACU | | AAAACACT | |
| 325 | AGUGUUUUGGCUG | 549 | UUGUGAGACCAGC | 773 | TTGTGAGACCAGC | Intron 51 |
| | GUCUCACAA | | CAAAACACU | | CAAAACACT | |
| 326 | AGUGUUUUGGCUG | 550 | AUUGUGAGACCAG | 774 | ATTGTGAGACCAG | Intron 51 |
| | GUCUCACAAU | | CCAAAACACU | | CCAAAACACT | |
| 327 | GUGUUUUGGCUGG | 551 | AUUGUGAGACCAG | 775 | ATTGTGAGACCAG | Intron 51 |
| | UCUCACAAU | | CCAAAACAC | | CCAAAACAC | |
| 328 | GUUUUGGCUGGUC | 552 | GUACAAUUGUGAG | 776 | GTACAATTGTGAG | Intron 51 |
| | UCACAAUUGUAC | | ACCAGCCAAAAC | | ACCAGCCAAAAC | |
| 329 | UUUGGCUGGUCUC | 553 | GUACAAUUGUGAG | 777 | GTACAATTGTGAG | Intron 51 |
| | ACAAUUGUAC | | ACCAGCCAAA | | ACCAGCCAAA | |
| 330 | UUGGCUGGUCUCA | 554 | GUACAAUUGUGAG | 778 | GTACAATTGTGAG | Intron 51 |
| | CAAUUGUAC | | ACCAGCCAA | | ACCAGCCAA | |
| 331 | UUGGCUGGUCUCA | 555 | AGUACAAUUGUGA | 779 | AGTACAATTGTGA | Intron 51 |
| | CAAUUGUACU | | GACCAGCCAA | | GACCAGCCAA | |
| 332 | UGGCUGGUCUCAC | 556 | GUACAAUUGUGAG | 780 | GTACAATTGTGAG | Intron 51 |
| | AAUUGUAC | | ACCAGCCA | | ACCAGCCA | |
| 333 | UGGCUGGUCUCAC | 557 | AGUACAAUUGUGA | 781 | AGTACAATTGTGA | Intron 51 |
| | AAUUGUACU | | GACCAGCCA | | GACCAGCCA | |
| 334 | UGGCUGGUCUCAC | 558 | AAGUACAAUUGUG | 782 | AAGTACAATTGTG | Intron 51 |
| | AAUUGUACUU | | AGACCAGCCA | | AGACCAGCCA | |
| 335 | UGGCUGGUCUCAC | 559 | AAAGUACAAUUGU | 783 | AAAGTACAATTGT | Intron 51 |
| | AAUUGUACUUU | | GAGACCAGCCA | | GAGACCAGCCA | |
| 336 | GGCUGGUCUCACA | 560 | GUACAAUUGUGAG | 784 | GTACAATTGTGAG | Intron 51 |
| | AUUGUAC | | ACCAGCC | | ACCAGCC | |
| 337 | GGCUGGUCUCACA | 561 | AGUACAAUUGUGA | 785 | AGTACAATTGTGA | Intron 51 |
| | AUUGUACU | | GACCAGCC | | GACCAGCC | |
| 338 | GGCUGGUCUCACA | 562 | AAGUACAAUUGUG | 786 | AAGTACAATTGTG | Intron 51 |
| | AUUGUACUU | | AGACCAGCC | | AGACCAGCC | |
| 339 | GGCUGGUCUCACA | 563 | AAAGUACAAUUGU | 787 | AAAGTACAATTGT | Intron 51 |
| | AUUGUACUUU | | GAGACCAGCC | | GAGACCAGCC | |
| 340 | GGCUGGUCUCACA | 564 | GUAAAGUACAAUU | 788 | GTAAAGTACAATT | Intron 51 |
| | AUUGUACUUUAC | | GUGAGACCAGCC | | GTGAGACCAGCC | |
| 341 | GCUGGUCUCACAA | 565 | GUAAAGUACAAUU | 789 | GTAAAGTACAATT | Intron 51 |
| | UUGUACUUUAC | | GUGAGACCAGC | | GTGAGACCAGC | |
| 342 | GCUGGUCUCACAA | 566 | AGUAAAGUACAAU | 790 | AGTAAAGTACAAT | Intron 51 |
| | UUGUACUUUACU | | UGUGAGACCAGC | | TGTGAGACCAGC | |
| 343 | UGUAAAAGGAAUA | 567 | UCAGCGUUGUGUA | 791 | TCAGCGTTGTGTA | Intron 51 |
| | CACAACGCUGA | | UUCCUUUUACA | | TTCCTTTTACA | |
| 344 | UGUAAAAGGAAUA | 568 | UUCAGCGUUGUGU | 792 | TTCAGCGTTGTGT | Intron 51 |
| | CACAACGCUGAA | | AUUCCUUUUACA | | ATTCCTTTTACA | |
| 345 | GUAAAAGGAAUAC | 569 | UCAGCGUUGUGUA | 793 | TCAGCGTTGTGTA | Intron 51 |
| | ACAACGCUGA | | UUCCUUUUAC | | TTCCTTTTAC | |
| 346 | GUAAAAGGAAUAC | 570 | UUCAGCGUUGUGU | 794 | TTCAGCGTTGTGT | Intron 51 |
| | ACAACGCUGAA | | AUUCCUUUUAC | | ATTCCTTTTAC | |
| 347 | GUAAAAGGAAUAC | 571 | CUUCAGCGUUGUG | 795 | CTTCAGCGTTGTG | Intron 51 |
| | ACAACGCUGAAG | | UAUUCCUUUUAC | | TATTCCTTTTAC | |
| 348 | UAAAAGGAAUACA | 572 | UCAGCGUUGUGUA | 796 | TCAGCGTTGTGTA | Intron 51 |
| | CAACGCUGA | | UUCCUUUUA | | TTCCTTTTA | |
| 349 | UAAAAGGAAUACA | 573 | UUCAGCGUUGUGU | 797 | TTCAGCGTTGTGT | Intron 51 |
| | CAACGCUGAA | | AUUCCUUUUA | | ATTCCTTTTA | |
| 350 | UAAAAGGAAUACA | 574 | CUUCAGCGUUGUG | 798 | CTTCAGCGTTGTG | Intron 51 |
| | CAACGCUGAAG | | UAUUCCUUUUA | | TATTCCTTTTA | |
| 351 | UAAAAGGAAUACA | 575 | UCUUCAGCGUUGU | 799 | TCTTCAGCGTTGT | Intron 51 |
| | CAACGCUGAAGA | | GUAUUCCUUUUA | | GTATTCCTTTTA | |
| 352 | AAAAGGAAUACAC | 576 | UCAGCGUUGUGUA | 800 | TCAGCGTTGTGTA | Intron 51 |
| | AACGCUGA | | UUCCUUUU | | TTCCTTTT | |
| 353 | AAAAGGAAUACAC | 577 | UUCAGCGUUGUGU | 801 | TTCAGCGTTGTGT | Intron 51 |
| | AACGCUGAA | | AUUCCUUUU | | ATTCCTTTT | |
| 354 | AAAAGGAAUACAC | 578 | CUUCAGCGUUGUG | 802 | CTTCAGCGTTGTG | Intron 51 |
| | AACGCUGAAG | | UAUUCCUUUU | | TATTCCTTTT | |
| 355 | AAAAGGAAUACAC | 579 | UCUUCAGCGUUGU | 803 | TCTTCAGCGTTGT | Intron 51 |
| | AACGCUGAAGA | | GUAUUCCUUUU | | GTATTCCTTTT | |
| 356 | AAAAGGAAUACAC | 580 | UUCUUCAGCGUUG | 804 | TTCTTCAGCGTTG | Intron 51 |
| | AACGCUGAAGAA | | UGUAUUCCUUUU | | TGTATTCCTTTT | |
| 357 | AAAGGAAUACACA | 581 | UCAGCGUUGUGUA | 805 | TCAGCGTTGTGTA | Intron 51 |
| | ACGCUGA | | UUCCUUU | | TTCCTTT | |
| 358 | AAAGGAAUACACA | 582 | UUCAGCGUUGUGU | 806 | TTCAGCGTTGTGT | Intron 51 |
| | ACGCUGAA | | AUUCCUUU | | ATTCCTTT | |
| 359 | AAAGGAAUACACA | 583 | CUUCAGCGUUGUG | 807 | CTTCAGCGTTGTG | Intron 51 |
| | ACGCUGAAG | | UAUUCCUUU | | TATTCCTTT | |
| 360 | AAAGGAAUACACA | 584 | UCUUCAGCGUUGU | 808 | TCTTCAGCGTTGT | Intron 51 |
| | ACGCUGAAGA | | GUAUUCCUUU | | GTATTCCTTT | |
| 361 | AAAGGAAUACACA | 585 | UUCUUCAGCGUUG | 809 | TTCTTCAGCGTTG | Intron 51 |
| | ACGCUGAAGAA | | UGUAUUCCUUU | | TGTATTCCTTT | |
| 362 | AAAGGAAUACACA | 586 | GUUCUUCAGCGUU | 810 | GTTCTTCAGCGTT | Intron 51 |
| | ACGCUGAAGAAC | | GUGUAUUCCUUU | | GTGTATTCCTTT | |
| 363 | AAGGAAUACACAA | 587 | UUCAGCGUUGUGU | 811 | TTCAGCGTTGTGT | Intron 51 |
| | CGCUGAA | | AUUCCUU | | ATTCCTT | |
| 364 | AAGGAAUACACAA | 588 | CUUCAGCGUUGUG | 812 | CTTCAGCGTTGTG | Intron 51 |
| | CGCUGAAG | | UAUUCCUU | | TATTCCTT | |
| 365 | AAGGAAUACACAA | 589 | UCUUCAGCGUUGU | 813 | TCTTCAGCGTTGT | Intron 51 |
| | CGCUGAAGA | | GUAUUCCUU | | GTATTCCTT | |
| 366 | AAGGAAUACACAA | 590 | UUCUUCAGCGUUG | 814 | TTCTTCAGCGTTG | Intron 51 |
| | CGCUGAAGAA | | UGUAUUCCUU | | TGTATTCCTT | |
| 367 | AAGGAAUACACAA | 591 | GUUCUUCAGCGUU | 815 | GTTCTTCAGCGTT | Intron 51 |
| | CGCUGAAGAAC | | GUGUAUUCCUU | | GTGTATTCCTT | |
| 368 | AGGAAUACACAAC | 592 | CUUCAGCGUUGUG | 816 | CTTCAGCGTTGTG | Intron 51 |
| | GCUGAAG | | UAUUCCU | | TATTCCT | |
| 369 | AGGAAUACACAAC | 593 | UCUUCAGCGUUGU | 817 | TCTTCAGCGTTGT | Intron 51 |
| | GCUGAAGA | | GUAUUCCU | | GTATTCCT | |
| 370 | AGGAAUACACAAC | 594 | UUCUUCAGCGUUG | 818 | TTCTTCAGCGTTG | Intron 51 |
| | GCUGAAGAA | | UGUAUUCCU | | TGTATTCCT | |
| 371 | AGGAAUACACAAC | 595 | GUUCUUCAGCGUU | 819 | GTTCTTCAGCGTT | Intron 51 |
| | GCUGAAGAAC | | GUGUAUUCCU | | GTGTATTCCT | |
| 372 | GGAAUACACAACG | 596 | UCUUCAGCGUUGU | 820 | TCTTCAGCGTTGT | Intron 51 |
| | CUGAAGA | | GUAUUCC | | GTATTCC | |
| 373 | GGAAUACACAACG | 597 | UUCUUCAGCGUUG | 821 | TTCTTCAGCGTTG | Intron 51 |
| | CUGAAGAA | | UGUAUUCC | | TGTATTCC | |
| 374 | GGAAUACACAACG | 598 | GUUCUUCAGCGUU | 822 | GTTCTTCAGCGTT | Intron 51 |
| | CUGAAGAAC | | GUGUAUUCC | | GTGTATTCC | |
| 375 | GGAAUACACAACG | 599 | GGGUUCUUCAGCG | 823 | GGGTTCTTCAGCG | Intron 51 |
| | CUGAAGAACCC | | UUGUGUAUUCC | | TTGTGTATTCC | |
| 376 | GGAAUACACAACG | 600 | AGGGUUCUUCAGC | 824 | AGGGTTCTTCAGC | Intron 51 |
| | CUGAAGAACCCU | | GUUGUGUAUUCC | | GTTGTGTATTCC | |
| 377 | GAAUACACAACGC | 601 | GUUCUUCAGCGUU | 825 | GTTCTTCAGCGTT | Intron 51 |
| | UGAAGAAC | | GUGUAUUC | | GTGTATTC | |
| 378 | GAAUACACAACGC | 602 | GGGUUCUUCAGCG | 826 | GGGTTCTTCAGCG | Intron 51 |
| | UGAAGAACCC | | UUGUGUAUUC | | TTGTGTATTC | |
| 379 | GAAUACACAACGC | 603 | AGGGUUCUUCAGC | 827 | AGGGTTCTTCAGC | Intron 51 |
| | UGAAGAACCCU | | GUUGUGUAUUC | | GTTGTGTATTC | |
| 380 | AAUACACAACGCU | 604 | GGGUUCUUCAGCG | 828 | GGGTTCTTCAGCG | Intron 51 |
| | GAAGAACCC | | UUGUGUAUU | | TTGTGTATT | |
| 381 | AUACACAACGCUG | 605 | GGGUUCUUCAGCG | 829 | GGGTTCTTCAGCG | Intron 51 |
| | AAGAACCC | | UUGUGUAU | | TTGTGTAT | |
| 382 | UACACAACGCUGA | 606 | GGGUUCUUCAGCG | 830 | GGGTTCTTCAGCG | Intron 51 |
| | AGAACCC | | UUGUGUA | | TTGTGTA | |
| 383 | ACACAACGCUGAA | 607 | AUCAGGGUUCUUC | 831 | ATCAGGGTTCTTC | Intron 51 |
| | GAACCCUGAU | | AGCGUUGUGU | | AGCGTTGTGT | |

^{†}Each thymine base (T) in any one of the oligonucleotides and/or target sequences provided in Table 8 may
independently and optionally be replaced with a uracil base (U), and/or each U may independently and optionally
be replaced with a T. Target sequences listed in Table 8 contain U's, but binding of a DMD-targeting
oligonucleotide to RNA and/or DNA is contemplated.

In some embodiments, an oligonucleotide useful for targeting DMD (e.g., for exon skipping) targets a region of a DMD sequence. In some embodiments, an oligonucleotide useful for targeting DMD (e.g., for exon skipping) targets a region of a DMD RNA (e.g., the Dp427m transcript of SEQ ID NO: 130). In some embodiments, an oligonucleotide useful for targeting DMD (e.g., for exon skipping) comprises a region of complementarity to a DMD RNA (e.g., the Dp427m transcript of SEQ ID NO: 130). In some embodiments, an oligonucleotide useful for targeting DMD (e.g., for exon skipping) comprises a region of complementarity to an exon of a DMD RNA (e.g., SEQ ID NO: 131, 838, or 854). In some embodiments, an oligonucleotide useful for targeting DMD (e.g., for exon skipping) comprises a region of complementarity to an intron of a DMD RNA (e.g., SEQ ID NO: 834 or 846). In some embodiments, an oligonucleotide useful for targeting DMD (e.g., for exon skipping) comprises a region of complementarity to a portion of a DMD sequence (e.g., a sequence provided by any one of SEQ ID NOs: 832, 844, 833, 845, 835, 836, 847-852, 837, 853, 839-843). Examples of DMD sequences are provided below. Each of the DMD sequences provided below include thymine nucleotides (T's), but it should be understood that each sequence can represent a DNA sequence or an RNA sequence in which any or all of the T's would be replaced with uracil nucleotides (U's).

Homo sapiens dystrophin (DMD), transcript variant Dp427m, mRNA (NCBI Reference Sequence: NM_004006.2)

(SEQ ID NO: 130)
TCCTGGCATCAGTTACTGTGTTGACTCACTCAGTGTTGGGATCACTCAC
TTTCCCCCTACAGGACTCAGATCTGGGAGGCAATTACCTTCGGAGAAAA
ACGAATAGGAAAAACTGAAGTGTTACTTTTTTTAAAGCTGCTGAAGTTT
GTTGGTTTCTCATTGTTTTTAAGCCTACTGGAGCAATAAAGTTTGAAGA
ACTTTTACCAGGTTTTTTTTATCGCTGCCTTGATATACACTTTTCAAAA
TGCTTTGGTGGGAAGAAGTAGAGGACTGTTATGAAAGAGAAGATGTTCA
AAAGAAAACATTCACAAAATGGGTAAATGCACAATTTTCTAAGTTTGGG
AAGCAGCATATTGAGAACCTCTTCAGTGACCTACAGGATGGGAGGCGCC
TCCTAGACCTCCTCGAAGGCCTGACAGGGCAAAAACTGCCAAAAGAAAA
AGGATCCACAAGAGTTCATGCCCTGAACAATGTCAACAAGGCACTGCGG
GTTTTGCAGAACAATAATGTTGATTTAGTGAATATTGGAAGTACTGACA
TCGTAGATGGAAATCATAAACTGACTCTTGGTTTGATTTGGAATATAAT
CCTCCACTGGCAGGTCAAAAATGTAATGAAAAATATCATGGCTGGATTG
CAACAAACCAACAGTGAAAAGATTCTCCTGAGCTGGGTCCGACAATCAA
CTCGTAATTATCCACAGGTTAATGTAATCAACTTCACCACCAGCTGGTC
TGATGGCCTGGCTTTGAATGCTCTCATCCATAGTCATAGGCCAGACCTA
TTTGACTGGAATAGTGTGGTTTGCCAGCAGTCAGCCACACAACGACTGG
AACATGCATTCAACATCGCCAGATATCAATTAGGCATAGAGAAACTACT
CGATCCTGAAGATGTTGATACCACCTATCCAGATAAGAAGTCCATCTTA
ATGTACATCACATCACTCTTCCAAGTTTTGCCTCAACAAGTGAGCATTG
AAGCCATCCAGGAAGTGGAAATGTTGCCAAGGCCACCTAAAGTGACTAA
AGAAGAACATTTTCAGTTACATCATCAAATGCACTATTCTCAACAGATC
ACGGTCAGTCTAGCACAGGGATATGAGAGAACTTCTTCCCCTAAGCCTC
GATTCAAGAGCTATGCCTACACACAGGCTGCTTATGTCACCACCTCTGA
CCCTACACGGAGCCCATTTCCTTCACAGCATTTGGAAGCTCCTGAAGAC
AAGTCATTTGGCAGTTCATTGATGGAGAGTGAAGTAAACCTGGACCGTT
ATCAAACAGCTTTAGAAGAAGTATTATCGTGGCTTCTTTCTGCTGAGGA
CACATTGCAAGCACAAGGAGAGATTTCTAATGATGTGGAAGTGGTGAAA
GACCAGTTTCATACTCATGAGGGGTACATGATGGATTTGACAGCCCATC
AGGGCCGGGTTGGTAATATTCTACAATTGGGAAGTAAGCTGATTGGAAC
AGGAAAATTATCAGAAGATGAAGAAACTGAAGTACAAGAGCAGATGAAT
CTCCTAAATTCAAGATGGGAATGCCTCAGGGTAGCTAGCATGGAAAAAC
AAAGCAATTTACATAGAGTTTTAATGGATCTCCAGAATCAGAAACTGAA
AGAGTTGAATGACTGGCTAACAAAAACAGAAGAAAGAACAAGGAAAATG
GAGGAAGAGCCTCTTGGACCTGATCTTGAAGACCTAAAACGCCAAGTAC
AACAACATAAGGTGCTTCAAGAAGATCTAGAACAAGAACAAGTCAGGGT
CAATTCTCTCACTCACATGGTGGTGGTAGTTGATGAATCTAGTGGAGAT
CACGCAACTGCTGCTTTGGAAGAACAACTTAAGGTATTGGGAGATCGAT
GGGCAAACATCTGTAGATGGACAGAAGACCGCTGGGTTCTTTTACAAGA
CATCCTTCTCAAATGGCAACGTCTTACTGAAGAACAGTGCCTTTTTAGT
GCATGGCTTTCAGAAAAAGAAGATGCAGTGAACAAGATTCACACAACTG
GCTTTAAAGATCAAAATGAAATGTTATCAAGTCTTCAAAAACTGGCCGT
TTTAAAAGCGGATCTAGAAAAGAAAAAGCAATCCATGGGCAAACTGTAT
TCACTCAAACAAGATCTTCTTTCAACACTGAAGAATAAGTCAGTGACCC
AGAAGACGGAAGCATGGCTGGATAACTTTGCCCGGTGTTGGGATAATTT
AGTCCAAAAACTTGAAAAGAGTACAGCACAGATTTCACAGGCTGTCACC
ACCACTCAGCCATCACTAACACAGACAACTGTAATGGAAACAGTAACTA
CGGTGACCACAAGGGAACAGATCCTGGTAAAGCATGCTCAAGAGGAACT
TCCACCACCACCTCCCCAAAAGAAGAGGCAGATTACTGTGGATTCTGAA
ATTAGGAAAAGGTTGGATGTTGATATAACTGAACTTCACAGCTGGATTA
CTCGCTCAGAAGCTGTGTTGCAGAGTCCTGAATTTGCAATCTTTCGGAA
GGAAGGCAACTTCTCAGACTTAAAAGAAAAAGTCAATGCCATAGAGCGA
GAAAAAGCTGAGAAGTTCAGAAAACTGCAAGATGCCAGCAGATCAGCTC
AGGCCCTGGTGGAACAGATGGTGAATGAGGGTGTTAATGCAGATAGCAT
CAAACAAGCCTCAGAACAACTGAACAGCCGGTGGATCGAATTCTGCCAG
TTGCTAAGTGAGAGACTTAACTGGCTGGAGTATCAGAACAACATCATCG
CTTTCTATAATCAGCTACAACAATTGGAGCAGATGACAACTACTGCTGA
AAACTGGTTGAAAATCCAACCCACCACCCCATCAGAGCCAACAGCAATT
AAAAGTCAGTTAAAAATTTGTAAGGATGAAGTCAACCGGCTATCAGGTC
TTCAACCTCAAATTGAACGATTAAAAATTCAAAGCATAGCCCTGAAAGA
GAAAGGACAAGGACCCATGTTCCTGGATGCAGACTTTGTGGCCTTTACA
AATCATTTTAAGCAAGTCTTTTCTGATGTGCAGGCCAGAGAGAAAGAGC
TACAGACAATTTTTGACACTTTGCCACCAATGCGCTATCAGGAGACCAT
GAGTGCCATCAGGACATGGGTCCAGCAGTCAGAAACCAAACTCTCCATA
CCTCAACTTAGTGTCACCGACTATGAAATCATGGAGCAGAGACTCGGGG
AATTGCAGGCTTTACAAAGTTCTCTGCAAGAGCAACAAAGTGGCCTATA
CTATCTCAGCACCACTGTGAAAGAGATGTCGAAGAAAGCGCCCTCTGAA
ATTAGCCGGAAATATCAATCAGAATTTGAAGAAATTGAGGGACGCTGGA
AGAAGCTCTCCTCCCAGCTGGTTGAGCATTGTCAAAAGCTAGAGGAGCA
AATGAATAAACTCCGAAAAATTCAGAATCACATACAAACCCTGAAGAAA
TGGATGGCTGAAGTTGATGTTTTTCTGAAGGAGGAATGGCCTGCCCTTG
GGGATTCAGAAATTCTAAAAAAGCAGCTGAAACAGTGCAGACTTTTAGT
CAGTGATATTCAGACAATTCAGCCCAGTCTAAACAGTGTCAATGAAGGT
GGGCAGAAGATAAAGAATGAAGCAGAGCCAGAGTTTGCTTCGAGACTTG
AGACAGAACTCAAAGAACTTAACACTCAGTGGGATCACATGTGCCAACA
GGTCTATGCCAGAAAGGAGGCCTTGAAGGGAGGTTTGGAGAAAACTGTA
AGCCTCCAGAAAGATCTATCAGAGATGCACGAATGGATGACACAAGCTG
AAGAAGAGTATCTTGAGAGAGATTTTGAATATAAAACTCCAGATGAATT
ACAGAAAGCAGTTGAAGAGATGAAGAGAGCTAAAGAAGAGGCCCAACAA
AAAGAAGCGAAAGTGAAACTCCTTACTGAGTCTGTAAATAGTGTCATAG
CTCAAGCTCCACCTGTAGCACAAGAGGCCTTAAAAAAGGAACTTGAAAC
TCTAACCACCAACTACCAGTGGCTCTGCACTAGGCTGAATGGGAAATGC
AAGACTTTGGAAGAAGTTTGGGCATGTTGGCATGAGTTATTGTCATACT
TGGAGAAAGCAAACAAGTGGCTAAATGAAGTAGAATTTAAACTTAAAAC
CACTGAAAACATTCCTGGCGGAGCTGAGGAAATCTCTGAGGTGCTAGAT
TCACTTGAAAATTTGATGCGACATTCAGAGGATAACCCAAATCAGATTC
GCATATTGGCACAGACCCTAACAGATGGCGGAGTCATGGATGAGCTAAT
CAATGAGGAACTTGAGACATTTAATTCTCGTTGGAGGGAACTACATGAA
GAGGCTGTAAGGAGGCAAAAGTTGCTTGAACAGAGCATCCAGTCTGCCC
AGGAGACTGAAAAATCCTTACACTTAATCCAGGAGTCCCTCACATTCAT
TGACAAGCAGTTGGCAGCTTATATTGCAGACAAGGTGGACGCAGCTCAA
ATGCCTCAGGAAGCCCAGAAAATCCAATCTGATTTGACAAGTCATGAGA
TCAGTTTAGAAGAAATGAAGAAACATAATCAGGGGAAGGAGGCTGCCCA
AAGAGTCCTGTCTCAGATTGATGTTGCACAGAAAAAATTACAAGATGTC
TCCATGAAGTTTCGATTATTCCAGAAACCAGCCAATTTTGAGCAGCGTC
TACAAGAAAGTAAGATGATTTTAGATGAAGTGAAGATGCACTTGCCTGC
ATTGGAAACAAAGAGTGTGGAACAGGAAGTAGTACAGTCACAGCTAAAT
CATTGTGTGAACTTGTATAAAAGTCTGAGTGAAGTGAAGTCTGAAGTGG
AAATGGTGATAAAGACTGGACGTCAGATTGTACAGAAAAAGCAGACGGA
AAATCCCAAAGAACTTGATGAAAGAGTAACAGCTTTGAAATTGCATTAT
AATGAGCTGGGAGCAAAGGTAACAGAAAGAAAGCAACAGTTGGAGAAAT
GCTTGAAATTGTCCCGTAAGATGCGAAAGGAAATGAATGTCTTGACAGA
ATGGCTGGCAGCTACAGATATGGAATTGACAAAGAGATCAGCAGTTGAA
GGAATGCCTAGTAATTTGGATTCTGAAGTTGCCTGGGGAAAGGCTACTC
AAAAAGAGATTGAGAAACAGAAGGTGCACCTGAAGAGTATCACAGAGGT
AGGAGAGGCCTTGAAAACAGTTTTGGGCAAGAAGGAGACGTTGGTGGAA
GATAAACTCAGTCTTCTGAATAGTAACTGGATAGCTGTCACCTCCCGAG
CAGAAGAGTGGTTAAATCTTTTGTTGGAATACCAGAAACACATGGAAAC
TTTTGACCAGAATGTGGACCACATCACAAAGTGGATCATTCAGGCTGAC
ACACTTTTGGATGAATCAGAGAAAAAGAAACCCCAGCAAAAAGAAGACG
TGCTTAAGCGTTTAAAGGCAGAACTGAATGACATACGCCCAAAGGTGGA
CTCTACACGTGACCAAGCAGCAAACTTGATGGCAAACCGCGGTGACCAC
TGCAGGAAATTAGTAGAGCCCCAAATCTCAGAGCTCAACCATCGATTTG
CAGCCATTTCACACAGAATTAAGACTGGAAAGGCCTCCATTCCTTTGAA
GGAATTGGAGCAGTTTAACTCAGATATACAAAAATTGCTTGAACCACTG
GAGGCTGAAATTCAGCAGGGGGTGAATCTGAAAGAGGAAGACTTCAATA
AAGATATGAATGAAGACAATGAGGGTACTGTAAAAGAATTGTTGCAAAG
AGGAGACAACTTACAACAAAGAATCACAGATGAGAGAAAGCGAGAGGAA
ATAAAGATAAAACAGCAGCTGTTACAGACAAAACATAATGCTCTCAAGG
ATTTGAGGTCTCAAAGAAGAAAAAAGGCTCTAGAAATTTCTCATCAGTG
GTATCAGTACAAGAGGCAGGCTGATGATCTCCTGAAATGCTTGGATGAC
ATTGAAAAAAAATTAGCCAGCCTACCTGAGCCCAGAGATGAAAGGAAAA
TAAAGGAAATTGATCGGGAATTGCAGAAGAAGAAAGAGGAGCTGAATGC
AGTGCGTAGGCAAGCTGAGGGCTTGTCTGAGGATGGGGCCGCAATGGCA
GTGGAGCCAACTCAGATCCAGCTCAGCAAGCGCTGGCGGGAAATTGAGA
GCAAATTTGCTCAGTTTCGAAGACTCAACTTTGCACAAATTCACACTGT
CCGTGAAGAAACGATGATGGTGATGACTGAAGACATGCCTTTGGAAATT
TCTTATGTGCCTTCTACTTATTTGACTGAAATCACTCATGTCTCACAAG
CCCTATTAGAAGTGGAACAACTTCTCAATGCTCCTGACCTCTGTGCTAA
GGACTTTGAAGATCTCTTTAAGCAAGAGGAGTCTCTGAAGAATATAAAA
GATAGTCTACAACAAAGCTCAGGTCGGATTGACATTATTCATAGCAAGA
AGACAGCAGCATTGCAAAGTGCAACGCCTGTGGAAAGGGTGAAGCTACA
GGAAGCTCTCTCCCAGCTTGATTTCCAATGGGAAAAAGTTAACAAAATG
TACAAGGACCGACAAGGGCGATTTGACAGATCTGTTGAGAAATGGCGGC
GTTTTCATTATGATATAAAGATATTTAATCAGTGGCTAACAGAAGCTGA
ACAGTTTCTCAGAAAGACACAAATTCCTGAGAATTGGGAACATGCTAAA
TACAAATGGTATCTTAAGGAACTCCAGGATGGCATTGGGCAGCGGCAAA
CTGTTGTCAGAACATTGAATGCAACTGGGGAAGAAATAATTCAGCAATC
CTCAAAAACAGATGCCAGTATTCTACAGGAAAAATTGGGAAGCCTGAAT
CTGCGGTGGCAGGAGGTCTGCAAACAGCTGTCAGACAGAAAAAAGAGGC
TAGAAGAACAAAAGAATATCTTGTCAGAATTTCAAAGAGATTTAAATGA
ATTTGTTTTATGGTTGGAGGAAGCAGATAACATTGCTAGTATCCCACTT
GAACCTGGAAAAGAGCAGCAACTAAAAGAAAAGCTTGAGCAAGTCAAGT
TACTGGTGGAAGAGTTGCCCCTGCGCCAGGGAATTCTCAAACAATTAAA
TGAAACTGGAGGACCCGTGCTTGTAAGTGCTCCCATAAGCCCAGAAGAG
CAAGATAAACTTGAAAATAAGCTCAAGCAGACAAATCTCCAGTGGATAA
AGGTTTCCAGAGCTTTACCTGAGAAACAAGGAGAAATTGAAGCTCAAAT
AAAAGACCTTGGGCAGCTTGAAAAAAAGCTTGAAGACCTTGAAGAGCAG
TTAAATCATCTGCTGCTGTGGTTATCTCCTATTAGGAATCAGTTGGAAA
TTTATAACCAACCAAACCAAGAAGGACCATTTGACGTTCAGGAAACTGA
AATAGCAGTTCAAGCTAAACAACCGGATGTGGAAGAGATTTTGTCTAAA
GGGCAGCATTTGTACAAGGAAAAACCAGCCACTCAGCCAGTGAAGAGGA
AGTTAGAAGATCTGAGCTCTGAGTGGAAGGCGGTAAACCGTTTACTTCA
AGAGCTGAGGGCAAAGCAGCCTGACCTAGCTCCTGGACTGACCACTATT
GGAGCCTCTCCTACTCAGACTGTTACTCTGGTGACACAACCTGTGGTTA
CTAAGGAAACTGCCATCTCCAAACTAGAAATGCCATCTTCCTTGATGTT
GGAGGTACCTGCTCTGGCAGATTTCAACCGGGCTTGGACAGAACTTACC
GACTGGCTTTCTCTGCTTGATCAAGTTATAAAATCACAGAGGGTGATGG
TGGGTGACCTTGAGGATATCAACGAGATGATCATCAAGCAGAAGGCAAC
AATGCAGGATTTGGAACAGAGGCGTCCCCAGTTGGAAGAACTCATTACC
GCTGCCCAAAATTTGAAAAACAAGACCAGCAATCAAGAGGCTAGAACAA
TCATTACGGATCGAATTGAAAGAATTCAGAATCAGTGGGATGAAGTACA
AGAACACCTTCAGAACCGGAGGCAACAGTTGAATGAAATGTTAAAGGAT
TCAACACAATGGCTGGAAGCTAAGGAAGAAGCTGAGCAGGTCTTAGGAC
AGGCCAGAGCCAAGCTTGAGTCATGGAAGGAGGGTCCCTATACAGTAGA
TGCAATCCAAAAGAAAATCACAGAAACCAAGCAGTTGGCCAAAGACCTC
CGCCAGTGGCAGACAAATGTAGATGTGGCAAATGACTTGGCCCTGAAAC
TTCTCCGGGATTATTCTGCAGATGATACCAGAAAAGTCCACATGATAAC
AGAGAATATCAATGCCTCTTGGAGAAGCATTCATAAAAGGGTGAGTGAG
CGAGAGGCTGCTTTGGAAGAAACTCATAGATTACTGCAACAGTTCCCCC
TGGACCTGGAAAAGTTTCTTGCCTGGCTTACAGAAGCTGAAACAACTGC
CAATGTCCTACAGGATGCTACCCGTAAGGAAAGGCTCCTAGAAGACTCC
AAGGGAGTAAAAGAGCTGATGAAACAATGGCAAGACCTCCAAGGTGAAA
TTGAAGCTCACACAGATGTTTATCACAACCTGGATGAAAACAGCCAAAA
AATCCTGAGATCCCTGGAAGGTTCCGATGATGCAGTCCTGTTACAAAGA
CGTTTGGATAACATGAACTTCAAGTGGAGTGAACTTCGGAAAAAGTCTC
TCAACATTAGGTCCCATTTGGAAGCCAGTTCTGACCAGTGGAAGCGTCT
GCACCTTTCTCTGCAGGAACTTCTGGTGTGGCTACAGCTGAAAGATGAT
GAATTAAGCCGGCAGGCACCTATTGGAGGCGACTTTCCAGCAGTTCAGA
AGCAGAACGATGTACATAGGGCCTTCAAGAGGGAATTGAAAACTAAAGA
ACCTGTAATCATGAGTACTCTTGAGACTGTACGAATATTTCTGACAGAG
CAGCCTTTGGAAGGACTAGAGAAACTCTACCAGGAGCCCAGAGAGCTGC
CTCCTGAGGAGAGAGCCCAGAATGTCACTCGGCTTCTACGAAAGCAGGC
TGAGGAGGTCAATACTGAGTGGGAAAAATTGAACCTGCACTCCGCTGAC
TGGCAGAGAAAAATAGATGAGACCCTTGAAAGACTCCAGGAACTTCAAG
AGGCCACGGATGAGCTGGACCTCAAGCTGCGCCAAGCTGAGGTGATCAA
GGGATCCTGGCAGCCCGTGGGCGATCTCCTCATTGACTCTCTCCAAGAT
CACCTCGAGAAAGTCAAGGCACTTCGAGGAGAAATTGCGCCTCTGAAAG
AGAACGTGAGCCACGTCAATGACCTTGCTCGCCAGCTTACCACTTTGGG
CATTCAGCTCTCACCGTATAACCTCAGCACTCTGGAAGACCTGAACACC
AGATGGAAGCTTCTGCAGGTGGCCGTCGAGGACCGAGTCAGGCAGCTGC
ATGAAGCCCACAGGGACTTTGGTCCAGCATCTCAGCACTTTCTTTCCAC
GTCTGTCCAGGGTCCCTGGGAGAGAGCCATCTCGCCAAACAAAGTGCCC
TACTATATCAACCACGAGACTCAAACAACTTGCTGGGACCATCCCAAAA
TGACAGAGCTCTACCAGTCTTTAGCTGACCTGAATAATGTCAGATTCTC
AGCTTATAGGACTGCCATGAAACTCCGAAGACTGCAGAAGGCCCTTTGC
TTGGATCTCTTGAGCCTGTCAGCTGCATGTGATGCCTTGGACCAGCACA
ACCTCAAGCAAAATGACCAGCCCATGGATATCCTGCAGATTATTAATTG
TTTGACCACTATTTATGACCGCCTGGAGCAAGAGCACAACAATTTGGTC
AACGTCCCTCTCTGCGTGGATATGTGTCTGAACTGGCTGCTGAATGTTT
ATGATACGGGACGAACAGGGAGGATCCGTGTCCTGTCTTTTAAAACTGG
CATCATTTCCCTGTGTAAAGCACATTTGGAAGACAAGTACAGATACCTT
TTCAAGCAAGTGGCAAGTTCAACAGGATTTTGTGACCAGCGCAGGCTGG
GCCTCCTTCTGCATGATTCTATCCAAATTCCAAGACAGTTGGGTGAAGT
TGCATCCTTTGGGGGCAGTAACATTGAGCCAAGTGTCCGGAGCTGCTTC
CAATTTGCTAATAATAAGCCAGAGATCGAAGCGGCCCTCTTCCTAGACT
GGATGAGACTGGAACCCCAGTCCATGGTGTGGCTGCCCGTCCTGCACAG
AGTGGCTGCTGCAGAAACTGCCAAGCATCAGGCCAAATGTAACATCTGC
AAAGAGTGTCCAATCATTGGATTCAGGTACAGGAGTCTAAAGCACTTTA
ATTATGACATCTGCCAAAGCTGCTTTTTTTCTGGTCGAGTTGCAAAAGG
CCATAAAATGCACTATCCCATGGTGGAATATTGCACTCCGACTACATCA
GGAGAAGATGTTCGAGACTTTGCCAAGGTACTAAAAAACAAATTTCGAA
CCAAAAGGTATTTTGCGAAGCATCCCCGAATGGGCTACCTGCCAGTGCA
GACTGTCTTAGAGGGGGACAACATGGAAACTCCCGTTACTCTGATCAAC
TTCTGGCCAGTAGATTCTGCGCCTGCCTCGTCCCCTCAGCTTTCACACG
ATGATACTCATTCACGCATTGAACATTATGCTAGCAGGCTAGCAGAAAT
GGAAAACAGCAATGGATCTTATCTAAATGATAGCATCTCTCCTAATGAG
AGCATAGATGATGAACATTTGTTAATCCAGCATTACTGCCAAAGTTTGA
ACCAGGACTCCCCCCTGAGCCAGCCTCGTAGTCCTGCCCAGATCTTGAT
TTCCTTAGAGAGTGAGGAAAGAGGGGAGCTAGAGAGAATCCTAGCAGAT
CTTGAGGAAGAAAACAGGAATCTGCAAGCAGAATATGACCGTCTAAAGC
AGCAGCACGAACATAAAGGCCTGTCCCCACTGCCGTCCCCTCCTGAAAT
GATGCCCACCTCTCCCCAGAGTCCCCGGGATGCTGAGCTCATTGCTGAG
GCCAAGCTACTGCGTCAACACAAAGGCCGCCTGGAAGCCAGGATGCAAA
TCCTGGAAGACCACAATAAACAGCTGGAGTCACAGTTACACAGGCTAAG
GCAGCTGCTGGAGCAACCCCAGGCAGAGGCCAAAGTGAATGGCACAACG
GTGTCCTCTCCTTCTACCTCTCTACAGAGGTCCGACAGCAGTCAGCCTA
TGCTGCTCCGAGTGGTTGGCAGTCAAACTTCGGACTCCATGGGTGAGGA
AGATCTTCTCAGTCCTCCCCAGGACACAAGCACAGGGTTAGAGGAGGTG
ATGGAGCAACTCAACAACTCCTTCCCTAGTTCAAGAGGAAGAAATACCC
CTGGAAAGCCAATGAGAGAGGACACAATGTAGGAAGTCTTTTCCA
CATGGCAGATGATTTGGGCAGAGCGATGGAGTCCTTAGTATCAGTCATG
ACAGATGAAGAAGGAGCAGAATAAATGTTTTACAACTCCTGATTCCCGC
ATGGTTTTTATAATATTCATACAACAAAGAGGATTAGACAGTAAGAGTT
TACAAGAAATAAATCTATATTTTTGTGAAGGGTAGTGGTATTATACTGT
AGATTTCAGTAGTTTCTAAGTCTGTTATTGTTTTGTTAACAATGGCAGG
TTTTACACGTCTATGCAATTGTACAAAAAAGTTATAAGAAAACTACATG
TAAAATCTTGATAGCTAAATAACTTGCCATTTCTTTATATGGAACGCAT
TTTGGGTTGTTTAAAAATTTATAACAGTTATAAAGAAAGATTGTAAACT
AAAGTGTGCTTTATAAAAAAAAGTTGTTTATAAAAACCCCTAAAAACAA
AACAAACACACACACACACACATACACACACACACACAAAACTTTGAGG
CAGCGCATTGTTTTGCATCCTTTTGGCGTGATATCCATATGAAATTCAT
GGCTTTTTCTTTTTTTGCATATTAAAGATAAGACTTCCTCTACCACCAC
ACCAAATGACTACTACACACTGCTCATTTGAGAACTGTCAGCTGAGTGG
GGCAGGCTTGAGTTTTCATTTCATATATCTATATGTCTATAAGTATATA
AATACTATAGTTATATAGATAAAGAGATACGAATTTCTATAGACTGACT
TTTTCCATTTTTTAAATGTTCATGTCACATCCTAATAGAAAGAAATTAC
TTCTAGTCAGTCATCCAGGCTTACCTGCTTGGTCTAGAATGGATTTTTC
CCGGAGCCGGAAGCCAGGAGGAAACTACACCACACTAAAACATTGTCTA
CAGCTCCAGATGTTTCTCATTTTAAACAACTTTCCACTGACAACGAAAG
TAAAGTAAAGTATTGGATTTTTTTAAAGGGAACATGTGAATGAATACAC
AGGACTTATTATATCAGAGTGAGTAATCGGTTGGTTGGTTGATTGATTG
ATTGATTGATACATTCAGCTTCCTGCTGCTAGCAATGCCACGATTTAGA
TTTAATGATGCTTCAGTGGAAATCAATCAGAAGGTATTCTGACCTTGTG
AACATCAGAAGGTATTTTTTAACTCCCAAGCAGTAGCAGGACGATGATA
GGGCTGGAGGGCTATGGATTCCCAGCCCATCCCTGTGAAGGAGTAGGCC
ACTCTTTAAGTGAAGGATTGGATGATTGTTCATAATACATAAAGTTCTC
TGTAATTACAACTAAATTATTATGCCCTCTTCTCACAGTCAAAAGGAAC
TGGGTGGTTTGGTTTTTGTTGCTTTTTTAGATTTATTGTCCCATGTGGG
ATGAGTTTTTAAATGCCACAAGACATAATTTAAAATAAATAAACTTTGG
GAAAAGGTGTAAAACAGTAGCCCCATCACATTTGTGATACTGACAGGTA
TCAACCCAGAAGCCCATGAACTGTGTTTCCATCCTTTGCATTTCTCTGC
GAGTAGTTCCACACAGGTTTGTAAGTAAGTAAGAAAGAAGGCAAATTGA
TTCAAATGTTACAAAAAAACCCTTCTTGGTGGATTAGACAGGTTAAATA
TATAAACAAACAAACAAAAATTGCTCAAAAAAGAGGAGAAAAGCTCAAG
AGGAAAAGCTAAGGACTGGTAGGAAAAAGCTTTACTCTTTCATGCCATT
TTATTTCTTTTTGATTTTTAAATCATTCATTCAATAGATACCACCGTGT
GACCTATAATTTTGCAAATCTGTTACCTCTGACATCAAGTGTAATTAGC
TTTTGGAGAGTGGGCTGACATCAAGTGTAATTAGCTTTTGGAGAGTGGG
TTTTGTCCATTATTAATAATTAATTAATTAACATCAAACACGGCTTCTC
ATGCTATTTCTACCTCACTTTGGTTTTGGGGTGTTCCTGATAATTGTGC
ACACCTGAGTTCACAGCTTCACCACTTGTCCATTGCGTTATTTTCTTTT
TCCTTTATAATTCTTTCTTTTTCCTTCATAATTTTCAAAAGAAAACCCA
AAGCTCTAAGGTAACAAATTACCAAATTACATGAAGATTTGGTTTTTGT
CTTGCATTTTTTTCCTTTATGTGACGCTGGACCTTTTCTTTACCCAAGG
ATTTTTAAAACTCAGATTTAAAACAAGGGGTTACTTTACATCCTACTAA
GAAGTTTAAGTAAGTAAGTTTCATTCTAAAATCAGAGGTAAATAGAGTG
CATAAATAATTTTGTTTTAATCTTTTTGTTTTTCTTTTAGACACATTAG
CTCTGGAGTGAGTCTGTCATAATATTTGAACAAAAATTGAGAGCTTTAT
TGCTGCATTTTAAGCATAATTAATTTGGACATTATTTCGTGTTGTGTTC
TTTATAACCACCAAGTATTAAACTGTAAATCATAATGTAACTGAAGCAT
AAACATCACATGGCATGTTTTGTCATTGTTTTCAGGTACTGAGTTCTTA
CTTGAGTATCATAATATATTGTGTTTTAACACCAACACTGTAACATTTA
CGAATTATTTTTTTAAACTTCAGTTTTACTGCATTTTCACAACATATCA
GACTTCACCAAATATATGCCTTACTATTGTATTATAGTACTGCTTTACT
GTGTATCTCAATAAAGCACGCAGTTATGTTAC

Homo sapiens dystrophin (DMD), transcript variant Dp427m, exon 50 (nucleotide positions 7445-7553 of NCBI Reference Sequence: NM_004006.2; nucleotide positions 1524527-1524635 of NCBI Reference Sequence: NG_012232.1)

(SEQ ID NO: 131)
AGGAAGTTAGAAGATCTGAGCTCTGAGTGGAAGGCGGTAAACCGTTTAC
TTCAAGAGCTGAGGGCAAAGCAGCCTGACCTAGCTCCTGGACTGACCAC
TATTGGAGCCT

Homo sapiens dystrophin (DMD) exon 50/intron 50 junction (nucleotide positions 1524606-1524665 of NCBI Reference Sequence: NG_012232.1) TAGCTCCTGGACTGACCACTATTGGAGCCTGTAAGTATACTGGATCCCATTCTCTTTGGC (SEQ ID NO: 832)

(SEQ ID NO: 832)
TAGCTCCTGGACTGACCACTATTGGAGCCTGTAAGTATACTGGATCCCA
TTCTCTTTGGC

Homo sapiens dystrophin (DMD) exon 50/intron 50 junction target sequence 1 (nucleotide positions 1524626-1524677 of NCBI Reference Sequence: NG_012232.1)

(SEQ ID NO: 833)
ATTGGAGCCTGTAAGTATACTGGATCCCATTCTCTTTGGCTCTAGCTAT
TTG

Homo sapiens dystrophin (DMD), intron 50 (nucleotide positions 1524636-1570417 of NCBI Reference Sequence: NG_012232.1)

(SEQ ID NO: 834)
GTAAGTATACTGGATCCCATTCTCTTTGGCTCTAGCTATTTGTTCAAAA
GTGCAACTATGAAGTGATGACTGGGTGAGAGAGAAAATTTGTTTCAATT
CTAAAGATAGAGATAAACCTTTGTGTTATTGACTGTGCAAAAAGTCTTA
GAGTACATTCCTTGGAAATTGACTCTGATTCAAAGTGTTGCATGACAAC
GGGATATGGGGAGTGTTCTCTGGAGATACACCCACAAGGAAGAGAAGAG
CACAAGGGAGATTGTGGGAGAGTCTGAAATGTGATTTGTCTGCAGCAGA
GGCCTAAGCCAGTCTCGCAGGAGCCCTACATCTGGGCTGGCTGTGCAGA
GCTGTCCTGAATTGCAGGCAGTGGGCCTGGCCCTTGTATTCCTGATCCA
GCCAGCCATTGGCCAGGGGCTGGCTGCTGCCTGAGAGTGGAAGGACAAC
TTGGACAAGTTTTCTGAGGCCGAAGGCAATTCTTAGTAAGGAACACCAT
TAACAACCAATATTCCTAGCATCCAGGGATGTGTGCATTGTTCCTGAAG
AGGGACAAGTATGTCTACAAAAATCACAGAAACCACAGAAACACACACA
GTCCTACTAGCACCTCTCCCTGTCCCATTTGCAAACAATTTAAGAGCTC
TCCCATTTTTAGTTCAAGAAAAAGAAAAATGGATTGGGAGGACCACAAG
CTGACTTGGGGGAGGAATATTTCCTCATTTAGCTGTAGTTTTAACTTTT
GTTTTCACTGCATATTTTCAGTCTATTTTATTTTCTTTCCTCTTCAGTT
GTTGATAGAAGGTATTCATAAATTCTCATGGCAATGTTAATGCTGGCTT
TGACTCTCAGGGGAAAGAGGCCAGAAAACTTCTTTGCTGTACCATTCCA
TAATTAGGCAGAACTAAAAACATCTTTGGGTGTTGTTTTTTGTTTTTGT
TTTTTTTTTTGCCTTGTCTGCTTTTCAAAGATCAAATGATTGAAGCATT
AAAGCATGGTGACTGGTTCTTCAGGTAAAGTTGATTTTTATTTTATGTC
AAGTAGAAAAATACTGAACTGGAAGAATCACAGCTGGGGTAGCACAATC
ATAATTCATTAGAAGGCATAAATAGTGCTTGGATTAAAAGAAGCCCTAC
AATCTGGGGACAGTGCATCTCATGTGCCCTCTGGGATTACTCGGCAGTC
ATCAGAGTTAGATTTAACGACTTTGGAGACTTAAGCATTATGGTTTTTT
TTTTTTGTCAATCTGGGACACTGAAATTGCTGTATCAGGGTTATACTCA
ACTGTGTCAGGTTTATTTGTTTTTATGAGCTGTAATTTTTGGTTCCCTC
AGCGCATATGCATAGTTTGTTCCTATGTTATCATTTATTGGTGTCTGTT
TTCTGGCTGTCTCTGGTAGGTTCAGCCTCAGACTCTGTAACTCCATGAA
GAGATTATGTTCCAATGATGTTTTATAAGTTTGTTAAACTCTGAACTCA
TGAGTTTATGTCCCATATAAGCCACGTTACACATGGTAGGAAGGCTCCA
AAACCAGGGCGCCGAAATCCATTTAACGTGTAACTTACCTAAATGTAAC
AATGTTTATAAGAAAAATACATTGGAAGTTCCAGTTTTGACTTCCAGCA
ACATATATAATTCATCCACTTTATTTATTAACTTCCATGTGTTGAGCAT
CATACTGGTGCTGCGAGTACAGCATAGAATAAAAGTCTCTCCTTTCATA
AAACATATATTGTAATTGAAAGAGAAAGACAATAAACTAATGAAGAAAA
TATATACTGTCTCAATAATTATAAGTGCTGTAGAGTGTAGTCTACAGAT
TGATGTCAATGGGTATTTGTTAGACACACAGAATCTGAGGCCCCTATCC
TAGACCCACTGAATCACAATCTGCATTTTAATAGAATCCCCAGGTGAAT
CCTGTGCACACTGACATTTGAGAAACACCATTACAGAGAACAACTAAGC
AGGGAGATGGGATGAGGGTATTATTGTCTATAGTGTGGTCAGGGAAGCT
TGTCTGTTAAGAGAACATCAGAAAACTGATGTAAGTGAGGAAGTGAGCC
TGGTGTATTTCTGGGAAAATTATTCCAGGCAGGGAGAGAAAAGACTGAG
CAACGATACTGAAGTAGGAACAAGATGACGGAATATTAAGGAGATCAGT
GAGACTAGAGGAGTGGGTCAGGGGAAGTGTGATGGAAGCCATGAGAGAT
ACTCATCTTTCATAGCACTGCCCTACTTCCTTCTCCCCAACATGAGGGT
CTCATCACCCCCCACCACTCTTGTCTTCTCCTATGTCCTCCACATTGCT
GCCAGTATGGAGAGTCTGGGAATGCCCTCAGCTCAAAGCTGTTTGGTGA
TAGCTGGCAGAGTTGTGGTAGTAGCTAAAAAAGAATTAAGGGAAAGAGG
AATTTTCTCAAAAGCAGGTGCTTTTCATCCTCTTTAGCAAACCGAAACA
GATCTGAGCATTAAGTCAAGATGTTAAATACACAAATGTTGAATGAAAA
AAAAAACAAAAGGTAGTCATTTAAATTCAGAGCTGCTTTTATTAAAATA
AGATTTTCTTTTTTCTTTACTGTGGTAGTTCAAATATCAGAATAAAGAA
TTGTTTCTATTCCCGACTTCCTGACTTGCAGGAAGTTAATCAGAAATAA
ATGCAATATAAAAAAAGAAAATCTAATTTGTATTATGCTTCTTGTATAT
GTTTATTATTTCATGTACTGTATTACAATGTAATAGAATTTATAATTCA
TTATAGCAGATTGTTTCCATTGCATTCCTACTATTAAATATGTAGAAGC
TACACATATACTTGTAGCTTTAACATATATGTCTTTATCCTCAAAATAA
CTGCAAAGAACATATAGATAATTTTTAAAGATTAAGGAGCCTGAGGTTT
AGAGGGGAGATAGCTAGATTAAGGCCACACAGCTAGAAAGCAAGCAAGC
AAGGGTTTCAGTCCACATGTCAAGCTCCACAGCCTGTGTTTTGTTTTGT
GGCTGTGCTTTACACTATCTCTCTGTCCAAGAACCTAATGGAAAATTAC
AGATACAGATGCAGCTGGCCAGCAGTTAATATAATTTAACTCAATCTTA
AATTTATCTGGAGTAAAAGTGATACAAGTTTCCGTGTTTTTTCTTTTCT
TTCTTTCTTTTTTCTTTGTGTGTGTGTGTGTGTGTGTGTGTGTGACAGA
ATCTTGCTCTGTTGCCCAGGCTGGAGTGCAGTGGTGCGATCTCGGTTTA
CTACAACCTCTGCCTTTCAGGTTCAAGCGATTCTCCTGCCTCAGCCTCC
CAAGTAGCTGGGATACAGGTGCGCACCACCATGCCCAGCTAATTTTTGT
AGTTTTAGTAAAGACGGGGTTGCTCCATGTTGATCAGGCTGGTCTTAAA
CTCCTGACCTCAAGTGATCCGCCTGCCTCAGCCTCTCAAAGTGCTGGGA
TTACAGGCATGAGCCACCTTGACTGGCCTGTTTCTGTGTTTTTTCTACT
TAAGAAGTAGAAAAAATTGGTTCACTCTATTTGAATTTCTTAGAACCAT
AGAAATCCAAACTTGGAATAATTATTGCAATTATTATCTAGTTGAGTAT
TCTTCTTTTATAGATGTAGAAGCTGAGGCCTAAAGTTGCTTGTTTTGTT
TTATGAACACTAAACCAAACTACCTTGTGGCTGCTTACTTAAATTATAA
ATTATAATGGGGTGGCCTTGCCTGAGCTGTATAAATTGTTTTAATTATC
AGGACAAATCAACATACTGGAAAAAAAAAGCAAAACTTGCAATTGTTGT
TGCTTAGACACCTGTCCATATCAGTTTCTATTGTTGCATAGAAGCACCC
CAAAACTTAACAGTTCAAAACGAGTGATTTATTGCTCATAATTCTGTGT
GTCTGAAGTTTGCTCTGTGATTAGCTGGATAGTTATTCTGATGGTCTTG
CTTGGTGTTACTTATGTGGATGCAGTTATCTAGCAAGTGAACAGGAGCT
AGATGGTCTAAAATGCACTCTCTCAGATATCTGACAGCTGGCTGTTGGT
TGCAGAACCTTGATTCTTCATAAGGCCACTCATCATCCTGTAGGCTAGA
TTAGGCTTCATTACATGGTATTCTCAGGGCAGTTTTCCAAGAGAGGGCG
GGTGGAAGCTACAAGACCTTCTGATGCCTAGGCTTTGAAACGTGTAGGT
TACTTCTGCTAAGTTATATTGGTCAAAGCACCTCAAAAGATCAGCCCAG
ATTCAAGAGATGAGGAAATTACTTCATCTCGTGAAAGGAGGAGATGCCA
CATCGCATATCAAAGGGGTATGCATATTGGGGATAGAAGGTTTTATTGT
CACCGTATTTATACACAAATCACTACATTGGGGAAAAGGAGGGAAACTG
GAGATCAAAAGTGTTGGTCTTAATCCAACTTAATTCTCAAAAATTACCA
TGCGTTAGAACCACCCAGTTCTTCGCAAAGTATAGATTACTGGGCTTCA
ACCCCAGAGTTTCTGATTTACTAGCTCTGGGGTGAGACCTGCATCTCTC
TCTCTCTCTTTTTTTTTGAGACTGAGTCTTGCTCTGTCACCCAGGCTGG
AGTGCAGTGGTGCAATCTCGGCTCACAGCAACCTCTGCCTCCTGGGTTG
AAGCGGTTCTCCTGCCTCAGCCTCCTGAGTAGCTAGGATTATAGGCACC
CGCCACCACGCCTGGCTAATTTTTGTATTTTTAGTAGAGACAGGGTTTC
ACCATGTTGGTCAGGCTGGTCTCGAACTCCTGACCTCAGGTGATCCACC
TGCCTCAGCCTCCCATAGTGCTGGGATTACAGGCGTGAGCCACTGTGCC
CAGCCAAGACTTGCATCTCTGAAAAGTTCCTAGGTTATGCTGATGCTGG
CCTATGCTTTGAGAACTACTACCACAGACATACAGTGAGTGGGGAAGAA
TAAATTCATCCCTTCTGCTGTGTGCAGCAAGGAGTGGGATTCCAATGAG
ATCCAGTGCTGTGAATGCTAAAGGGAAATCCATCTTATTTTAGCACCTC
TACTCCCCATCTCCCCACCCCGAGGATGTTATAGCTTAGAAGTTCAAGG
AGATGGACAACACACTAAACCAGGCAGTATTTGCCCTGCAGAGCTGTTC
AGTGTTCCTGGATGAGACCTCTGAGAAGAAAAGCCATAAGTTCCTCTAG
AGACTTTCACAATCATTTAGGTAGACAGGACTTTGCATGGGTCTGAAGG
CTTGCATGGCAGATGGAGGCAAAGAGCCAGCAAATCTGGTTGTAAATGT
CAATGTGAATCCTTTCTTATCCACAAGCTGCTGGGCCTGAGAACATTAA
TGTTCTACAATACCCGATTTAGCATTTTTGAAAGAAATTGCATATAGAC
ATGCTTAATGTGAAGACTCCAAATCAGGATATTTGATTCAAATGTCTCT
TGGTAATAACTATGGAATGAATAACCCATTGTATATGGACATATAGAAG
AGCCAGTTAACAGAGTTTTCTTTTTTTTTTTTTTTTTTTTTTTTTTTTT
TTTTGAGACGGAGTCTCACTCTGTCGCCCAGGCTGGAGTGCAGTGGCGC
GATCTCGGCTCACTGCAAGCTCCGCCTCCCGGGTTCACGCCATTCTCCT
GCCTCAGCCTCCCCCGTAGCTGGGACTACAGGCGCCCGCCACCACGCCC
GGCTAATTTTTTTGTGTTTTTTAGTAGAGACGGGGTTTCACTGTGTTAG
CCAGGATGGTCTCGATCTCCTGACCTCGTGATCCGCCCGCCTCGGCCTT
CCAAAGTGCTGGGATTACAGGCGTGAGCCACCGCGCCCGGCCCAGAGTT
TTCTAGTTGATTAAACACTGGTAAAATCATCTCTTCCTGTAATTAAGTT
TGGAGAGAGCAAGTCTCAAGTGTAATTAGGAAAGCACAGTATTGGAGGT
CAAGAAGCCTAAATTCTGATATCTCAGTTGTGCTACCAACTATATAAGG
GATCAACAGACTGTTTCTGCAAAGGACCGATACTAGATATTTTCTGCTT
TATGGGTCATGTGGTCAACTCTGTCATTGTATACTAAAAGCCACAGACA
ATACCTAAATAAAGGGACATGGCTGTGCTCCAGTGACACTTTATTTACA
GAAACGAGGCAGGTAAGATAGATTTGACCCTTAGTGGGCTACCCATTTC
CCCTTAGCACTCAGCCTTCCTATAAACCATGAAGCGTCTAACTACAAGT
ACCTGTGAGTCTTCCTGAGTCCTTTCTCTGCCTGTAGGAGTAGCTCTCC
CACTTGCAGAGCAGGCTGGAAGCTGGGGAGGAGATTATCTCTGGAATAG
CACTTAACCAATGGACAAAAGCTGGGGGATAAAGGTGTTGGTCTTCATA
GTTTTCATACTGCTATGAAGAAATACCCGAGACTGGATAATTTATACAG
GAAAAAGAAGTTTAATGGACTCACAGTTCCACATGGCTGGAGAGGCCTC
ACAATCATGGCAGAAGGCGAAGGAAGAGCAGAGGCACTTCTTACATGAC
AGCAGGCAAGAGAGCATGTGCAGGGAAACTGCTCTTTATAAAACCATCA
GATCTCATGAGACTTATTCACTATCAGGAGAACAGCACAGGAAAACCCC
ACCCCCATGATTCAGTTACCTCCCACATGGTCCCTCCCACAACACGTGG
GGATTATTGGAGTTACAATTCAAGATGAGATTTGGGTGAGAACACAGCC
AAACCATATCAATAAATATCCCACCTTCTTCTCCCCTTGGGTAGGACAC
CTCCAATGCATGTTCCACACTAAAAATCTCCAGTGAATTGGGCAGTTGA
CCACAATGATAAGCATACTTATTAGCATGCCTCGTATAGGCTTCCTTCC
GATGTGTAGGCTTTCTTCCCATTTTGGCAGAATCTGGACCCAACCCCAC
ATCTCCTGAGTTTGATTTCAGTCCTCTTCAGTATTCTCATATATCTCAA
CTTTGTTCCCTGCTGAGATACAACTGACAGATATATATGTATCTTCAGC
TCTGCAGCTCTATCTTCTCTTCTACCATGGCTTTCTGGGATCAGCTCCC
AATTGAATTATTTGTATTTAAATCCTTGTCTCAGGGTCTACTTCTGAGC
AAACCTAAATTAATACAAGTCTTAGAATTAGTGCCACAGTTATACCTTT
TGAGTAAGTTATGTTGCAATTCCCATTTTATAGATGAGATAACCAAGGG
TCAGAAAGGTTAAATAATTTGTCCAAGGTTACAGAGCTAGTCAGTGACA
GAGTGGGTATTCAAAAATAAGTCTCTATGATTCCAGAGCTTATGCTATT
AATCAGTGCACCATGTATCAAGCAGTGATTTAGTTATCTTCATTTGAAT
TTTTTGAAGTCTCTATTTAAATTGAATATCAAATCCTCACATATAAGGT
TATTTATACCTTTTTATTTGTTTTATTTATTTATTTATTTTTGCTTAAC
TTTTTTTTATTATACTTTAAGTTTTAGGGTACATGTGCACAATGTGCAG
GTCTGTTACATATATATACATGTGCCATGTTGGTGTGCTGCACCCATTA
ACTCGTCATTGAACATTAGGTATATCTCCTAATGCTATCCCTCCCCCCT
CCCCCCACCCCACAGCAGGCCCCAGTGTGTGATGTTCCCCTTCCTGTGA
CCATGTGTTCTCATTGTTCAATTCCCACCTGTGAGTGAGAATATGCGGT
GTTTGGTTTTTTGTCCTTGCGATAGTTTGCTGAGAATGATGGTTTACAG
CTTCATCCATATCCCTACAAAGGACATGAACTCATCATTTTTTATGGCT
GCATAGTATTCCATGGTGATTTATACATTTTAATTTGAACTTCACTCAC
CTCTACAAAGTTAAATAAAGCATTCCATTTCCATTTCAATAATACTTTA
AATATCTAGGCAGTTAAAGCATGAGGTTACTTGGCACTTAAATGTACTC
TTGTCATACTACTTTTGTTGGCTTAAATTGCAAAAAAAAAAAATGGTTA
ATTTATTGTGACAATACCCTACATTTATCTGCAGTGATCATTTTTTTTG
TGAAAATGGCCTTCGTTTATCTGCAGCAAAGGAAAAAGAGGATGGCAAT
TAGTTCTTGCATTCTTATTCCTCTCTTGGGTCCTGATCCTTCTCATTAA
TAGAAACATGGCAGGGGAGGGGTATATAACCCACACCCTTTCCTGTTGT
GGTTATGTTTCCACTGTTGATTCTGCTTCAGGTGAACCTTTAGGATTAG
GCAAATAAATTTCCGTGAGGCCAAATCTTTTTCTTCCTCATTAACAGAT
GATTTCTCTGCTAAAAACACTTACGACATGGCTATACTATTGCCGGTTT
TATAGTTACAGGCTCTAAACCTTGAAAACTTCCTCAAAGTCTAATACGT
CAGGAGCAAGCTTTTGTACAAAAAATGTGAAGACCCTTAATCAGTTCCA
ATAACAAAATAAATCCATTTTAAACCCTATCCCAAGATACTGCAAGGCC
TTGGAGCAGCTGGAGAGACTCCTTAACTCTTGACATTAATTAATTAATT
TAAAAATTCATATTTGTATGTATCAGTGAGGTAAGAGTGCTTGAAATAT
AATGAGATGTGTCACACTGTAGAAAGGGGAGTGACAACAGAAAGCCCTG
GCTGGTGAGGCCCCAGCACTTCTCACACTCATCAGAAGGAAGTCTTTCC
ATGAAGGCAGTAGGGGGTCCCTGGTCCCAGGCCAGGTCCTTCAACAATG
CTCACTGGTTAACAGGAAAGGCACTACAGTGGCATCATTGTTAACATCC
AAGACAGTGATAGAATGTGAAACTTCTACTGTTTAGTATTTAGTATTCA
GCATTTAGATGTTAATTATCCATTTTGTGATGAAGTTCCCTTTCTTCTC
CCTCTCTTAACCTTTTGGTAGTTTTATTGCATGGTTACCATTTCCAGTT
AGGGTTGTGCTTTGGGGTCTGAACTGATGAAGGAAGAGAAACTCTAGTT
CATCATTTCTAGGAAAAAGAGAAGGCTAACATCAATTCTGATGATTAGA
GATTTTTTGATTACCTATGTCCTGCATTTTAACAGAAATAAAATGTTAA
TTACAGTAAACTTACTTTAACCTTCTTATGCTATTTAACACTCTTCAAG
AAAGGACTTTTGCTTGAAATCACTACAGAGTATACCATAATCTTGACTG
TCATACTCTGACCAAGAAACCAGAACTATTTAGGCATATTTATGAGAGT
AAGTACCCTACCGCTCAAAATGGAAGGCTTCAACATGTATTCCTAATAT
TTCCTAAAACTTCACTGAATAGTTTTAAAATTGAATATAACTTTACCTT
CAGAGAGAAACAAGATTTCGAAAGGAAATTGTAGCAGTTTTGTACTTCA
ATTGTCGATTTTAAGATTGTGGTCTGAAAAGTATTTGAACAGTCATTCT
TTCTTTTCCACTATCTCAGGAAAGGCTCTCATTCTATTAGAAGCAATCT
TAGAAGCGTAACTGCCAACTCTCTTCTTAAAAAGTGAAGAGCAGATGGA
GTTCTACCAATCTGCTATACTTGACAGTTTTTCAACTACCTAACCTAAG
TTCCAGTTACTGTCTAAATTATTTTTATTCGAATAGGAAAAATACATTT
ATGCTTACTGTACATTTCGACAGTGTTCATTCTTTGAGGTCTGTTATTT
AATCTCCTTCCCACTTTTCTTTTCTATCTTAAATCAGATGAGTCCCCAT
GGTCTGGCAACAAAACAGCTGTTTCTCAAATTTTTAACGTCTCTGTGGC
CCTCCTCCTACCCTTCCAAGTGTTTTCCATACTATTTTTCTGTTGTGGG
CTTCAGAATTGGATGCTGTTCCATAATTACTGGTCTTCCCATGACTCTG
TCACATCATAAAGTCTATATATACCCTTTTCCTACGTTTACATGCCAAA
TACCAGGTGTGTCTAGCTTTAATTCAATGACTAGCATATGGTAGGTATT
GCACTAATATTTTTTGGTCAAGTGAACATATATTTTACAGATAGATCAA
ATACATCAACCTTCCCATGTAATAATAATAATACCAAGAAGATTAAATG
ATCGCGTGCTTGACCAGCTGTTTAGTGGCAGAATCTGGACCCAACCCCA
CATCTCCTGAGTTTAATTTCAGTGCTCTTCAGCATTCTTATATATCTCA
ACTTTGTTCCCTGCTGAGACACAGCTGACAGATATATATATATATCTTC
ACAATCTCAGTAGTAAGCCCTATTTTTTTAAGATTACAGTGCTGATCAA
AGAGGGATATTCTATGGCATTGATGCAAATCTTCCAGGCCAGATATAAC
CCCTATCTCCTTAAACCCTCATTCTTGACTGTGTCTTGAAATCAAATTT
GAGAACCTCTGCTTTACATGCTACCTTCCTCTTAAAATTCATAAGGTTT
ATTCCTTCTCTCATCTTCAGTATTTTTATGAAAATATGTTTTACTCTTA
TTTCCAAAGTGCTTGCCAGCCCATGCTGAGTTCATTAGACAAACAATCT
AGGTATCCTAATATTTAGGTAAAATTGCTAGCAGCACTAACTATACCAG
TACCCACTTAAATTGCAATATAAACCTACAATAGGAAAAAAAAAGTCAA
AATTATACTACTTTCAATTCCTACTTCTGGAATAATTATCACACCTTCA
AAAAAACTCATAATTGTTCTCCAATATTAAAAACCAGGAACTAAATTAC
ATCACTATATATATATAGCTATATAGACATATAACTGTATATAGTTTAT
ATATATATTACATGTCACTATATATGGTTATATATACAGATAGCTATAT
CTCTATATAGTTTTTATATAGGAAACTATATATATAAACTCATATATAT
TTATATATAAACTCATATATATATAAACTCATATATATTTATATATAAA
CTCATATATATTTATATATAAACTCATATATATATAAGAGATTTTATAT
GTATATATGAGAGACAATATTGAAATAAAACAAACTAGCCAATCTCCAA
TGTCCCTTCATTTTCCCAGGACTCTTCTTTATTCTCAAAGAAATGTATG
AATACACAATATAAATAAATGTTCTATAATGTTCATTATGGATTATAAT
AGTTTTCTTGTCATACTGAAGTGAATGGGAATTGTTTCTTCGATTATTA
GCATACTTTTCATAACTATGTGATTTGGTGATAGGCACTTTTGCTTACT
AAGTTCAGCATGATCAAAACAGAACTCCTATTATTTTTATTGTTGATTC
TTATTTCTTTAAGGTAAAATATGCCCTTTTCCTTTGAACCTGCACACTG
TTTAACAAATAAGCACATAACTCAGGATTGAATTGTACACTTCGATTTG
AGCTTTTTTTTCAAGGTCAGGACCCAGTTTCACAAGAAGTTTTATTTTT
TCCAATACAACTGACATCCACTCCCACCAGCTGAAAGACAAGAAAAACT
TGTCTAATAAAGCTTTCAGATTCAATTTGCTGCCTGCATACAGCTTGAG
GAATCTCTGGAGGTCACTCACAGCATGTGTTGCAACCCCAACAGGGAGA
AGTAATGAAAAGATTCTAGTTAAAAAGCTGACACTGCCCCTTCCAACCT
CTTTGAATGTGAATATAATAAGCCAGTTTACAGACGCAAATCTCTATGA
TTCTGGGGATTTCCATCTTGATCTCTGACTCCAAGGAACATTTGAATGC
ATGGATTTGTATCCATTATCTGGGTGAATAAATGCTTCATATTGAAAAA
AGGGGTGCTTTAACAACATAAGTCTGATGTAAATCAGGCAAAACAACAT
TGTCACTTCATGTTTAACTCTCCTGGAGGGTCTCTAAGGTCTCACAGTT
TGGTTCTATTCCAGTAATATATAGGCCTATCATAGCCATTTTCAAAAAT
AATACCTGCTTTCATTTCGATTATTCCCCCTAGCTTTTGCATTGACCCG
AACATACCTAATATTTATCTTAGGGCTAACACGCATTAATGCCTTGCTC
TGTACCAGGCATTTTGCCAAGTATTCTTTGTGCATTTTTCTGTTTAATC
TTACAGCAGCCTTATGAAATAGGTACTACATTATTATTATTTTTCACCA
TGAGAGGAAATGAAAGCCTAGAGAGAATGGTTATCCAAAAACACCCAGC
TACTAAGTGGGCACAGCATGGCCTTGAACCTGAGTCTTTATAAAGTTCA
TGCCTGTCTTTTACCTTTATGTTAAACATACTGAATCTTGGTCATGCAG
TCTATGAATGAAGACTCCATATACTCTAGGACCAATTCTACCATATTGT
GCATGCTTTTGTACATATTTTCCAAATGAAATTAAACAACAATACTCTC
TCTTCCCCTTTCTTTTCCGTTTTGCATGACATCTAAATCTTTTATTAAA
TCTCCGTGGGTGGAATTGGGCCTTAAGTAGTAGTACCTTTGAAGCTTAA
TACTATAACCTCAGAGTTACGGAAGTGGTTTCAATATGAAGAAATATAT
ACGTTCTTTTCTTTTCTTTTCTTTTCTTTTTTTGGCTCCTCTAGAATAG
AAGGCAATCAGGAGAGAAAAAGACATTAAGATGATAGGCTTGATTCTCC
CACAGTGTTACTACTTAGCTTTATTTATTCTCCCACATCTGTAACTGTC
AATTCAAGGTCAAAAAGCCAGTACAGCAGCAGAATTACAAAAAGTAGAT
CTGGAAAATCTATAGGGTACCATAGTCCAGCACCCTGCTGCAAGTCAAA
ATCAATTAAATAAATATGTTTAAACAGCACTTCATTTAATCTTAAGGGC
ACCACGACTCCCTTGGAAAATTCTTTTTTGTGTTCAGACCTGATTCTCT
GAAAGTATTTTCTAGTGTTTTCTTTGTTCGGTGGCTGTGGTGAATAAGT
GGACCCAATTGCCTACAGCATGAAATCGTAGAAATGAATATGGGCTGGA
ACTTCAATCAATCACACAAACCAGAAACATAGAGTTCATCACCTCTTCT
GGAAAGCGTCCCCTTCGGCCGTCAAAAGGGTAGAGTTTCCTCCAACATA
TGTTCATACTTCCCTTTGCCTCCCATCTGCAATTTCAACCAGTCTGTTG
CTCTCGCCCTTTTTAGGCTTCACATACCGGATTTCTTCTTAGGAGCTCA
CTTGAAAAAAGGGTTGTATGCTTTAAAAATACTAAAAGTCACTGGACTA
GATGATAATAAAATTTCTGTAAAATAAAAAGGGAGTTAACAGAGGTGTT
ACCACTTATCATAATGGATATTGTTGACCTTTCCTCTAGCCAGTGTCCC
ACTTAAGCATGCTTCTTGGCTGGAATTTATTTTTCTCATCTGGATAAAT
CTGAAGCATTATAGTAAAACCCAGTGTGAGCAAGGCCATGGATGCAAGT
GGATTGAGATGAATGAGTGGATAGACCTGCGTGGGCAGAAATAATGAGG
TCAGCAATAAGCCATACCAAGGGATGCACACTCAAGGAATAAATTGGAG
GTAGCAGAATAACTGAACAAATGATACATTTGATCAGACTGTCACATGA
ATATCAACTGAAACAAGTTATGACTGTAGTTAGTGAACCCTAGAGTGAA
GAAAACAAAATATTGAAATCCATCTGATCAAAAAATAATAAAATTGTTT
ACATGTATATATTATCTAAAATTTCCCTTAAGAATGAAGATTATAAATT
CCCGCAGTTGAGGAAACTGAATAAGAGGAGAATCAGGACAGAATCTTTT
CTCTTCGTATTCCTAGTCCATTTTTCTTCTAACTACAGTAGTGAGAACA
AGAATTTATTCACCAAATCATAATACATTGCAATTAGGGGATGCCATTT
GAATCTTGGAAGACACGATTTGAGGCAAATAAAAAATCCATATTTATAA
AGTCAATTATTGGTTTATATAATTTACTGTTTCAGCGGGAGGTATAGGT
TAGTAATGTAAGTCACTTCAAGAAGGTTTTGGTCGAAGTTTTGCCAATT
TTTTCTGTAAAAAGCCAGATAGTAAATATTTTGGGTTTTACAAGCCAGA
AGATCTTTGTTGAAGTTACTCAACTCTGCTAATGAGGTGCAAAGCAGCC
ATAGGCAGTTTGTAAATGCATAAGTGTGGCTGTGTTCCAATAAAAGTGT
GTTTGCAAGAACAGGCAGTTGGTTGGATTTGGCCCACAAGCCGTAGTTC
ACTGACCCCTGTTTTAGATAAATACTAGTCAATAGATTCCTTGTAGCTA
ATAAAGGGAATATAGATTGTTTAATGTGTTTCCCTAATCTTCTTAAGGC
TTCCTCCAGGGGAAAAAATATTTCTTATCATCAGCAGTGTTATTAATGC
TTACTCAGAACAAAGAATACCACTTACTTAGCATAGAATGGACATTTAT
AAAGCCACTCATGGAAAGAATAAAGAGATGTGAAATACCTGGTACAACT
CTTAAGAGTGTTCCACTATCATTTGAAAAAATGATGAGTAGTAGTTATA
TAAAAGGACTCGAGCCTAAAACATTTGTTCTCACTTTGGATAACGTTTT
CCTATCTCAGCATCAAAAAATACAAAGAAAGGGGATAACTAGGTCTTAG
ATTTTCTAAATTCTCATGACTATAAGTCCCATATATTTAGATATTGAAG
TACCCTTAAATATACCTAAGTAGAAGTAATAATTTCGATATTATGGGAA
ATCTAATTACTGCTCATAGTTCTCGAGATTAAAATGTTATACACCACAT
CTCAGCTTTCTTTGAAACTTGAAATGGCATACTTGTACAAAGGTAGAAT
GGTCTCCCAAAGCAGCGTCATAGATTAGCAGAAAAGAGCTATGATAATG
CCACTTCAACTGACTATCATTTAAATAATGAATTAGTTAGGAAGAGTTA
AATTATATAGTTACAGGCAGCAGGAGATGTCACCATCAGGGGGATAAGA
AGCCATTTTCCAGTACACTATTTGACTTAACTGGTGCAGTTCCTTCTTT
AACTGTAGGTTACTCATTTGTATCCTTAATTTATCAATATACTAATCTG
ACAAATAATTTATTAAGGATTACTGTGTGCCCTCCTGTGTGTTAGATTC
TAGGGATACAATATGGATCAAGATAAGCGTGATTCCTAGACTCCTAGAA
AGTAAGAAAACTAAATAACTGTACGTGACAAATACTAGGATGTAGAATA
CTAGGGAAGAATCAGGAAGAATCACCTCACCCAGTCTGGAGTATTGCAA
CAGAGCGATGTCAGAGGAAAAATTCTGGAAAAAAAAAAAAAAAAGGAAA
TTTGAAGATGAGACTTGAAGGATAAGTAGGTGTACCTAGGCAAACAAGT
AGGGGAGAGTGTTCCAGGCAAGAGAAACAAGTAACAGTTGGAGGCAAAA
TGGAGCCTAGCACGTTTTAAAAATATCAGTATGGCTGAAGCACTGAGTT
GGGGTTGGGAATGGGTGAGGAGCGATGTGTCAGGGAGGACATTCCAGAT
GATGCTGGAAATGCATGCATGTTCGCAAACTGCATTTTAAGACATGTTA
GAGAAATCAAACACATTTTATTTGAAGGGTAATTGAAAGCCACTGAAGC
ATTGTAGAGAGACTAGTGACATGAGCATATTTGGGTTTTAGAGGGATTA
CTCTGGCTGAAGAATAGGTAACTGATTGGATAAGAGCAACTTTGGAGGC
TATTCCAGTGGTTCAGGTGATAGATAATAATGACCTAAACTAAAGAAGT
AATAAAGGGAATGGAGACAAAGTAGACAGATTCAAGTGATACTTGGCAC
GTGGAAACAACAGGCCCTGGTAACTGATTGGACATAAAATGGAGAGATA
CAGAGAACGCGAATAGAACACCCAGGCATTTGGCTTGATAAACCGAGTA
GACCGTGGTAGAAATTACTAAACTATGGAGTATTTTAGGGTTCAGGGGA
ACTGTGAGTTCAATTTTAGACATCTAGTTTTGCAAGGCCACTGAGGTAA
CTGAGCATATGAGTCAGGAGAGAAGTTTAGGCTAAAAATATGGATGCCA
GAGTTAACCAGCGTTATGATAAAACAAAGCTGAAGTCTCGAGGGTAAGA
ACACCTAAACATCTAGGCTGGAGAAGGCAGCAGGTAAAAAGTAAAAGGA
GAGGCCAGGCACGGTGGCTCACGCCTGTAATCCCAACACTTTGGGGGGC
CGAGGTGGGTGGATCACCTGAGGTCGGGAGTTTGAGACCAGCCTGACCA
ACATGGAGAAACCGTGTCTCTACTAAAAATACAAAATCAGCCAGGCATG
GTGGCGCATGCCTGTAATCCCAGCTACTCGGGAGGCTGAGGCAGGAGAA
TCGCTTGAACCCAGGAGGTGGAGGTTGCGGTGAGCCAAGATTGTGCCAT
TGCGCTCCAGCCTGGGCAACAAGAGTGAAACTCCGTCTCAAAAAATAAA
AAGAAAGAGAGAGAGAGAGAGAGAGAGAGAAACAGAGAAAGAGAGAGAG
GAAGACAAAGAAAGGAAGGAAGGGAGGGAGGGAGGGAGGGAGGGAGGGA
AAGAAAAGAAAAGGAGAAAAATTCAGGAGAATGGTTACTTCCAGGGAGA
TGGAGGCGATTGTGCTGTGGGGAACACAAGGGTGGGGTCAAGGTATTAG
CAGTATTCTATTTCTTGATTGGGGTTGTATTTACATAAAGTGTTGCTTT
ATAATTATTCTTCACACTTTATGTGTACGTTCTATGTAATCATCTATAG
ATAAGACAGATTTCACTGTAAAAGAAAATAAAAGCTTCCAAAAGATTAT
CATCACAATTGTAACAGATTCCCCTGGTGCCTGGAGTCACACGCCATTT
TCCTGCACTGCAGTTGCAGCTGCAGTGGACAGCCCTGTGTGAGTTCAGA
CTTGCCTTTAGCTGACAGCATCCCATGTCAAGGGAATGGCTCCCATTTT
TCTACTTTCTATCTAAGGGACTTCTCTGACATCCCAGGAGCCCACAGAT
TTTGTGAGCTTTCTCACCCTTGAAGTTTTAGTGAGTGAGCAACCTTCAA
CCAATGGAGATGGGAGCCCATGGATATATTTTTAACCACTATTCCTTCC
GGGGGCAAGGGGAATTCTCTGTGATTCTCAGGAACATACAAAAGTTCTG
TCAAAATACAGTCCCCATGGTCCATAAGCATTACCTTGATGATAATACA
TTTGATTGGCATTTCCTCCCCCTCTGTCTCACTCTTTTGGTTTTTCATT
CTTGCTTCCTAGGGATCAGCTTCCAAATGAGCTATCTGTACCCAAGTCC
TCATCCAGGCCCTGTTTTCAGGGGACCCAAAGACAACAATAAGAAAAAT
GGAACTGAAAGAGGAGAAGACTTTAAGGCATGAAAAAAGTTCCTCTGTA
TTCCATACTGCATATTTAACTGCCTACTCAACAGTTCCACTTAGATGTC
TCAAAAATAATCTCATGATCTTTTACTGCTATGAAATGCATGACCTTCC
CCTGATATATTCCTTTCTTCAGGTTTTGTAGCACCACTTAGCTATCCAG
TAACAAAATCTTGGGGGTCATTCTTAAAACCTTCCCACCTCACCCCTGC
TGGACATCCACCACTAAGTTCAGTTGATTTTTTTGCTCCTAAATATTTC
TTGGTTCAGCTTTCATTTTTAGATTTATCCTGCATACCCCTGTACAATC
TCATCATCTCTTTCCTGGACTGTTACAGTAGTCTTATATTAGGATGATC
ATAGTCTTGATTTGCCTAGAGAAAACATGCTTTATGTCATTGCCTCAGA
GTGACTAATAGTCCCACCTTTCACTTGTAACACTATGCAGGTTATTGGG
TTAAATAATACTGTCATCCAAACCTAATCTCTCCTCATTCATTTCTTCT
CTCCTATGCATCTTAGCTGTGTGAAAATTCAAAATATAAATTTGATGAC
AGACAGAACTCTGTTTAAAATGCTCCAGTACCTTACCATTTATCTCAAA
ATAAAAATTTCAAAAAAAGAAATGATATCCCACAGGATCTTGTATAGTG
TGACCCTTTCCATTTCATCATCCTTATACCAGATACTGTGAACTACTTA
CTCTCTTATCTCCACATTGACCTCCCTCCTAGTTTTGTTTTGCTTATGA
AAGTGCTTATTTTCTGTTTTTTAAAGCCATTGCACATATTGGTCTCTCT
GATTGAGACACTATCCTTTTGAATTTTTGACCTCATACCTACTCACTTT
TCAGGTCTCAGCTCAAATGTTATACTCAGGAAAGACGCTCCTTACCTCC
CAAACTAGGTTAGTGTAAATGGCACTATTTATACTCCTCTTCCAGAGCA
CACACCAAATCTTATATTTATTTGTGGGACAATTTGAATTCATGTTGTT
CCCCCTACTCAATTGTGAGCTTCTTGAGGACATACTCTGCCTCCTCCTT
ACCTAGCTTTATTGAAGTATAATTGAGAAATAAAAACTGTATGTATTCA
AGGTATACAACATGATGATTTTATATGACTATATTGTGAGATGATTACC
ACAATCAAATTAATTAATACATCTAGCACAAGAAATAGTTACTATTGTG
TGTGTGTTGGGGGGGGGGGATGAGGACACTTAAGATCCAGTCTTGTAGC
AAATTTCAAGTAAACAGTAAAGTATTATTAACTATAGTAACCATAATGT
ACATTAGATCCCCAGACATCTTATAACTGAAAGTTTGTGCCCTTTGACC
AATGATATGGTTTGGCTGTGTCCCCACCCAAATCTCACCTTGAATTGTA
ATCCCTGTAATTCCCATGTGTTGTGGGAGGGACCCAGGGGGAAGTAATT
GAATCATGGGTTTGTTTCCCCCCATGCTGTTGTCGTGATAGTGAATGAG
TTCTCATGAGATCTGATGGTTTTATAAGCATCTGGCATTTCCCTTGCTG
GCACTCATTCTCTCTCCTATCACCCTGTGAAAAGGTGCCTTCCTCCATG
ATTGTAAGTTTCCGGAGGCCTCTGAAGCCATGCGGAACTGTGAGTCTAT
TAAACCTCTTTTCTTTATAAATTACCCGGTCTTGGGTATTTCTTCATAG
CAGCATGAGAACGGACTAATACAGTAAATTGGTACTGCAGAGAGTGGGG
TATTGCTGTAAATATACCTGAAAATGTGGAAGTGACTTGGAACTGGGTA
AGAGGCACAGGTTGGAACAGTTTGGAGGGCTAGAAGATGACAGGAAAAT
GTGGGAAAATTTGAAACTTCCTAGAGACTTGTTGAATGGTTTTGACCAA
AATGCCGATGGTGATGTGGACGATGAAGTCCAGGCTGAGGTGGTCTCAG
ATGGAGATTAGGAACTTCTTGGGAACTGGAGCAAAGGACACTGTTGCTA
AGCTTTAGCAAAGAGACTGGCAGCATTTTCCCCTGGCCTAGAGATCTGT
GGAAATTTGAACTTGAGAGAGATGATCTGAAATTGGAACTTTGTTTTAA
AGGGAAGCAGAGCATCAAAGTTTGGAAAATCTGCAGCCTAACAATGTGA
TAGAAAAGAAAAACCCATTTTCTGAGAAATACAAGCTGGCTGCAGAACT
TTGCTTAAGTAAAGGAGCCAAATGTTAAGCGCCAAGACAATGGGGAAGA
TGTCTCCAGGGCATGTCAGAGGTCTTAATGGCAGCCCCTCCCATCACAA
GCCCAGAGGCCTAAAAGGAAAACATGGTTTCACGGGCCAGGCACAGGGC
CGTGCTGCTTTGTGGAGTCTCAGGACTTCGTGCCCTGCATACCAGCTGT
GGCTCAAAGAAGCCAAAGTACAGCTCAGGCTGTTGCTTCAGAGGGTGCA
GGCCTTAGTGGCTTACATGTGGTGTTGGGCCTGGGGTTGGACAGAAGTC
AAGAATTGAGGTTTGCAACCTCTGCCTAGAATTCAGAGGATGTATGGAA
AAGCCTAGGTGTCCAGGCAGAAGTTTGCTGCAGGGGCAGGGCCCTCATG
GAAAACCTCTCTGCTGGGACAGTGCAGAAGGAAAATGTGGGGTCGGAGC
TCCACACAGAGTCCCCACTGGGGCACTGCCTAGTGGAGCTGTGAGAAGA
GGACCACCATCATCCAAACCCCAGAATAGTCAGAAATGCTGATAGTTTG
AACCATGCATCTAGAAAAGCTGCAGATACTCAGTGCTAGCCATGATAGC
AGCTGGGAGGGGGCTGTACCCTGCAAAGCCACAAGGGCAGAGCTGCCCA
AGGCCATGGGAGCCCACCTCTTGCATCAGCATGACCTGGATGTGAGACA
TGTAGTCAAAGGAGATCATTTGGGCAGTTCAAAGTGTAATGACTGCCCT
ATTGGATTTAAGACTTGCATGTGGCCTGTAACCCCTTTATTTTGGCCAA
TTTCTCCCATTCGGAACAGGTGTATTTACCCAATGCCTGTACCCCCCAT
TGTATCCTGGAAGTAACTAACTTGTTTTGGATTTTCAGGCTCATAGGCG
GATGGGACTTGCTTTGCCTTAGATGAAACTTTGGACTTGGACTTTTGGG
TTAATGTTGGAATGAGTTAAGACTTTGGGTGACTGTTGGGAAGGCATTA
TTGTGTTTTGAAATGTGATGACATGAGATTTGGGAGGGGCCAGGAGCAG
AATGATATGTTTTGGCTGTGTCCCCACCCAAATCTCACCTTGAATTGTA
ATCTCCAAAATCCCCAGGTGTCATGGGAGGGACCCAGTGAGAGGTAATT
GAATCATGGAGGCAGTTTCCCCCATGCTATTCTCATGATAGTGGGTGAT
TTCACATGAGATCTGATGGTTTTATAAGTGTCTGGCGTTTCCCCTGCTG
GCACTCATTCTCCCTCCTGCCACCCTGTGAAGAGGTGCCTTCTGCCATG
ATTGTAAGTTTCCTGAGGCCTCCCCAGCCATGCAGAATGGTGAATCAAT
TAAAACTGTTTTCTTCATAAATTACCCAGTCTCGGGTATTTCTTCATAG
CAGCATGAGAACAGACTAATACAACCAACATCCCCTCTTGTCGATTTTC
ATTGAATCCCCATTACCTGGGAATGAATGAGGAAGGCAGAAATAGAAAT
CAGCGTATTGTTCAATATAGAAATCCTGAAGTTACACAAGATAATTACG
AGCAAGACTCAGTACAGAGAAGGAACCCTTGAGCTGGGGATCAGAGTCT
TCTGGAAACAAGGAGTACCAAAGGGGAGTGAAAAAGAGAAGTGGAGAAT
GAGTAGTAGCAGTAAAAGAGGAAGTATGTAAAGAAATGGAAATAAAGAT
GAAATAAACTGTCATCTTGTTCCAGAGACAAAGCTAGCTAGGGGAGCTA
AGGAAATTTAGAGGGATAAAAAGTCTCATCCAAACCTTCATAATGAGCT
AAGGGCTAGTTTTTCATAGACCCTAGCCCAACTTGCAACCCTCTCACTC
TCTTACTTTTTTCCTGGCTCTGTTTTGCTTTACTGCCTTTCATATAATC
TAAAATGATGTTAAATGTATATTTCCTGTCTCTTTTCTATATCACAATA
TAAATTCCATGAGGCCATTGCCTGTGCCACATTTACCACTGTACTCCCA
GTGCCTAGAATGGTGCCCAGATCAAGTAGACTCCCAGAGTGTCTGTTGT
CAAATGATTCTCAGTCCAGTGATGTTTGCACTACATGGTGCTGCCACTT
TTTTTTTTTTTTTTACTTCATTCCATTTGGAATTTGGATCATTTTGCCA
ACTTTAGGGTTTATTCCTCTTTATTTCCCCTGTGTTTGCTGAATATCTG
ACTATCTATTTTTCTCAGCTCTCTTCTCCTCACACTTTGCCTCCAACTT
CCTCTCTGCTACAAATTATACTTAGCACAAGACTGGAGCCACTTTTCAT
TGATTTTGATGTCAGTGCTTGAGTGACAACTCCATGTGGAGAACTTTCA
ACCTCCTGCTGTCTCGTTGTTTGCTTTGAAAAAAAAAAATACTCATCTT
CTTCCTGAACCCACTGGGTAAATTTATGCTCACAAAAAAGACAAAACTT
CTAGAACAAAAACTGTATGAGCTTCAAAAGTATTAGTTTTCCCTTGATT
TTGAGACTCAAAAGTACCATAGAAATTTTAAAAGGGGGATGAAGTAATT
TTGTCATTCAACCCGATATATTTTACAATTTGAATGTCAGAACCATGAA
ATGTCTTTTCAGTGTACTATCATCCTCACTATTTCCACCTATAAAAGAA
AAGCAAAAATATTGTTTGAGAACAAAAAAATATTGCAGTAGCGGAAAAA
TTCACTTTAAGTCTTTTTTTGGAGGTTGCCACATCACGCCTGTCTTCAG
CTCCTCTCACTGAAGATTCTTTGTTTTCTTGAAACCAGTTGGTCACTTC
CAATTCTTACCACCTATAAATCTTCCTGGTTCTTTATATTTCACTTTAT
ATGTCCCTTATCTCCTCTGATTTTTGTTCCAGTCTTTCATTTTTTACAT
TCTTTGTCATCCTATATGTCAGAAAGAATCTGCCACTCCAAATTCCTTA
ATATATTTATCTACCAGTGTTTATTTGCCTGTCACATGTACTCTTCAGT
AATCCATTGACTTGAAAACTTTCTACTCTGTCTTTCTTACTATAGTTTG
CTTCTTGAGTTCAAATTATTAATATATTAGGTCAAGTAATTCTATTTTT
ATGCACATTTTACCTAACATTCTTGGTAAATATAATTGTTTTTTAAATT
GCATCACATGGTGAGTATTAATTTAAGACGTAATTAGTATTAATCTACT
GGACAAGATTTTATTTATTAACTGTTTTGCCCTTTGCTAGGGTTCTGTA
ATCTCACCCTCAGTGCCTTTAAATAGTCTTACCCAAGGATCACCTATCC
CTCTTCAATTTAAATTCTCTATTGTGTTAGTTTCTGAGATTGCACATTC
TCTGGTTTGGTTTGGTTTTTCAGTGATCTCTGTGGTCACTTCCAACCAT
TTCTACTTAGTTTACTTTTTTCCCTGTTAGCCCTCTTCAGTGTTAATAT
CACCTGGAAGTTAACCCATTCCTAGTCTGCATTGCTCTATGGCCAGCAT
CTTGGCTGATTGGCCATCACCTATCCTGTACTTATTGTCAATTTTTTTA
AATTTTTTATTGTGGCAAACAGCACATAACGGAAAATTTACCATCTGTC
TTTGTTCATGTATGTTGCTATAAAGGAATGCCAGAGACTGGGCAATTTA
TAAAGAATGGAAGGTTTATTTGGCTCATGGTTCTGCAGGCTGTACAAAA
AAGCATGGCACCACTACCAACTTCTCATGAGGGCCTCAGGCTTCTTCCA
TTCATGGTGAAGGGCAAAGGGGAGCTGGTGTGTAGAGATCACATGGTTA
GAGAGCATAAACAAGAGAGAGAGGGGAAGTGCCAGGCTGTTATTTGGCA
ACTAGCTCTTGCAGAACCTAACAGAATGAGCAGTCACTCAACCTGCCCC
CAGGAAGGGCATTAAGCCATTCAAGAAGGATCCACCCCCATGACCCCAC
ACTTCCCGTTAAGCCCCACCTCCAATATTGAGGATCAAATTTCAACATG
AGATTTGGAGGGGACAACATCTGAACTATAGCATTATCTTAATTCAAAT
AATTTTACTACCAATTCCATTTATTAGAGAATCTAAGATTAAGACTACT
GAAACTATGCCAATGATTATCTCTAGTTCAGGCTTCTCTCTTCAATTCC
AAACTCATATTTTCACCTGCTGACAAGCTACCACGTAGATGTTCCACAG
GTATTTCTAACTCAGGATATGTAAAATTGATGTTATCATTATTTTCTGA
AAATCTATTCCTCTTACTATATTCCCTATTTTTGTGAATGACACAATCT
AATGGACTGGCCATGTTAAAAATCAGGAAGTTTTTCTGGCACTTTCTTT
ATTCCTTAATATTAAGCCCCACGATAAATTACCAATCCTTTCGATCTTG
TTAAGACAAATAAGCTTGAAATTTCTTATCTCTTATTTAGCACTGCAAT
TTTGTTTTCTCATTCATTCAGATAGCATCTTCTACAAAACCTCTTCACT
AGTTTTTCTGTCTCTCAGCTCATCTTGTACACTGCAAATCCATTAAAAC
TACACTTTTTAACATGCCAATCATAATGTATTAGTAAAGGTTGTATAAT
AGTTCTCTTTCTCTTCTAGGATAAAAGCTAAATTTTTCAGCTTGGTACA
CAAGGCCGTTCATGGATTTGGTACCTACTAGTCTATCTAGGGTATTTCC
TACATCTTCCCTCCTCTCTCTTGAAAAATCAAGTAATACTAAACTATTT
GTAACTCCCTAATCACCAATTGCTGCCTTTATACTTTAACATATATTCT
TCCTTTTGCTAGAAATACATTTGTCATAATAGGCTCTTTGGTAAACTTA
CCATTACATTCTCAGTTTATAATATTATTTGTGTAAGTACCTTTTTTCC
TACTTAGTCCTCCAGGGGAATGCATGTGCTTCTCTCTCCATATTATCAT
TTTAAAATTTACTCCTATTTCAGCAAGTATCATAATATTTTACTGAGAT
TAATTGTTGGCATATTTATGTTACCCTGGTGACATATTTAATAGTTAAG
ATGCTTTGGGCTGCAAGAAAGAGAATGCACTTTCAAAGTGGCTTCAAAA
ATAAGAATAATTCAACTCACATAACTGGAAGTTCAGAGGTTAAAGCTGG
CTCCAGATGAAGTACAAGCAGAGCTCTGTCACTCTTTCTCTATGACTTT
CTTTGTCCTGATTTTTTTCTCTGCGTCAGCTTTGACCTCCTCACTAGTT
GCCCTCCAGGTTCCAAGATGACTGCCAGCAACAAATGGGTAACATAATT
TCTTGTTACAGTGAGAGACATAAACACTTACCTCACAGATTCAGAGATT
TCACCCTCAGATAAGAACAATCAAAATTTTAATTGTGTATCTACTGTAT
CATTAGTATATGTATTAGGTATTCATATATATCTACTATTGTACTAAAG
ATGATTCAGAATGGTTTCATAAGCTGGTTGATTTAAACTTTTCTTCCTG
AACCAAGAGGATGAGATTATCTTGACTGGCTTAGAATCGAAGATTTAGG
TGAATCTAAATCCATCTCCAGAACTGAGGATGGATTCCGTAGAAACTTG
AAGACAATTGGAATTCTCCTAGAAAGCAGAATTTCAGAGTGGGTGTAGG
GATATCTTTGAATGGAAGTTGTTTAAGCCACCAACAATATTCACTCCAT
TGGGGTGACACTGGGCCCAAAGGTACCCAGTTAATATTTGTTCAACGAA
CCTATAGGAAGACAATATTATCTGCAAGCTATTTATAAACTAAATGACA
ACGAGAAGTATTTTTAGAAAAGTTAATCATATACAATTTAGTCATTCAC
ATTGGGAGGCTCTTCCTAAATTTCGTTTCTGATCCTTATCTTGATCTTT
GACGCAATCATATCTTGGCCTCCATCTAAAAGCCAGTAGAAGCCTCTGG
CCATTTTTTAAACAATTGGTTTAACAACATTAATTGGAATCTATATAGC
TCAACTTAAAAAAAAAAAAAAGAGGAACTAGCTTATCATAATACAATAT
TGTCAAGGCAAGTGATACTTCCATGCAGCTATTCAGCATTTGGTGTATT
CTTGGTTTCTGCAGACCTAATAAAAAGAGATTATCTCAGCGTCCTCTAG
GTTTAATTTTAAAAAACAAGAAACCATGAACAATGAAAATGATTTCAAA
ACATCTTCACTCACATCTAAAATATGAAATAGCTAGATCAGAGGTTCTT
AATAATTTAATCGGTGAAGCATAACTGAAGGATTTAGAAAAAGTGCCTA
AACTATTAAAAGATTCTTCAGCTGCTGCTTAATGTTGATATCAAGTTAT
TTTTTACACTGGCAAACAGCTAAGCCATTCCCTGGTCACATCCACTTTT
CAGCATTTAGCGCTCCTCTCAATCATCCCGATCACAGCCCCAGGAATGT
TTCATGGCATCTCCGCAATAATAGTATATTACTATTGGGTCCTGAATTT
TGAGGTAGTTTATCTTTCAGAAAGGATATGAATAGAGCAATAAGTCCAA
GTAGAGTGGGCTTTATTAATGATGCCCAAGATTTTAGACGTATACTATA
ATCTTCAATCAATTTTAACACCACTTCCCTTACCCTAGTTTTAGTCTAA
ACCAGTCCAACTGTGCTGTAGTCTCAGATAATACAATGTTAGATTTTTT
TTTTCAAGATATAGAATTGTCGGGAAAACTTGTATGTAACCTATACCAA
TCTAAATTTCTTAGCATTTAACTCTAAAGAGTAAAGCTTTTAGCACTTC
CTGTGTAATACAAGGGCAAGTGCTGATTTCTCTGAATATAATTTTCCTT
TGTTAGCACATATGTTCACTGTTTAATAAAAATAAGAATGCTTCAATTA
TCTCCTTTTGTCAAGGGCCTGAGAAATAAAGAAATATACAGAGGGTCCC
AATCTAAAAATGTTTTGATTTACAATGGTTCGGCTTAGGATATTTTTAT
TTTACAATGGTGTGAAAGTAATATGCACTCAGTAGAAATCATACTTCAA
GTACTCATACAACTGTTCTGCTTTTCATGTTCAATACAGTATTCAATAA
ATTACATGAGATGTTCAACACCTTTAATAAAATAGGCTTTGTGTTAGAT
GATTTTGCCCAACTGTAGGCTAATGTAAGTGTTCTGACCATGTTGAAGG
TAGGCTAGGCTAAGCAATGATGTTCAGTAGGTTAGGTTTATTAAATGCA
TTTTTCAATTTATGATGTTTTCAACTTATGATGGGTTTTTCAATTTATG
ATGTTTTCAACGTATGATGGGTTTATCAGGAAATAATCCCATTGTACGT
TGAGGAGCACCTGTATCAATATAGGCATTTACACAACTCTCGTACTGAT
AGCGGCAGGAGGCAGAGAAGCTCTAGGCAGAAAAGGGATGGTCCCCAGC
GAAAACCCCACCCTCAAGCCAAAAAGCCTGAAACCGCAGCTCAAAGTGG
GAACTTATATCCCAGTTTTCCTGCTCGAATGTTGCCTTTTTCTAAACCA
CCCATGGCCCCACCCCACCCCATCCTGTGCCTATAAAAACCCCAGACTC
AGCTGGTAGACAGGACTACAGCTGGACATCAGAGAGAAGCAGCTTGACT
TCAGAGGGACAACTTGATGGCATAACTTTAGAGAAGAATCCGGCTGGAC
TTCAGGGGAAGATTACTTGCCACCCCCATCCCCTTTTCAGCTCCCCTTC
CCACTGAGAGCCACTTTCATCGGCAGTACAATCCCTCACATTTACAATC
CTTCAATTTGTTCATGTGACCTCATTTTCCCTAGATGCTGGACAAGAGC
TCAGGAGCCACAAGTGTGAATACAAAAGGCCCTTTGCCCTTGCTGGTGG
AGGGCAGCTGCCTCCTGTGAAAAGACAAATGGCCCACTGAGCTGTTAAC
GCCTAAGCTGTCCGTGGATGGCAGAGCTAACAGAGCACTGTAACACACC
CTCTGGGGCTTCAGGGGTCGCAGACGCCTCCACCTAGATGCTGCTCCAG
TGCTCATGCACTCCAGTTCCCACCTCGTTTGCTTGCACACTCCCTCCAG
TGAGGAGTTGAGAGCAGTGGGCTAAGTAAATAAGGCACCCCTGTTGCGA
GTTCCACAAAGGGGTCAGGGAAATATCCTGCTTCGTTACTAAGTAGGTA
TAAACCCTTTACTAAGTTAGTAATTATTCAGTAATAATACTTAAATGAA
AGCTATTCTGTTAGACTAAATTCAGATTATCAAAGACACGACAGAAAAC
AGCTTTCTCATTCATGAACCTATTATTTCCTTTTGTAAAATATGTTAAA
TGAAAATACAGCCGTCATGCTAATTTCTAATGGTAGAACATATTTTGAA
AACTCCTTTATGTTTGGAAGATTTTGCTTTAGTGCAGATAATCAGAATG
ATGTGATATACTAAGAAATAATTTTTAAAATGAGATGTGACATTTCTCA
TAATCTAAATAAGAAATGGCAAAACATTGTCCTAAGCTAAATAACTCAT
ATGAAGTGATAAAATATTGCTTTCTAAAGGTCCATGATATGTAGTGATT
TCTAATGTGTTTAATAGCATTGATCCTATTGGGTAATTGGGTGGTTCTA
ACATGTAAGGAAGGCCTCCAGTACTAATTTCATGTACTGGGAAACTACT
GGGCAGGGATGAATCCCTTAACTCCTAAGTAAGACTATCAGATCATATA
AATCTGCTTTTTGATTTGCAAAGACTCTTAGGCATACCTCTTGAGAATA
TTAAACATCTACTAAATTATATGTAAAGCATTTCAGTCTAAGATTTACA
ATGCTCAAAGGAGAAAGATTTTAAAGTTCAGCATTGGAATTTCCATAAT
TTCCTTCCAATTGTAGAATTTTACAATTGAGATAGCAAATAATAATATA
AATAAATATGTAAGACTGAAATCAACATAGGAGTTTGGAAAGAAGACTG
TATGGGTTAACTAGAGTTGTCAACAAAGACTTCACAGAAGATTGTGATC
GTAGACATTAGTAAGGAATAAAATGGGTGCAAAAAAAAGTCTAAGAAAC
AACAAGGATAAATTTATTTTGTAAAGAGTGAAAGCATATTGGGTATTAC
ATAAAGAGACTGGCTAGTTTGACACAATGTGTACTTTTGGGGTAAGCAG
TGGGAAATCACTTTCTGTAAGTAAAGTGGAAGAGAATTCAGGTATTGGA
TATGGTCATATAGGATTTTTCAACCTTTGTTTTGCCCCCATCCCTATCC
TACCAGTATCATCACAGATGTTGGCAATATTCACTCACTTGCCTGAAGA
TAGATATTCATGTCAGGGGCTACAGATATGTAAAGGTGAATGAAGTTAA
ATAGCAAAATTCAAGCACACTAGTTCTGACTGTGCACCCTCTCTCACTC
AGAAAAGCACTTTGGAATGCGAGAGTACTCTTATATTGACAACCTTTAG
TTGGTTCTAATTTATAAAAAAATATAAAAATATTTAGACAATTTGGATA
TTTTATATCTATGCCACAACCCATTGTCAAATGATTGAGATATCATTGT
TTGGCAACAATGAAACTGAAATCAACTCTAAGAGTAAAGAGTACGACTT
TTAAAGATATTTGGTTAAGAGTAATGTTGCAAGGCAGAGACCAACATTA
TAGAAGTTCTCAACCTGTGTGCTGGATATAGCTAAAATTTCCCAGGACC
ATTGAAAGAAATTCTCAAATCCACAACCACCTCAAGTGTTGTCTGTGAA
TCAAAGGAGTGATGCATTACCCACAAACATGTAGTAGACATTTTTTAAG
TGATGTAGATGCTACTGCAATTAATAAATGCAAAATTATATTACAATAT
TACTGAATTCAGAATAAGTTGATTTTGTCACTATTTTTCTCAATCTACG
CATGCTAGATATAAAGTTATTATATAGAGACGTAACATTTTTAATATTT
CAGAAGCATGCTCATGATATTGTAAACCAGGCACTGTTAAAAGATCACT
GGATTGGGAATTAATAAACCTGGGATCTAGTCCTCATTTGATCTCTTGT
TGGCTATGGTTTGGGGGTTTTGGGCAAACTATTCAACCTTTTCTGCCTT
GGTTTAATTGATAAATGATGGTGTTTTCCCAGATAAATCCCTTCCCAAC
TTAATATTACCAGATGTAGCATCTATGGTTTAGAATGTACAGTATAAAT
TAACCTTCCTGAAGATCTTCACAAGTTACTATAACCTATATTTTCATGG
CACTGAAAACTAAGTTTTTGATAGCTTACGTTTTTATAAATAATTTACT
TACTCATTTTTATCATAATAATAAATCTGATTCACTAATCACCAAAATA
TCATTTTTGAAAATAGATGCATGAAAGGATCCGAACTTGTTATGGTTTT
ATCTGTTCAAGTCACCTAATTTTGGTAGCCACAGGCCCCCATTGTCAAT
AGGGGAAGATTATCCATTTTAGCAATACAGTCTCTGATAACTTCAGCCC
ATATGCCCTTCAATTCCACTTTTACGTCAGATTGAATGGGAATGTGAGG
CCCCCAGTAGTAAACTAACCCTACTTTCTCTTTGGAAATTGGTCTTTCT
ACCTAGCTCTTTGCCTCTGTTCACGTTCTTGTATTCCACAGAAAATATA
TACATTAGGTGTTAAAATCACAATGATTAACAATTTTTAAGTAGAAATA
GTTATTAAGTATAGCATAATCATGCCTTTGAATTAGTACAAAAGTAGGA
AACAGAGCTTTAGTGACTTTTTTCATTCTTTCCACCATTTACAGGGCAA
AAATGAAGAATTTTACCAATTCAAAACTATGCACATGTATAGTTTCCAC
CAGTATTTAGTAGTTATGTTTCTCAAGATGTATAATTCCTTTCCTTTCT
GTTTTCTGTAGTTTGATAAACCCTAGATAGGAGTTAATGTTGTTTCAGT
TAGGTTTATTATTTCTTTTATGTGGTTTAATTTCATGCAATAAGCTAGA
GATTTTGTAACATAACTTGATAAAAATTTTCTCCCTTCGTATCTTTTTG
TTTTAAAATAATGGATTAATATAGATTGTAATTTTACAGTAGAGGAAAT
ACATTTTACTTTTAGTTCTTATCTAGATATCTTAGGAAAAAAGAAACAT
CATTTTTAAGGATTATTATTTTTCTACTAATGAAAAAAATAGCATGATT
TTCCATTCTGGACTTTGTAATTAACTTTACCTTGGAATAATTGAATCTT
AAATATAACTTCATTGAAAATTTTATTTTCAAGTAACATTTTAAAATGC
AAAATTTGTGTGATCTCTACTAAAAAGAACTCTCACATCCCAATGTGCT
TATAGCTACAATAATTGGTTAAAAGAGACAAATTATAAAGAAGATATAG
GTTGTGACATTGAAATTACAAAATGTGGATTAGGGAAGTAAGCGTGTAG
AGTTTTGTATGCAATCAAAGTTAAGTTATTACCAGCTTAAAAAACCCTT
ACGTAAGATGTAAAGAAAAGCAAAAACCTATAGTAGCAAAAGCAAAAAC
CTATAGTAGATATACAAAAGGTAAAAAGTAAGGAATCAAAGTATACTAC
TGAGCAAAACAGTCAAACCATAAAAGAAGACAACAAAAAAAGGCATATA
GAACAAAGGATCTACAAAACAATTAGAAAACAACTTTTTAAATGGCAGT
AGTAAATTCCTACCTATCAATATTTACTTTGAATGTAAATGGATTAAAT
TCACCAGTCTAAAGATAGAGTGGCCAAATGGATTAAAACAACAAGACTC
AACTACATGCTGCCCATAAGAGACTCACGTCATCTTTTAGGACACATGT
TGACTGAAATAGAAGAGATAGAAAAAGATATTTTGGTTTCCATGCAAAT
GGAAACCAAAAGAGAATGGGGATAGCCATACTTCTATTAGACAAAATAG
GCTTTAAAAATCAAAAACTAAAAAGACACAAAGAAGGTCATTAAATAAT
GATAAAAGGATCAATTCATCAAGAAGGTATAACAATTGTAAATATATAT
GCACCCAACATTGGAGTACCTAAATATATAAAGCAAATATAAAGTGATA
AAAAGAAAGAGACAAACTACAGTACAATAATAGTAGGGGACTTCTACCC
CAAATTCAACAATAGACAGAAAATCCACATTAAAAAATCAATAAGAATG
CATTGGACTTTATGCTTTAGATCAAATAGACCTAGCAGACATATACAGT
ACATCTCATCTAACAGCAGGAGAATATACATTCTTATCAAGTGCACAAG
AAACAATTCTTCAGGGTAGATCATATGTTAGGCCACAAAATGAGTCCTA
ACAAATTTAACAAGATTGAAAGCATGTATTTTATGTAAAACAACGCCAT
CATGGAAAAAGTACTAGTGTAAACAAGGTTTAAATATGATTTACTGATT
GTTTAAAAAGGAATTATCTTAGCCCTGATCTGATGGGATTTCCCCTTTG
TAAGCAGCAAAAATAAGTTCATAATGAAGCAACTGTAATAATACAGCTT
CACAGATCTTTCTGAATAAACAGAGTTGGATATGTTTCTACTTCAGAAA
CCATTTACTGTGGGCTCACAGCTTTTCCATACACTCTTTACACTCTTAA
TTTTAAACCCATTCATCAAAAGGATTAAGACAATGAGATTCAAGTCCAA
GACAATAGGAAGTATGTGCATCAAAACTGTCATGCTAATGCTCTGAGGA
ACATTGTTATTTCAATAGCATAATTTAAAACCACTGAAACCATGTTTTA
TTTATGATTACCTTTCATACGTTCAAAAAGAATTTGAGATGGTTTGGCA
GGGATGTCTTAAAGAAACAAACACCAAATTTTTATTTGTTGTGCTTCTA
ACAAGCAATTTTTCCTACGTAAGTGTTACCTGTTTTCTCCCCTTGATTT
TGATCTCTTTTGTATGGTTGGTAGTTGTCGCACTTCTGGGCATAGTATG
GTTTTCTACAAGTTAAATAAAAACATAAATTGATTTTGATGTAGCTAGT
TCCACTATTTCAGTTAGTGTGTTTTCCATACCTGCAATTCATAACATGT
TTACTGACCCAGAACGTTAAAACTCAAGTTTAGTTCAATACAGTCACTT
CAAACCAACTTAAGATAGCAGTTACATTTCCAAGGTCATTATGAAGACG
GAGTTCAAGACTTCAGCAGTTGAATTTGTCAAGCTCATGGGTCTTTTAG
TTACAGGAAGTGTGCATATTTCACATAAGAAACAGCAACTCTGATGATA
CATTGAAACTCAAATATACCCAAGGAGTGTAAAACTACTTTATAAGCCC
TTAAACAATAAATATGCCAACAATCCTCTGCATACTTTTTGTCATTTTT
TAGAGCATTCAATTGAATTATATAACATGTGATACCAATAAATAATTAA
CTTTTTATTTATTTATTTAGAGAATGATTCTTGCTTTGTCTCCCAGGCT
GGAGTGCAATGGCATGATCTCAGCTCACTGCAACCTCCACCTCTTAGTT
TCAAGTGATTCTCCTGCCTCAGCCTCCCAAGTAGCTGGGATTACAGCCA
TGCACCACCACGCCTGGCTAATTTTGTACTTTTAGTAGAGACAGGGTTT
CACCATAATGATCAGGCTGGCCTTGAACTCCGGACCTCAGGTGTTCTAC
CACCTCAGCCTCCCAAAGTGCTGGTATTACAGGCATGAGCCACAGCGCC
TGGCTTATAATTAACTTTAAAAAATATTCTACTATCGAATGCCTGAAAA
AATCATATTACTTCTATGTATTAAAAACAAACTATTACGTAAATAGACC
AGAGATACGGTGTCAGAGATGAGATTCCTTGGCAACAGTCTCTGAGACA
AAAAGTTGCACACAGAAAGTATTTTGAGAAGTATTGTTGCTATTTATAA
GGAAGTGAAGGAGGCAGTATTGGGCAGAGAGAAAAGCTCATCCACACTG
CAGTTGCTACTGAGGCTTCAGCCCATGTGACAGGGACACTGGAGCTGGG
ATGGTCTTTCAGAGTTCTACCAAATTGAGGCAAATGGGCGAGACTTTTG
TATCTTTGCACTAGCCAAGAGCAGGCACCAGGGAGAAATGCAGCTGTGA
TCCTTTTGTAGATATTATTATTCTGGAGTCAATGCAACCACACCACAAA
TACTAGGAATAATATTGGTAGTGTGGGTGCATCGACTCCAGAAGAGGAT
CTTGATGAAGCATTACAGTATCCACTACAGAGAGGCACTGGCATATTCT
GCTTACCCCACAAATATTGCAATGAGGATGCAAGTAGGAGTAGTGAGGA
CATAAAATAAATCGATTATTTTCCACTGGGCCTTAATATATCAGAACCA
TTGGAATTTACAGGATGATATTTATTATAGTACTCAAAAAAATCTCTTT
TAAATCTCCTTAACTCAGAAGGGAATTTTAAAAAGTCCACAATTCACCT
GGTCTATGAATTCCCTTTTAAAATAAAATGTGGTTTAATCAAATTTACA
AGAAAAAAACAAACAACCCCATCAAAAAGTGGGGGAAGGATATAAACAG
ACTCTTCTCAAAAGAAGACATTTATGTGGCCAAAAGACACATGAAAAAA
AGCTCATCATCACTGGTCATTAGAGAAATGCAAATCAAAACCGCAATGA
GATACCATCTCACATCAGTTAGAAAGGCGATCATTAAAAAGTCAGGAAA
CAACAGATGCTGGAGAGGATGTGGAGAAATAGGAACGCTTTTACACTGT
TGGTGGGAGTGTGAATTACTTCAACCATTGTGGAAGACCGTGTGGCGAT
TCCTCAAGGATCTACAACCAGAAATACCATTTAACTCAGCCATCCCATT
ACTGGGTATATACCCAAAGGATTATAAATCATTCTACTGTAAAGACACA
TGCACACGTACGTTTGTTGCAGCACTGTTTACAATAGCAAAGACTTGGA
ACCAACCCAAATGTCCATCAATAATAGACTGGATACAGAAAATGTGGCA
CATATACACCATAGAATACTATGCAGCCATAAAAAAGGATGCGTTCGTG
TCCTTTGTAAGGACATGGATGAAGCTGGAAACCATCATTCTCAGCAAAC
TAACACAGGAACAGAAAACCAAACACCGCATGTTCTCACTCATAAGTGG
GAGTTGAACGATGAGAACACATGGAGATGGGGGAGGGGAACATCACACA
CCAGTTGGGGGATTGGGGGGCAGAGGGAGGGATAACGTTAGGAGAAATA
CCTAATGTAGATGAAGGGTTGATGGGTACAGCAAACCACCATGGCACGT
GTATACCTACGTAGCAAACCTGTACATTCTGCACATGTATCCCAGAACT
TAAAGTATAATTTAAAAAATGTGGTTGAAAAACAAAACCCACATAATAC
AAACTTTGCCAGCTTAACTGTTTTATGTGTACAAACCAGTAGTGTTAAC
TATACATACATTGTTATTCAACAGATCTCTAGAATGTTTTCATCTCTCA
AATCCGAAACTCCAAACCCACTGAAGAGCTCCCATTGCTCCCTGTACCC
CAGCACTGGCAATATGACCACTCTACTTTCTGTCTCTAAAGAGTTTAAC
TACTTTAGATACATCATATAAAAGGAAGCATGCAGTATTTGTCATTTTA
TGACTGGCTTATTTCACTTAGCATAATGTCTTCAAGGTACACCCATGTT
GTAGCATAGGAAAGGATTTCCTTCTTTTTTTGTGGCCGAGTAATAATAT
TCCGTTGTGTCCTATACCACATTTTTTAATCCGTTTATCAATCAATGGA
CATTTTAGTTACTTCAATTTTTGGCTATTGTGAATTATGCCGCAGTTAA
TATGAGTGTGCAAATATCTCTTTAAGATCCTGTTTTTAATTCTTTTGGA
TATACAGATGCTCATTGATTTACACTGGGATTACATCCCAATCAGCCCA
TCATAAGTTGAAAATATCATGTCAAAAATTAATTTAATATGCCTAAGCT
ACCAAACATCATTGCTTAGTCTAGCATACTTTAAATGTTATCAGAACAT
GTAGATTACAGTACATCTGGGCAAAATCATCTTGCAACACAGTACATAG
TAGAGTATCAATTATTTATCCTCATGATAAATATCATGCTGCAACCCAA
TATGTCAGAAGAGAGTATCATACTGCATATTTACTAGCCCTAGAAAAGA
TCAAAATTCAAAATTTGAAGTATGGTTTCTTAATGAATGCATATCGCTT
TCACACCATTGTAAAGTCCAAAAATTACAAGTTAGACCTTAGTAAGTTG
GGGACTAACCGTACACCCAGAAATGGGATTGCTGGATTATATGATAATT
CTATTTTTATTTTTTGAGGACTGGATTATATGATAATTCTATTTTTATG
TTTTGAGGAACTTCCATACGGTTTTCCATAGTGGCTACACCTTTTCACA
TTCCCACCAACAATGCAGAAGTGTTTCAGTTTCTCCACATCCTTGCCAG
CACATGTTATTTTCTGTTTATGATCGTGGTCATCCTCATGGTTGTGAGG
TAATATCTCATTGTGGTTTTCGTTCTTCATTTGCATTTCCCCGATGATT
GACGATGTTGAGTATGTTTTCAGATGCTTGTTGGCTGTGTATATATTTT
CTTTGGAGAAATGTCTATTTAAATCCTTGCCCATTTTAAAATCAGGTTA
ATTGTTTTTGGTGAATTCCTTATTAATTACTCACCTCAGAGCCTTTCTT
CTTAAAATACAGATTTCTCAGAACCTTTCATCTAATTTACTAAATGTTG
ATCTTGAAGAAGTATTTTAATTTATCTGAATTTAGTTTCCCGTTCTATA
AATTGATAGTAATCATGTTTTCCCTATCCACACCAGAGTGGTATGATGA
GGAACCAATGCAGAAAATGACTAAAAAGGCATTTTTATGCTGTCCAGAT
CTTGTGCAAATATATTATAATTGATTTGAACCAAAAGAATCTACATTTT
AAGACTATTTAATATTGTCGCATTTACATTGCACTAATGGTTCCTCTTT
TTTCTCACTGATGAAGTTCTAGATTGAAACTCTGAGGAAACTGTAAATC
ACAGTTACATAACTCTGTTATATTAATTTATATAACTTCTCTGTCTCTC
TCTAAATATATATATTTAGACTATATATACATATGCATATATATATTTA
GAGAGAGAACGAGATTACACTTAAGGACTCTGCTTAGTACATTGCAACT
TGCACCTCATTCTAATTGTGAAAACAACAACAAATTTTGATTGAGACCC
TGTTATTTTTCAGACCTTTGGTCTTGTGAGGAAGGAAAAGATGATTCTG
ATACTTATCTCAAATAGCTTATCATCTCCTCTGGTGGATGAGGCTTGTG
AGGGCCTGACACATGGTAGAGGATCTTATTCATTATATAACAACTTTCA
AAACACATCACTAATTATTTTTCTGCTTAAGGTGAAAACGTAGCTCTCC
CAAACAAATAAGAGATTTAGACTGAATTCTGTAGAAAACACTGCTTACC
TCATTTTGCTTAGTTTTCACTGCAGAGTCTTGTCTCTAACATTCAGGTA
GGAGGAACTCTATTTTAAAAAATTGAATAAACAAACCCAAATCAGAATA
AATTATTTAATTCAGGAGTTTTTTAAGGTGCTCAGAATTTTGGTAGAAT
AGCAGTGTATTTTCTGTAATAAGAACTACACAATGTTCTCTAGCTGGTT
ATACTCAAATGGTTATATGGATTTTATTCTGATATCAGTTTTGCAATTG
ATGACCACCCTTCATCAATAGCGTGGTCTTTATTATGGTTTCATCTGGC
TTTTTCTTCCCTTGTCTTCTGCACTGGGCTGCCTACTTCTTGGCTCTTA
TTATTACATACTCTACTTTTTTCATATACAAAGGTCTTAGTGCATTAAT
AGTGAGTTTCTGCTAAAAGTGTTACTTTCTTCAAATATCCTGTCAAATG
CTGGCTGCCTGATTTATTGACCTCATAGAGTGACTATGTGAATCTTCCA
TTCTCTGTGGAATTCCATTCCACATTTTAACTTAAAGACTCCGTCTTTT
CCAGCTGTGTTCTCTCTGCTGGATAAGCATTTTTCTTTTAATTTCTGTG
GTAATGGGAAGGGAATTTTAAATCCTGTTTTGCGTACAAGTGTTTAACA
GGTAAAAATGGATGCTGTGTCAGCTATCTATTGCTGTGTAACAAGCTAC
CCCAAATGTAGAGACTTAAAATAAGAATCATTTTTTTTTTTTAGCTCAT
GATTCTTGATTCTGCTTGGGGCTTGGTTCATCGAGGCGATTCTTTTGCT
AGACTCAGCTGTGCTCATTCATGCACTCGTGGTCAGCTGGTAGTTGATG
ACCACATGTGGCATTCATATGTCTGGTATAACTTTTTGGTTATAAGTTG
AGACGGTGTGGGTGACAGGATCACTTGCCTGTAATTATTCATCAGGCTA
GCATAGTTCCATAGCGGTGGAAGAGTTCCAAGCATAGCCCAACACCCAA
GCATTGTTAAGCCTCTGCTTATGTCATGTTTGCTGATACCTCATTAGCC
GAAACAAGTGACTTGACCAATCCATATTCAAGGAGTGGAGAAACAGACT
ATGGCTCTTAATGGGAGAATCTGGAATATCTCTTGACAAAAGTGTAGAT
GCAAGAGGAGAATCTGTGGACATTTACTCTCCAGCAGAGATGTTGTCAT
TTTTGTCACTAAGACAGTTCCTGTGTAATATTATTTATTATTCCTTGGC
AGCCATAAGTTTGTGGTTTACTTATGGATACTTCAGTGCCTGCCCAATA
TCATGTTGGAAAGAGAAGCCCTTAACAATTTCTCTTAATTTCTACTTCT
GAGGAATGAGAAAAAAAAAATTGGATAGGCATAAATGATTTCCAGCACA
AATTCTAAGCAGCTATTTGAGAGGTGGGTGGGTAGGGAGAGATGGAAAA
TCCTTTTAATTGAGACATGCTCAACCATAAGCAATTTTTCTTTCCTCAG
GAGACATTTGGTAATGTCAGGAGACATTTTTGGTTGTACATCTAGGAGG
GAGCTCTGGCCAGAGATACTGACAAGCAATTCTGATCCTACAATGCACA
GGACAGCCACTCACGACAAAAAATTATCTGGGACAAAAATGTCAATAGT
GCTGAGATTGAGAAACCCTGCTTTTCACTGAGCGCATGTAGATGAATTC
ATATAATGTTAGTTATAGAAGGGCACTTAGATGATGCCAACTAAAACAG
GAAAAGGCAATGATTATTTCTCTATTCAAGTTAGTGAAGGAAGGATATG
TTTAAAGTCAGGTTGGAGATATTTCTCAAGGGTAATATTTAAGTTGTGA
GTGGATGCCAGATGCGCTTGATTGTTACAATCCATATTTTTATAGCTAT
ATCAAATGATCTTCTCTCCCAAATTAAAATGGATATTAGATATATTTCA
TGGATACCATATATTATAAGCTATAGCTCTTAGAAGTATTTGAGTGATT
AATAATTCTTGCATATTGAATGAGACATGTAAGAAAGATACCTGATGTC
CTATGATTCTTAGAAATATGTGCAGTGGTTAAGCACATTGTCTTGGAAA
CATCACTTGTTAATGTGAGATCCTGAACAAGTTTCGTTACCTTGGTAAA
AGGGGGATGATAACATCTACCCTGAAGATGGTTTGGTAAAGGTGAAATA
AAAGAGGGTTTATGAAGAACCTTGCCTAGCACATAAGAATCACTGAGTA
AATGATGGGGGTGATGGCAGTGTTGGTGGCAATTATCTTACTTGTAAGT
AGTAGCAAATATTTATTTGGATTATATTTTTAATAGCAAGAAAGAATCT
ATTAAAATGTAAGCAAATAGCACCATTAAGCTTTAAAAATCCATAGCTC
CCAAACTTAACATTTTTTTTTCTTAAATCCAGAAATACAAATGGTTATC
ATGAATCCTTGAGTTCCAGTACTCCGGCCTTGCCTATGTAGAGTACAAG
CATTTATTTTTCTAAAATGAGAATTATTGATGTTAGGTGTGTCATATAT
TTTATTTACTCTTTAGAGTGACAGAAAAGAAAGCAGACAAAAATAAGTG
TTTATGTGTTCATAGTGTTTGTTTCTAATTTTCCAACTTGTATGTGCAG
ACTGTAAATATTTAGAGGGAAGACATGAAATTCTTCCTTACCTACACAA
TAGATTGTCTTTCTTAGAATCTAGACATGATTTCAAATTTTCTTCAGAA
GTTCTCTTGAATTCTTACGGAATTCATTAATCCAAATGCCATCCTTTCA
AATATTCAAAAAGAAAATATTCTCCAATGTTGCTTACATGGAGGAGAGT
TATTTTATCTACTAGTGAGATGGAAGTCGCTAACAATCTTTCTTATACC
CCATAGTATTAAATAGTAATGCAATTTGATCACATATAACATTGCTCTC
CTGTTGACATGGAATGATAAGGAGGAAGGGAATATCTAACAGCACTTTT
TAGATATTCTCTCATAGAATTGTTAATGTGAGCCCAATAACTTATTTTG
TAAGTTGAGTGATTTTTCAAATATAAAAGTGAAGCAAACAAAAAAAGTT
ACCCACATTCATTCAACAAAGCTAGAAATAGAGTATGTGCTTCTTGCTT
TCTTATCAACCAAAGGAGACCTGTGAATTACAGGAAAACCATGTGTGAA
TACTTCAAAGGAAATATGCCAAACACATTGTGTGTGTGTTTTAAAAGAT
TTAGTAGCTCCTTTAAAGCTCTAAGTAAAGCTGTTAGTCTGACTAATCA
TGTACCCCTGAAACCAATTAAATTTTCAGACTAGAGAAGTGTTCTTCAA
ACACTTGAAAAAAATAAGAGTTCCTTCCTAAAGCCAACACCTTTTAATT
AACAAAATACTCAGAGCAAAGCTGTGGTTTGCAATAGTATATTACACCT
ACTGTTATTCATGCTACCATTCTGGTGTGCCAAACTCAATTCATTTCAA
CTATTTTGAGCTTGATTTTCTGTCTGCTTAACTATGGAGGGAAAACCAC
AATTTTAGTAGAGTCATTTTAATTTTCTAATGAATGCATGAGAAAGGTA
TTATGCTACATAGTATGTCCTCAGACTATTCAGATGCCCTCTCTCTCTC
TTGCTCTGTCTCTGTCTGTCTCTCTTTACACACACACACACACACACAC
ACACACACACAATGTGTGTATCTATCTGTCTACCTAGATATACAGTTTA
ATTTTGTACATTGTAATACATCTGGTTCTGGCATAAGGTGATTGAGCAA
AAAACAAACAAACAAAACCCACCCAAATCTGTTACTGGAGGAACCTTTA
ATAGCATAACATCAGAAAGACTATTTTCAGTAAGGACCCTAGGCAGGAA
AAGACAATTCCTCCAATGGACTTGAGAAGAATACCCTTAGTCTCTGAGG
TGGTTAGAGAGGCTGAGATCTGAACCAGCTAATACCAGAGCCAGCTTCT
TGTAACCCAAGATAGGGGCATAAGAAACGGGGTGAATGCCCAGGCATGA
ACAGACTTGGGGTTAAGATTAAATAGGGTACACGGGAAAGGATTTCCGC
CAGCTGGAGGGGAAGCCATTTCCCATGCTCCTTAGGGATGACAACAGGA
GACCCAGCCCAGAACATCATTGAAAATTCTATGTAATAATTTATATATC
CTAACTCTTTACCCGTTCGTTCTAGATTATGGATTCAGGCACTCTTAGC
AAAACAAATCCTCTTTCAGTCTTTTAAAATATTGACTTAACAGGAAATA
TCCTTGGCACAACAATACGACCCTGCCTGACCCTTAGAACTGTGAACAG
GTTGCTCAGACTGTACAAAACCACTAACAAATCTGACCAGCCTACACTT
CACTGAAAAAGGTGATTTGCATATATTTCTAGTACTATGACATGGTGGA
AAAGAAAGAACCGCATATGTGTTAGATAAAGGTAAACGCATCTGAAAGT
GCAGTAAGAATCAATGTGATATATACAATAAAATAAGATAAATAGGCCG
GGCATGGTGGCTCATGCCTGTAATCCCAGCACTTTGGGAGGCCGAGGCA
GGCAGATCACGAGGTCAGGAGATCGAGACCATCCTGGCCAACATGGTGA
AACCCCGTCTCCACTAAAGTACAAAAAATTAGTTGGGCATGGTGGCAAA
TGCCTGTAGTCCCAGATACTCAGGAGGCTGAGGCAGGGGAATCGCTTGA
ACCCGCGAGGCAGAGATTGCAGTGAGCTGAGATCACGCCACTGCACTCC
AGCCTGGCAACAGAGCAACACTCTGTCTCAAAAAAAAAAAAAAAAAAAA
AAAATTTCAGGACTTCAAAACTTCTGTCCTTTGAAACACTTTGATTAAA
AAATGAAAAAAATATATACATGCCTGAATCCAGAGGCAAGTCTTTTTAG
AAAACATAAAATATTTTAAGTAATGTTTTTCTGAACAGAAATGGCTCTA
ATGTGAGAGGTCGATGAAGATTAGCCTCTTGCTTCTTTATGTTTTAAAG
TATATTCCTTGAGACCCAAAGTTTCTGACTTTAGAATCCAACTCTCAAC
TACTATCAGCTATCCTCTAGACACTTTCAAAATCCTCCAAAACCTAATA
GTGGTGCAAGATTTTGTCCTTCCTAGTGATAACTAGATTTAAGTAAGAA
AACGTTAAAGACTAAAAAGTGTTTAGGACCCACAAGAAAGGTGTAATTC
TGTTCCTCATGGAGATATTTTAGTTACACACTTCTTATTCTGGTCCTCC
CCAAAACACAGAAATCTAGAGTTGACATTTAAAGAGAAAATGAGATTTT
TGTCTGGAAACAGGAGAGAAAAAGTTTTCAAGAAGTATAGAAAGCGTAT
GAAAGGACACAGGTTTGAAATGGCCTGTGCTCATGAGAGAATGCACAAT
AGACATGGGTGTGGAGGAGCCTGGAAAGAATGGAGAGAGGTAAGTCTGG
AGGGTCAAATCTGGTTCAGAGTACAGAGCATAAAATCCCATACGAATGT
AGAACATGAGGAGCCAATAACCATTGTGTGAGAAATGCAGGAAAAAGAG
GAAGAAAAAAATCCACATAGTGTACTTACAGTCAGAACAATCCTAGGAG
GTGACTTACATGTATGATGTCATAAAATCCGTAACACAATTCCATTAGA
TTGGTACTGTTTTCATAGTTACACAGGTGAAAACTTTTGAGGGTCAAGA
TGTTCATTGTAGCAGGCATAGTATAATTCCTTACCTCAAGATGCCTCCA
ATGTCAGTGAACATTACTTTTCCAAGATTGTCTATTTAGGAAAAAGTAA
GGCCAGTTGTCTATGAAGGAAGTACTATTATAATCGCCACTTTACAGAG
GAGTAAATGGAGATTCAGAGAGGTTAAGTCATCTGACTATAGTTACACA
GCTAAAAAAAAAAAAAATTATAGAGCAAGGATTCAAATACAACAATCTG
GTCCTAGAAAGCCTAAATATTCTACACCCTGCTTTCTGATTCAGAGCTA
TTTCCTGCTTTTCTCCCTTCGATAGGAAAAAAAATGTATGTGTGTGGGG
TTGGGGGCAAATAAATTGACTGGCTGTATTTTCTCTCTTTAATATTTGA
TCTTTCATTGTCTGCTCCAAGCAGTGGGTCTTCCTCCATTATCTTCTCG
TGAAAATAGCTAAAATCACCTAACTAGTAAAAACCTAGGCATGTTTTGT
GTTCTAGCCTCCATTCTTCCATTCTAATGGGTGGCTGTTAAATTTTAGT
ACTCATTGTATGTAGAAGACCTCTAAGTTCACTAAAGAGCTTACTGCAC
ATTGACTTTTCTTTAGATTCAAAGGGAATCAATTGAGCACCTCCTATAC
TCCAGGACATGTGCAAGATGCAAGAGATAGAGCAGTGAGAAAGTCTGAT
AAGGTTTCCCCTCCCTTTCCCAGCAGTGTACAGATAGATGTTCTTCAGG
TGGTGCATGCTGGAGAAGAGAAGACCAGAAATACTGGCCTGACGGGTCT
ATAGATGAGATTATCTGCCCATGACTGGCGCAGGGATGGCCGCATTGCT
AGACCTCTCCATCATCCTCTGAATATGCTTCAGCTAATTTATCATCCCA
ATGGCATATTTAGTCATCATGAACCATTCTCTCTTTTTGAGTCTCAGGC
CCTGGCCTTGCTTTTCAATAGACTTCCAATAGATTCCTTCCTCCTTCCT
TCCCTTACTGCCTGGCTTCCTGACTTCCTTCCTTCCTGCCTTCCTGCCT
TGTCTTGCCTTGCTTTTCAATAGATTCCCTCCCTCCCTCCCTCCCTCCT
TCCTCCCTCCCTCCCTCCCTGACTTTCTTCTTTCCTTCTTCCTTTCTGT
CTTTTTCCAAACGTGCTTTTCAGGAAACAGTGGTCTGCTTGTTGAAGTC
TGATAATTCTCTAGTTCCTCATCCTTCACTTTATTGAAATTTAGTGTGA
CATGATCATTTCCACCTCATTAGGTACTTCTTTCATGGTCAGAAGTAAG
TCTGGAGAAAAAAAAAAAATCTCTTGTCTCTGCTCTTATTTGAGGGTCA
AATTTTCAATAAGTCACTTTTTAAAAAAGCATTTTCTGACACTTCCCAT
AAGCTTGAAATCCTCCTAAATTGCTGTATATTGCCTCAGTATACCTTGT
GTATCTATTTTAGGAACACTCCATCCACATTTGCCAGTCAGCCTGGTGT
TCTGCAGTTAGTTCCTACAGTGATATTTTAATTTAGCTCTCTTTTCATC
CTCACACATGCATCCTCTCTGGATATTTAGCTCCTTTCCTGGAATCCCT
TTTAAGATTTCTAGATCTTTTTGCTTCCATGATTTCTTCTCCTGGACTG
TGACACAAAATGCTATTTCTTCTTTACATTACATTTAATTCTTTCTAGA
AAGAGCCTCAGAGTAGTCAGATAGCTTTGAGAAACAAAACTTTTTCTTT
ATTGCCTCACTGTTACTGCCTTTCAATCATTGTTTCGTGACACAAATTT
TTTTATTCTCTCTGACAATTAAAACACTATTTTTTTCTGTCTGCATTGA
TCAAAATTAGTTCCTTCATTCATAGAAAACTCTTGGTGTCCCTGAGAAG
CTTGAGAGACAAGAAACATTCTTCCATTCTACTCATCTTCTTCTCTAAT
GAGGAGACAACCTTAAAAGCACAGTTACATAGCCATAAAAATTAATGAT
TGGCTACCTCAGAATGAAAATTCAATGTCTCATTTTTTTTTAATATTCT
TAGAATCGTTCACTGGTTGTCCAGTGTGAGTCTCCTGTTGAGATGTCTT
TTGCAGCTTTCCTTGAAACCTTTCATTCCAAACTACATAGTCCAATAAT
TTTGCCACCAATCTTCTGGTTATATTATGCTCTTGAGTCTGTTGTCTAT
AAACTTGATTAGGCATTCCTTCCCCTCACCACTCACCTCTGATAACCCA
GCTGTGTGTTGGTATTTAGTATCAATTCACACCAGCAAGTTCAGCCCTC
TTCAATCAATATAGGGCCACACACGGACTTTTGACTGACTACTCCCCAA
GTATTTCACATTTTGGGGCCTTATCTCCAGTTTCTCACCACAGTTGTTC
ATCACTGTGTTTCTTACTAGCCAGGCGTTTATAAAAACAGTAATACCTA
ACACTATTGATCACCTACTATAGTGTCAGGCGCTGTAATAATATTATTG
TGATGATGATGATTATGCTGCTCTTTCTGGCATTGTCATACGTGTATTG
CTTGTACTACTCACTGAATCTACACAACTGCCCTTATGACATTTACCCT
GTTATTATTCCTCTTTTAAGGTAAATACATGAAAAATGCTTCCCACTTT
GCCTTGCTTACTGCTTATTGCTAGTACTGAACAAATGTTAGAACTGAAA
CTTAGAGAGGTTATGTGGCTTTACCAAGGTCCCAGAGTTCCTAGGGCAG
AGAACAGGATTGTCTACCAGACATTTTAATTCTAGTACTATGCATCTTA
ACCATTACCATAGGCTGACTTACTCTACAGTGTCCAACACTATTCATAT
TAAGATTTATTTAATGACTTTGAAACAGTATTTCATGTCTAAATAGAAA
AACTACTAACTCGCATTTTTAAGAAAATATTGTATCTTGGTTTTTCTTC
ACTGCTGGCCAGTTTACTAACAATCTGAAATAAAAAGAAAAAAATATGA
TAAACTGCTCCCAGTATAAAATACAGAGCTAAGACAAGAACGTTTCATT
GGCTTTGATTTCCCTAGGGTCCAGCTTCAAATTAATTTACTTCCTATTC
AAGGGAATTTTAAATCAGAAAGAAGATCTTATCCCATCTTGTTTTGCCT
TTGTTTTTTCTTGAATAAAAAAAAAATAAGTAAAATTTATTTCCCTGGC
AAGGTCTGAAAACTTTTGTTTTCTTTACCACTTCCACAATGTATATGAT
TGTTACTGAGAAGGCTTATTTAACTTAAGTTACTTGTCCAGGCATGAGA
ATGAGCAAAATCGTTTTTTAAAAAATTGTTAAATGTATATTAATGAAAA
GGTTGAATCTTTTCATTTTCTACCATGTATTGCTAAACAAAGTATCCAC
ATTGTTAGAAAAAGATATATAATGTCATGAATAAGAGTTTGGCTCAAAT
TGTTACTCTTCAATTAAATTTGACTTATTGTTATTGAAATTGGCTCTTT
AGCTTGTGTTTCTAATTTTTCTTTTTCTTCTTTTTTCCTTTTTGCAAAA
ACCCAAAATATTTTAG

Homo sapiens dystrophin (DMD), intron 50 target sequence 1 (nucleotide positions 1524636-1524685 of NCBI Reference Sequence: NG_012232.1) GTAAGTATACTGGATCCCATTCTCTTTGGCTCTAGCTATTTGTTCAAAAG (SEQ ID NO: 835)

(SEQ ID NO: 835)
GTAAGTATACTGGATCCCATTCTCTTTGGCTCTAGCTATTTGTTCAAAA
G

Homo sapiens dystrophin (DMD), intron 50 target sequence 2 (nucleotide positions 1570168-1570417 of NCBI Reference Sequence: NG_012232.1)

(SEQ ID NO: 836)
CGTTTTTTAAAAAATTGTTAAATGTATATTAATGAAAAGGTTGAATCTT
TTCATTTTCTACCATGTATTGCTAAACAAAGTATCCACATTGTTAGAAA
AAGATATATAATGTCATGAATAAGAGTTTGGCTCAAATTGTTACTCTTC
AATTAAATTTGACTTATTGTTATTGAAATTGGCTCTTTAGCTTGTGTTT
CTAATTTTTCTTTTTCTTCTTTTTTCCTTTTTGCAAAAACCCAAAATAT
TTTAG

Homo sapiens dystrophin (DMD) intron 50/exon 51 junction (nucleotide positions 1570388-1570447 of NCBI Reference Sequence: NG_012232.1)

(SEQ ID NO: 837)
TCCTTTTTGCAAAAACCCAAAATATTTTAGCTCCTACTCAGACTGTTAC
TCTGGTGACAC

Homo sapiens dystrophin (DMD), transcript variant Dp427m, exon 51 (nucleotide positions 7554-7786 of NCBI Reference Sequence: NM_004006.2; nucleotide positions 1570418-1570650 of NCBI Reference Sequence: NG_012232.1)

(SEQ ID NO: 838)
CTCCTACTCAGACTGTTACTCTGGTGACACAACCTGTGGTTACTAAGGA
AACTGCCATCTCCAAACTAGAAATGCCATCTTCCTTGATGTTGGAGGTA
CCTGCTCTGGCAGATTTCAACCGGGCTTGGACAGAACTTACCGACTGGC
TTTCTCTGCTTGATCAAGTTATAAAATCACAGAGGGTGATGGTGGGTGA
CCTTGAGGATATCAACGAGATGATCATCAAGCAGAAG

Homo sapiens dystrophin (DMD), exon 51 target sequence 1 (nucleotide positions 1570442-1570487 of NCBI Reference Sequence: NG_012232.1)

(SEQ ID NO: 839)
TGACACAACCTGTGGTTACTAAGGAAACTGCCATCTCCAAACTAGA

Homo sapiens dystrophin (DMD), exon 51 target sequence 2 (nucleotide positions 1570455-1570498 of NCBI Reference Sequence: NG_012232.1)

(SEQ ID NO: 840)
GGTTACTAAGGAAACTGCCATCTCCAAACTAGAAATGCCATCTT

Homo sapiens dystrophin (DMD), exon 51 target sequence 3 (nucleotide positions 1570465-1570506 of NCBI Reference Sequence: NG_012232.1)

| (SEQ ID NO: 841) | |
|---|---|
| | GAAACTGCCATCTCCAAACTAGAAATGCCATCTTCCTTGATG |

Homo sapiens dystrophin (DMD), exon 51 target sequence 4 (nucleotide positions 1570442-1570506 of NCBI Reference Sequence: NG_012232.1)

(SEQ ID NO: 842)
TGACACAACCTGTGGTTACTAAGGAAACTGCCATCTCCAAACTAGAAATG
CCATCTTCCTTGATG

Homo sapiens dystrophin (DMD), exon 51 target sequence 5 (nucleotide positions 1570518-1570567 of NCBI Reference Sequence: NG_012232.1)

(SEQ ID NO: 843)
TGCTCTGGCAGATTTCAACCGGGCTTGGACAGAACTTACCGACTGGCTTT

Homo sapiens dystrophin (DMD) exon 51/intron 51 junction (nucleotide positions 1570621-1570680 of NCBI Reference Sequence: NG_012232.1)

(SEQ ID NO: 844)
GATATCAACGAGATGATCATCAAGCAGAAGGTATGAGAAAAAATGATAAA
AGTTGGCAGA

Homo sapiens dystrophin (DMD) exon 51/intron 51 junction target 1 (nucleotide positions 1570623-1570674 of NCBI Reference Sequence: NG_012232.1)

(SEQ ID NO: 845)
TATCAACGAGATGATCATCAAGCAGAAGGTATGAGAAAAAATGATAAAAG
TT

Homo sapiens dystrophin (DMD), intron 51 (nucleotide positions 1570651-1614861 of NCBI Reference Sequence: NG_012232.1)

(SEQ ID NO: 846)
GTATGAGAAAAAATGATAAAAGTTGGCAGAAGTTTTTCTTTAAAATGAAGATTTTCCACCAATCACTTTA
CTCTCCTAGACCATTTCCCACCAGTTCTTAGGCAACTGTTTCTCTCTCAGCAAACACATTACTCTCACTA
TTCAGCCTAAGTATAATCAAGGATATAAATTAATGCAAATAACAAAAGTAGCCATACATTAAAAAGGAAA
TATACAAAAAAAAAAAAAAAAAAAAGCAGAAACCTTACAAGAATAGTIGTCTCAGTTAAATTTACTAAAC
AACCTGGTATTTTAAAAATCTATTTTATACCAAATAAGTCACTCAACTGAGCTATTTACATTTAAACTGT
TTGTTTTGGCACTACGCAGCCCAACATATTGCAGAATCAAATATAATAGTCTGGGAATTGATTATTATCC
ACTCTTCTAAGTIGTCTGTGCCAATTTGCCTTCTCCAATGATAAGGATAATTGAAAGAGAGCTATAACTT
AAAAAGAGAAAAGTAACAAAACATAAGATATTTAAAATTACCCTAGATCTTAAAGTTGGCATTTATGCAA
TGCCATGTTCAAATGAACATGTTTTTAATACAAATAGTGCATTTTTCAGCCTCAGTGTAATCCATTTGGT
AAAATTATGACATCAACTAGAAACATTAGAATACATTGATGTAAATATGGTTTACCTAGCTAGATCAAAT
ATACTATATATCTTTTATATTTGTGAATGATTAAGAAAAATAATGTTGGAATTGTTATACATTAAAGTTT
TTTCACTTGTAACAGCTTTCAAGCCTTTCTAAAGAAATACAAAGTTGTGCTGAAGGTATTTAGGTATTAA
AGTACTACCTTTTGAAAAAACAAGAAGTGAGGCAGACAGAGTAAGGGGAATTTCTTTGTAAAATAAACTT
CACCAATTCCATAGGAATAAAAGTAATTTGATAGTAAACAACCTGCATTTAAAGGCCTTGAGCTTGAATA
CAGAAGACCTGAATTCAGTGCCATTTGCAAATGATGATTGTGGTCAAGCCATCTCTGGATCTTCGTTTCC
TATTCTGAGTACAGAGCATACAGAGTACACATTCACATTCACAATATAGTTATGGATATGGATGTATATA
AATATATGTAAATACTACATATATGTACCTAAAATTTGTTTTACTTCTGCTTTAAAAAAAGTAATTATAG
CCACATTTTTCAGAAAAAGTAACTGAGGCTCATAGATGTCAAATTCCCAGTAAGTAGCAGAACAAGGATT
CAAATCCAAGTCCATTTGATTCCTAAGCTTGTGTTATTACTTGCTACTGCAGAGAGTATACGTAGCAAGT
AATATATGTACTGCAAGCAATACATACTATTGCTGCGGTAATAACTGTAACTGCAGTTACTATTTAGTGA
TTTGTATGTAGATGTAGATGTAGTCTATGTCAGACACTATGCTGAGCATTTTATGGTTGCTATGTACTGA
TACATACAGAAACAAGAGGTACGTTCTTTTACAATACCATATTGAGTTATATAATACTCCCAGGACTTTT
ATTTACCAAAGGAAACAATATTTTATAATGTTTAAAGCCCAGGTTTTGAAGTTACATTGTCTGGGTTCAA
AGCTTGGCTCCCAAGCTGTGTGACCTTGAGTAAGTTATTCTGCCTACCTGAGCCCAAGTTTATCTAGCTA
TAAAATGGGGATAGTTGTACTATCTGCCTTGCAGTTTGTCATCAGGATTAAGTTGGTTGGTACATGAAAA
ATGCTTCCCACTTTGCCTTGCTTACTGCTTACTGCTAGTATTGAACAAATGTTAGTAATTATATTTGGTT
CCACCACGAACTCTAGAAATCTAACCAATGATGGCATTTGTATTATGCAAACTGTATATCACATCATAAT
ATTATATGGAAATGAGAGCTTGTTTCCGCTTCTGTAGCCTAGTCTACCATTGACATAGCTTCCTGCAGAA
GTTACCAGATAATAGATTGGGAGAGAAAGTCCACACTTCCTTGTGACGGGTTTGTGAGTCCAGCATTTAG
GGAAGCCATTGATGTGCTCAGTAGTCTCCAGAGTTCTCTAAATAAATGTGTCCTTTTCAGAAAGGACTAC
TGATTTGATGCCCCCTCACAGAGATCGTCTTTAAATATAGGTCAAAAACTAATGTAGAGGGCCAGGTGCA
ATGTTTCACGCCTGCACTCCCAGCGCTTTGGGAGGCTGAGGCAGGTGGATCACTTGAGGTCAGGAGTTTG
AGACCAGCCTGGTCAACATGGCCAAACCCCATCTCTACTGAAAATAAAAAAATTAGCTGGTGTGGTGGCC
CATGCCTATAATCCCAGCTACTAGGGAGGCTGAGGCAGGAGAATCACTTGAATCCTGGACCAGAGGTTCC
ATTGAGCTGAGATCACACCATTGCACTCCAGACTGAGTGACAGAGTGAGACTCCATCTCAGAAAAAAAAA
AATTTAGGGGGAAAAATCAAAAGCCATTTCTGAGACACAAAAATACAGGATTTATAAATTATATATGGTA
TATATAAAAATATTTTTAAAATAGTATATATAGCATATTATATATAATGATATGTAATGTTCATATATTA
CATATTTATAAAAAAATCTAATCTCCCTTCTCTTGCTTGCTGAATAGGGGGATGCTTTGCCTGCCTCTTC
CTCTTATATTAAAAAATAATTCTTAAAGACATTGTCAGTTCTTGGCTTTTATAGCCTCAATCACCAAATT
GTCGGTAAAATGGCCCTAAATAATCATTAAACAAATGTGTGTGAGAGGGGAAATAAGAAGGATAAGTAAG
TATGGGGAGGATTTTGTTATAATTTCAGGAAATCAATATCAATTTTATGTAAAGTTTTAAATAAAGCAAT
CCCAACTTTAATGTTTGATGTGTGAAAAATTAGGCAAAATTCCAAAAGGGCTTTATAAACTGAAAAAAAC
TTTACTAACACCTATCCATTTTTATTATTTTAACCAACTTCTATTGAGCTGCCACTAAGTACCTGGGAAA
CATAAAGTTGTACAACATAGAATGTGCAGGTAAAAGAGGTTGAAGGAAGAAAATAATAACACTATGATAG
AGATAAATTTTAGGATAATAGCTAACACATATGATATGCCAGTCATTGATCTAAGTACTTCACGTGAATT
CTTTAATGCTTACAACATTACTGTGAGGTAGATAGAGAGGCACAGTAAGGATAATAACCTGCCTGAGATC
GAGGAAGAAAGACAATGATGAGATGTGAACTCAGGCAGTTTGGTTCCAGAGTCCTCTCCCTTAAACCTCA
TAGTTTTCAACTTCTCTGATATTGTGTGGGTGATGCTGTTGGGGCTTTCTTCAGGGAAAACTAAGCCAGG
AGAGAGAATGGATGCTAGTGAGATATTCCTGAAGAAGGAAAAACTTAAGCCAGGCATTAAAGAATGAGTT
GGAATTACCTAGCTAGATAAAACGAGAAGGGCAATCCAGGCAGAGGGAACAGACTGTGCTTTTCACTGAG
GTGGAAAAAAAACAGAGTATATCAGAGGAATTGTGATTCCATATGGCTGAAGTTAAGGGTATATGATGAG
GAAGAAATTGATGAGGTTGAATAGAGAGGACTGGGGCTAAATAATGGGAATCCTTTGTTGCCAGACTGAG
GAATTTTGATGATGGCCTACAGGCAGTGGCAACTCTGAAAGGATTGTAAACAGGAAAATAAAATCATCAC
ATATAGTTTAGTTGCCTATCAATTAGAGCTCTCTGGATGCAAGCAACAGAAATCATTCTCTGATTAAATC
AGGCAGAAAGTAAATGTGCTGTAATTAGCACAAAGGCATTGGAACAAAACTTACAAAAGGAAAAAGAATC
TGAGCATGCCTTTCTGGGCATGTGGCTAGCAAGAAGTATTCCAGTCTGTTTGTGATACTCTCTTTTCTCC
ATCCTGTGTGTAACTCTGTTCAAATTTTAAAGTCTTAAAAGAGAGTCCAGTTCACCTTGTTTGGGTCACA
TGTTAATACATGAGCTAGAAGGGAGCAGAAAACTTTGATTTAAATCCCTCTCCTCCCAAAGTCTCAAAAT
TAGGGAAAGGCAATTCTCCTGAATAGAAACTGGGTTCTATTGACAATAGAAGAAGGAAATGATTCTGACC
AACCACTAAACAATAATTGTCCACTGAACTCAGTCAAGAACATGTAGAATAAGTTGGAGGATAGAGCAAA
TAAAGGAGATTTGTAGGAGGTAATTATTATGATCTAAAGCAAGCTTGTTCAACTCATGGCCTGTGAGCCA
CATGCGTCCCAGGATGGCTTTGAATGTGGCCCAACACAAATTTGTAAACTTTCTTAAAACATGAGATACT
TTTTGTGACTTTTTTTTGCTCATCAGCTATCATTAGTGTTAGTGTATTTTATGTGTGGCCCAGGACAATT
CTTCTTCCAGTGTGGCCCAGGGAAGCCAAAAGATTGGATACAGCTGATCTAAAGCAACAGGTTCATCTAC
TCAACTTCACAACGTGTAGACCTGAAATAAAGACCATTCATATACCAATACCTGAAATATAAATTTGTTT
GACCATGACACGTACAGTAATTGGTTCTCAATAAATGTGGATAGCTTGATGGATAATGTGAATGCAATGT
GATAAGGAAACTTCATATTCAACAAAGACTGGAATGTGAGGATTATAATTCCAAAGCACCAGAAGATAGA
TAAGATAATGCAATGAGACATTTTATGACTCAAGGCAAAGTTAGTTATGAGATTCAGACCAAACCTTAGA
CGTGCAGTAATTGAAATATTTGCCACAGAAGGGGTATAAGGACATGACATTCAAGTAAGCTAACCTTTCA
CTAGCTTTAGACTTTGAACTCAGAAAACATATTTGGTGAAAAGCTTATGGTCCCCTTTAGTATGTATTGC
TTGATTAAAGTATTATTTTAGAAAATGGTGAGCTGCTTCCATTTTGAAATAAAAATAATTTTTACTAAGT
GAATTATATTCAGTGAAAAAAATGGAAGCTACAATTACAACTTTAATTTTTTTAAGTTTTAAGAATACAG
CCATTTAAAAAAATTAAGCAAATCTGCTTCATTTTAGACAGTAGAAAATATACCATTATCTTTTAGAAGA
ATAGAGATGTGAAATATGCAAATTAAGCCTTTAGAAGTAAAGCACACATGAAGTTCAAAGTTTAATTTCT
AGAATTGTGAATCAATAGCAGTGGATGATTTGTACTTTATAGCTTAGTGTCGGAGAAATCTGATTAAAAA
ATGCTTTTTCTGTTTCATCACATAAACATAAGTAAAATTGCTCTGAAACAACAATATTTGACAAGAATTA
GCAGTTTTCTTTTTTGACATAATCTATCAAATGAAGGGAAAAATATGTCCTGGGTTTTGCTTTGAGAGTG
ATTACTAAATCTGACCCTTAAGGAAAGGAAGGAGAGAACAAAGAAGGGAGGAAAGAAAGGGAAGGAAGGA
GGAGGAAAGGGAAAAAAAGAAGGAAGAGAGGAAGGAAAGCAGGAAAAAGGGGAAGGAGAGAGAAAAGACA
AAAGAAAGGTAGCAAGGAAAGAAAAAAAGACAAGAAAGGAATATTAAAGAGGACAAAAGAGGAGTGAGGA
AAGGAGGAAATGGAAGAGGGATGGTGGGAGACAGGAGGGAGAAAGGTGGAGGGGGAAATATGAAGAGAGG
TTCCCAGCAGTGGAGACTAGTGTTGCTATCAACAAATAGAATTTAGATGGCCATATGATATTATTTTTCA
TAATACTGGTGTCTGATTGCCTGTGCTGAGTTAATTGTAGTCTTTTTTTTCAATTCCGTTTGGCCAGGTG
TTCAGGATAATTCACCACAAAATCTCAACCACTGCACTTGTATTGAATAAAGAATTGAGTTGGCAAAGGC
ATTTTATCCTCCAGTAAGACCTTTCCAGATTGGGGTTGAGACAAATTGGCCAATCTGGACAAGATGATAA
TAGCATTGTTCAAGATTAATTTTTAACCACACATTGCACTGTTACCTGGGAGATTTCATTATCTAAAAAT
TGAATGAGCAGTTTTAGTGGGTATAGTGTATATTTAAATGGGACATAATTACTTGAATGAGTTTAATTTT
TGTTGTTGTTGTTAAGGTCAAAGTACTTAAAAATTATGATTTTTTAAAACTCTGTCTATACACAAAAAGC
ATTTGAATTAGCTACAGAATAATTCTGATTATAACTTTTGGTGAATAGATTCAGTCAAAATCTGATTACT
AAACAACTTGTGTAGTATAGCCCTGGAAGAATTGATGGGACAATGTGTGGGTAAAGTGGCATTGGCTATT
TAAACTAAAAGCAATACAAAACAGAATGTTTCTTGGTTTTATTCTGTTGTCACAAACCCAGCAGAAAGTG
GCTATTACAATAGTTTCCCTTATTCAACAAATGAGAGAAGTTATAGACAATTTAGTTAATTGATCTAAAG
TCACTTAGTAAATGTAATTGTCCTAACATAAACCCAGACCCCCAGACCTCTTGGGAATAGATAATGTTTC
TTACTTCTTTTCTATTTCCTCAGCCACCCCCCTCAACTTCTTACACATCTCATTTCTCCATCCAAATTAT
AACAAAACAAAGCAAACATGGTTTATTTCCATGGGCATCAAATGGATTTCACGAGGTTGGGTGACAGTCA
TCTTAGGGTGAGGAGATTGATTATTCTGTTTTTCTCTTTCATCGATCAACAATCCAGCCCTTCTCATCTC
ATCATTTCATTTCTGCACAAACTTGTTTAAGAAATACCAATTAAGAAATTAATTAAGAAATTAATGTTGT
AATCTGTTTGGCTGAAGATATTTACAAATTTTGTGCTTTAATTATCTTCCAACAAATGTACATGTCTCTG
GTAGACAGCTTGCGACCATCTGGATGACTGATCCATATTTATATAATTTTCTTTCTTTACCTAATGAGAC
CAAATCCACTATTATCTTCAACGAAGGATGTAAAGATATGTCAGTGTCAGTAATGTGACTTATTTTATAT
TCTCTGGTCATAACAAAAATAAACCGCCCCTTAAATAAAAAGGTCATAGAGTTGCAAACACACACACACA
CACACACACACACACAAAATCATATTTTCTAAGTCTCCTAATTACCTTTTTATGGAAAATGATACCATAT
GCTTTTTTCTTAAAGAAACTACATAAACTTATAAACTATACTAAACTACACATTTCAAAGTCTATGAATG
GAAATGTGTATCTTATTATATTTTAATTCAATTCACTGTAAACTTTTCTGTCAAAATCTTATCAAGCAAA
ACTGATCCAGGATATTTACATGAATTCTGATGGAAGTCACTGTACTGTGTTTTCCATAAAATACCAGTGG
GATTCTGATAAGGAAGTTTATGTTTGCCATTGTGTTTAAATAGAGAATTCTGGGCCGGGCATGGTAGCTC
ACGCCTGTAATCCCAGCACTTTGGGAGGCTGAGGCGGGTGTATCACCTGAGGTCAGGAGTTTGAGACCAG
CCTGACCAACATGGAGAAACCCCGTCTCTACTAAAAATACAAAATTAGCCGGGCGTGGTGGCGCATGCCT
GTAGCCCCAGCTACTCCTGAGGCTGAGGCAGGAGAATCGCTTGAACCCGGGAGGCAGATGTTGCAGTGAG
CCGAGATCACACCATTGCACTCCAGCCTGGGCAACAAGAGCGAAACTCCGTCTCAAATAAATAAATAATT
AGAGAATTCTATTTACAAATTTCCTTTCTTGGATCTAGTTAGGGCTCCTTTATATGGAGTGATTTTTATT
GTTTTCATAGAAATACGTAGAATCTGGGTCTTCTCTAACTTTCTTACAGGAAAGCAATGTAATAAGGTTT
TTTTTTAATTTTCTGAAAGTTATATAATGTTATTGTTCCCTAAAGTTTAGGACCTGCCTTTTAGGCTTTC
CATTTCACCATAACTTTTGGTCCTTAAAGTCTGTAATTGAAGTTACAGTGTGTTATGATGTAAATTTTTC
TTATTATTACCTTTAATGTTAGGGTAATGTTAACTAATGTTAATGTTAGGTATATGGTTGTTTTTTTCAT
TCCTTCGTTCAACAAATTGTCTTTGAAACCCATGTTACAAAGCACTCTAGAGTCAGGTTGAAGACTATTA
AGAAAGGAGAATAGAAAGAGACACTAGAGTAATAATTTGGATTTAAATTTGATTTCCTTGTGTATGATAG
TGAATAAGTGTGAATAAGATGAGGCAGTGATCACACATCACTATTAGTAAAAGTGTTTCTGTACCTGTAT
CCACACTTTTATGTATATGGTTACTTATGTTAAAGTGATACATATTATATAAAATTAACGTATACATTAA
GTAGATATTTTAATAGTCTGTAATTAAATACTACTAGTATTTTCTTTCCTCCTTCAAGTGCTTACTTTTG
ATACCTCGAGTTACAGTGTCATAAAGATTCTTTAGAAATATATTGACTGTCTTTTAAGAGCTTTTGATAC
AATACTGAGTTTACATTCATCTGTTATTTATTGAACACTTGCTGGTGAAAGGCATCAGTGTTATCTGCTC
TTAGGGAACAAAAATTAAAAAGGGATAGGCCCTAATTTTAGAGTGTATCCTCTATAAGAAAAACATAAAA
GATAGGGCAGTCATGGCCACAAAAGAAAAAAGTGTTATGGTGGTTTCAATCATATATGTATTAGAATGAA
TAAATCAACTGATCAATTGTGATTTCTTATTCTAAATATGTGCCTGCCTTTTTCATATAGATGAAAATTA
AGCTATGTTTATCTTTCCAGGGATCTTGTTGATTTTTATTCAATAACTTGGGAGTGAAAGTTGATTTTTG
CATATGTTTTAATGTTTTTAAATTTCATAAATGAATTGATCAGTAATTTCCAAGGTAGTAATGGCTGCAT
TGTTTTTGAAAAAAAAAAAGCAACAGGATTTGATTGTGCTTTTATGATTTTTAAAGAATTCATTAAAAAT
AATGCCACGGTTTCTAAAATGATTTGAGTCAATTTCTTATTCGATTTATAAAAATAACTTTGAATACAAT
TTTAGTAATTCACAAATGCTTTCAGTTCCCTTACCTTTATATTTTATATTCTGTGTAAACAAGTGACATA
ATATTTAAGAATTATATATCTCCTATGATTTATTCAAGAAAAGAATATATACTGTATTATTTATTTCAAG
AACAGAAATGCTTTGATTTAACTGTCATCTTCTCTCTTCAATTATGGAAGCAAAATAAACTGTAATGACC
AATGTAACCCCTCCCCCATATCAAGTTAATCTATGTTCAACTCCAGAATTATTTTTGAACACTCAAACTA
GAAATTAAAAAAAATTAAATCCATGAAGACGATTTTTGCCAAAAGCATATAGATAAATTGAGTTGATTCT
ATACTTAAGAAAGTGGAGAGGAGAGAGTAATTTGGAGAGAGTAATTTACTCTTAATCCCATATTTTTTCC
CTAAATGTGAAAGAAGTAGATTGTAGTGAGAGGGAAAATAACCTGTAGCAACTTCATTGAGGCTAAGCTT
TCTGTCATGTTATATTATACGAAAGTAATGAAATGCTTCCACAGATAGAATCAGAAGTCCCCTCTGAGAA
ATTCTACATAAAAATTAGCCTGCCACTTTACCACACTTACTCAAGTTTGATTTTTTTAAGTTATGTAATA
GATGTTAGGCACTAGAAGAGGACATTTACTGGGGGCAAAGATCAGTAGTTGGAAAGAATGCAAGCAGGCA
AGAAGCTATATATAATGAGATTTTACAGTACAATTGTTTTCTAAATGAAAATGAGGACGGGTCCAGACAC
AATGGCTCACACTTGTAATATCAGTGCCAGGATGGAGGATCCCTTGAGGCCAGAAGTTCAAGAGCAACCT
GGGCAACAGAGTGAGACTTCATCTCTACAAAAAAATGAAATAAAAAGTTAGCTGGCTGTGATGGTGTGGG
CTTGTAGCCTTAGCTACTCAGGAGGCTGAGGTGACATGATCTCTTGGGCCCAGGAGTTCGAGGCTGCAGT
GAGCTATGATAGCGCCACTGGATTCCAACCTGGGCAATGGAACAAAACTCCATTTCTAAAAAAGAATAAA
ATATAAAACTAAAATAATAAATAAATAAAAATGAGGATATATTTTATTTTAACATTTGGAAACTTTGTAG
GTGAGGACCATGCAAACATTCAAGGTGTGAGTTCTGACCAAATCCAATTATTAACCATACCAATGACTTA
AGGTTTCTTCACACTCCTTAAAGTTGATTAATATAATGATTATATAGTTGACTGGTATGTCACAGCTTGA
AGCCITTGAGATTTATTCCTGCCTTTTCTGTAAAGGTTGTTTTGTTAATTCCAGTATGTACTGGTCGTTT
TTGTTTTGTTTTGTTTTTGTTTTTGTTTTGTTTTTTTGAGATGGAGTCTCGCACTGTTGCCCGGGCTGGA
GTGCAGTGGCACGATCTCGGCTCACTGCAACCTCCGCCACCTAGGTTCAAGCGATTCTCCTGCTTCAGCC
TCCTGAGTAGCTGGGATTACAGGCACTCACCACCACACCCGGCTAATTTTTTTTATTTTTAGTAGAGATG
GGGTTTCACTATGGTGGCCAGGCTGGTCTCAAATACCTGACCTCATGATCCACCTGCCTTGGCCTCCCAA
AGTGCGGGGATTACAGGCGTGAGCCACCGTGCCCGGCTGCCAATATGTATTGGTCTTTTTCATCAATGAT
TCAGTCCAAAATCATTTTGTCCITTAACTATATATTTTCTTGTAAAGCTGCTTCTGTTGTCTTGAACTTT
TCTTTTCAAATGTATGTTGTCATTTGACTTTTTAGATTGTTATTTTCTGGTCCTCGAAATAAATTTAAAT
TTCCTGTAAAGGAAGGTGTAATATTCTATTTGACATAGCCGCTAAAGATGTACTAGGTGCTTTATAAATA
TTGTTGATTTACTTTATCTTCACAGATTACTAGTTTTACTTAGTATTTGGAATATGACAACATTTTATAG
AGCTATATTCATATATATGTTTATCTTAACTGTTAAATGCAATATGATTCATGTCTTGTTTTGGTCAATG
ATGAATGAAAGTCTCCTGAGAATTAAATTTACTGCATCGATGCAAAAACAATCATAATTTTAGACACTCT
AAGAATTTTAGAAATTAAAGGATTTTTTTTTTCCAGTTTACTCTGTTAAGATTGTGTTTAGCTATGCGTG
ACAGCATTCTCACTACAGTGGCTTATCCAGATAGTTTCTTTTTCTCATAGAGCAAGACTTCCAGAATTAT
GTGTTCCAGGGTCAGTGCAGCACCTCCAAAACCGTATGTCCCAACTTTTTCCTCCAACCCCAGTCATCTC
CAACATGAGACTTTCTTTTTGTTTTGTTTTGTTGTTTTTGTTTTTGTTTTTGTTTTGAGATGGAGTCTCT
GTCGCCAGGCTGGAGTGCAGTGGCGCGATCTCGACTCACTGCAACCTCTGACTCCCTGGTTCAAGGGATT
CTCCTGCCTCAGCCTCCTGAGTAGCTGGGATTACGGGAACGCACCACCACGCCCAGCTAATTTTTGTATT
TTTAGTAGAGACGGGGTTTCACCATGTTGGCCAGGATGGTCTCGATCTCCTGACCTCATGATCTGCCTGC
CTCAGCCTCCCAAAGTGGTGGGATTACAGGCGTGAGCCACCGTGCCCAGCAAGACTTTTTTTCCTGTGGT
CTCAACATGGCTACTCTGCCTCCAGGCACTATGTCTGTACTTTAAAATGGAAGAAGGGAAAATGGGGAAA
GTAAAAGCATATTCCAGCTGTGTCAGCTCCTGTTTGTAAGGAAAACCAGTGCTTTTCTGGCAGCCCCACA
CAGAAGAGTTTCTACTTGAACAGTGCATTAACCAGAAATGTGTCACGTGACCATTCCTAACTTCTAAGGA
TCTTGGGAGGATTGAGTGTTTTAACTGAATAGGTGTGTTTCTTTCTTCATAATTCAAAATGTGAAAATTG
GTAACTTAGTTATAAAACCTTGCTAGTCTGAACAGAATTTGGATTTTTTTAGCTAAGAAGGAAAGAAAGG
GTATCGGATAGGCAGCTGGCTATGCTAGCCAAGATACTCTTAATAATGCACATTTTTCTTCTTTGGACAT
AAGCAGTTTTAACTTAGCTAAATATGATGTGATTGTTTTCCTGTCTTCTTAGTTCTGTTTAAATTTGTTT
CAGAAATCAAGGAAATAAAATGGAGAAAAAACTCTATTATTCATGTCTATCTTTCTGCCTCTGAATATTT
TTATGTTGGAGAAAGAGAAAGCAGTAACTTTCATAATAGCTTACATAGTCTGACAAATTCTAAACATGTC
CGTTAGCATCAATATACGTGGTATTAGGTCATCAGTTTTTATATTCTGAATTATTAAGACCCAAATAAAC
CCACTGAGTTCAAGAGAAAGTATACACTGAGCAATAAAAACATTACCAGTTCTAGCAATGATAATCAAAC
AAGAAAACAAGTAATATAGTCTGTTTAGAATAACATGTTTTAAAGATCAAGTTTTTCTTCCTTACCAATG
TTGCCTTTCTTGTAACACTTTTTTTTCCTTCTTGAGATAGGCTTTCCTATCTTTTGTCACAAACCCCAAT
ATTTACATGGCCATTCGTAGTCTATTCATAGCAGCACCACCCCATGGCCCAAACTTGTAGATATTGCCCT
CCTTCTATGGTTGTTCTAATAAGAATAACCACTCTTGTCTCTCATAATCTCAGCTGTTTTGTGCCGTTAA
AATGGAAAATAATGAGTATTAAGATACTAACTAGGGGCCAGGCGCGGTGGCTCACGCCTGTAATCCCAGC
ACTTTGGGAGGATGAGGCGGGCGGATCACGAGGTCAGAAGATTGAGACCATCCTGGCTAACACGGTGAAA
CCACGTCTCTACTAAAAATACAAAAAATTAGCCAGGCGTGGTGGTGGGTGCCTGTGGTCCCAGCTACTCG
GGAGGCTACTGCACTCCAGCCTGGGCGACAGAGCAAGACTCCATCTCAAAAAAAAAAAAAAAAAAAATAC
TAACTAGGTTTCAGTCATATGAGATGAATAAGTCCTACAAATCTGTACAGCCTAGGGCTTGTAGTTAACA
ATGTTTTATATTTAAAACTTTGCTAAGAGAGTAAATCTTTTTTTACCTGTTATTATCGTACATGCAAAAA
TAATAGTAAATAAGGAAGGTAAGAGAAAAATTTTGGAGGTGATGCATAGGTTTATGGCATAGATTCTGGA
CATGATTTCACAGGGGTATACAGTCATGCATTGCTCAACAACAGATATACATTCGGAGAAATGCATGGTT
AGCTGATTGATCTTGTTGTGCAAACATCATACAGTGTACTTACACAAACCTAGATGGTACATCCTACTAT
ACATCTAGGTTGTATGGTATAGCCTATTGCTCCTAGGCTATAAACCAGTACAGCATGTGACTGTACTGAA
TACTTCAGGCAATTGGAACACGATGGCAAGTATTTGTGTATCTAATCACATCTAAACATAGAAAAGTTAC
AGAAAAATATTATAATCTTATAGGACCACTGTCCTACATGTATACAGACTGTGCACATGATAGTGGTCCA
TTGATCAAAATGTCCTTATGCAGCACATAACTGTAATTATCTCGAAACTCATCAAGCTGTGTTCATTAAA
TGTGTATAGCTTTTTATGTCAATAAAGTGGTTAAGAAATCAATAAAGTGGTTAAAAAATATTTTGACTAG
GAAATATACTATCATTTCTAGTTGATAAAAGATCTCAACATTTCCAAAATTGTCCTACAGAAAACCAGGT
TCATCAGGTGTTCATACATGATCCTCATGAAAAGGTCAAATAAGCTGAAAAACATGCATAGACGTTGCCT
ATCCTAGCAATCTATGATGTACATCTCCATAGTAAGGTCACTGAAAAGTCTTTTAGGAATGTTAGTATTG
TTAGCTCAGTATTTCTCAGTGATTTCTCCATGGAAACCATTIGTGTAGAGCATCTTGAGGAGCACAGCTG
AGAGAACATTATCTTAGITGGATGTGTATGTCCCTCTGAGTCACTTGATTTCTCTACATATGCTTTTACC
AAATTAATCTTTTAGAAATCTTTTCTCTTCGCACTATGTCTATAATTTGTGAAGTTGTTACCAGGATAAC
ATTTGTGCCTCTCACCATGATGTACCTACCAGGGTCCAAGCAGCCATTCCTTCTCTAGAGCAACTGTCTG
AGGGAAAGAATTTAACACAGCATTCTACGAAATCATTTTATTTATAAAAATAGATTACTGCTTTACATAT
AGTAATTTATATTTAGAATATTGATTAATTATTAAAATCTGCATGAGAGCTTTAAAGAGTAGTACATAAT
ATATAGCAGTTTGTACTCAAACTGTCTTCTAAAAAGGATTCACTTTTTGTTTGTATTCTATTGTCCTATT
CGTTGATAGTGTTACGTAAGTAATTATAAAACTTAAAATCTGGAAAGAGAATGTGGACTCAGAATGCCAT
CTCTTTTGTTATTTCAAATGGATTAGAAATGAACATACATATTCATTTTCTTTCATTACACATCCAGAGA
AATAGAATGGATTTTATAAATATGTAAAAGCAAGGATTTTGATCACTGATAAAAAGGGAAGGTTTGGTCA
CTACCTTATTTCATTCCTTTTTTCTTATCCTTTTTTTTTTTTTTGTCAATTATTTGATGACATCTCTGAA
CATCACCTTTTATTCATGACAAGAATTGGGTATCATGGTAAAGAACACTGTTAATATAATTCAGTTACTT
CACCCCCTCCTGAAATATAGAGAAGCTTTAAGACTATGTGAATATTTTTTTCTGGTTTTCTTGTATTTGT
AGAAATAGCATGAGCTTTGTTTAAAGTCAGGCATCTAAAACCTTGCCCTGTATGTTATTGACAACCTGCA
CAAATTTTAGGATCTATTCTATTACAGTTTGTTCAACTGTAAAACTAGGATAGCAAACTCTATGTCATAT
TTTCGTTATCAGAATTTAAAAAGCATGTTTTAAGATCTTAGTAAATAATAAATCTCTACTCTGTAGTTGA
ATTTGTTCTATATTCTTTAAGAAATTCCCTTTGATGGTTATGCCAACCTCTGTATTACTTTTCTTCACAC
TTTAACTTTGCGCTGAAATCATAGTAGTATTTTACGTTATCAGTCAAAATAACAGTCATCCTTAAAACAA
ATATGAATTTTAGATGATTAAATAGATTTGTATGGAGGTTCTTCTTGCTAATCATAGCAGTTATCCTTGG
TGAAAAATGATAGACACTTGAAAAAACCAATTAATCATGATGGCTATTTTTGCATCATAAATAAAGCTTT
CAAATTTGAGAGGGAATCAAAAGGGCAATGGTAGTATAGTGTCTCAAAGCCCCTTTCCAATTGATGGTAC
AAATTTAAAAAGAGAGAGAGAGAGAGAAACATGTTTCACTGTAATTGTTTTCTAAGAGCTTCCAAAAAAG
CGTATTTTCTTAATAGATTCAAATTTTTCAGTTGGATTGAAAGGGAAGTCTTGGAGTGTAGTGAGGAGGG
CACCTTCTGTTGAGAGGTGTTCAGACGACAGAGTGTGCCCAAGGCCAAAGATGAGATGGTTTTGCGAAAG
TCAGTGGCCACAAACAGGTGTGTTTGACCCCTGAGAGATATGCAGGAAGTCTACCCCACTTTAATTCTTC
CAAATATTCTTTACCTTAATTCCCAAGTACTTGATAAAGGAGCAATGGGGAGAAAATATGCACACTATTA
TGGAAAAGTTTTGACCTACACTTTGGAGAGTTTTAGATTAAGAGCATTCTAGAAATCAGTCCCAAATGCC
TAGGGTTTACTTACTTAAAGATAATATCATAGTTTGGGTGACTGGGAAGCATACCCTGAGATTGAGGTGA
GCATGCAGTATGTCTATTTAGGAGTGTTCTTGGGGTCAACGTGTAGGGGCAGAGGGAGAAGTTGAGCTCT
GACGCAGTCTTAGTAAGGGCCTCAGCTGACCGTTCAGGGAGTTCTTAAGCTGGAATGACCCTTCAGAAGT
GCTAGGAAACGAAGAAAGGGGACTGGATCTTTATAACCCCGTGTCAAGTCATGCACTGGATGTGGGCTAC
TCCAGGAAGGCAACGAACTTTAGCAAGATGATTCTCTTTAGCCACGGGAATTTCCATAAGGGGGCTGCTA
TGGTCTGAATGTTTTTGTCCCTCCAAAATGTGTATGTTGAAACCTAACACTCAAGGTGATGGTATTAGAA
GGTGGGGGTTTGGGGGGGTGATTAGGTCATGCGGGCTCTGCCTTCAGAAACAGGATCAGTGCCCTTATAA
AAGCGGCTCCAGAAAGCTTCCTTGCCCTCCCACCATGTAAGGACACACCGAAGATGCCATTTAACAGGAG
TGGGCCCTCACCAGACAATGAATCTGCTGATGTCTTGATCTTGGAATTCCCAGCCTCCAGAACTATAAGC
AATAAATTCTGTTGTTTATAAATTACCCAGTCTAAGGTATTTAGCTATAGCGGCCCAGACTAAGACAAGG
GCTGACAGCTGAAGGCTGTCTACCAGCAGCACTCCTAGCAGCTGGGGAACTAAGTCCTTCATTTCCAAAG
GGGAATCTAGGCAGCATATTTACAGCTTTTCACTACAGATAAGCTCATTATTTCAAATAGGGACTAGCAG
GAAAAAATTAAATTGCCCAAAATTTAGTGGGATGCTGAAATAGATTGTGGTGTGTAAATTGGAGTATAGT
GAGGAGAGCACCTTCAAACCAGTATGTACTACATGATATTGTTTTTGTTGCAATATTTATTATATACCCA
AACACACATATATTACTTTTAGAAACACACACCACATATATATCTATGAATATTTTATATACACATAGGG
AAGGATTGTTGATGTTATTTATGCTATTTTAAAGATCGATGTTTTCATATAATTATGTATTGGTTATATA
TTATTTCTTGATATAAGGTAAAAAAAAAAAGCAAAACAAACTTTAAGTGATCACTATGAAAAGAATCCCA
ATGCTGCACATTTAGGTTTATCCAACTCTTCCCATTAAAATATTAAATAGTAGAAATAATTGTGAATAAG
AAAGAGCAGATTTTGAAAAATGGAAAGAAATGCTTAAAGACATAGCATTGTTGCCCAACCATCATTATTT
AAACATACAGTGTTTGGCTTTGACCAAATTGCCTTCAAACACTTCCTTTTGGCCCAAAATGTTAGGTCAT
ATATACTACCATAAAATTCATGATGCTTACCATGCATTAATTTCTAGTATATACCAGGCATTGTGCTATG
CATATCATATTCAATATTTCTAATCCTCTCAAAAGTGGTACAAGCTAACTGGCGTTTTTCTTGTTTTGAA
AGGGAGAAACTCAGAGAGGTTAAGTGACTTGCCCAAGGCAATGCCATTGATAAGTGCCAGATTCTATCAC
AGGTTTATTGGCAACAAACCATATGTGCGCGTGCATGCGCGTGTGTGTGTGTGTGTGTGTGTGTGTACAC
ATACACGAATAACATATATGGTATAAATACGTGGAAACATAATAAACTGCATTGAGCTGCGTTTATAATT
AGTATTTAGGACATGTTTGGCAAATAAAAACAGTGGAGATTGAAATGGATTTGCTTAGGAAAAATGATAC
ATTAAAATAGGCTTTATTATGAGTCTTCAACTATTCTGTGAAAATAGATACCCAGGGAAGAAATAATAGA
GAATATGAATCTTGAGCAGGCAACTGAGAACTTGTCGAAGAGCCAAGATAAAAATGTCAGAGAGGAGAAT
ATTTTGGCAGCTCAGATGAGCCCCCAGAGGGTGGGAGGCAATGATCTCACCGCAGTCTCGTATCTGAACC
CCAGGTTTTTGCATCTCCATAAAGTAATTTCTTACACCCCTCAATAATGATCGGGCTTACTCTCAATCTC
TCGCTCTCTCTCTGTGTCTCTCTCACGCACACAAACATGCAGAACATTTCTTGCACATGCATAACTCATA
AGACGATTATGTAAATACCAGCCTTTTTATTTCATAACTAAATTACAAGGCCTGGTTATTGTTTGGACTG
TGAAAAAATAATTATGTGAATAGGTGCCTCAAGATGAAAGACAAGGCAAGATTGTGAAATTATTCATATG
ATAGTAATAGTATGCAAAAAATAACACAATCTTTAAAGATCTTTAACGACCTAGTTATAAAACCATGCTT
TATAACAAATATAACCATGAGGAAATAAAAAGAAAAATGTAATAATATACTCCAAGAATAAAGTCAAATG
TATTGTTGAATGTAAGGAGTTGGTTACACTTCCTTATAGTGGAGGTTATTTTAAAATTTGTGGCTTACGT
GGTGTTATGAATTGCCCTAGATCAACACTATTATGCAAGGCCAACTATTAGGTTATTTTTGGTAGATAAC
CACAGCAAAACTTTAGTATAATAGGTAAAGGTTAGCTACACTCCCATACCCTCACTCTCAGGTGTTGTCA
TACTCCGTATAAAAGGTTCAATCAAGGGAGACATGAGAATATTCCAGAATCTAGAGGCAGGATGCAGTTA
ACCTTAGAGAAGGCATCAGACAACTAGAATCTTCGGATTCAATGTGGAAACAAAGCATAGTTTAGGCATT
AAATCTTGGGCACCATTCCAAAGAATACAGGTTCCATAACTTACTATATTTTTATACCTAGCAAGCTAGA
GATGAGGAATTGCTCTCAAATATTTTAACCAAAGCATGTATCTTAAGTAACACTAATCTCATAAGTGAAA
ACTCATTTCTAATATTCATTTTGCTCATTAGCAAGGCCTCTAGTGTTGACTGTGATAAAAAATAGTTCAA
ATGCTGGTAGAACCCACCCCAGGAGACTGGCCTTTCTGATTAAATTCTAACTCTATCCCCACGTGAATTC
CTGACTTAAGTAACTGAGTTCCTGCACATCAGAATATAAGTATATTATAGATATAAAAACATATGTAATT
AATAAATATTTTAAGTGAGACACTTCTTTCATCTTTATGGCTTAACTATATCAGACATTTGATTATTTTT
AGCGGTCTAACTACAAAACAAAACACAAAGCCCACAACTAAAAATTTCTTTGTATATATTGCAAAGAGGC
AACCATTTGGTGTCAATTCAATCATGAGTGAAATGCTATTATACGAGTACATCTCCCTGGCTTGTATGGG
GGTAATAGGGCATGGAATTTACAGATTCACAATAACTGAGATATTCACAATAACAAAGATATCAATATGT
AGCTTTTCCCATAACTTTGTGTAATGAAATCCTCAGTTTGTGCTGTGTAAAAAGCTTATTGTTTACTTCT
CATGAAAATCATCTTAGTTTTTATCTTTATTTAATAGTCTGTAATTTGGGGGTAATACATTCGTTTTGTT
GATACTATGTGAAGTGGCAAGCAGAAAATTCTAACAGGAATAGATAAGCAAGTATCCTATAAATCAGAGT
CAGTGTCTCTCTCTCTCTCTTTTAATGAGTCAGTCTGTCTCTCTCTCTTTTTCCCTGCCTGGCTATCTAT
CTGTATTTTTCAGTTTTGCTTTGCAAATAAGAGAATTGTGTGTTGTAAACCAACCAACTTACCATTAATT
TTCTCTGAATTCAAAAGCAATTACAAGCGGACTCTTGAGTTTGTGCTGCCTGGTTGTCTGCATATAGGCC
AGATGTCTAGAATAGGATCTTTATTTACTATTTTTACCCTCCTAATTTCATGGTAACTCCAAGGTAGATG
ATATTTGTAATCGTACACTACTTGTCAGAAATCTTTCTAATAACACTGCTATTTTATAAAAATAAACATT
AATTCAGTATAAAATTTTATTTTAAATTGTTAATTCAAGCAAATCAGTGAGGTAACTTTTACACTGCCGA
GCGTACGTGTGTGTGGATTAGTACAGCCATGCCATAGACTTCACTTGTAATCTTTTCTTTATATTTTTTA
TACACCTGAAATGTTCATCATTGTGCTGTAGAAAACAATCTCATTGTGTTTTTAAAAGCTAGAGTGGGTA
TTGAGAAGGGGAAGAGGATCATAGAAAAAGTTGGTTAACATGCTACTTAACACTTCAAATCTTTACTCGA
TGTCATCATCAGCAACATTTTAAATTTATGCTTCTACTAGTTTGCAGTTCTTTTCCTTTGATTATTCTTA
TGATACAAGCCTTTCCACACAAAATTTATGTACAGGAATTGTGTAGAATTTTTCTTTGGAAAATATGGTG
ATTTATTACAATTTGGGCAACATCATCATTTTAAAAATTCAGAATTTGATTTTTCTCAGAATCATCAGAA
AGAATAAAGCATATATATGGTTCATGTCAGGAGAATTAGAACATGAGAATTAATATATCTCTGATCTTTT
AAAATATTTTCATGTTTGTGAATCAGCAGATTTTTCCTAGTTTGAGATTTAAAAAATCTAGATATAATTA
AAATCTCACTGATGTTTCACCATCAGATGATTTTATATTTGTATTTTCTTCCACTTCATAACTTGTATAG
AGAAGAATAGAAGAAAGAAAAAGGGAGGATTGATAATCTTTCTCTCTCAGTTCTTATAGCACTTCATTTT
TTAAACTTATTACTTCCTTCTGCCTGCTTTGTTTGTCTACATGTTTGTATTTCATGATTTCTTAGAAATC
CATCTACTGCCATTCTGAAGGTCATTTACCTGAAAATGATAGAAAGCAGCATATATTCAAACAACTGCAG
AGTAATTGTCTATATCAGTTATCATTGTTCATTACTTTTCTGTTTTAGGATTGAGGGGCTGCCTCGCCAC
CTCCCTCACACCCCCAGCATATTATCACAAAGCCTACTGATTCATTCACATCCCTGGGCTGAATTTGCCA
CCCACTGTGTGTTCCTGTTGTTTTGTGTATGGAAGTGAAAAGATTTAATTTGATGTTGTTGAAAAGACAC
AGAGGCTAACTTTCAATTTTCATATGTAGTTCTTCCCTCTCCCTCTGCACCACCTCCTTTACTTGTTGAG
AAAATTGCCCTCTCCATGGTAACAATAGAAGAAGCTTTCAGATTTTAGTAGTAGTTGTTGCAGAGAAAAG
AATTCAAAAAGTAGATGAAGTTTAAAAATGAAAAAGAGAGAGGAAGACAGCTGGGAAGAAGGCTTAATGT
TTATGAGTGGGTGTGGAGGGGAAGAACTAAGTTGAATGAACAAAGCTGAGCTAAGGGGAAGATGGTTTTT
CTGCATCCCAGAAGGCAATACCCTAGCCTTTCCTGCAGCCTTCACTCCCCAAAAGATAAGAGCTTTATCT
GAAATTCTTATAGGATTCATTCCTGAAGAGCAGCTTGTCACCAAACAGAAACACTGTGATTTCCTCAGGG
AGTCACAGITTATTATTATTTTTTTAATGTAACGCTTTTGTGAACTCCAGTTTCCACCTCAATTCAAATG
GTCTTTTGGTTACAGGGTGAAAGAGACCCAACAATACACCTTTCCCACTTCCGGAGGCCTTTGGTTAAAC
CATGTCTGCCACAAGGACACAGGAGCCTGGTATGACTGGTTGTTTTTTGTTTGCTTTTTTGCCTCCTGTG
CTTTCTAGATTGTGAGATACTGTAACTCTTGTCGATGACACATAGTACCGAACCCACCCGAAGAAGTATG
TCAGTATGTCACATTGTGACAAACAGCTTCTCATGCTAAGTAAATGCAGAACCATTGTGAAAGGTTTAAT
AATGCCCACTCCTCCCCCGCCAAAGATGTCCATATCCTAATCCCAGGAACCTGTGAATATGTTACCTTAC
ATGGCAAAAGGCTTTGTATTAACAGATGTGGTTAAGTTAAAAATCTTGACACGGAGAGATAGCCTGGGTT
ACCCCAGTGCGCCCAATGTAATCACAAGAGTCCTCCTAAGAGAGAAGGAGGTGATGATACAAGCAGAGTA
AAAGAGAGATTGGAAGATGCTACACTACTGGCATTGAAGATGAAGGACAGGGCCAAGAGCCAAGAAATGC
AGGCAGGCTCTAAAAGCTGGAAAAGGCATGGAAAAGAATCCTCCCCTACATCCCTTAGAGGGAATGCAAG
CTCTGCCAACACATTGTTTCTAGCTTGTGAGACCCATTTTTTGGACTTTGGACCTCCAAAATTGTAAGAT
AATAAATTTGGGTTGTTTTAAGCCATTAAGTCTGTAATCATTTGTTACAACAGCCACAGGCAGCTAATAC
AGCCATGAACATTTAGTAATGACTAACTTTGCACAATTTTAATACAAGCTTCTTATTAAGGTTTATTTTT
TCTTAATTACAAGGAATAAAAGTGGGGTCTGGGGGCAATGTCATGGTCCACTCCGTTTTAGCCATATGAA
TTTGTATTTCCAGCATTAGAACAAAAGGTGACAAATCTGAATGTATTTGTGTGAAATAATAATAAAGCAG
AACAAAAAGGGAAAAGTGTCCAGCTGGAAATGAAGTTAGAGAAAGATGAGGAGAAGCAAGCCAATTGTGT
AGTTTTCCCTTCTGCTTTTTAAAATCATGATTTGTTTAACCCACTGAATTCTATTTTAGAAACAGGACTG
CAAGGAAGTGTTGATGGATTTGGTGGCATGAGAACCAGAGTCACAGAGGCAGGAAAGTAAGGAATAAGTG
TTAGAATAGGAAGCAGAGTTGCTTGGGAAGAGACCTTATGACATGTGGACAGGGCTAGACTTAGGAGTCA
GAAAGACCTGAGTTCAAATGCTATCCTTTAGTATAGTTTGAAGTCAGGTAGCGTGATGCCTCCAGCTTTG
TTCTTTTGGCTTAGGATTGACTTGGCGATGCGGGCTCTTTTTTGGTTCCATATGAACTTTAAAGTAGTTT
TTTCCAATTCTGTGAAGAAAGTCATTGGTAGCTTGATGGGGATGGCATTGAATCTGTAAATTACCTTGGG
CAGTATGGCCATTTACACGATATTGATTCTTCCTACCCATGAGCACGGAATGTTCTTCCATTTGTTTGTG
TCCTCTTTTATTTCCTTGAGCAGTGGTTTGTAGTTCTCCTTGAAGAGGTCCTTCACATCGCTTGTAAGTT
GGATTCCTAGGTATTTTATTCTCTTTGAAGCAATTGTGAATGGGAGTTCACTCATGATTTGGCTCTCTGT
TTGTCTGTCGTTGGTGTATAAGAATGCTTGTGATTTTTGTACATTGATTTTGTATCCTGAGACTTTGCTG
AAGTTGCTTATCAGCTTAAGGAGATTTTGGGCTGAGACAATGGGGTTTTCTAGATATACAATCATGTCGT
CTGCAAACAGGGACAATTTGACTTCCTCTTCTCCTAATTGAATACCCTTTATTTCCTTCTCCTGCCTGAT
TGCCCTGGCCAGAACTTCCAACACTATGTTGAATAGGAGTGGTGAGAGAGGGCATCCCTGTCTTGTGCCA
GTTTTCAAAGGGAATGCTTCTATAGTACAAGGCTACAGTAACCAAAACAGCATGGTACTGGTACCAAAAC
AGACATATAGATCAATGGAACAGAACAGAGCCCTCAGAAGTAACGCCGCATATCTACCACTATCTGATCT
TTGACAAACCTGAGAAAAACAAGCAATGGGGAAAGGATTCCCTATTTAATAAATGGTGCTGGGAAAACTG
GCTAGCCATATGTAGAAAGCTGAAACTGGATCCCTTCCTTACACCTTATACAAAAATCAATTCAAGATGG
ATTAAAGACTTAAACGTTAGACCTAAAACCATAAAAACCCTAGAAGAAAACCTAGGCATTACCATTCAGG
ACATAGGCATGGGCAAGGACTTCATGTCTAAAACACCAAAAGCAAGGGCAACAAAAGCCAAAATTGACAA
ATGGGATCTAACTAAACTAAAGAGCTTCTGCACAGCAAAAGAAACTACCATCAGAGTGAACAGGCAACCT
ACAACATGGGAGAAAATTTTCGCAACCTGCTTATCTGACAAAGAGCTAATATCCAGAATCTACAATGAAC
TCCAACAAATTTACAAGAAAAAAACAAACAACCCCATCCAAAAGTGGGCGAAGGACATGAACAGACACTT
CTCAAAAGAAGACATTTATGCAGCCAAAAGACACATGAAAAAATGCTCACCATCACTGGCCATCAGAGAA
ATGCAAATCAAAACCACAATGAGATACCATCTCACACCAGTTAGAATGGCAATCATTAAAAAGTCAGGAA
ACAACAGGTGCTGGAGAGGATGTGGAGAAATAGGAACACTCTTACACTGTTGGTGGGACTGTAAACTAGT
TCAACCATTGTGGAAGTCAGTGTGGCGATTCCTCAGGGATCTAGAACTAGAAATACCATTTGACCCAGCC
ATCCCATTACTGGGTATATACCCAAAGGACTATAAATCATGCTGCTATAAAGACACATGCACATGTATGT
TTATTGAGGCACTATTTACAATAGCAAAGACTTGGAACCAACCCAAATGTCCAACAATGATAGACTGGAT
TAAGAAAATGTGGCACATATACACCATGGAATACTATGCAGCCATAAAAAAGGATGAGTTCATGTCCTTT
GTAGGGACATGGATGAAATTGGAAATCATCATTCTCAGTAAACTATTGCAAGAACAAAAAACCAAACACC
GCATATTCTCATTCATAGGTGGGAATTGAACAATGAGAACACATGGACACAGGAAGGGGAACATCACACT
CTGGGGACTGTTGTGGGGTGGGGGGAGGGGGGAGGGATGGCATTGGGAGATATACCTAATGCTAGATGAC
GGGTTAGTGGGTGCAGCGTGCCAGCATGGCACATGTATACATATGTAACTAACCTGCACATTGTGCACAT
GTACCCTAAAACTTAAAGTATAATAATAATAATAATAATAAAATCTCAAAATAATTAAAAAAAGAAACAA
ACAAATGCTATCCTGATCCTAACTGGCTGGCTGTCTTTGGGGAAGTTGGTAATCTTTTCTGTGCTTATTT
CCTCATGTGTAAAAAAATGAATATAGTACCCAGCTAGGTAGAGTTGTTGTTGGGATTAAATGATGACTAT
AAAGCATCTAGCCCAGCTTCGGCTACATTATAGCTGCTTACGAAATTGTAGTTACGATGTAAAAGAGAAA
AACACTGGAAAAGGAGGATATGGGCCATTTTATTCCACCTTCACCACCTTTTAGCTTGGTGACCTTGGGC
AAATTATGCTTCATTCCGTGCTTCATTTTCCTTGTCTATAAAAGGGTGTAAGTACAGAACCATTGAGGGG
TGGTCATTATTAACCTACCTCAAATGGTGTCTGTAAGTTAATATATATTGTGCTTTTCCTATGTACAATA
TCTAGCACATAATTACAAATCAAATCCATCCCATGTGCAATATCTAGCACATAGGAAAAGCACAATAACT
AGTTATTACTCTTGTTGTAGTAATTGCTACGCTGTAGGAGTTTGAATTGTAAGGCAGTGGAGAGTCACTG
ACCTTTACGAGAAAGTGTAGCAGAACATTTGAGTAGATAGTAATGGGGAATATTACATAAATGGATAGAT
ATTAGGGGCAGATATTACTATTAAAATATTACAGCATGGATATTTATTAAGGCCAAACTGGTTAATTAGT
TGCATCTCTCAGGTTCCTAATGTTGCTTAATTTTTTAACCTCCCATTTTGTGCTGCCCTTTGTACGAATA
TTTAATGCTCCCAACACCTCTTCAGTAGCACATGTACTGIGAGTTTGTTTTGTTATTACTTGTGTGTATT
AGCATTCCTTTGTGAACCAAAAGCATGGAATTAGCTGTTGCCTCTAGGCTACCTAGTTTTGTAGTTTGGA
TTGAAGCCTTCACCTCAGTAACACCTATTCTGTCTACTATCTTACAGAAAACTTGTAAAATTAAGACAGA
TCATTAATATAGCAGAAAGAGACAAAGGGCAGAGAACATTGAGATACTGGATATTGGAACCACCCAATAG
TGTTGATTTATTTATGATTATCAGTTTTTGTCTCTGCCTAGCCTCATGCCACTAAAGTCTCTGAGGCAAC
AAAGAATAAGCAATTTTGCTCACCTTATACAAATAAAACACAGAAAAAGGAATCACTAGAGAAATGGTAC
TGCAGCCTTTCTGCAGGGATTACTGCTTATTTTTAAATTACTTAAAAGGTATTGAAATTATTGTTCATAA
TGAGAAACCTGCCTAATAAAACAGAAAATTAAACTTAACACTTCCCTATAATGTAAACAGCTCGGTTAGG
AACACAACATTACAGAAACCACTTAAGAATTGATTGTACTTGTTCTTGGAGCAGAACTAGAAGCTCACCG
TTTAGAAGCTGTGCACATTTCCCTATCAAACAGTACATAAAGTTTCCATATTCCTCAGAATCGGCTTCAT
TTGTGCCATGTGTTTGCTTGGAACTATGCCACAGAAAGCAGTTCTCCCCCTCAAGCTGGGCTCCTTTCAT
GCCGCAGTGCAAGTGTGTGATATACTGGCACCATGTGCTAATGTAGACCCATTTTTATATGATAAGAATT
AGTACGGCCTAGGGAATAGACAAGTATGTCTAAAATCCTCCCCATAGAATATGTCCCTTCCTTTAAAAGC
TGTCATACTGTAAGTTCCAGCTGAGTTAAAGGCCACTGTGCTCCTATAGGGAAATATATTCTATTGTAAT
TTTTACGTTCTCCAATAACAGTCTGTTCTTTGTTTACTGAAGAGAGCTTTCATGTCATAAAATGGTGTTT
TTTGACAGAGAAGCAGAATCATTGTTTTATTATAGAAATTTGCTCTTACAACAGCAAAAATAAATAGCTC
ATCTCTTAAGCTCCTGATCAATGTCTAACACCTCCTACCCCCAGCAACACTTCACTGCAAGTATATTAAC
ACTCTATAATAGCAATTCCACTCACCTACCAAGAAATGATCTTCACAAATGATTTACAGCTAAACCAGAG
CTTAAACACATAGCACCCAATCAAGGGCAGATTTTTATCTTTTTCCCAGTCATATAAGTTCTGAGAAGAA
ATAGATTAATGTTGATCTCCCAGACAACTGCTGAGAAAATGTACAAAGGATGTTGTTTATTTTGAAGAAT
GAGACCTAGTTGTTAAGCACTTTTTCCCCTTATATGTACGTCCAAAGGTAACCATTACACCATTTTGATG
CAAATTTAGGATATATATTTATTCATACCTCTCTTCTCCATTCGGATGTTGTCTGTGTGAGTGCTCACAG
ACACATGCACACATACACACATGCACTCCTGTTTCACACTTATTTGTAAAACTCACAAGGATTTCCAAGC
CATTAATATAGCATTGTTTAAGGTGAACACATGGTTGTTCACCATCCATATGTATCTTCACTTTGTAGCA
CTCAGAATTTGGCAAAATCAGAAGGCTGAAACCTCATGGATTAAATATATTCTATATAACATATGTCTTA
ATTGCTGTTACTGTAAAGAAACCTGGACTAGCCATATTTGACTAATTTCTACCTAAGGTATTTGAATTCT
TATAAATAGATTCATTGCTTTAATCACACAAGAGTGGTTTATATGAATGTAATTATCTCCACTTTATAGC
TGAATAAACTGAGCCTGATTCTATCCCTATATGGGAAACATGAATTGAACAGCTGTGCCAATTATTTTGA
TAATTCAAATTTCACATCTACCATGTGAAGACAGCAGAAGAGGGTTAGGGGGCTTGAATTATTCTGATTA
ACTGTGTTCATGAGTGTAATCGCCTCTAGATAATCACTCATTTCTTCACTTCACTTCCCATCTACAGGTA
GTATCAGCGAATGGTAACATCTCTTGCTTTGCCTCAGTTTATTGATGGCTGCCGTTAGAAATAAAAAGCA
TGGTTTTGTTTCAGTACTTAAATGAATAATATCTTCAAAATGTTTTTAAAACGTGAAAAGGTTGAATGCA
TTTTTAACAAATGTTTTGTTAGCTTTGACTTTTATTTTTGAACAGATGAGCACAATACCCCAGTACTCCT
TTCCTAGAAATAGGAGTACTACCTGAAGACTTATTTCCCAAAGAAAAATATCAGGTCTAGTGCAGCAATA
CGTATTAAGGGCATTGAAAAGTTATATTCACAAAATGTGACATCATAACATATGTTAATACTTCTTATCA
CTGATAATAATCCTTGAAGTTGTATTTCCAGAGAGATCTCAATTTCTTCTCACACTCTGAAAGTCTCTGT
TTATCCTTTAGAGTAGGAATGTAAGAATTTAACAAAACATTCTGAATGTTTACCTTTTTTCTAAACTGAA
ACTACAATCCCTTTTTACCCCTATATAGTAAAATATAATATTACAAGTAGAATCAGCAAATTTGTTTAAT
AATTCTTTGGGACAGTTTTTACAAGCAATGGGGTTGAATTTATGTTCCTTGTGTGCAGTGGAGTTATTAT
ATTCTTCTTAAAAAGATGCATGAAGGTAAATTAGAAATGTTTTACATGTTTTCATGACAGGACATTTAAT
CAAAGAGGAGATACAAGAGGCTTTTCTTGGGTTACTGCAATTTAATTTTCCATTTCTTTCTTGGAAGGGA
CATTGGATGCAGTTGTACGAGGTTAATATTTCTAACATGCCACTTTTATTGTGGCATTCTTCTGCTCTCA
AACCTCTGATGATTTCCCATGGCCTTCAACATGATGTCCTTATTTGGGCATCTAGTGTTCTGAGCCCTCA
CATTCTGGCCCCAGCTTCCCTTCTCAACTTGATCACAGCCATCGTAATTCTGCTAACTAATTACACAGCT
GCCCTTCCCACTCTTTCTCAACCACTCTGCTGTATTCTGACTTCTACACTTTAGTTTTAAAGCTACTCCT
TGTCCTGAAATTCCTTCTCCCATTAGGTCATTATTAATTGAGTTCATCCTCTAAGTTACAGTTCATGTTG
CTGTTCCTCTATGGCACCTTCCCTGAACCTGGTCCCATATAAAATCCCATCTCTCAGAATCCCATTAGGG
TAGGATATGTGGTCTGTAGACTATTACGTTTGTCATTTACATATTGTCTTCTATTATTGGGTAACTGCGT
GTGCGAGCATGCATGGGCTGGCCTGAAGGTCACCTCCCCAACTGCATTGAAAGTTCATCACAAGTTCAAT
TATTTCACTAGAGAGTCTCTTTCATGTCATCCATGAGGCACATTCCCTACTGTGATGTGTTATGCATACA
ATTATTTCAATAAATATTTTCTAGCTCTTTGATTGACCAAAGCTTAATTACCTGTCAACTCTAGCCTCTT
GTATCTGGAATTTCTACAGTCTTTGGATAGTATCTTTAGGATGCAAAATTAGGAGGAGTATGTACCAGGC
AAACTATTACAAATAATGCCCTCAAATAGTTACATTTCACTATTCATGTCTTGTAATTTATCTTCTGCTT
TGGTATTTTAGTACACTTATGATTCAATTTGCTGTATAGATTCCTCTGAATAGGGACAAGAGAATTCGTC
TTGATAAGTGGAAGTTCGAAGGATTCCAAAATGATGTTATTCAAGGTAGAACAAGAAATTAATACTGAAA
AAATTGAGGAGTAATAATCCCCAAATATGTACATGCGTATCTCGTTTTATTGGGTTTCACTTTATTGCAC
TTTGCAGATATTTCACTTTTTGTAAATTGAAAGTTTGTGGCAAGGCTGCATTGAGCAAGTCCGTCGGGCA
CAATTTTTCCAACAGCATGTGCTCGCTTTACGTCTCTGTGTCACGTTTTGGTAATTTGCTCAATATTTCA
AACATTATTATTATTATTATATTTGTTATGATCTGTGATCAGTGACCTTTGATGTTACTATTGTAATTGT
TTTGAGATGTCATGAACTGCACTCATATGAGATGGCAAACTTTGTGGGGTGCAGTGGCTCACACCTGTAA
TCCTAACACTTTGGGAGGCCAAGGCAGGAGGATCGTGTTAGCCCAGAAGTTTGAGACCAGTCTGGGAAAC
AAAGTGAGACCCTGTCTTTAAAATATATATGTAGAAAAATTAACTGGGCATGGTGGCACATGGCTGTAAG
GAGCCCTGCCAGCTGCATGGGAGGCTGACACAGGAGGATCACTTGAGCCCAGGAGGTCAAGGCGGCAGTA
AGCCATGTTCACTCCAGTGCCCTCCAGCCAGAATGACAGAGCAAGACCCTGTGTGGAAAAAAAAAAAAAG
ACAAACATTTTTTCAACTTTGATTTTAGATTCAGGGAGTACATGTGTAGGTTTATTACCTTGATATATTA
CATGATGCCGAGGTTTGGAGTACAAATGATACTGTCACCCAGGTACTGAGCATAGTAACCGATAGTTAGT
TTTTCAACCCTTGTTTCCCTCCCTCCCCACTCTAGTAGTCCTCCGTTTCTATTGTTGCCATCATTATGTC
CATGACAACGCACTGTTTAGCTCCACATGAGAACACGTGGTATTTGGTATTGTGTTTCTGCATTAATTCA
CTTAGAATAATGGCCTCCAGCTGCATCCATGTTGCTGCAAAGGACATGATTTTGTTCTTTTTTATGGCTG
CATAGTATTCCATGGTGTATATGCACCGTATTTTCTTTCTCCAGTCTGCCACTGATGGGCACCTAGGCTG
ACTTCATACCTTTGCTATTGTGAATAGTGCTGAAATGAGGATGAGAATACATGTGGTTTTTTAGTAAAGC
AATTTGTTTTATTTGGGCTATATGCCCAGTAATGGGATCACTAGGTTGAACGATAGTTGTGTTTTAAGTC
CTTTGAGAAATCTTCAAACTGTTTCACTGTGGCTGAACTAATTTGCATTCCCAGCAACAGTGTATCAGAG
TTCCCTCTTCTCTACAGCGTCAGCAGCATCTGTCATTTTTTTGACTTTTTAATAATAGCCAGTATGAATG
GTGTGAGACGGTATCTCGTTGTGGTTTTGATTTGCATTTCTCTGATGTTGAGTGATGTGGAGCATTTTTT
CATGTTTGTTGGCCACTTGTATGTCTTCTTTTGAGTAGTGTCTGTTTATGTCTTTTGCCCATTTTTTTTG
ATGGGGTTATTTGTTTTTGACTTGTTGAATTGTTTAAGTTCCCTATAGATTCTGAATATTAGACCTTTGT
CAGATGCATAGTTTGCAAATATATTCTCTTATTCTGTAGACTGTCTGTTTACTCTGTTGATAAATTCTTT
CACTGTGAAGAGCTCTTTAGTTTAATTAAGTTCCACTTGTCAATTTTTGGTTTTGTTATAATTGCTTTTG
AGGACTTAGTTATAAATTCTTTCCCAAGTCTGATGTCCAGAGTGGTGTTTCCTAGGCTTTCTTATAGGAT
TCTTATAATTTGAGATCTAATGTTTAAACCTTTATTCCATCTTGAGTTAATTTTTGTATATGGTGTAAGG
AGGGGGTCCAGTTGCATTCTTCTGCATATGGCTAACCAGCCATCCCAGCATCATGTCTTAAATACAGAGT
CCTTTTCCCATTACTTATTTTCATAAGATGGCAAACTTAATCAATCAATGTTTTGTGTGTTCTGGCTACT
CCACTGATCAGCCATTCCCTCATCTCTCTTCCTCTCCTTGGGCCTCCCTATTCCCTGAGACACAACAATA
TTGAAATTATGCCAGTCAGTAACCCTACAATGTCCTCTAAGTGTTCATGGGAAAAAAAAGAGTCACATGT
TTGTCACTTTAAATCAAAAGTCAGAAATGATTAAGATTGGTGAGGAAGGCATGTCAAAAGCCAAGACAGG
CTGAAAGCCAGACCTCTTGTGCCAGTTGGCCAAGTIGTGAATGCAAAGGAAACGTTCTTGAAGAAAATTA
AAAGTGCTACTCTGTTGAACACAGGAATAAGAAAGTGGAACGGCCTTATTTTTAATATGGAGAATGTCTT
AGTGGTCTGGATAGAGGATCAAAACAGCCACACCATTATCTTAAGCAAAAGGCTAATCTAAAGCAAAGGA
CTAATTCTCTGCAATTCTGTGAATGCCGAGAGAGGTGAGGAAGCTGCAGCAGAAAAGTTGGAAGCTAGCA
GAAGTATGTTCATGAGCCTTAATGAATAAGCCCTCTCTATAACATAAAAGTGCAAGGCAGAGCAACAAGT
GCTGATGGAGAAGCTGCAGCAAACTATCCAGAAGATCAAACTAACATCTAAGTTAATAAAGGTGGCAATA
CTAAACAACACATTTTCAATATAGACGAAACAGCCTTCTATTGGAAAAGGATGCCATCTAGGACTTTCAT
AGCTAGAGAGAAGTCAATGCCTGGCTTCAAAAGTTCAAAGGACAGGCTGACTGTCTTGTGAGGGGCTAAT
GCAACTGGTGACTTTCAGTTGAAGCCAGTGCTCGTTTACCATTCCAAAAATCCTAGGGCCCTTCAGGTTA
TGCTAGATCTAGTCTGCCTATGCTCTGTAAATCAAACAACAAAGACTAGATGACTGCACATCTCTTTACA
GCATGGTTTGATGAACATTTTGAGCCCTCTGTTGAGACCTACTTCTCAAAAAAATGTGTCTTTCAAAATA
TTACTGCTCTTTGACAATGTCCCTGGTCACCCAAGAGCCCTGAGGAATATGTCCAAAGAGAATGATTTTA
TTTTCATACCTGCTAACACAACATCCATTCTGCAGTCCTTGGATCAAGAAGTCACTTCAACTTTCAAGTC
TTATTACTTAAATCATACATTTCATAAAGGTATAGCTGCTATATTGTGGTGAGTCCACTGATGGATCTGG
ATAAAGTAAACTGAAAACGGAAAGTCCTCACCATTCCAGATGCCATGAAGAAAATTCATGATTGAGGGGA
GGAGGTCAAAATATCAACATTATCAGGAGTTTGGAAGTAGTTAATTCCAACACTCATCAATGACTTTGAG
GGTCCAAGACTTCAGTAGAAGAAGTCACTGCAGATGTGGTAGAAATAGCATGAGAACCAGAATGAGAAGT
GGAGTCAGAAGGTGTGACTGAATAGCTGTAATCTCAAGGTAAAACTTCAGCGGATCCAGAGTTGCTGCTT
ATGGATGAGCAAAGATTGTGGTTTCATGAGATGCACTCTACTCTTGGTGATGATGCTGTGAACATTGTTG
AAATGACAACAAAGGATTTAGAATATTCCATAAACTTAGTTGATAAATTAGCATCAGGGTTTGAGAAGAT
TGACTCAAATTTTGAAAGAAGTTCTACTGAGTAAAATGCTATCAAACAGCATCACATGCTACAAAGAAAT
CATTGGCTATAAAGGAGAGCCAATCGATGCAGCAAACTTCGTTGTTGTCTTATTTTAAGAAATTGCTACA
GCCATCCCGACCTTCAGCAACCGCCACCCTGATCAATCAGCAGCCATCCTCACTGAGGCAAGAGCCTGTA
CCAGCAAAAAGATTATGACTCCCTGCAGGCTCAGATGATTGTTACCATTTTTTTTAGGAGTAATGTATTT
TCAAATTAAGGTATGTACATTTTTTAGATACAATGCTATTGCACACTTGACAGATGATAGTAGAGTGTAA
ACATAACATTTAATGCACTGGGGAACCAAAAATATTCGTGTGCCTCATTTTATTGTGATATTTACTTTAT
TGCAGCAGTCTGGAACCAAACACACAATATTCCAAGGCAGTCCTATATATTGTTTCATGTCTCTCTTTCT
TCTAAAATGTGTGTAGGAGAAAAAATTATATCACTTATGTTACTGGGCCATAATATCAAAAGCCTGCGAA
TCTGATGCACATGATAAAAACTGGTCTTAAACCTGTCTTGATTATCCTTTGTTAATATGCCAAATTTATA
GAACAATAGAGTTTCAAGAAATGTGAACAATGTAGAATAACTAAAAGATCTAACGTTGAATAGCTAAAAT
TATCAACGCCCTTTATATCACTTTAGAAATGCGTTTGCAAATCATTATCAACAAATTGTAGCATAAAATT
CTTTTTTTTTCTTGACTTTGAAATTGTATTTCAAGATCCGCAATTTACACCACATTATTTACTTACTGGC
TTGTGAAAGTGAAAGGCATTTTCATTTTTGGATGTTAAGGGTTTTAGTAAATGACAAGTAAATCAATCCT
TTAAGTTCCGTGTGGTATAATAGCTTGGAAAGGACCATCTTAATCTTTTTTTCAACACAGGGAGATAATT
TATTTTAAAAATAACACACTAATAGTAGATTAATATTATTACCATTTCAAAGAAGTCACCAAATTTGGTA
GGCTTAAGAGGTTTTTTTTTTTTTTTTGAGATTACTATGCTCTTTTTTTATTTTATTTATTTTATATTTA
TTTATTTATTTTTTAGTTTTTTAATTTTACTTTAAGTTCTGGGATACATGTGCAGAACGTGCAGGTTTGT
TACCTAGGTATACATGTATCATGGTGGTTTGCTATACTCATCAACCCAACATCCAGGTTTTAAGCCCCCA
ATGCATTAGGTATTTGTCCTAATGCTCTCTGTCCCCTTGCCCCTCACCCCTTGACAGGCCCCGGTGTGTG
ATGTTCCCCTCCCTGTGTCCATGTGTTCTCATTGTTCAATTCCCACTTACGAGTGAGAACATGCAGTGCT
AGGCTTAAGAGTTTTTTTAACTCCCCAAAATATCAACAAATTGAAACATTACTACAAAGAAATAGAGAAA
TAAATTTCACTGACTGTCTTATTGTTTTTTAAGTTTGAATAGCTAATAATGATTTCTTAAACAGCTATCA
TATTTTTTATTTTTAAAGCTAGCCAAATGATCAGTGATTTTTATAATACTGATAAATACTGCTTAGAAAA
GGAACATGTGTTCTAGCAATTTCCACACATTTCTGATTCTAATTACTTGTTTCTTTTTGTTTTATTTTTA
TATTTTTAAGTTTTGTGAGTACCTAGCAGGTGTATATATTTATTTGGTACTTGAGATGTTTTGATACAGG
CATGCAATGTGTAGTAAGCACATCATGTAAAATGGGGTATTCAACCCCTCAAGCATTTATTCTTTATGTT
CCAAACAATCCAATTATACTCTTTTAATTAATGTAAAATGTACAATTAAATCATTATTGGTTATAGTCCC
CCTGTTGTGCTATCAAATAGTAGGTCTTACTCATTCTTTCTAACTAATTTTTTGTACCCAGTAACTATAC
CTACACCACCCCCACCTCCCCACGACCCTTCCCATCCTCAGGTAACCATCCTTCTACTCTCTATGTCCAT
GAGTTCAATTGTTTTGAATTTTAAATCCCACAAATAAGTGAGAACATGCAATGTTTGTCTTTCTGTGTCT
GATTTATTTCACCTAGCATACTGACCTCCATTTCCAACAATGTTGTTGCAGATGACAGAATCTCATTCTT
TTTTGTGGCTGAGTAGTATTCCATTGTGCATAGGTACCACATGTTCTTTGTCCATTCATCTACTGATGGA
CACTTAGGTTGCTTCCAAATCTTGGCTATTGTGAATAGCGCTGCAATAAACATGGGAGTGCAGATATCTC
TTCAATATACTGATTTATTTTCTTTTGGGTATATACCCAGCAGCGGGAATGCTATATTATATGGTAGCTC
TATTTTTAGTTTTTTGAGGAACCTCCAAACGGTTCTCCATAGTGGTTGTGCTAATTTACATTTCCACCAA
CAGTATACAAGGGTTCCTTTTTCTTCACATACTTGTCAACATTGGTTATTGCCTGTCTTTTGGATATAAG
CCATTTTAACGGGAGTGAGGTAATATGTCATTGTAATTTTGATTTTCATTTCTCTGGTTATCAATGATTC
AGTGATGTTGAGCACCTTTTCATATGCTTGTTTGCCATTTGTATGTATTCTTGATCTGCCACAAGTCTCT
AATTACTTGTTTCTTGTACCACTGITTCCCTTCATTGTAGTCAGTGATTTGGTCAATCAACACTTTTTAC
GTATGGGTATCTGTTAACTGTATTTCTAGGAATGGACCAAGAATTGAGAAATTTATCCCAACCAGAAAGA
ACAAAATCTATGGAGGCACAGGATTACTGAAAGCTTTGCTCCTATAGCCTCAGTTTTTTTCTGCAAGTTC
GCTGCTTCCCAGTTGTCCTTGTGATAAAATTCAAAACCTCAAACTGATTTTTAAAAAGCCAACTATATAC
TGCTTCTACTATGTCTACCTAAACTTATTTGCCATTATTTGTAAAGAATTCCAGGCTCTAACCAGCCCAT
TTGAGGCTTTATTTTACGTTCATGCCTTTGTTCATGTCTGTCTTTCCCTTATAATGCTCTTCTCATTCTC
TAGCCAAATCTGCCAAAGTTCAGCTGTAGTGCCATACTTGATATGAAGTCTTTGCTGAGAATTCCCATAT
GTGGTCATTTCCTTCACTGATTTGTTTCAATAGTTGTTGCCATTATTATTATTTTTTTTATACTGTTGCT
GGATACTTTCGCAGATCCAATCTCTAACCTGTTGTTCTACCCTAGGAGGTTGACCAATATGGCAGTAGCA
TTGGGCTTGCTTGTCTATTGGCTTGGTCCTTGTGTTGGGAGGCATCAGCAGATCAATGGATGAGAGGAGA
GTGAGTCGAGGGATGGCTAGGTTCCTTTACTCACAGCCCCAGCTCCTGTCAAGAGGTCTTGTCCCTGCAG
CCACTTCTCAGATTCTACTAACTATACCCTCCCCTTAGCCTTTCAGGCCTTGAGGAGGAAAGCCTCCTCA
TCCCACTTTGAATGAACTATTTATTGCAAGAACCATGACTGATTCAGAATATAATCTGTCCTTGAATGAA
ATAGTTAATTTCCCCTTTTGTATTCTCGTTTCATATCCCTGTCCCTTTATCCATTCCTATATTATATCAT
ACTATCATATAGCTACCTTATATTATACTATGACATGAGTACCTTGTAAGTATATCATGCTATATTATAG
TTCTTAAGTGTGTGGGCTTTGGGGCCAACTTACCTATGTTAGAAGTTCTGCCACTTATTACCATGTGATC
CTAGACAAATTGTTTAACTTCTTTTTCATTCATTTTCTTTACTAGTAAAATAGGGGCAATATTAGTACCC
ACCTCACAGAGTAATATGATAATTACATTGACTGATAGTTTTTATTTAAAACTTTTATTGTTCTTACTAT
GAAATGGGAAATGTTCTAAACACCTTACAAATATTAACTCTATTAATTCTCATAATGAATCTGAGAGGTA
GAGACTCAGTATCTCCATTTCACAGTGCTGGAAATGATACAGACAGCATTTAAGTAACATACTGAAAATA
GTAAACCGAATAGGTGGCAGCCAGTATGCAAATTGGGAAAGCCTGACTCCAGAGTTCATTCTTTTAACTA
GTATGCTGTGATAGACTCTTTTGGCATATGGTATGCACTCAATAAACAACTATTGTTGTTGTCGTTAGCA
TTACTTCCCATTGTATTATTACACAAAAAATAATTGTTTATGAGTATTTGTTTGCTAGATTATAAGTTCC
TTGAAAAAAGAAGACAAGTTTTATTTGTCTATGTGTACTAGCTGAGTGCTTGGCAAATAGTTGCAGTTTA
GTAAATGTTTCTAAAACAAATTATTAGTTGTTTCTTATGTATTTCCCAAGTCTATCCTAGCCTTGGAAAC
AGCTAACACTTAGCTAAACCTAGAAATGTCATTTGAGATTTCAGCAGCCATCATTGTTGCTGAAGCCACA
GGTTCTCCTTTTTATCTCCAATTTCTTCCTCAGATGATTCACCACTTTCTTTGTCGTTTCCCTACTTCAT
TTTTCCTATAGTGACTTTTGTTCTCCAATAACCATTGATAGTGATGACAGTCCCACCTTGGAACCACTTC
TAACTGTCCTGTTCAGCTTTTCCTGAGGCCAGATTTCCCCCAAAATACATTTTTTAGCTTCATAACTGCT
CCTTCCAGAAATTTGAAACGCATTCTCAGGAATTAAGCAAGAGGCATTTGATTGACGTCATTTAATTTGT
GTTTAAATGTGTTCTTCCCATCAACGATTAACCAGTGCTTCAGCCAAGATACATAACTTTTATTCTCCAC
TAACCTTAGGGTTGAGGTCACAATCAGGTACGTAATCTGTAGAGCCCAGTGAAAAATGAAAACGCAGGGC
CCTTGGTTAAAAAAAAATTAAGAATTTCAAGATGGCAACAGTAGAGCATGAAACCAAGTATGAATCCCTT
CTAATGCAGATCCTTGTGTAACTAACTGCACAAGTTATACGTTCAAGAAGCTGGTCCTGGTTGAGGTCTT
TGTCTACCTAGGGCACTTCTCACTCAGAGAGGTGGAATAATCTTAACTTTCTGTCTTCCGTCATCTGAAA
ACTTGCCCCAAAGTTCCTTTTGCCATCTCTTGCAGCAAACCTAGTGGTTGTTATCTGTTAAGGATTCCTG
GTGGCTTGCTTTAGTTTCCTACAGTTCTTGAGCTGCATGTGTTGAATGGAACTCCACCATTACCATGTAA
CACATCTGAATACTCTTATTTTCCTCCAGTTAAATTCTGCGCTCCTTGGAAATAATGGTCATGCACCCTA
CCTTAGTGATTTCTAAATAGAGGTAGTATGTAAAAATACATATTTACTTGAATTGCATCTTAAGAGGCCA
GCCCATGGGAGGCATGAAGAAACCTATGTTACTAATTAATATTCAATAATTGACAGTTACACATTCAGGT
AGTAGTGTCATGGAACCATCACGTTACATGGAACTGAAGAGCTGATGAGAGGTTTAGGGTCTAGGAGGAC
AAAAGTGAGTGTATTAGTCAGGGCCTGAGGTTATATGTACCCAGACAGGATAAAATGGGACAATATTTTA
TTAGTGGAAATATCTATGTGAAAGAAAAGAAGGAGGAAGCCAAGGAAGGTGCAAGAGATGTTAGATCAAG
ATGCAATTCTGACTCTGCAAGGAAGAGAGAGGGAGAGAAGGCTAGGCAGAAGCATCCCAGTGTGCGGTCT
AGTGGAAGGAAATTTTGGCAAAGCTGTTGGGAAGTCATTGAGGCAGAGCCAGGCAAAGAAGTCCCATGTC
TCCCAAGGAAGGCTCTCTGCCTTAGTATTCCCACCACACCCAATCATTGGGTGAGGAGAAGTCTGTGAGA
AGCTTGGCTTTGGTGCAGTGCAATCATGGATTTCAAAATGCAGTAACAGGAGTCCTCAGTCAGTTAAGAC
CCAATAATAGAAGGCCTGCATATTCTCATGGTGGCCACTTGGGTATGAGGAGCAGTGGTGTTTAAAACTG
TTTTGTATAATTTAAATAAAGAAAAGCTGAGGACTACTTAAGCTTGATTCCTTCAGAAGACAGTCTTTGG
CCTTTATATTTCATGTGATATCTTTGCCATATGCTCTGATAACTCTCCATGTTTCCCCTACCATAATACC
TCAGGTTGTTGCAATTGCTTTTTTATTGCCTGTCATCACTACTCGTTTGTTTTCTCTTTGAGAACAGGAA
TTATTTTCTCCTTCACTGCTACAGCCCCTGCACCTAGCTCAATGAGTGACACAACAGAAGCACTTAAAAA
ATTGCTATAACTCAAATTTGGGGGATATTCTACAAAATACCTGGCCTCTGTTGGTCAAAACTGTAAGTGT
TATAAAGTTCAAAGAAAGGCAAATAACTATTTCAACTTAAAAAAGACTAAGGAGATACGACAACTAAAAG
CAATGCGTGAGTCTGGACCAGACCAGGAAATAAAAATATAGCTATAAAGTTCATTAATGGGGTAATTGTC
ATACTCTGAATATAGAGTATGGATAAGATTATGGTACTAAAGCCACGCTAAATTTCCTGACTTTGAAACT
GAGCTGAGGTTATTTAAGAGAATGTCTTTGCTATTAAGAAATAAAACCTAAAGTATTTAAGGGTAAAGGG
GCATGATATCTGCAAATTATTCTCAAATGATTCAGAAAAAAAATATATATATATAAAACATTACATATAC
AGTTATATATCTATACACACAGAGAGAATGATAAAATGATAAATAATTAAAAATAATGTGGCAAAATGTT
AACAAATGGTGAATCCGGGCAAGGGTTACCTGGGAGTTCTTTATCTTGCAAATATTCTGCAAGCTTGAAA
TTATATAAAAATAAAATGCATCTTATAAAGTATTTATTCAGTGAATAAAGAACAAAAAAGGCTAACTGCA
GTTGGAAGATATTTATGAAGTTGGTTATGAAGATTCTTAAGAAGTTACCGATGGAGGTGCTAGTAGAACA
TTCAAAAAAGAAGTAGGGAAGGTTGACCAAGTAGGAAAATATCAATATCCAAGCCAGTATAAAATGGAAA
TGAGAAGTGAGGAAGTAAGTGGAAGACAATGAGGATGGTTTTCGATTTGGCCACTCTATTTGGAGAGGCA
GCCGAATGCAAGAATAGAAGCCAGAAGAAGATGCTCAGTGAGATGGTTGAAAGCTAGATAGATTACAGCA
TCCTCACCAGTAAAACCCTTTCGGTAACTAGAAAGGCTACAATTTAGTACCTTCCTGACTTCTATGCTTA
TTTTCTTCAATACATAAAATGGTTCCGTAAACTCTTTTACCTTCTGAATTCITTATATTAATTTTTTGAA
GTTGTAAATAAAATAGCATCAGTTCTACATTGTTACATTTCAGCTTAATTCATATTCATTTACTGAAAAT
GGGAACATTTGAAAAATCATCATGGGCATTTATGCTATGTAGATTGTTGATTTTTATAGAAAAATATAAA
AATATGACCAGTTTGATTTTCAAAGTCTTTTCTTAGACATGTAAATACTAAGCATTCAACTCAACATATA
GAGTTTTTATTTGAGTATTATTTAGGTGGAATTCTATTTTAATGAATACAATAAAAAATTGTAATTTTGT
CTAAAAGCCTAAAATGCCCTAGTTATAATATGTATGATTTCACTGTTTAACTTCCTATTTCATAGGGTTG
CTATTTATAACCACTTCACTCAACTCTGGGGGGACTTAGTGAGATTAAAGACTTCTGATTCACTTTGTAT
TTGAAGAATTTTTTTTCCTCCATCTTTGCTCAGCTAGTGGAATCCATGATGAATTCTCATCTCCAAGGGG
TAAGCAGTTTTTAGTAAAGCCCAGTAGCTGACTTATGACTCCTTAGAAATAGCATTGATTCCTTCCTTCT
CCTGTGTTTTGTTTCCTCTAGAATGATAGAATCCATGTAGACACGATCCATTATCATGCTTAGGTACTGG
TAAGCATGTAATGATTTTAGTTTTGTTCGCTTTAAGTTATTTGTGTCACAAATATCTGGGATCATATCAG
AGAAATAAATAAGCACAATTAGCATTCTACTTGTTTGTTATGACTAAAGCTAGGTTGAGGAAACAGAAAA
GGACCAGAGGTCATATGAGGATGAAGATAATACTAGGAACAGCATGTTTGGGAGAGTAACATCTGGTAGG
GGTAGCAGATTGGGGGCAGAGAACAGAATTTTATAGATGGATATTTTGGAGGCAAGTAGTTTGAGTAATG
ATTAGATCTAAGGTGTTTTCTCATCTGTGGGTGGCTCGAAGGAATAGAGGTGAAGGTCAGTTTATTTGAG
AAGTTCTGGAATTATAAAACTAAGTTGAAGTCAAAGAAAGTATAGTAGCAAATAAATAGAATACCCTTAA
AAGGAAACCAAATGAAAAATAATCGTTACTCTCACCATATGCTTGTGTTCTTATTAGCAAGAAATTCTTT
TAACCACTGTTTTTATAATATCTTAATGAAAAAATACTGAAGCGTATGCCATATTAAATCCCTCTCTTTA
TTTCTAGAAAGGGAATCAAAGGAGAAAATTCCCATTCTGCTATACTAAAAGACCACTAAGTAAAGAGCCT
ATTAGTGTATGATAAATCCCATAGCAATATACATTATCATTTTACAGCTTCTTTGTTGAAATGAATGTTT
GTATGTGTTGACCATAGAGTGGGATAAAAAGTTGAAATTTTGTTTTGAAATATTTTAGAAATGCATAGTT
GTACTGCAGTTGTGAACCTCCTTAGATTTTTAAGGAGGCTGCTTCAAAGGATCTCATTAATAATCTTCTC
AGGTGCTTACAAAGCATGTGTCTGTCAGCAGAATTAGAGAATCACCCAACTAGAGAACAGGTTTCACAAT
ACCCTGAGACCTATTTTGTTCATTAGAGAGGAAAATGGCTTGTTTTGAGTCTAAGTTGACATGCTTGCTA
ATTTCAGCAATAAAAGCTGTTCATTGTGGTCAGGTTTAATTTAGAGCCTGGTAAGGTTCAGATTAAAGTT
GATCAACTTACTTTTACAACATACTTCTTAAATGAACTTTGAAATCTTAAAAGAAGGAAAAAAGTATAGC
AAACAGTGAATAATGTATCTAAAACTGAGAAGCAAAAAAAATCTGGTTATGTGAGAGTGAATTAAAAGAA
GAACAACCCAATAAAGATAATCTTTGTTATATAAAAATTTCCAAGTATCGAAAAGCACGATTTTTCATGT
GAGTTACACACTATACCGAATATATTTGTCCACTGCCACATGCATAGTCCCTAGAATAGTGCCTAGTGCT
GAAAAATATTTATTAAAATGAATGGATGAGTAAATGAATTTATGTATTTTGCCAGCCCTGTGTATTTAAA
GTTCTCTGTTAACTTTGAGGTGAAAATTTGACTTCATCTGAGGTTTCTGGGTAAGTCCGTTTTAAAAATT
CTATTGAACATATTCAAACATTTTAGGGTAGGCAATTCCAAAGCAACCTTTCAGCTTCCCATGTCACAGA
TGACCAGAGTTTCACATTCTAACACTGGAAAACATCTTATTTCATAAAATCTACCTGCTACTATATGGIT
CCTACTTTAAAATTTGTTCAGTACTCTCCAGCTGACTTATGCCACTTACTTCAATAGCTGTCTTTGGCAA
TTTGTTCCATATTTCAAACACTCTGTTGTGTAAAGAAACCATAAAAGTTAGAAACCTGAAAATTGGATTT
TTTTTCTGAGCAATCACAGCTTACAAATGTGGAAAATTTGTTAAAAGTTAGCCCCTCCAATTTTTCAATA
CAGAGAGGAGAAAGTGCCTAAAGTAGATGTACAATGTTTGGAAAAGTTTTTTGCATTATTTTACTATTAC
CAAAAGCAATTGAGTTTAAATCACAAAGCCTGTCTCCCTACCTCTTCACAGAAGAAACACTACAGAATGA
TCAAAATTTGGCCTTTCCAAAACCAAAATTCGTTAGAAAATCAGCAGGAGTCAAAGTACAGAGTAAATAA
CTAAGTTTCATATAAGTTTCAGATACATTACTATCTACCACTTTCATCTCTTCATCTTCATTGGCCTCAT
GTGGTAGAACATCATATTTAAAATTATACAAACTTGCTGGCTTGTTTACTAGTGTGGTTATTATAAGAAA
AAAATGAGAAAATATAGATAAAACATCTGTCACATATTGCTTATAAACTAACAGTAAATATTACTTGTAT
TTTCCCCAATTAAAATAAAATTTACTGAGTTTTAGAACCAGAGCTAGTCAGATGCCTTTTTTTCATAAAT
TTCTTCATAAATACCTCTGAGATTGTGGTCCTTAAATCTAGAGAGACTAAGATGACAGAGAAAATAGACA
CTGAAGAAAGGGAAGAATATCTTAATGATTTACATTACTACCTATAAATTAAAAATTGTTAACTTTATTA
TATTTGTATTTTTATTTAAAATAGTGCTATATTAAAGTCATTTATAATACAGGGGAATAGGAATACTAAC
CTGTAATCTGATGCTCTCCAAACTTGCCTAAATCATAAAAGTTAATTAGATAATTTATTTAAAATGCAAG
ATTTGCAGCCCTTTCCACTACATATTCATTAGTTCTGGAGGGAGGCAAAAAGGTTTGGTGATTCTTACGG
ACAGGCAGCTTAGGAGAAAAGCTGATTTAGCTCGTCTACTTCACCTTTTCATTTGACAGGTGAGAAATCT
CAGGGGTACAATGAAGTTAAATAAGTAATATCTCTTAAATCGGTTCTGTGCTTTTTCTGTTTTTAAAATA
AATATACCTTAATTTTGACGTCACACAGAATGATATTATAAGTATAAATAGTTATCTATCTTTTAAATAC
ATTGTCGTAATTCAGAATAACATTTCTTACTCAAGGCATTCAGACAGTGGTTTAAGTAATCCGAGGTACT
CCGGAATGTCTCCATTTGAGCCTTTAAATGAAGAAAATCTATAGTCAAGATTTTCATTTGAAATATTTTT
GATATCTAAGAATGAAACATATTTCCTGTTAAATTGTTTTCTATAAACCCTTATACAGTAACATCTTTTT
TATTTCTAAAAGTGTTTTGGCTGGTCTCACAATTGTACTTTACTTTGTATTATGTAAAAGGAATACACAA
CGCTGAAGAACCCTGATACTAAGGGATATTTGTTCTTACAG

Homo sapiens dystrophin (DMD), intron 51 target sequence 1 (nucleotide positions 1570651-1570700 of NCBI Reference Sequence: NG_012232.1)

(SEQ ID NO: 847)
GTATGAGAAAAAATGATAAAAGTTGGCAGAAGTTTTTCTTTAAAATGAAG

Homo sapiens dystrophin (DMD), intron 51 target sequence 2 (nucleotide positions 1570651-1570693 of NCBI Reference Sequence: NG_012232.1)

(SEQ ID NO: 848)

GTATGAGAAAAAATGATAAAAGTTGGCAGAAGTTTTTCTTTAA

Homo sapiens dystrophin (DMD), intron 51 target sequence 3 (nucleotide positions 1570703-1570765 of NCBI Reference Sequence: NG_012232.1)

(SEQ ID NO: 849)
TTTCCACCAATCACTTTACTCTCCTAGACCATTTCCCACCAGTTCTTAGG
CAACTGTTTCTCT

Homo sapiens dystrophin (DMD), intron 51 target sequence 4 (nucleotide positions 1614751-1614804 of NCBI Reference Sequence: NG_012232.1)

(SEQ ID NO: 850)
TATTTCTAAAAGTGTTTTGGCTGGTCTCACAATTGTACTTTACTTTGTAT
TATG

Homo sapiens dystrophin (DMD), intron 51 target sequence 5 (nucleotide positions 1614793-1614847 of NCBI Reference Sequence: NG_012232.1)

(SEQ ID NO: 851)
CTTTGTATTATGTAAAAGGAATACACAACGCTGAAGAACCCTGATACTAA
GGGAT

Homo sapiens dystrophin (DMD), intron 51 target sequence 6 (nucleotide positions 1614612-1614861 of NCBI Reference Sequence: NG_012232.1)

(SEQ ID NO: 852)
CGGAATGTCTCCATTTGAGCCTTTAAATGAAGAAAATCTATAGTCAAGAT
TTTCATTTGAAATATTTTTGATATCTAAGAATGAAACATATTTCCTGTTA
AATTGTTTTCTATAAACCCTTATACAGTAACATCTTTTTTATTTCTAAAA
GTGTTTTGGCTGGTCTCACAATTGTACTTTACTTTGTATTATGTAAAAGG
AATACACAACGCTGAAGAACCCTGATACTAAGGGATATTTGTTCTTACAG

Homo sapiens dystrophin (DMD) intron 51/exon 52 junction (nucleotide positions 1614832-1614891 of NCBI Reference Sequence: NG_012232.1)

(SEQ ID NO: 853)
CCTGATACTAAGGGATATTTGTTCTTACAGGCAACAATGCAGGATTTGGA
ACAGAGGCGT

Homo sapiens dystrophin (DMD), transcript variant Dp427m, exon 52 (nucleotide positions 7787⁻⁷⁹⁰⁴ of NCBI Reference Sequence: NM_004006.2; nucleotide positions 1614862-1614979 of NCBI Reference Sequence: NG_012232.1)

(SEQ ID NO: 854)
GCAACAATGCAGGATTTGGAACAGAGGCGTCCCCAGTTGGAAGAACTCAT
TACCGCTGCCCAAAATTTGAAAAACAAGACCAGCAATCAAGAGGCTAGAA
CAATCATTACGGATCGAA

In some embodiments, an oligonucleotide useful for targeting DMD (e.g., for exon skipping) targets a splicing feature in a DMD sequence (e.g., a DMD pre-mRNA). In some embodiments, a splicing feature in a DMD sequence is an exonic splicing enhancer (ESE), a branch point, a splice donor site, or a splice acceptor site in a DMD sequence. In some embodiments, an ESE is in exon 51 of a DMD sequence (e.g., a DMD pre-mRNA). In some embodiments, a branch point is in intron 50 or intron 51 of a DMD sequence (e.g., a DMD pre-mRNA). In some embodiments, a splice donor site is across the junction of exon 50 and intron 50, in intron 50, across the junction of exon 51 and intron 51, or in intron 51 of a DMD sequence (e.g., a DMD pre-mRNA). In some embodiments, a splice acceptor site is in intron 50, across the junction of intron 50 and exon 51, in intron 51, or across the junction of intron 51 and exon 52 of a DMD sequence (e.g., a DMD pre-mRNA). In some embodiments, the oligonucleotide useful for targeting DMD promotes skipping of exon 51, such as by targeting a splicing feature (e.g., an ESE, a branch point, a splice donor site, or a splice acceptor site) in a DMD sequence (e.g., a DMD pre-mRNA). Examples of ESEs, branch points, splice donor sites, and splice acceptor sites are provided in Table 9.

In some embodiments, an oligonucleotide useful for targeting DMD (e.g., for exon skipping) targets an exonic splicing enhancer (ESE) in a DMD sequence. In some embodiments, an oligonucleotide useful for targeting DMD (e.g., for exon skipping) targets an ESE in DMD exon 51 (e.g., an ESE listed in Table 9).

In some embodiments, an oligonucleotide useful for targeting DMD (e.g., for exon skipping, such as for skipping exon 51) comprises a region of complementarity to a target sequence comprising one or more full or partial ESEs of a DMD transcript (e.g., one or more full or partial ESEs listed in Table 9). In some embodiments, the oligonucleotide comprises a region of complementarity to a target sequence comprising one or more full or partial ESEs of DMD exon 51. In some embodiments, the oligonucleotide comprises a region of complementarity to a target sequence comprising one or more full or partial ESEs as set forth in SEQ ID NOs: 860-894. In some embodiments, the oligonucleotide comprises a region of complementarity to a target sequence comprising at least 4 (e.g., 4, 5, 6, 7, or 8) consecutive nucleotides of an ESE as set forth in any one of SEQ ID NOs: 860-894. In some embodiments, the oligonucleotide comprises at least 4 (e.g., 4, 5, 6, 7, or 8) consecutive nucleotides of an ESE antisense sequence as set forth in any one of SEQ ID NOs: 904-938.

In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) of DMD exon 51. In some embodiments, the oligonucleotide comprises a region of complementarity to a target sequence comprising at least 6 (e.g., 6, 7, 8, 9, 10, 11, 12, 13, 14, 15, 16, 17, 18, 19, 20, or more) nucleotides of one or more ESEs (e.g., 2, 3, 4, or more adjacent ESEs) as set forth in SEQ ID NOs: 860-894. In some embodiments, the oligonucleotide comprises at least 6 (e.g., 6, 7, 8, 9, 10, 11, 12, 13, 14, 15, 16, 17, 18, 19, 20, or more) nucleotides of one or more ESE antisense sequences (e.g., antisense sequences of 2, 3, 4, or more adjacent ESEs) as set forth in SEQ ID NOs: 904-938.

In some embodiments, an oligonucleotide useful for targeting DMD (e.g., for exon skipping, such as for skipping exon 51) is 18-35 nucleotides in length, and comprises a region of complementarity to a target sequence comprising at least 4 (e.g., 4, 5, 6, 7, or 8) consecutive nucleotides of an ESE as set forth in any one of SEQ ID NOs: 860-894. In some embodiments, an oligonucleotide useful for targeting DMD (e.g., for exon skipping, such as for skipping exon 51) is 20-30 (e.g., 20, 25, 30) nucleotides in length, and comprises a region of complementarity to a target sequence comprising at least 4 (e.g., 4, 5, 6, 7, or 8) consecutive nucleotides of an ESE as set forth in any one of SEQ ID NOs: 860-894. In some embodiments, an oligonucleotide useful for targeting DMD (e.g., for exon skipping, such as for skipping exon 51) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of an ESE as set forth in any one of SEQ ID NOs: 860-894. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, or 8) consecutive nucleotides of an ESE as set forth in any one of SEQ ID NOs: 860-894.

In some embodiments, an oligonucleotide useful for targeting DMD (e.g., for exon skipping) targets a branch point in a DMD sequence. In some embodiments, an oligonucleotide useful for targeting DMD (e.g., for exon skipping) targets a branch point in DMD intron 50 or intron 51 (e.g., a branch point listed in Table 9).

In some embodiments, an oligonucleotide useful for targeting DMD (e.g., for exon skipping, such as for skipping exon 51) comprises a region of complementarity to a target sequence comprising a full or partial branch point of a DMD transcript (e.g., a full or partial branch point listed in Table 9). In some embodiments, the oligonucleotide comprises a region of complementarity to a target sequence comprising a full or partial branch point of DMD intron 50 or intron 51. In some embodiments, the oligonucleotide comprises a region of complementarity to a target sequence comprising a full or partial branch point as set forth in any one of SEQ ID NOs: 856-858, 896, and 897. In some embodiments, the oligonucleotide comprises a region of complementarity to a target sequence comprising at least 4 (e.g., 4, 5, 6, or 7) consecutive nucleotides of a branch point as set forth in any one of SEQ ID NOs: 856-858, 896, and 897. In some embodiments, the oligonucleotide comprises at least 4 (e.g., 4, 5, 6, or 7) consecutive nucleotides of a branch point antisense sequence as set forth in any one of SEQ ID NOs: 900-902, 940, and 941.

In some embodiments, an oligonucleotide useful for targeting DMD (e.g., for exon skipping, such as for skipping exon 51) is 18-35 nucleotides in length, and comprises a region of complementarity to a target sequence comprising at least 4 (e.g., 4, 5, 6, or 7) consecutive nucleotides of a branch point as set forth in any one of SEQ ID NOs: 856-858, 896, and 897. In some embodiments, an oligonucleotide useful for targeting DMD (e.g., for exon skipping, such as for skipping exon 51) is 20-30 (e.g., 20, 25, 30) nucleotides in length, and comprises a region of complementarity to a target sequence comprising at least 4 (e.g., 4, 5, 6, or 7) consecutive nucleotides of a branch point as set forth in any one of SEQ ID NOs: 856-858, 896, and 897. In some embodiments, an oligonucleotide useful for targeting DMD (e.g., for exon skipping, such as for skipping exon 51) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a branch point as set forth in any one of SEQ ID NOs: 856-858, 896, and 897. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a branch point as set forth in any one of SEQ ID NOs: 856-858, 896, and 897.

In some embodiments, an oligonucleotide useful for targeting DMD (e.g., for exon skipping) targets a splice donor site in a DMD sequence. In some embodiments, an oligonucleotide useful for targeting DMD (e.g., for exon skipping) targets a splice donor site across the junction of exon 50 and intron 50, in intron 50, across the junction of exon 51 and intron 51, or in intron 51 (e.g., a splice donor site listed in Table 9).

In some embodiments, an oligonucleotide useful for targeting DMD (e.g., for exon skipping, such as for skipping exon 51) comprises a region of complementarity to a target sequence comprising a full or partial splice donor site of a DMD transcript (e.g., a full or partial splice donor site listed in Table 9). In some embodiments, the oligonucleotide comprises a region of complementarity to a target sequence comprising a full or partial splice donor site across the junction of exon 50 and intron 50, in intron 50, across the junction of exon 51 and intron 51, or in intron 51 of DMD. In some embodiments, the oligonucleotide comprises a region of complementarity to a target sequence comprising a full or partial splice donor site as set forth in SEQ ID NO: 855 or 895. In some embodiments, the oligonucleotide comprises a region of complementarity to a target sequence comprising at least 4 (e.g., 4, 5, 6, or 7) consecutive nucleotides of a splice donor site as set forth in SEQ ID NO: 855 or 895. In some embodiments, the oligonucleotide comprises at least 4 (e.g., 4, 5, 6, or 7) consecutive nucleotides of a splice donor site antisense sequence as set forth in SEQ ID NO: 899 or 939.

In some embodiments, an oligonucleotide useful for targeting DMD (e.g., for exon skipping, such as for skipping exon 51) is 18-35 nucleotides in length, and comprises a region of complementarity to a target sequence comprising at least 4 (e.g., 4, 5, 6, or 7) consecutive nucleotides of a splice donor site as set forth in SEQ ID NO: 855 or 895. In some embodiments, an oligonucleotide useful for targeting DMD (e.g., for exon skipping, such as for skipping exon 51) is 20-30 (e.g., 20, 25, 30) nucleotides in length, and comprises a region of complementarity to a target sequence comprising at least 4 (e.g., 4, 5, 6, or 7) consecutive nucleotides of a splice donor site as set forth in SEQ ID NO: 855 or 895. In some embodiments, an oligonucleotide useful for targeting DMD (e.g., for exon skipping, such as for skipping exon 51) is 20-25 (i.e., 20, 21, 22, 23, 24, or 25) nucleotides in length, and comprises a region of complementarity to a target sequence comprising at least 4 (e.g., 4, 5, 6, or 7) consecutive nucleotides of a splice donor site as set forth in SEQ ID NO: 855 or 895. In some embodiments, an oligonucleotide useful for targeting DMD (e.g., for exon skipping) is 30 nucleotides in length, and comprises a region of complementarity to a target sequence comprising at least 4 (e.g., 4, 5, 6, or 7) consecutive nucleotides of a splice donor site as set forth in SEQ ID NO: 855 or 895.

In some embodiments, an oligonucleotide useful for targeting DMD (e.g., for exon skipping) targets a splice acceptor site in a DMD sequence. In some embodiments, an oligonucleotide useful for targeting DMD (e.g., for exon skipping) targets a splice acceptor site in intron 50, across the junction of intron 50 and exon 51, in intron 51, or across the junction of intron 51 and exon 52 (e.g., a splice acceptor site listed in Table 9).

In some embodiments, an oligonucleotide useful for targeting DMD (e.g., for exon skipping, such as for skipping exon 51) comprises a region of complementarity to a target sequence comprising a full or partial splice acceptor site of a DMD transcript (e.g., a full or partial splice acceptor site listed in Table 9). In some embodiments, the oligonucleotide comprises a region of complementarity to a target sequence comprising a full or partial splice acceptor site in intron 50, across the junction of intron 50 and exon 51, in intron 51, or across the junction of intron 51 and exon 52 of DMD. In some embodiments, the oligonucleotide comprises a region of complementarity to a target sequence comprising a full or partial splice acceptor site as set forth in SEQ ID NO: 859 or 898. In some embodiments, the oligonucleotide comprises a region of complementarity to a target sequence comprising at least 4 (e.g., 4, 5, 6, 7, 8, or 9) consecutive nucleotides of a splice acceptor site as set forth in SEQ ID NO: 859 or 898. In some embodiments, the oligonucleotide comprises at least 4 (e.g., 4, 5, 6, 7, 8, or 9) consecutive nucleotides of a splice acceptor site antisense sequence as set forth in SEQ ID NO: 903 or 942.

In some embodiments, an oligonucleotide useful for targeting DMD (e.g., for exon skipping, such as for skipping exon 51) is 18-35 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 859 or 898. In some embodiments, an oligonucleotide useful for targeting DMD (e.g., for exon skipping, such as for skipping exon 51) is 20-30 (e.g., 20, 25, 30) nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 859 or 898. In some embodiments, an oligonucleotide useful for targeting DMD (e.g., for exon skipping, such as for skipping exon 51) is 20-25 (i.e., 20, 21, 22, 23, 24, or 25) nucleotides in length, and comprises a region of complementarity to a target sequence comprising at least 4 (e.g., 4, 5, 6, 7, or 8) consecutive nucleotides of a splice acceptor site as set forth in SEQ ID NO: 859 or 898. In some embodiments, an oligonucleotide useful for targeting DMD (e.g., for exon skipping) is 30 nucleotides in length, and comprises a region of complementarity to a target sequence comprising at least 4 (e.g., 4, 5, 6, 7, 8, or 9) consecutive nucleotides of a splice acceptor site as set forth in SEQ ID NO: 859 or 898.

In some embodiments, an oligonucleotide useful for targeting DMD (e.g., for exon skipping) comprises a region of complementarity to a junction of an exon and an intron of a DMD RNA (e.g., any one of the exon/intron junctions provided by SEQ ID NOs: 832, 833, 837, 844, 845, and 853). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a junction of an exon and an intron of a DMD RNA (e.g., any one of the exon/intron junctions provided by SEQ ID NOs: 832, 833, 837, 844, 845, and 853). In some embodiments, an oligonucleotide useful for targeting DMD (e.g., for exon skipping) is complementary to any one of SEQ ID NOs: 832, 833, 837, 844, 845, and 853.

In some embodiments, an oligonucleotide useful for targeting DMD (e.g., for exon skipping) comprises a region of complementarity to a target sequence of a DMD RNA (e.g., a target sequence provided by any one of SEQ ID NOs: 833, 835-837, 845, 847-853, and 839-843). In some embodiments, an oligonucleotide useful for targeting DMD (e.g., for exon skipping) comprises a region of complementarity to at least 10 (e.g., 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 833, 835-837, 845, 847-853, and 839-843). In some embodiments, an oligonucleotide useful for targeting DMD (e.g., for exon skipping) is complementary to any one of SEQ ID NOs: 833, 835-837, 845, 847-853, and 839-843.

TABLE 9
Example target sequence motifs

| | | SEQ | | SEQ | Motif |
|---|---|---|---|---|---|
| Location | | ID | Motif | ID | Antisense |
| in DMD | Type | NO: | Sequence^{†} | NO: | Sequence^{†} |
| Across | Splice | 855 | CTGTAAG | 899 | CTTACAG |
| exon 50/ | Donor | | | | |
| intron 50 | | | | | |
| junction | | | | | |
| Intron 50 | Branch | 856 | TATTAAT | 900 | ATTAATA |
| | Point | | | | |
| Intron 50 | Branch | 857 | TTGAC | 901 | GTCAA |
| | Point | | | | |
| Intron 50 | Branch | 858 | TTCTAAT | 902 | ATTAGAA |
| | Point | | | | |
| Across | Splice | 859 | TATTTTAGC | 903 | GCTAAAATA |
| intron 50/ | Acceptor | | | | |
| exon 51 | | | | | |
| junction | | | | | |
| Exon 51 | ESE | 860 | CTACTCA | 904 | TGAGTAG |
| Exon 51 | ESE | 861 | CAGACTG | 905 | CAGTCTG |
| Exon 51 | ESE | 862 | GACTGTTA | 906 | TAACAGTC |
| Exon 51 | ESE | 863 | TTACTCT | 907 | AGAGTAA |
| Exon 51 | ESE | 864 | ACTCTGG | 908 | CCAGAGT |
| Exon 51 | ESE | 865 | CTCTGGT | 909 | ACCAGAG |
| Exon 51 | ESE | 866 | TGACACA | 910 | TGTGTCA |
| Exon 51 | ESE | 867 | GACACAA | 911 | TTGTGTC |
| Exon 51 | ESE | 868 | ACACAAC | 912 | GTTGTGT |
| Exon 51 | ESE | 869 | AACCTGTG | 913 | CACAGGTT |
| Exon 51 | ESE | 870 | CCTGTGG | 914 | CCACAGG |
| Exon 51 | ESE | 871 | CTGTGGT | 915 | ACCACAG |
| Exon 51 | ESE | 872 | GGTTACTA | 916 | TAGTAACC |
| Exon 51 | ESE | 873 | TTACTAA | 917 | TTAGTAA |
| Exon 51 | ESE | 874 | CTAAGGA | 918 | TCCTTAG |
| Exon 51 | ESE | 875 | CTGCCAT | 919 | ATGGCAG |
| Exon 51 | ESE | 876 | CAAACTA | 920 | TAGTTTG |
| Exon 51 | ESE | 877 | TGGAGGT | 921 | ACCTCCA |
| Exon 51 | ESE | 878 | GGTACCTG | 922 | CAGGTACC |
| Exon 51 | ESE | 879 | GATTTCAA | 923 | TTGAAATC |
| Exon 51 | ESE | 880 | TTTCAAC | 924 | GTTGAAA |
| Exon 51 | ESE | 881 | TCAACCG | 925 | CGGTTGA |
| Exon 51 | ESE | 882 | CAACCGG | 926 | CCGGTTG |
| Exon 51 | ESE | 883 | GGACAGAA | 927 | TTCTGTCC |
| Exon 51 | ESE | 884 | CCGACTG | 928 | CAGTCGG |
| Exon 51 | ESE | 885 | CGACTGG | 929 | CCAGTCG |
| Exon 51 | ESE | 886 | TTTCTCTG | 930 | CAGAGAAA |
| Exon 51 | ESE | 887 | TCTCTGC | 931 | GCAGAGA |
| Exon 51 | ESE | 888 | TCACAGA | 932 | TCTGTGA |
| Exon 51 | ESE | 889 | CACAGA | 933 | TCTGTG |
| Exon 51 | ESE | 890 | ACAGAGG | 934 | CCTCTGT |
| Exon 51 | ESE | 891 | CAGAGGG | 935 | CCCTCTG |
| Exon 51 | ESE | 892 | CTTGAGG | 936 | CCTCAAG |
| Exon 51 | ESE | 893 | TGAGGA | 937 | TCCTCA |
| Exon 51 | ESE | 894 | GAGATGA | 938 | TCATCTC |
| Across | Splice | 895 | AGGTATG | 939 | CATACCT |
| exon 51/ | Donor | | | | |
| intron 51 | | | | | |
| junction | | | | | |
| Intron 51 | Branch | 896 | TTTTGAT | 940 | ATCAAAA |
| | Point | | | | |
| Intron 51 | Branch | 897 | CCCTGAT | 941 | ATCAGGG |
| | Point | | | | |
| Across | Splice | 898 | TCTTACAGG | 942 | CCTGTAAGA |
| intron 51/ | Acceptor | | | | |
| exon 52 | | | | | |
| junction | | | | | |
| Across | Splice | 855 | CTGTAAG | 899 | CTTACAG |
| exon 50/ | Donor | | | | |
| intron 50 | | | | | |
| junction | | | | | |

^{†}Each thymine base (T) in any one of the sequences provided in Table 9 may independently and optionally be replaced with a uracil base (U). Motif sequences and antisense sequences listed in Table 9 contain T's, but binding of a motif sequence in RNA and/or DNA is contemplated.

In some embodiments, any one of the oligonucleotides useful for targeting DMD (e.g., for exon skipping) is a phosphorodiamidate morpholino oligomer (PMO).

In some embodiments, the oligonucleotide may have region of complementarity to a mutant DMD allele, for example, a DMD allele with at least one mutation in any of exons 1-79 of DMD in humans that leads to a frameshift and improper RNA splicing/processing.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(RA)-, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of the any one of the oligonucleotides described herein (e.g., the oligonucleotides listed in Table 8). In some embodiments, the oligonucleotide is complementary (e.g., at least 85% at least 90%, at least 95%, or 100%) to a target sequence of the any one of the oligonucleotides provided by SEQ ID NO: 384-831. In some embodiments, such target sequence is 100% complementary to an oligonucleotide listed in Table 8. In some embodiments, such target sequence is 100% complementary to an oligonucleotide provided by SEQ ID NO: 384-831. In some embodiments, the oligonucleotide is complementary (e.g., at least 85% at least 90%, at least 95%, or 100%) to a target sequence provided herein (e.g., a target sequence listed in Table 8). In some embodiments, the oligonucleotide is complementary (e.g., at least 85% at least 90%, at least 95%, or 100%) to any one of SEQ ID NO: 160-383.

In some embodiments, an oligonucleotide useful for targeting DMD (e.g., for exon skipping) comprises a region of complementarity to a target sequence of a DMD RNA (e.g., a target sequence provided by any one of SEQ ID NOs: 160-383). In some embodiments, an oligonucleotide useful for targeting DMD (e.g., for exon skipping) comprises a region of complementarity to at least 8 (e.g., 8, 9, 10, 11, 12, 13, 14, 15, 16, 17, 18, 19, 20, 21, 22, 23, 24, 25, or more) consecutive nucleosides of a target sequence of a DMD RNA (e.g., a target sequence provided by any one of SEQ ID NOs: 160-383). In some embodiments, an oligonucleotide useful for targeting DMD (e.g., for exon skipping) is complementary to any one of SEQ ID NOs: 160-383.

In some embodiments, an oligonucleotide useful for targeting DMD (e.g., for exon skipping) comprises a sequence comprising at least 8 (e.g., 8, 9, 10, 11, 12, 13, 14, 15, 16, 17, 18, 19, 20, 21, 22, 23, 24, 25, or more) consecutive nucleobases of a DMD-targeting sequence provided herein (e.g., an antisense sequence listed in Table 8). In some embodiments, the oligonucleotide comprises a sequence comprising at least 8 (e.g., 8, 9, 10, 11, 12, 13, 14, 15, 16, 17, 18, 19, 20, 21, 22, 23, 24, 25, or more) consecutive nucleobases of any one of SEQ ID NOs: 384-831. In some embodiments, the oligonucleotide comprises the sequence of any one of SEQ ID NOs: 384-831.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Table 8) may independently and optionally be uracil bases (U's), and/or any one or more of the U's in the oligonucleotides provided herein may independently and optionally be T's. In some embodiments, any one or more of the thymine bases (T's) in any one of the oligonucleotides provided by SEQ ID NOs: 608-831 or in an oligonucleotide complementary to any one of SEQ ID NOs: 160-383 may optionally be uracil bases (U's), and/or any one or more of the U's in the oligonucleotides may optionally be T's. In some embodiments, any one or more of the uracil bases (U's) in any one of the oligonucleotides provided by SEQ ID NOs: 384-607 or in an oligonucleotide complementary to any one of SEQ ID NOs: 160-383 may optionally be thymine bases (T's), and/or any one or more of the T's in the oligonucleotides may optionally be U's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′—O-methyl (2′—O—Me), 2′—O-methoxyethyl (2′-MOE), 2′—O-aminopropyl (2′—O-AP), 2′—O-dimethylaminoethyl (2′—O-DMAOE), 2′—O-dimethylaminopropyl (2′—O-DMAP), 2′—O-dimethylaminoethyloxyethyl (2′—O-DMAEOE), or 2′—O-N-methylacetamido (2′—O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′—O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′—O-Me modified nucleosides. An oligonucleotide may comprise a mix of 2′-fluoro modified nucleosides and 2′—O-Me modified nucleosides. An oligonucleotide may comprise a mix of 2′-4′ bicyclic nucleosides and 2′-MOE, 2′-fluoro, or 2′—O-Me modified nucleosides. An oligonucleotide may comprise a mix of non-bicyclic 2′-modified nucleosides (e.g., 2′-MOE, 2′-fluoro, or 2′—O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′—O-Me modified nucleosides. An oligonucleotide may comprise alternating 2′-fluoro modified nucleosides and 2′—O-Me modified nucleosides. An oligonucleotide may comprise alternating 2′-4′ bicyclic nucleosides and 2′-MOE, 2′-fluoro, or 2′—O-Me modified nucleosides. An oligonucleotide may comprise alternating non-bicyclic 2′-modified nucleosides (e.g., 2′-MOE, 2′-fluoro, or 2′—O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 Al, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleosides, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′—O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′—O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′—O-Methyl Mixmer Antisense Oligonucleotide, Molecules 2016, 21, 1582, the contents of each which are incorporated herein by reference.

i. Multimers

In some embodiments, molecular payloads may comprise multimers (e.g., concatemers) of 2 or more oligonucleotides connected by a linker. In this way, in some embodiments, the oligonucleotide loading of a complex can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via an oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include j-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

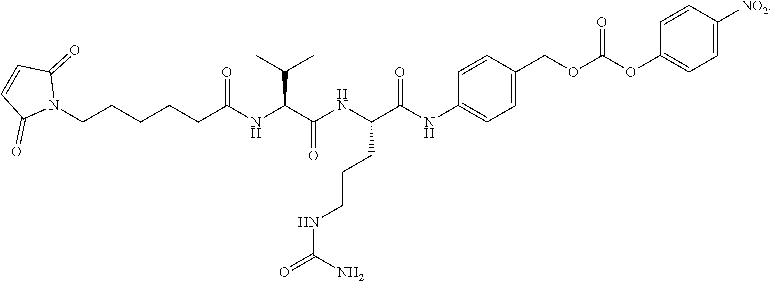 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

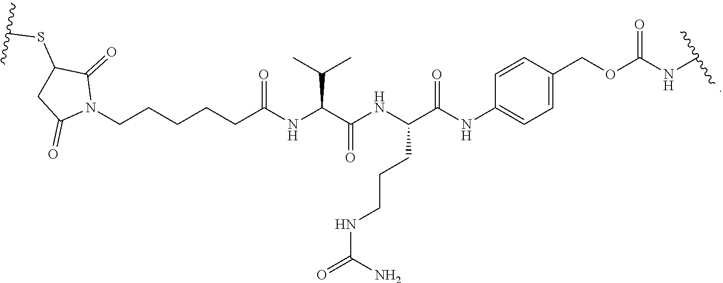 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of:

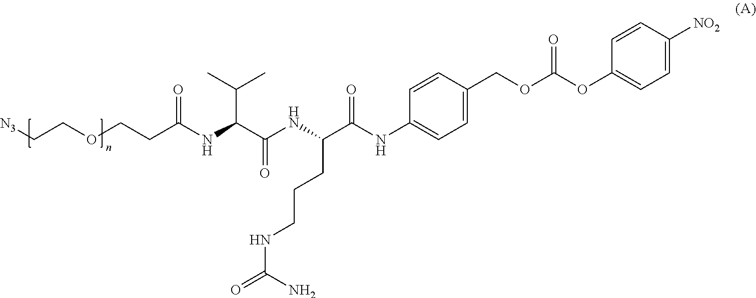

wherein n is any number from 0-10. In some embodiments, n is 3.

In some embodiments, a linker comprises a structure of:

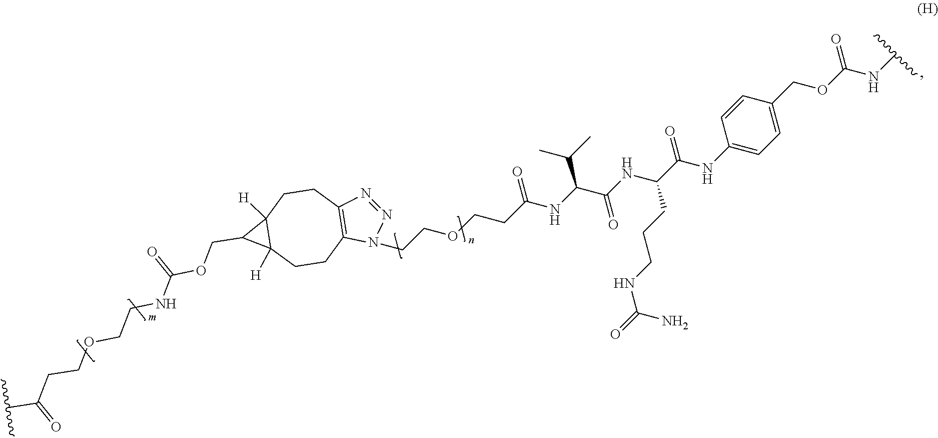

wherein n is any number from 0-10, wherein m is any number from 0-10. In some embodiments, n is 3 and/or (e.g., and) m is 4.

In some embodiments, a linker comprises a structure of:

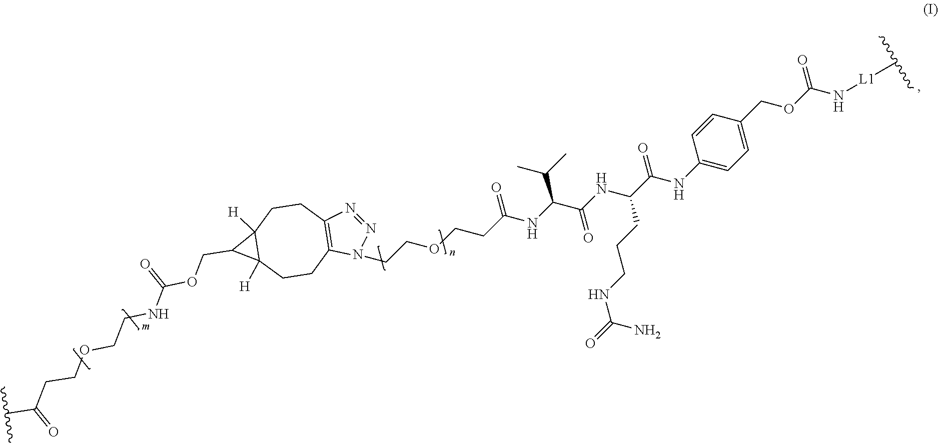

wherein n is any number from 0-10, wherein m is any number from 0-10. In some embodiments, n is 3 and/or (e.g., and) m is 4.
ii. Non-cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S, an imino, an optionally substituted nitrogen species, an optionally substituted oxygen species O, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody- Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reaction between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

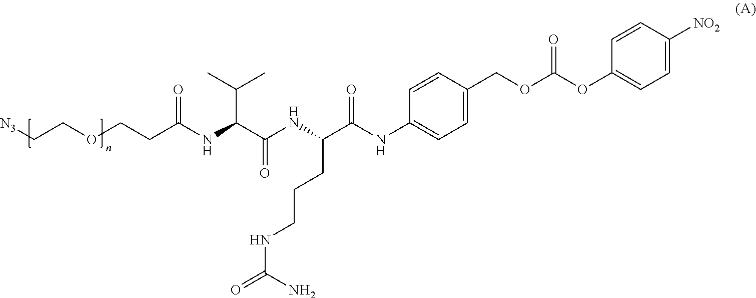

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

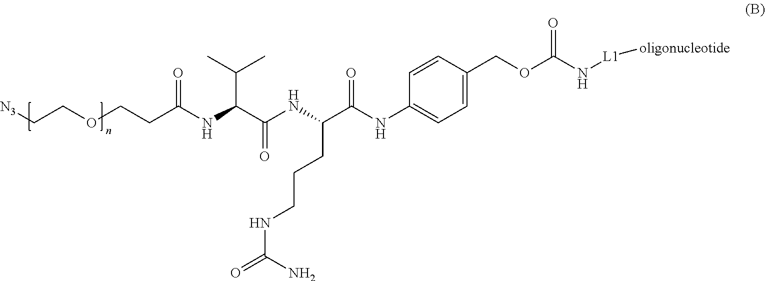

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

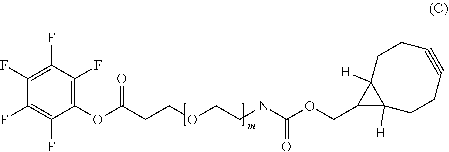

wherein m is any number from 0-10. In some embodiments, m is 4.

In some embodiments, the azide of the compound of structure (B) forms a triazole via a click reaction with the alkyne of the compound of structure (C), forming a compound comprising a structure of:

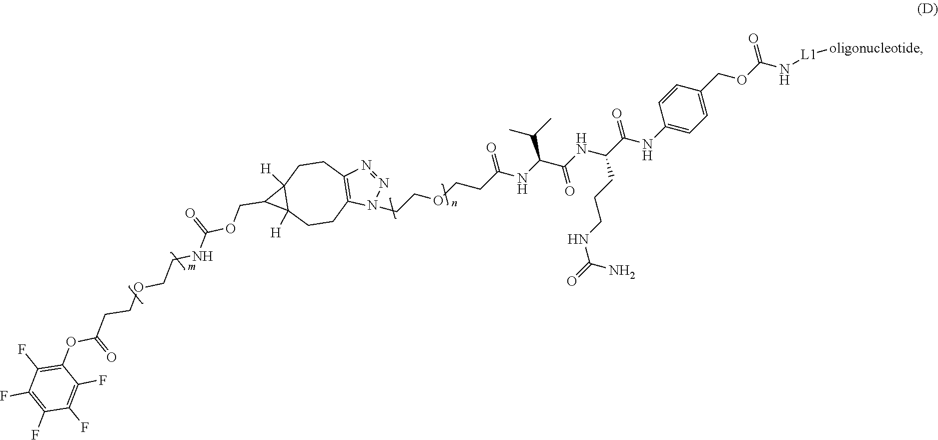

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

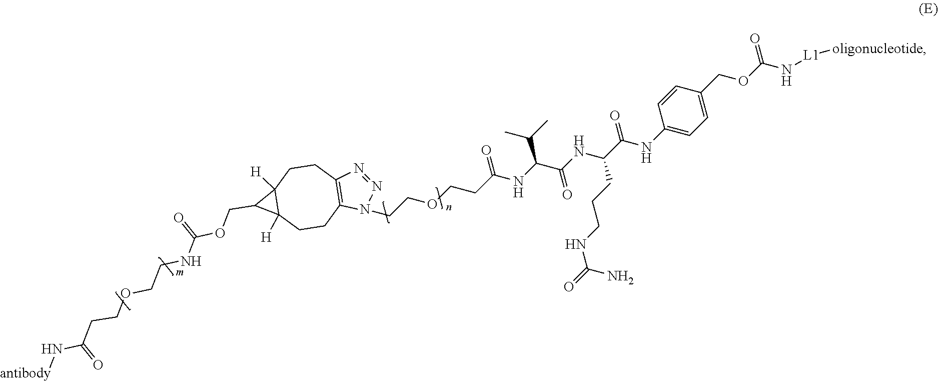

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

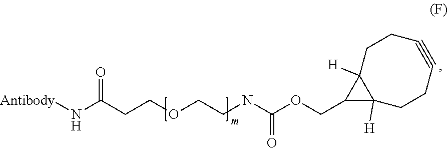

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

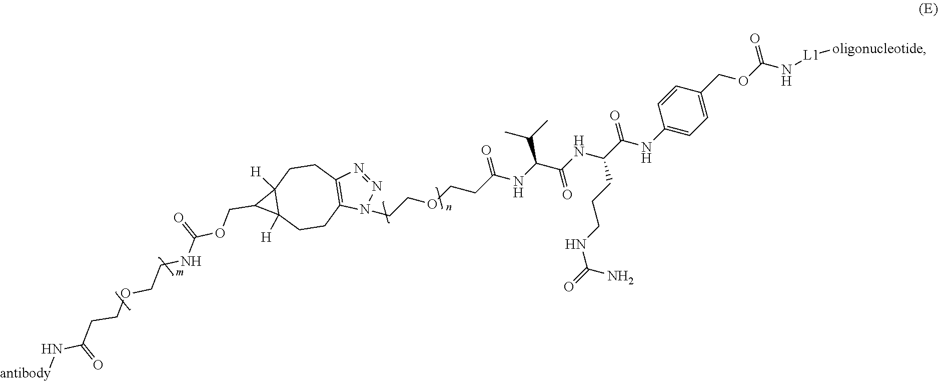

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

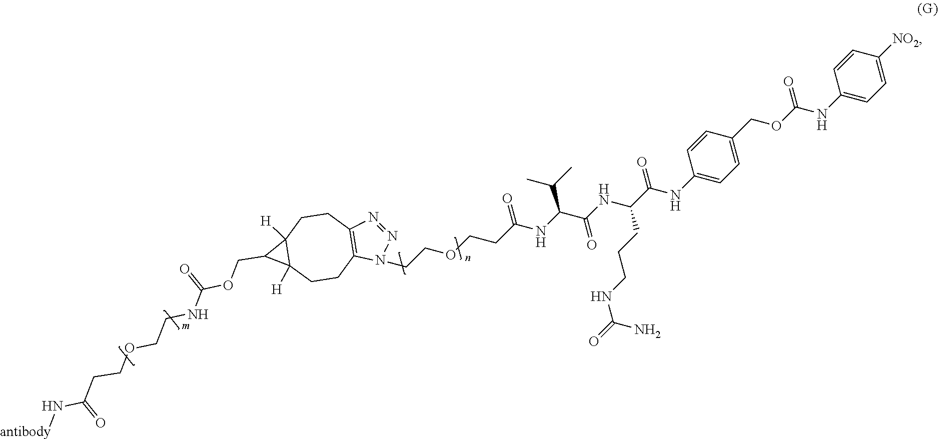

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

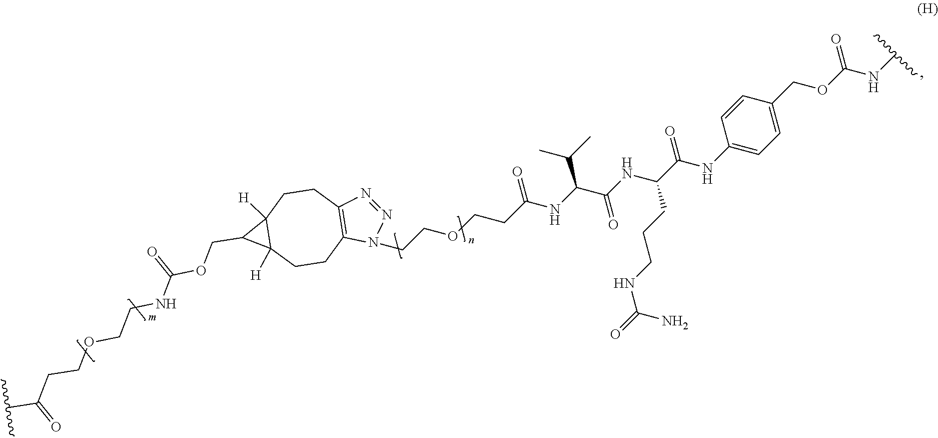

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

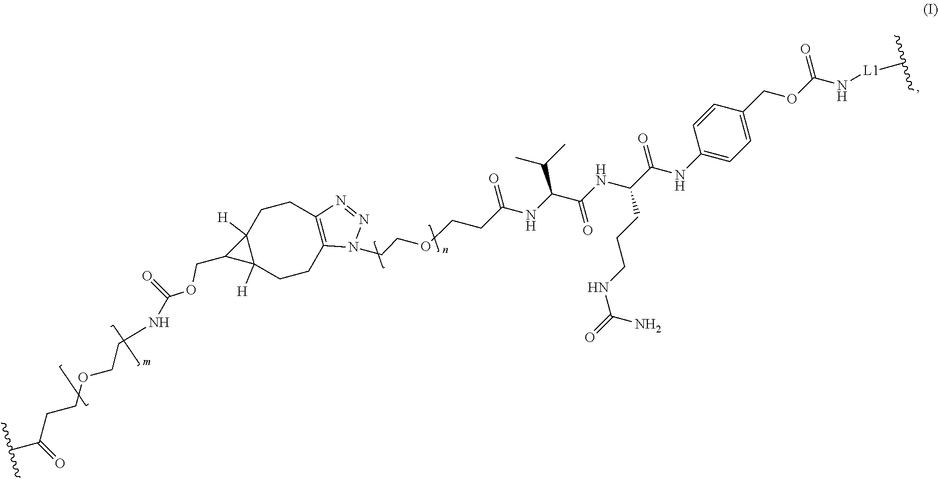

wherein n is any number from 0-10, wherein m is any number from 0-10. In some embodiments, n is 3 and/or (e.g., and) m is 4.

In some embodiments, in formulae (B), (D), (E), and (I), Li is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(RA)-, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

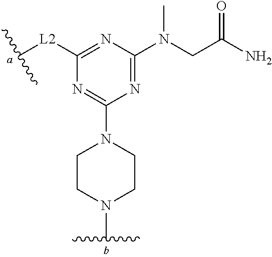

wherein L2 is

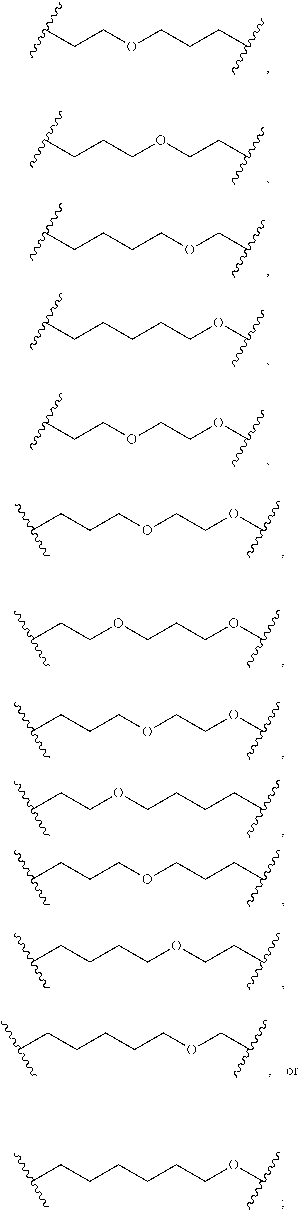

wherein a labels the site directly linked to the carbamate moiety of formulae (B), (D), (E), and (I); and b labels the site covalently linked (directly or via additional chemical moieties) to the oligonucleotide.

In some embodiments, L1 is:

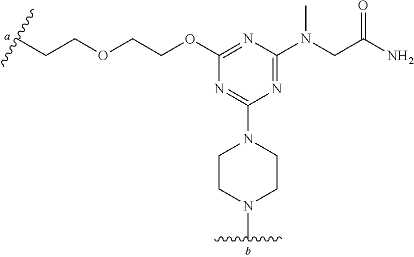 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

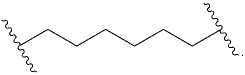 [SMILES: *CCCCCC*]

In some embodiments, L1 is linked to a 5′ phosphate of the oligonucleotide. In some embodiments, the phosphate is a phosphodiester. In some embodiments, L1 is linked to a 5′ phosphorothioate of the oligonucleotide. In some embodiments, L1 is linked to a 5′ phosphonoamidate of the oligonucleotide. In some embodiments, L1 is linked via a phosphorodiamidate linkage to the 5′ end of the oligonucleotide.

In some embodiments, L1 is optional (e.g., need not be present).

In some embodiments, any one of the complexes described herein has a structure of:

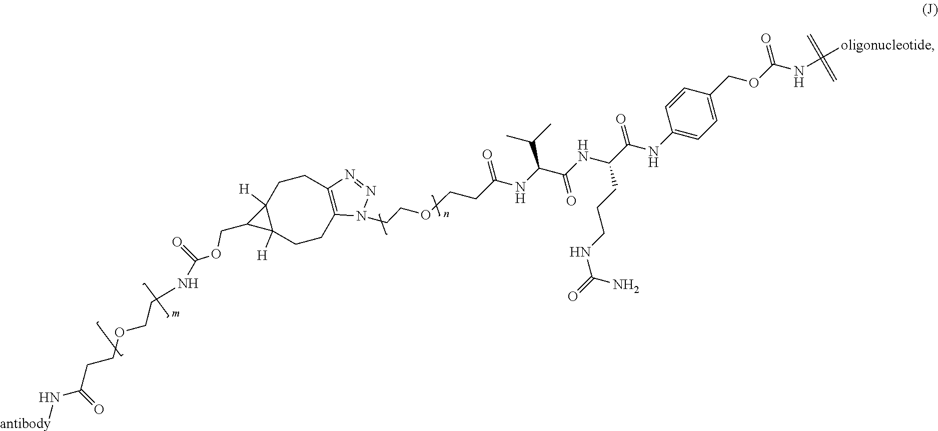

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

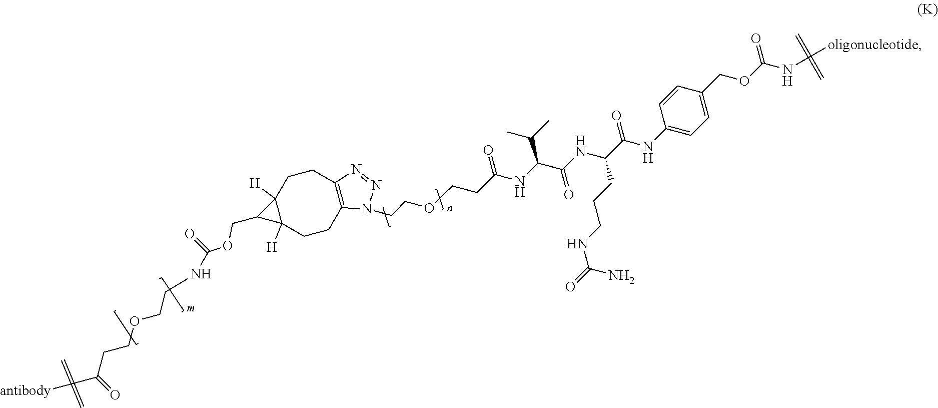

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8) via a linker. Any of the linkers described herein may be used. In some embodiments, if the molecular payload is an oligonucleotide, the linker is linked to the 5′ end of the oligonucleotide, the 3′ end of the oligonucleotide, or to an internal site of the oligonucleotide. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

An example of a structure of a complex comprising an anti-TfR1 antibody covalently linked to a molecular payload via a linker is provided below:

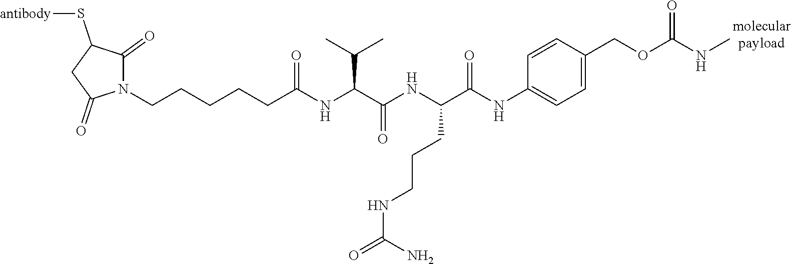

wherein the linker is linked to the antibody via a thiol-reactive linkage (e.g., via a cysteine in the antibody). In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

Another example of a structure of a complex comprising an anti-TfR1 antibody covalently linked to a molecular payload via a linker is provided below:

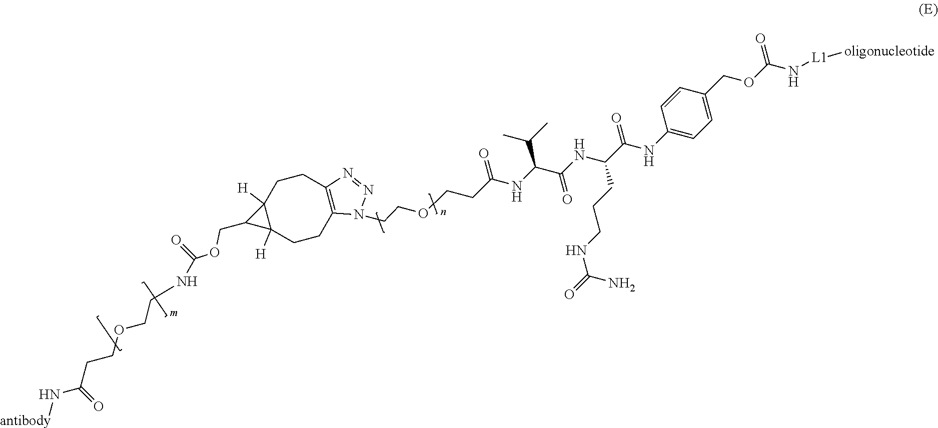

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383). It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. An average DAR of complexes in a mixture need not be an integer value. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 97, SEQ ID NO: 98, or SEQ ID NO: 99 and a light chain comprising the amino acid sequence of SEQ ID NO: 85. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is a DMD-targeting oligonucleotide (e.g., a DMD-targeting oligonucleotide listed in Table 8, provided by any one of SEQ ID NO: 384-831, or complementary to any one of SEQ ID NO: 160-383).

In any of the example complexes described herein, in some embodiments, the anti-TfR1 antibody is covalently linked to the molecular payload via a linker comprising a structure of:

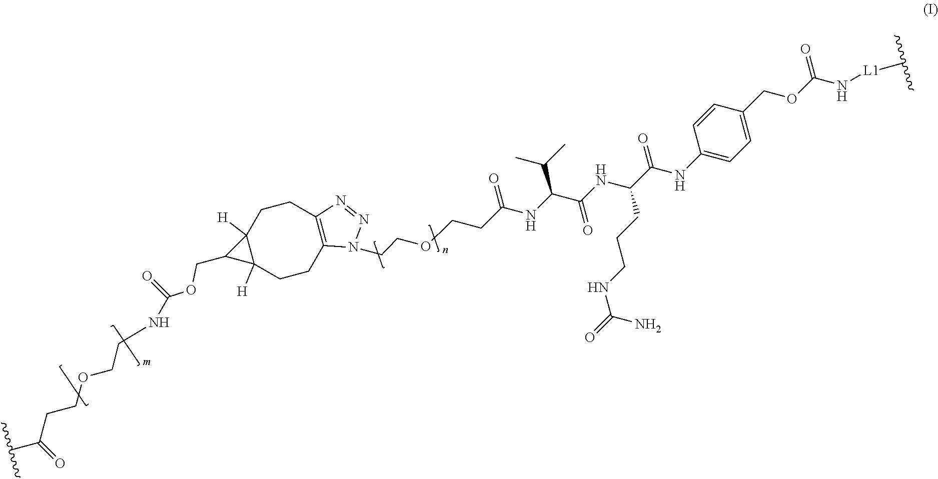

wherein n is 3, m is 4.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 384-831, or complementary to any one of SEQ ID NO: 160-383) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of:

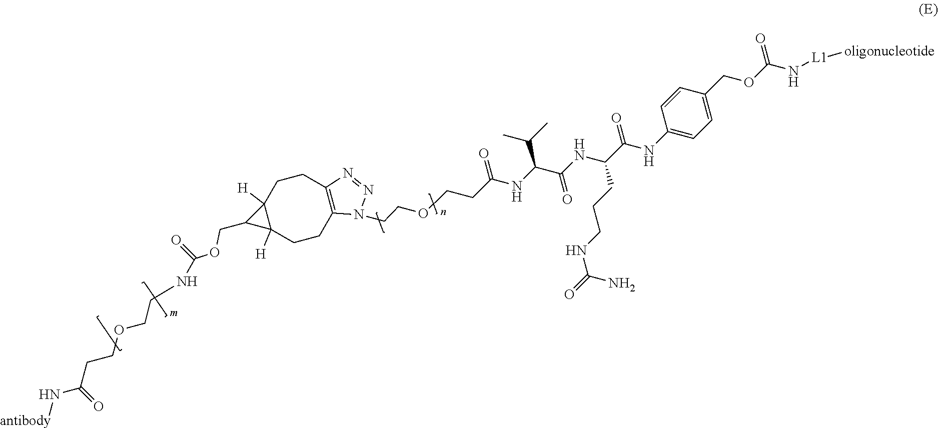

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 384-831, or complementary to any one of SEQ ID NO: 160-383) via a lysine in the anti-TfR1 antibody, wherein the anti-TfR1 antibody comprises a VH and VL of any one of the antibodies listed in Table 3, wherein the complex has a structure of:

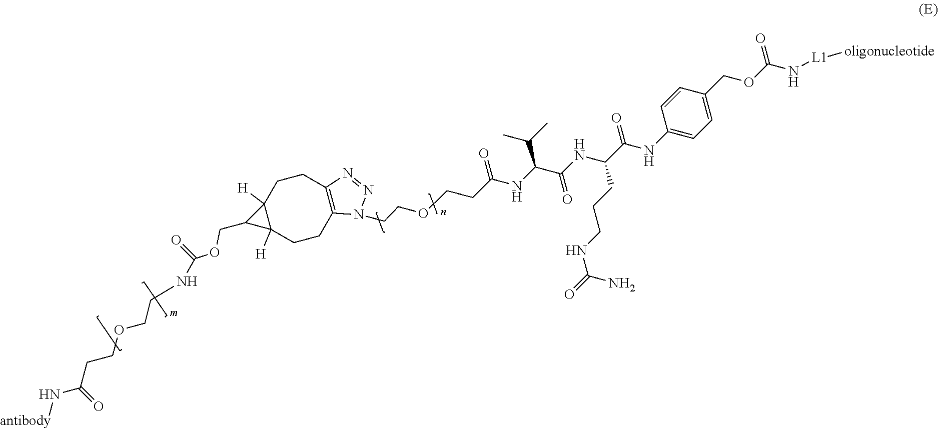

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMD-targeting oligonucleotide (e.g., a DMD-targeting oligonucleotide listed in Table 8, provided by any one of SEQ ID NO: 384-831, or complementary to any one of SEQ ID NO: 160-383) via a lysine in the anti-TfR1 antibody, wherein the anti-TfR1 antibody comprises a heavy chain and light chain of any one of the antibodies listed in Table 4, wherein the complex has a structure of:

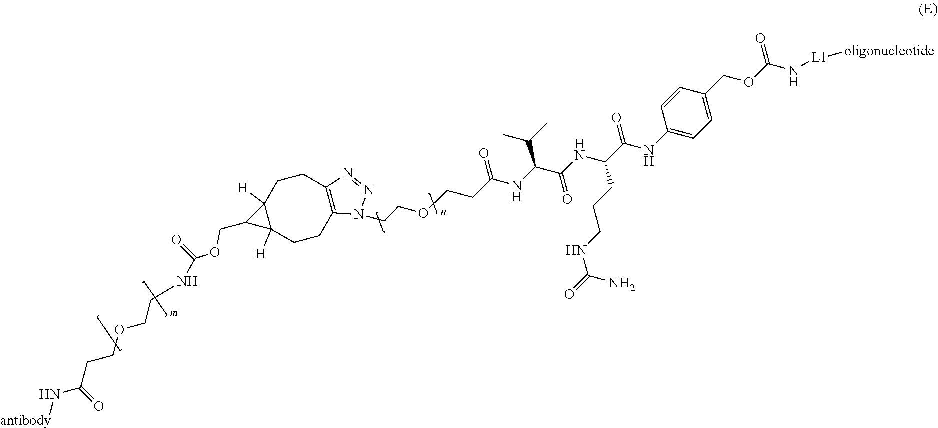

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 Fab covalently linked to the 5′ end of a DMD-targeting oligonucleotide (e.g., a DMD-targeting oligonucleotide listed in Table 8, provided by any one of SEQ ID NO: 384-831, or complementary to any one of SEQ ID NO: 160-383) via a lysine in the anti-TfR1 antibody, wherein the anti-TfR1 Fab comprises a heavy chain and light chain of any one of the antibodies listed in Table 5, wherein the complex has a structure of:

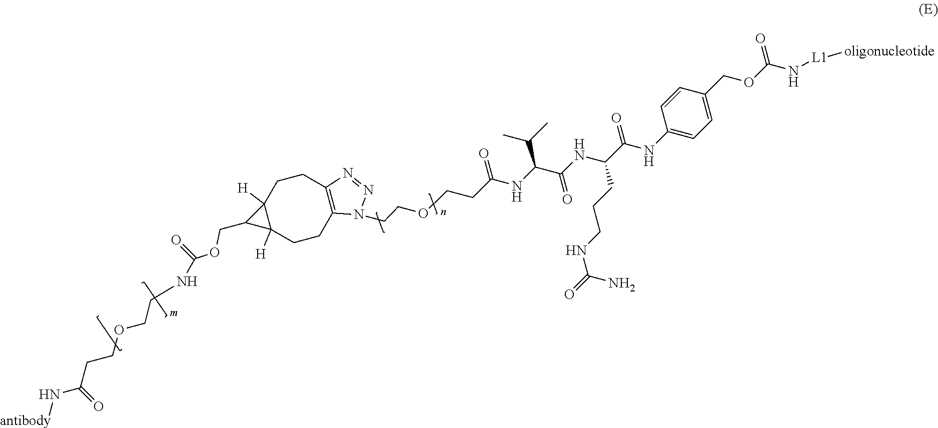

wherein n is 3 and m is 4. It should be understood that the amide shown adjacent the anti-TfR1 antibody in Formula (E) results from a reaction with an amine of the anti-TfR1 antibody, such as a lysine epsilon amine.

In some embodiments, in any one of the examples of complexes described herein, L1 is:

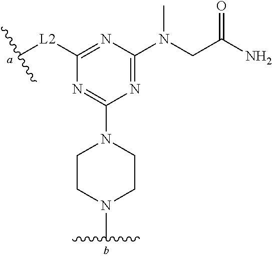

wherein L2 is

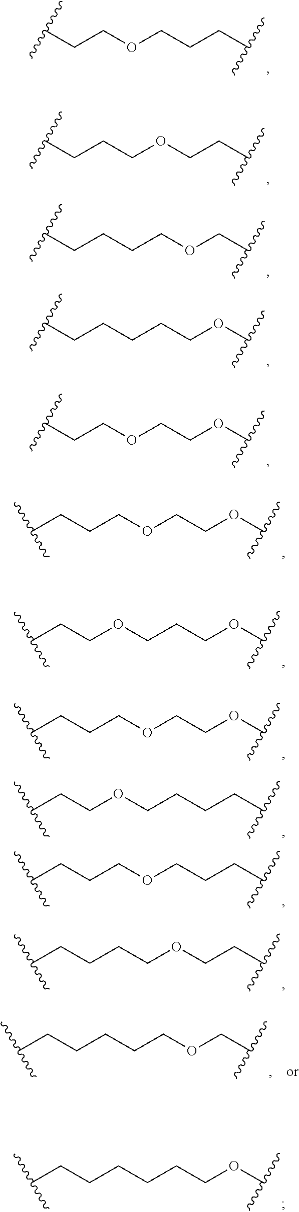

wherein a labels the site directly linked to the carbamate moiety of formulae (B), (D), (E), and (I); and b labels the site covalently linked (directly or via additional chemical moieties) to the oligonucleotide.

In some embodiments, L1 is:

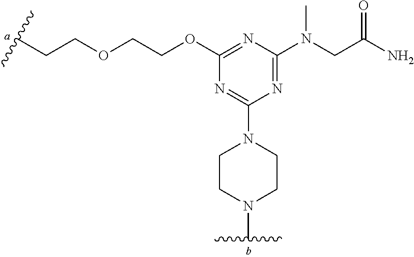 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, Li is linked to a 5′ phosphate of the oligonucleotide. In some embodiments, the phosphate is a phosphodiester. In some embodiments, L1 is linked to a 5′ phosphorothioate of the oligonucleotide. In some embodiments, Li is linked to a 5′ phosphonoamidate of the oligonucleotide. In some embodiments, L1 is linked via a phosphorodiamidate linkage to the 5′ end of the oligonucleotide.

In some embodiments, Li is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating a subject having a dystrophinopathy, e.g., Duchenne muscular dystrophy. In some embodiments, complexes comprise a molecular payload that is an oligonucleotide, e.g., an antisense oligonucleotide that facilitates exon skipping of a pre-mRNA expressed from a mutated DMD allele.

In some embodiments, a subject may be a human subject, a non-human primate subject, a rodent subject, or any suitable mammalian subject. In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g. muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy.

In some embodiments, a subject has a mutation in a DMD gene that is amenable to exon 51 skipping. In some embodiments, a complex comprising a muscle-targeting agent covalently linked to a molecular payload as described herein is effective in treating a subject having a mutation in a DMD gene that is amenable to exon 51 skipping. In some embodiments, a complex comprises a molecular payload that is an oligonucleotide, e.g., an antisense oligonucleotide that facilitates skipping of exon 51 of a pre-mRNA, such as in a pre-mRNA encoded from a mutated DMD gene (e.g., a mutated DMD gene that is amenable to exon 51 skipping).

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with a dystrophinopathy, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### Additional Embodiments

1. A complex comprising an anti-transferrin receptor 1 (TfR1) antibody covalently linked to a molecular payload configured for inducing skipping of exon 51 in a DMD pre-mRNA, wherein the anti-TfR1 antibody is an antibody identified in any one of Tables 2-7.
2. The complex of embodiment 1, wherein the anti-TfR1 antibody comprises:
   (i) a heavy chain complementarity determining region 1 (CDR-H1) of SEQ ID NO: 33, a heavy chain complementarity determining region 2 (CDR-H2) of SEQ ID NO: 34, a heavy chain complementarity determining region 3 (CDR-H3) of SEQ ID NO: 35, a light chain complementarity determining region 1 (CDR-L1) of SEQ ID NO: 36, a light chain complementarity determining region 2 (CDR-L2) of SEQ ID NO: 37, and a light chain complementarity determining region 3 (CDR-L3) of SEQ ID NO: 32;
   (ii) a CDR-H1 of SEQ ID NO: 7, a CDR-H2 of SEQ ID NO: 8, a CDR-H3 of SEQ ID NO: 9, a CDR-L1 of SEQ ID NO: 10, a CDR-L2 of SEQ ID NO: 11, and a CDR-L3 of SEQ ID NO: 6;
   (iii) a CDR-H1 of SEQ ID NO: 7, a CDR-H2 of SEQ ID NO: 20, a CDR-H3 of SEQ ID NO: 9, a CDR-L1 of SEQ ID NO: 10, a CDR-L2 of SEQ ID NO: 11, and a CDR-L3 of SEQ ID NO: 6;
   (iv) a CDR-H1 of SEQ ID NO: 7, a CDR-H2 of SEQ ID NO: 24, a CDR-H3 of SEQ ID NO: 9, a CDR-L1 of SEQ ID NO: 10, a CDR-L2 of SEQ ID NO: 11, and a CDR-L3 of SEQ ID NO: 6;
   (v) a CDR-H1 of SEQ ID NO: 51, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50;
   (vi) a CDR-H1 of SEQ ID NO: 64, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50; or
   (vii) a CDR-H1 of SEQ ID NO: 67, a CDR-H2 of SEQ ID NO: 52, a CDR-H3 of SEQ ID NO: 53, a CDR-L1 of SEQ ID NO: 54, a CDR-L2 of SEQ ID NO: 55, and a CDR-L3 of SEQ ID NO: 50.
3. The complex of embodiment 1 or embodiment 2, wherein the anti-TfR1 antibody comprises:
   (i) a heavy chain variable region (VH) comprising an amino acid sequence at least 85% identical to SEQ ID NO: 76; and/or a light chain variable region (VL) comprising an amino acid sequence at least 85% identical to SEQ ID NO: 75;
   (ii) a VH comprising an amino acid sequence at least 85% identical to SEQ ID NO: 69; and/or a VL comprising an amino acid sequence at least 85% identical to SEQ ID NO: 70;
   (iii) a VH comprising an amino acid sequence at least 85% identical to SEQ ID NO: 71; and/or a VL comprising an amino acid sequence at least 85% identical to SEQ ID NO: 70;
   (iv) a VH comprising an amino acid sequence at least 85% identical to SEQ ID NO: 72; and/or a VL comprising an amino acid sequence at least 85% identical to SEQ ID NO: 70;
   (v) a VH comprising an amino acid sequence at least 85% identical to SEQ ID NO: 73; and/or a VL comprising an amino acid sequence at least 85% identical to SEQ ID NO: 74;
   (vi) a VH comprising an amino acid sequence at least 85% identical to SEQ ID NO: 73; and/or a VL comprising an amino acid sequence at least 85% identical to SEQ ID NO: 75;
   (vii) a VH comprising an amino acid sequence at least 85% identical to SEQ ID NO: 76; and/or a VL comprising an amino acid sequence at least 85% identical to SEQ ID NO: 74;
   (viii) a VH comprising an amino acid sequence at least 85% identical to SEQ ID NO: 77; and/or a VL comprising an amino acid sequence at least 85% identical to SEQ ID NO: 78;
   (ix) a VH comprising an amino acid sequence at least 85% identical to SEQ ID NO: 79; and/or a VL comprising an amino acid sequence at least 85% identical to SEQ ID NO: 80; or
   (x) a VH comprising an amino acid sequence at least 85% identical to SEQ ID NO: 77; and/or a VL comprising an amino acid sequence at least 85% identical to SEQ ID NO: 80.
4. The complex of any one of embodiments 1 to 3, wherein the anti-TfR1 antibody comprises:
   (i) a VH comprising the amino acid sequence of SEQ ID NO: 76 and a VL comprising the amino acid sequence of SEQ ID NO: 75;
   (ii) a VH comprising the amino acid sequence of SEQ ID NO: 69 and a VL comprising the amino acid sequence of SEQ ID NO: 70;
   (iii) a VH comprising the amino acid sequence of SEQ ID NO: 71 and a VL comprising the amino acid sequence of SEQ ID NO: 70;
   (iv) a VH comprising the amino acid sequence of SEQ ID NO: 72 and a VL comprising the amino acid sequence of SEQ ID NO: 70;
   (v) a VH comprising the amino acid sequence of SEQ ID NO: 73 and a VL comprising the amino acid sequence of SEQ ID NO: 74;
   (vi) a VH comprising the amino acid sequence of SEQ ID NO: 73 and a VL comprising the amino acid sequence of SEQ ID NO: 75;
   (vii) a VH comprising the amino acid sequence of SEQ ID NO: 76 and a VL comprising the amino acid sequence of SEQ ID NO: 74;
   (viii) a VH comprising the amino acid sequence of SEQ ID NO: 77 and a VL comprising the amino acid sequence of SEQ ID NO: 78;
   (ix) a VH comprising the amino acid sequence of SEQ ID NO: 79 and a VL comprising the amino acid sequence of SEQ ID NO: 80; or
   (x) a VH comprising the amino acid sequence of SEQ ID NO: 77 and a VL comprising the amino acid sequence of SEQ ID NO: 80.
5. The complex of any one of embodiments 1 to 4, wherein the anti-TfR1 antibody is a Fab fragment, a Fab′ fragment, a F(ab′)₂ fragment, an scFv, an Fv, or a full-length IgG.
6. The complex of embodiment 5, wherein the anti-TfR1 antibody is a Fab fragment.
7. The complex of embodiment 6, wherein the anti-TfR1 antibody comprises:
   (i) a heavy chain comprising an amino acid sequence at least 85% identical to SEQ ID NO: 101; and/or a light chain comprising an amino acid sequence at least 85% identical to SEQ ID NO: 90;
   (ii) a heavy chain comprising an amino acid sequence at least 85% identical to SEQ ID NO: 97; and/or a light chain comprising an amino acid sequence at least 85% identical to SEQ ID NO: 85;
   (iii) a heavy chain comprising an amino acid sequence at least 85% identical to SEQ ID NO: 98; and/or a light chain comprising an amino acid sequence at least 85% identical to SEQ ID NO: 85;
   (iv) a heavy chain comprising an amino acid sequence at least 85% identical to SEQ ID NO: 99; and/or a light chain comprising an amino acid sequence at least 85% identical to SEQ ID NO: 85;
   (v) a heavy chain comprising an amino acid sequence at least 85% identical to SEQ ID NO: 100; and/or a light chain comprising an amino acid sequence at least 85% identical to SEQ ID NO: 89;
   (vi) a heavy chain comprising an amino acid sequence at least 85% identical to SEQ ID NO: 100; and/or a light chain comprising an amino acid sequence at least 85% identical to SEQ ID NO: 90;
   (vii) a heavy chain comprising an amino acid sequence at least 85% identical to SEQ ID NO: 101; and/or a light chain comprising an amino acid sequence at least 85% identical to SEQ ID NO: 89;
   (viii) a heavy chain comprising an amino acid sequence at least 85% identical to SEQ ID NO: 102; and/or a light chain comprising an amino acid sequence at least 85% identical to SEQ ID NO: 93;
   (ix) a heavy chain comprising an amino acid sequence at least 85% identical to SEQ ID NO: 103; and/or a light chain comprising an amino acid sequence at least 85% identical to SEQ ID NO: 95; or
   (x) a heavy chain comprising an amino acid sequence at least 85% identical to SEQ ID NO: 102; and/or a light chain comprising an amino acid sequence at least 85% identical to SEQ ID NO: 95.
8. The complex of embodiment 6 or embodiment 7, wherein the anti-TfR1 antibody comprises:
   (i) a heavy chain comprising the amino acid sequence of SEQ ID NO: 101; and a light chain comprising the amino acid sequence of SEQ ID NO: 90;
   (ii) a heavy chain comprising the amino acid sequence of SEQ ID NO: 97; and a light chain comprising the amino acid sequence of SEQ ID NO: 85;
   (iii) a heavy chain comprising the amino acid sequence of SEQ ID NO: 98; and a light chain comprising the amino acid sequence of SEQ ID NO: 85;
   (iv) a heavy chain comprising the amino acid sequence of SEQ ID NO: 99; and a light chain comprising the amino acid sequence of SEQ ID NO: 85;
   (v) a heavy chain comprising the amino acid sequence of SEQ ID NO: 100; and a light chain comprising the amino acid sequence of SEQ ID NO: 89;
   (vi) a heavy chain comprising the amino acid sequence of SEQ ID NO: 100; and a light chain comprising the amino acid sequence of SEQ ID NO: 90;
   (vii) a heavy chain comprising the amino acid sequence of SEQ ID NO: 101; and a light chain comprising the amino acid sequence of SEQ ID NO: 89;
   (viii) a heavy chain comprising the amino acid sequence of SEQ ID NO: 102; and a light chain comprising the amino acid sequence of SEQ ID NO: 93;
   (ix) a heavy chain comprising the amino acid sequence of SEQ ID NO: 103; and a light chain comprising the amino acid sequence of SEQ ID NO: 95; or
   (x) a heavy chain comprising the amino acid sequence of SEQ ID NO: 102; and a light chain comprising the amino acid sequence of SEQ ID NO: 95.
9. The complex of any one of embodiments 1 to 8, wherein the anti-TfR1 antibody does not specifically bind to the transferrin binding site of the transferrin receptor 1 and/or wherein the anti-TfR1 antibody does not inhibit binding of transferrin to the transferrin receptor 1.
10. The complex of any one of embodiments 1 to 9, wherein the molecular payload comprises an oligonucleotide.
11. The complex of embodiment 10, wherein the oligonucleotide promotes antisense-mediated exon skipping in the DMD pre-RNA.
12. The complex of embodiment 10 or 11, wherein the oligonucleotide comprises a region of complementarity to a splicing feature of the DMD pre-mRNA.
13. The complex of embodiment 12, wherein the splicing feature is an exonic splicing enhancer (ESE) of the DMD pre-mRNA.
14. The complex of embodiment 13, wherein the splicing feature is in exon 51 of the DMD pre-mRNA, optionally wherein the ESE comprises a sequence of any one of SEQ ID NOs: 860-894.
15. The complex of embodiment 12, wherein the splicing feature is a branch point, a splice donor site, or a splice acceptor site.
16. The complex of embodiment 15, wherein the splicing feature is across the junction of exon 50 and intron 50, in intron 50, across the junction of intron 50 and exon 51, across the junction of exon 51 and intron 51, in intron 51, or across the junction of intron 51 and exon 52 of the DMD pre-mRNA, optionally wherein the splicing feature comprises a sequence of any one of SEQ ID NOs: 855-8595 and 895-898.
17. The complex of any one of embodiments 12 to 16, wherein the region of complementarity comprises at least 4 consecutive nucleosides complementary to the splicing feature.
18. The complex of any one of embodiments 1 to 9, wherein the molecular payload comprises an oligonucleotide comprising a sequence complementary to any one of SEQ ID NOs: 160-383 or comprising a sequence of any one of SEQ ID NOs: 384-831, wherein each thymine base (T) may independently and optionally be replaced with a uracil base (U), and each U may independently and optionally be replaced with a T.
19. The complex of any one of embodiments 10 to 18, wherein the oligonucleotide comprises at least one modified internucleoside linkage.
20. The complex of embodiment 19, wherein the at least one modified internucleoside linkage is a phosphorothioate linkage.
21. The complex of any one of embodiments 10 to 20, wherein the oligonucleotide comprises one or more modified nucleosides.
22. The complex of embodiment 21, wherein the one or more modified nucleosides are 2′-modified nucleosides.
23. The complex of any one of embodiments 10 to 18, wherein the oligonucleotide comprises one or more phosphorodiamidate morpholinos, optionally wherein the oligonucleotide is a phosphorodiamidate morpholino oligomer (PMO).
24. The complex of any one of embodiments 1 to 23, wherein the anti-TfR1 antibody is covalently linked to the molecular payload via a cleavable linker.
25. The complex of embodiment 24, wherein the cleavable linker comprises a valine-citrulline sequence.
26. The complex of any one of embodiments 1 to 25, wherein the anti-TfR1 antibody is covalently linked to the molecular payload via conjugation to a lysine residue or a cysteine residue of the antibody.
27. A complex comprising an anti-TfR1 antibody covalently linked to an oligonucleotide configured for inducing skipping of exon 51 in a DMD pre-mRNA, wherein the oligonucleotide comprises a region of complementarity to any one of SEQ ID NOs: 160-383.
28. The complex of embodiment 27, wherein the anti-TfR1 antibody is an antibody identified in any one of Tables 2-7.
29. A complex comprising an anti-TfR1 antibody covalently linked to an oligonucleotide configured for inducing skipping of exon 51 in a DMD pre-mRNA, wherein the oligonucleotide comprises a region of complementarity to a splicing feature of the DMD pre-mRNA.
30. An oligonucleotide that targets DMD, wherein the oligonucleotide comprises a region of complementarity to any one of SEQ ID NOs: 160-383.
31. The oligonucleotide of embodiment 30, wherein the region of complementarity comprises at least 15 consecutive nucleosides complementary to any one of SEQ ID NOs: 160-383.
32. The oligonucleotide of embodiment 30 or 31, wherein the oligonucleotide comprises at least 15 consecutive nucleosides of any one of SEQ ID NOs: 384-831, optionally wherein the oligonucleotide comprises a sequence of any one of SEQ ID NOs: 384-831, wherein each thymine base (T) may independently and optionally be replaced with a uracil base (U), and each U may independently and optionally be replaced with a T.
33. A method of delivering a molecular payload to a cell, the method comprising contacting the cell with the complex of any one of embodiments 1 to 26.
34. A method of delivering an oligonucleotide to a cell, the method comprising contacting the cell with the complex of any one of embodiments 27 to 29.
35. A method of promoting the expression or activity of a dystrophin protein in a cell, the method comprising contacting the cell with the complex of any one of embodiments 1 to 26 in an amount effective for promoting internalization of the molecular payload to the cell, optionally wherein the cell is a muscle cell.
36. A method of promoting the expression or activity of a dystrophin protein in a cell, the method comprising contacting the cell with the complex of any one of embodiments 27 to 29 in an amount effective for promoting internalization of the oligonucleotide to the cell, optionally wherein the cell is a muscle cell.
37. The method of embodiment 35 or 36, wherein the cell is in vitro.
38. The method of embodiment 35 or 36, wherein the cell is in a subject.
39. The method of embodiment 38, wherein the subject is a human.
40. The method of embodiment 39, wherein the subject has a DMD gene that is amenable to skipping of exon 51.
41. The method of any one of embodiments 35 to 40, wherein the dystrophin protein is a truncated dystrophin protein.
42. A method of treating a subject having a mutated DMD allele that is associated with a dystrophinopathy, the method comprising administering to the subject an effective amount of the complex of any one of embodiments 1 to 29.
43. A method of promoting skipping of exon 51 of a DMD pre-mRNA transcript in a cell, the method comprising contacting the cell with an effective amount of the complex of any one of embodiments 1 to 29.
44. A method of treating a subject having a mutated DMD allele that is associated with a dystrophinopathy, the method comprising administering to the subject an effective amount of the complex of any one of embodiments 1 to 29.

### EXAMPLES

### Example 1. Exon-Skipping Activity of Anti-TfR1 Antibody Conjugates in Duchenne Muscular Dystrophy Patient Myotubes

In this study, the exon-skipping activities of anti-TfR1 antibody conjugates comprising an anti-TfR1 Fab (3M12 VH4/Vκ3) covalently linked to a DMD exon 51-skipping antisense oligonucleotide (ASO) were evaluated. The DMD exon 51-skipping ASO is a phosphorodiamidate morpholino oligomer (PMO) of 30 nucleotides in length and targets an ESE in DMD exon 51 having the sequence TGGAGGT (SEQ ID NO: 877). Immortalized human myoblasts bearing an exon 52 deletion in the DMD gene were thawed and seeded at a density of 1e6 cell/flask in Promocell Skeletal Cell Growth Media (with 5% FBS and lx Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50,000 cells/well. Cells were allowed to recover for 24 hours. Cells were induced to differentiate into myotubes by aspirating the growth media and replacing with differentiation media with no serum. Cells were then treated with the DMD exon 51-skipping oligonucleotide (not covalently linked to an antibody—“naked”) at 10 μM ASO or the anti-TfR1 Fab (3M12 VH4/Vκ3) covalently linked to the DMD exon 51-skipping oligonucleotide at 10 μM ASO equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in the cells. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + Unskipped Amplicon )} * 100 .$$

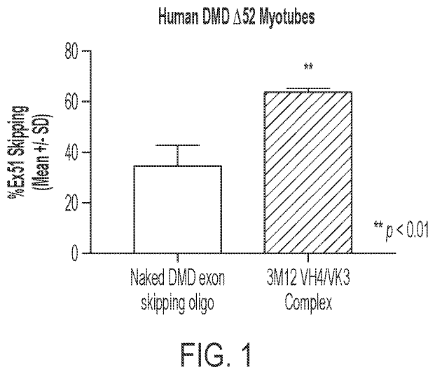

The results demonstrate that the conjugate resulted in enhanced exon skipping compared to the naked DMD exon 51-skipping oligonucleotide in patient myotubes (FIG. 1). This indicates that anti-TfR1 Fab 3M12 VH4/Vκ3 enabled cellular internalization of the conjugate into muscle cells resulting in activity of the exon 51-skipping oligonucleotide in the muscle cells. Similarly, an anti-TfR1 antibody (e.g., anti-TfR1 Fab 3M12 VH4/Vκ3) can enable internalization of a conjugate comprising the anti-TfR1 antibody covalently linked to other exon skipping oligonucleotides (e.g., an exon skipping oligonucleotide provided herein, such as an exon 51 skipping oligonucleotide) into muscle cells and facilitate activity of the exon skipping oligonucleotide in the muscle cells.

### Example 2. Exon Skipping Activity of Anti-TfR1 Fab-ASO Conjugate In Vivo in Cynomolgus Monkeys

Anti-TfR1 Fab 3M12 VH4/Vκ3 was covalently linked to the DMD exon 51-skipping antisense oligonucleotide (ASO) that was used in Example 1. The exon skipping activity of the conjugate was tested in vivo in healthy non-human primates. Naïve male cynomolgus monkeys (n=4-5 per group) were administered two doses of vehicle, 30 mg/kg naked ASO (i.e., not covalently linked to an antibody), or 122 mg/kg anti-TfR1 Fab (3M12 VH4/Vκ3) covalently linked to the DMD exon 51-skipping oligonucleotide (30 mg/kg ASO equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100 .$$

Calculated exon 51 skipping results are shown in Table 10.

TABLE 10
Exon 51 skipping of DMD mRNA in cynomolgus monkey

| | Time | | | | |
|---|---|---|---|---|---|
| | 2 weeks | | | 4 weeks | |
| | | Naked | | Naked | |
| Group | Vehicle | ASO^{a} | Conjugate | ASO^{a} | Conjugate |
| Conjugate dose^{b} | 0 | n/a | 122 | n/a | 122 |
| ASO Dose^{c} | 0 | 30 | 30 | 30 | 30 |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis anterior ^{d} | 0.00 | 0.699 | 1.042 | 0.384 | 0.816 |
| | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}ASO = antisense oligonucleotide.
^{b}Conjugate doses are listed as mg/kg of anti-TfR1 Fab 3M12 VH4/Vκ3-ASO conjugate.
^{c}ASO doses are listed as mg/kg ASO or ASO equivalent of the anti-TfR1 Fab 3M12 VH4/Vκ3-ASO dose.
^{d} Exon skipping values are mean % exon 51 skipping with standard deviations (n = 5) in parentheses.

Tissue ASO accumulation was also quantified using a hybridization ELISA with a probe complementary to the ASO sequence. A standard curve was generated and ASO levels (in ng/g) were derived from a linear regression of the standard curve. The ASO was distributed to all tissues evaluated at a higher level following the administration of the anti-TfR1 Fab VH4/VK3-ASO conjugate as compared to the administration of naked ASO. Intravenous administration of naked ASO resulted in levels of ASO that were close to background levels in all tissues evaluated at 2 and 4 weeks after the first does was administered. Administration of anti-TfR1 Fab VH4/VK3-ASO conjugate resulted in distribution of ASO through the tissues evaluated with a rank order of heart>diaphragm>bicep>quadriceps>gastrocnemius>tibialis anterior 2 weeks after first dosing. The duration of tissue concentration was also assessed. Concentrations of the ASO in quadriceps, bicep and diaphragm decreased by less than 50% over the time period evaluated (2 to 4 weeks), while levels of ASO in the heart, tibialis anterior, and gastrocnemius remained virtually unchanged (Table 11). This indicates that anti-TfR1 Fab 3M12 VH4/VK3 enabled cellular internalization of the conjugate into muscle cells in vivo, resulting in activity of the exon skipping oligonucleotide in the muscle cells. Similarly, an anti-TfR1 antibody (e.g., anti-TfR1 Fab 3M12 VH4/VK3) in vivo can enable internalization of a conjugate comprising the anti-TfR1 antibody covalently linked to other exon skipping oligonucleotides (e.g., an exon skipping oligonucleotide provided herein, such as an exon 51 skipping oligonucleotide) into muscle cells and facilitate activity of the exon skipping oligonucleotide in the muscle cells.

TABLE 11
Tissue distribution of DMD exon 51
skipping ASO in cynomolgus monkeys

| | Time | | | | |
|---|---|---|---|---|---|
| | 2 weeks | | | 4 weeks | |
| | | Naked | | Naked | |
| Group | Vehicle | ASO^{a} | Conjugate | ASO^{a} | Conjugate |
| Conjugate | 0 | n/a | 122 | n/a | 122 |
| Dose^{b} | | | | | |
| ASO Dose^{c} | 0 | 30 | 30 | 30 | 30 |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis | 0 | 564.71 | 1591 | 169 | 1087 |
| anterior ^{d} | (76.31) | (327.88) | (253.50) | (110) | (514) |
| Gastroc- | 0 | 705.47 | 2096 | 170 | 1265 |
| nemius ^{d} | (41.15) | (863.75) | (474.04) | (69) | (272) |

^{a}ASO = Antisense oligonucleotide.
^{b}Conjugate doses are listed as mg/kg of anti-TfR1 Fab 3M12 VH4/Vκ3-ASO conjugate.
^{c} ASO doses are listed as mg/kg ASO or ASO equivalent of the anti-TfR1 Fab 3M12 VH4/Vκ3-ASO conjugate dose.
^{d} ASO values are mean concentrations of ASO in tissue as ng/g with standard deviations (n = 5) in parentheses.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 (TfR1) antibody covalently linked to an oligonucleotide configured for inducing skipping of exon 51 in a DMD pre-mRNA, wherein the oligonucleotide comprises a region of complementarity that is complementary with at least 8 consecutive nucleotides of any one of SEQ ID NOs: 160-383.

2-4. (canceled)

5. The complex of claim 1, wherein the anti-TfR1 antibody is a Fab fragment, a Fab′ fragment, a F(ab′)2 fragment, an scFv, an Fv, or a full-length IgG.

6. The complex of claim 5, wherein the anti-TfR1 antibody is a Fab fragment.

7-8. (canceled)

9. The complex of claim 1, wherein the anti-TfR1 antibody does not specifically bind to the transferrin binding site of the transferrin receptor 1 and/or wherein the anti-TfR1 antibody does not inhibit binding of transferrin to the transferrin receptor 1.

10. The complex of claim 1, wherein the oligonucleotide comprises a region of complementarity to at least 4 consecutive nucleotides of a splicing feature of the DMD pre-mRNA.

11. The complex of claim 10, wherein the splicing feature is an exonic splicing enhancer (ESE) in exon 51 of the DMD pre-mRNA, optionally wherein the ESE comprises a sequence of any one of SEQ ID NOs: 860-894.

12. The complex of claim 10, wherein the splicing feature is a branch point, a splice donor site, or a splice acceptor site, optionally wherein the splicing feature is across the junction of exon 50 and intron 50, in intron 50, across the junction of intron 50 and exon 51, across the junction of exon 51 and intron 51, in intron 51, or across the junction of intron 51 and exon 52 of the DMD pre-mRNA, and further optionally wherein the splicing feature comprises a sequence of any one of SEQ ID NOs: 855-859 and 895-898.

13. The complex of claim 1, wherein the oligonucleotide comprises a sequence complementary to any one of SEQ ID NOs: 160-383 or comprises a sequence of any one of SEQ ID NOs: 384-831, wherein each thymine base (T) may independently and optionally be replaced with a uracil base (U), and each U may independently and optionally be replaced with a T.

14. The complex of claim 1, wherein the oligonucleotide comprises one or more phosphorodiamidate morpholinos, optionally wherein the oligonucleotide is a phosphorodiamidate morpholino oligomer (PMO).

15. The complex of claim 1, wherein the anti-TfR1 antibody is covalently linked to the oligonucleotide via a cleavable linker, optionally wherein the cleavable linker comprises a valine-citrulline sequence.

16. The complex of claim 1, wherein the anti-TfR1 antibody is covalently linked to the oligonucleotide via conjugation to a lysine residue or a cysteine residue of the antibody.

17. An oligonucleotide that targets DMD, wherein the oligonucleotide comprises a region of complementarity to any one of SEQ ID NOs: 160-383, optionally wherein the region of complementarity comprises at least 15 consecutive nucleosides complementary to any one of SEQ ID NOs: 160-383.

18. The oligonucleotide of claim 17, wherein the oligonucleotide comprises at least 15 consecutive nucleosides of any one of SEQ ID NOs: 384-831, optionally wherein the oligonucleotide comprises a sequence of any one of SEQ ID NOs: 384-831, wherein each thymine base (T) may independently and optionally be replaced with a uracil base (U), and each U may independently and optionally be replaced with a T.

19. A method of delivering an oligonucleotide to a cell, the method comprising contacting the cell with the complex of claim 1.

20. A method of promoting the expression or activity of a dystrophin protein in a cell, the method comprising contacting the cell with the complex of claim 1 in an amount effective for promoting internalization of the oligonucleotide to the cell, optionally wherein the cell is a muscle cell.


SEQUENCE LISTING (974 sequences)

SEQ ID NO: 1 - protein, 8 aa, synthetic construct
  REGION 1..8: note = Synthetic
GFNIKDDY

SEQ ID NO: 2 - protein, 8 aa, synthetic construct
  REGION 1..8: note = Synthetic
IDPENGDT

SEQ ID NO: 3 - protein, 10 aa, synthetic construct
  REGION 1..10: note = Synthetic
TLWLRRGLDY

SEQ ID NO: 4 - protein, 11 aa, synthetic construct
  REGION 1..11: note = Synthetic
KSLLHSNGYTY

SEQ ID NO: 5 - skipped

SEQ ID NO: 6 - protein, 9 aa, synthetic construct
  REGION 1..9: note = Synthetic
MQHLEYPFT

SEQ ID NO: 7 - protein, 5 aa, synthetic construct
  REGION 1..5: note = Synthetic
DDYMY

SEQ ID NO: 8 - protein, 17 aa, synthetic construct
  REGION 1..17: note = Synthetic
WIDPENGDTEYASKFQD

SEQ ID NO: 9 - protein, 8 aa, synthetic construct
  REGION 1..8: note = Synthetic
WLRRGLDY

SEQ ID NO: 10 - protein, 16 aa, synthetic construct
  REGION 1..16: note = Synthetic
RSSKSLLHSNGYTYLF

SEQ ID NO: 11 - protein, 7 aa, synthetic construct
  REGION 1..7: note = Synthetic
RMSNLAS

SEQ ID NO: 12 - protein, 7 aa, synthetic construct
  REGION 1..7: note = Synthetic
GFNIKDD

SEQ ID NO: 13 - skipped

SEQ ID NO: 14 - protein, 6 aa, synthetic construct
  REGION 1..6: note = Synthetic
LRRGLD

SEQ ID NO: 15 - protein, 12 aa, synthetic construct
  REGION 1..12: note = Synthetic
SKSLLHSNGYTY

SEQ ID NO: 16 - protein, 6 aa, synthetic construct
  REGION 1..6: note = Synthetic
HLEYPF

SEQ ID NO: 17 - protein, 117 aa, synthetic construct
  REGION 1..117: note = Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 18 - protein, 112 aa, synthetic construct
  REGION 1..112: note = Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 19 - protein, 8 aa, synthetic construct
  REGION 1..8: note = Synthetic
IDPETGDT

SEQ ID NO: 20 - protein, 17 aa, synthetic construct
  REGION 1..17: note = Synthetic
WIDPETGDTEYASKFQD

SEQ ID NO: 21 - skipped

SEQ ID NO: 22 - protein, 117 aa, synthetic construct
  REGION 1..117: note = Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSS

SEQ ID NO: 23 - protein, 8 aa, synthetic construct
  REGION 1..8: note = Synthetic
IDPESGDT

SEQ ID NO: 24 - protein, 17 aa, synthetic construct
  REGION 1..17: note = Synthetic
WIDPESGDTEYASKFQD

SEQ ID NO: 25 - skipped

SEQ ID NO: 26 - protein, 117 aa, synthetic construct
  REGION 1..117: note = Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSS

SEQ ID NO: 27 - protein, 9 aa, synthetic construct
  REGION 1..9: note = Synthetic
GYSITSGYY

SEQ ID NO: 28 - protein, 7 aa, synthetic construct
  REGION 1..7: note = Synthetic
ITFDGAN

SEQ ID NO: 29 - protein, 12 aa, synthetic construct
  REGION 1..12: note = Synthetic
TRSSYDYDVLDY

SEQ ID NO: 30 - protein, 6 aa, synthetic construct
  REGION 1..6: note = Synthetic
QDISNF

SEQ ID NO: 31 - skipped

SEQ ID NO: 32 - protein, 9 aa, synthetic construct
  REGION 1..9: note = Synthetic
QQGHTLPYT

SEQ ID NO: 33 - protein, 6 aa, synthetic construct
  REGION 1..6: note = Synthetic
SGYYWN

SEQ ID NO: 34 - protein, 16 aa, synthetic construct
  REGION 1..16: note = Synthetic
YITFDGANNYNPSLKN

SEQ ID NO: 35 - protein, 10 aa, synthetic construct
  REGION 1..10: note = Synthetic
SSYDYDVLDY

SEQ ID NO: 36 - protein, 11 aa, synthetic construct
  REGION 1..11: note = Synthetic
RASQDISNFLN

SEQ ID NO: 37 - protein, 7 aa, synthetic construct
  REGION 1..7: note = Synthetic
YTSRLHS

SEQ ID NO: 38 - protein, 8 aa, synthetic construct
  REGION 1..8: note = Synthetic
GYSITSGY

SEQ ID NO: 39 - skipped

SEQ ID NO: 40 - protein, 8 aa, synthetic construct
  REGION 1..8: note = Synthetic
SYDYDVLD

SEQ ID NO: 41 - protein, 7 aa, synthetic construct
  REGION 1..7: note = Synthetic
SQDISNF

SEQ ID NO: 42 - protein, 6 aa, synthetic construct
  REGION 1..6: note = Synthetic
GHTLPY

SEQ ID NO: 43 - protein, 119 aa, synthetic construct
  REGION 1..119: note = Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 44 - protein, 107 aa, synthetic construct
  REGION 1..107: note = Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 45 - protein, 8 aa, synthetic construct
  REGION 1..8: note = Synthetic
GYSFTDYC

SEQ ID NO: 46 - protein, 8 aa, synthetic construct
  REGION 1..8: note = Synthetic
IYPGSGNT

SEQ ID NO: 47 - protein, 13 aa, synthetic construct
  REGION 1..13: note = Synthetic
AREDYYPYHGMDY

SEQ ID NO: 48 - protein, 10 aa, synthetic construct
  REGION 1..10: note = Synthetic
ESVDGYDNSF

SEQ ID NO: 49 - skipped

SEQ ID NO: 50 - protein, 9 aa, synthetic construct
  REGION 1..9: note = Synthetic
QQSSEDPWT

SEQ ID NO: 51 - protein, 5 aa, synthetic construct
  REGION 1..5: note = Synthetic
DYCIN

SEQ ID NO: 52 - protein, 17 aa, synthetic construct
  REGION 1..17: note = Synthetic
WIYPGSGNTRYSERFKG

SEQ ID NO: 53 - protein, 11 aa, synthetic construct
  REGION 1..11: note = Synthetic
EDYYPYHGMDY

SEQ ID NO: 54 - protein, 15 aa, synthetic construct
  REGION 1..15: note = Synthetic
RASESVDGYDNSFMH

SEQ ID NO: 55 - protein, 7 aa, synthetic construct
  REGION 1..7: note = Synthetic
RASNLES

SEQ ID NO: 56 - protein, 7 aa, synthetic construct
  REGION 1..7: note = Synthetic
GYSFTDY

SEQ ID NO: 57 - skipped

SEQ ID NO: 58 - protein, 9 aa, synthetic construct
  REGION 1..9: note = Synthetic
DYYPYHGMD

SEQ ID NO: 59 - protein, 11 aa, synthetic construct
  REGION 1..11: note = Synthetic
SESVDGYDNSF

SEQ ID NO: 60 - protein, 6 aa, synthetic construct
  REGION 1..6: note = Synthetic
SSEDPW

SEQ ID NO: 61 - protein, 120 aa, synthetic construct
  REGION 1..120: note = Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 62 - protein, 111 aa, synthetic construct
  REGION 1..111: note = Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 63 - protein, 8 aa, synthetic construct
  REGION 1..8: note = Synthetic
GYSFTDYY

SEQ ID NO: 64 - protein, 5 aa, synthetic construct
  REGION 1..5: note = Synthetic
DYYIN

SEQ ID NO: 65 - protein, 120 aa, synthetic construct
  REGION 1..120: note = Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 66 - protein, 8 aa, synthetic construct
  REGION 1..8: note = Synthetic
GYSFTDYD

SEQ ID NO: 67 - protein, 5 aa, synthetic construct
  REGION 1..5: note = Synthetic
DYDIN

SEQ ID NO: 68 - protein, 120 aa, synthetic construct
  REGION 1..120: note = Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 69 - protein, 117 aa, synthetic construct
  REGION 1..117: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSS

SEQ ID NO: 70 - protein, 112 aa, synthetic construct
  REGION 1..112: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIK

SEQ ID NO: 71 - protein, 117 aa, synthetic construct
  REGION 1..117: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSS

SEQ ID NO: 72 - protein, 117 aa, synthetic construct
  REGION 1..117: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSS

SEQ ID NO: 73 - protein, 119 aa, synthetic construct
  REGION 1..119: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSS

SEQ ID NO: 74 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIK

SEQ ID NO: 75 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 76 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 77 - protein, 120 aa, synthetic construct
  REGION 1..120: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 78 - protein, 111 aa, synthetic construct
  REGION 1..111: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIK

SEQ ID NO: 79 - protein, 120 aa, synthetic construct
  REGION 1..120: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 80 - protein, 111 aa, synthetic construct
  REGION 1..111: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIK

SEQ ID NO: 81 - protein, 330 aa, Homo sapiens [sequence omitted]

SEQ ID NO: 82 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 107 aa, synthetic construct [sequence omitted]

SEQ ID NO: 84 - protein, 447 aa, synthetic construct
  REGION 1..447: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 219 aa, synthetic construct
  REGION 1..219: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 447 aa, synthetic construct
  REGION 1..447: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 447 aa, synthetic construct
  REGION 1..447: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  REGION 1..449: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 450 aa, synthetic construct
  REGION 1..450: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 218 aa, synthetic construct
  REGION 1..218: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, synthetic construct
  REGION 1..450: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 108 aa, synthetic construct
  REGION 1..108: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 225 aa, synthetic construct
  REGION 1..225: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 225 aa, synthetic construct
  REGION 1..225: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 225 aa, synthetic construct
  REGION 1..225: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 227 aa, synthetic construct
  REGION 1..227: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 228 aa, synthetic construct
  REGION 1..228: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 228 aa, synthetic construct
  REGION 1..228: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 19 aa, synthetic construct
  REGION 1..19: note = Synthetic
MGWSCIILFLVATATGVHS

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 107 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 108 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 197 aa, synthetic construct
  REGION 1..197: note = Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 110 - protein, 5 aa, synthetic construct
  REGION 1..5: note = Synthetic
SYWMH

SEQ ID NO: 111 - protein, 17 aa, synthetic construct
  REGION 1..17: note = Synthetic
EINPTNGRTNYIEKFKS

SEQ ID NO: 112 - protein, 7 aa, synthetic construct
  REGION 1..7: note = Synthetic
GTRAYHY

SEQ ID NO: 113 - protein, 11 aa, synthetic construct
  REGION 1..11: note = Synthetic
RASDNLYSNLA

SEQ ID NO: 114 - protein, 7 aa, synthetic construct
  REGION 1..7: note = Synthetic
DATNLAD

SEQ ID NO: 115 - protein, 9 aa, synthetic construct
  REGION 1..9: note = Synthetic
QHFWGTPLT

SEQ ID NO: 116 - protein, 7 aa, synthetic construct
  REGION 1..7: note = Synthetic
GYTFTSY

SEQ ID NO: 117 - protein, 6 aa, synthetic construct
  REGION 1..6: note = Synthetic
NPTNGR

SEQ ID NO: 118 - protein, 6 aa, synthetic construct
  REGION 1..6: note = Synthetic
TSYWMH

SEQ ID NO: 119 - protein, 13 aa, synthetic construct
  REGION 1..13: note = Synthetic
WIGEINPTNGRTN

SEQ ID NO: 120 - protein, 6 aa, synthetic construct
  REGION 1..6: note = Synthetic
ARGTRA

SEQ ID NO: 121 - protein, 7 aa, synthetic construct
  REGION 1..7: note = Synthetic
YSNLAWY

SEQ ID NO: 122 - protein, 10 aa, synthetic construct
  REGION 1..10: note = Synthetic
LLVYDATNLA

SEQ ID NO: 123 - protein, 8 aa, synthetic construct
  REGION 1..8: note = Synthetic
QHFWGTPL

SEQ ID NO: 124 - protein, 116 aa, Mus musculus
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 125 - protein, 107 aa, Mus musculus
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 126 - protein, 9 aa, synthetic construct
  REGION 1..9: note = Synthetic
QHFAGTPLT

SEQ ID NO: 127 - protein, 8 aa, synthetic construct
  REGION 1..8: note = Synthetic
QHFAGTPL

SEQ ID NO: 128 - protein, 116 aa, synthetic construct
  REGION 1..116: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 129 - protein, 107 aa, synthetic construct
  REGION 1..107: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 130 - protein, 13993 aa, Homo sapiens
TCCTGGCATCAGTTACTGTGTTGACTCACTCAGTGTTGGGATCACTCACTTTCCCCCTACAGGACTCAGATCTGGGAGGCAATTACCTTCGGAGAAAAACGAATAGGAAAAACTGAAGTGTTACTTTTTTTAAAGCTGCTGAAGTTTGTTGGTTTCTCATTGTTTTTAAGCCTACTGGAGCAATAAAGTTTGAAGAACTTTTACCAGGTTTTTTTTATCGCTGCCTTGATATACACTTTTCAAAATGCTTTGGTGGGAAGAAGTAGAGGACTGTTATGAAAGAGAAGATGTTCAAAAGAAAACATTCACAAAATGGGTAAATGCACAATTTTCTAAGTTTGGGAAGCAGCATATTGAGAACCTCTTCAGTGACCTACAGGATGGGAGGCGCCTCCTAGACCTCCTCGAAGGCCTGACAGGGCAAAAACTGCCAAAAGAAAAAGGATCCACAAGAGTTCATGCCCTGAACAATGTCAACAAGGCACTGCGGGTTTTGCAGAACAATAATGTTGATTTAGTGAATATTGGAAGTACTGACATCGTAGATGGAAATCATAAACTGACTCTTGGTTTGATTTGGAATATAATCCTCCACTGGCAGGTCAAAAATGTAATGAAAAATATCATGGCTGGATTGCAACAAACCAACAGTGAAAAGATTCTCCTGAGCTGGGTCCGACAATCAACTCGTAATTATCCACAGGTTAATGTAATCAACTTCACCACCAGCTGGTCTGATGGCCTGGCTTTGAATGCTCTCATCCATAGTCATAGGCCAGACCTATTTGACTGGAATAGTGTGGTTTGCCAGCAGTCAGCCACACAACGACTGGAACATGCATTCAACATCGCCAGATATCAATTAGGCATAGAGAAACTACTCGATCCTGAAGATGTTGATACCACCTATCCAGATAAGAAGTCCATCTTAATGTACATCACATCACTCTTCCAAGTTTTGCCTCAACAAGTGAGCATTGAAGCCATCCAGGAAGTGGAAATGTTGCCAAGGCCACCTAAAGTGACTAAAGAAGAACATTTTCAGTTACATCATCAAATGCACTATTCTCAACAGATCACGGTCAGTCTAGCACAGGGATATGAGAGAACTTCTTCCCCTAAGCCTCGATTCAAGAGCTATGCCTACACACAGGCTGCTTATGTCACCACCTCTGACCCTACACGGAGCCCATTTCCTTCACAGCATTTGGAAGCTCCTGAAGACAAGTCATTTGGCAGTTCATTGATGGAGAGTGAAGTAAACCTGGACCGTTATCAAACAGCTTTAGAAGAAGTATTATCGTGGCTTCTTTCTGCTGAGGACACATTGCAAGCACAAGGAGAGATTTCTAATGATGTGGAAGTGGTGAAAGACCAGTTTCATACTCATGAGGGGTACATGATGGATTTGACAGCCCATCAGGGCCGGGTTGGTAATATTCTACAATTGGGAAGTAAGCTGATTGGAACAGGAAAATTATCAGAAGATGAAGAAACTGAAGTACAAGAGCAGATGAATCTCCTAAATTCAAGATGGGAATGCCTCAGGGTAGCTAGCATGGAAAAACAAAGCAATTTACATAGAGTTTTAATGGATCTCCAGAATCAGAAACTGAAAGAGTTGAATGACTGGCTAACAAAAACAGAAGAAAGAACAAGGAAAATGGAGGAAGAGCCTCTTGGACCTGATCTTGAAGACCTAAAACGCCAAGTACAACAACATAAGGTGCTTCAAGAAGATCTAGAACAAGAACAAGTCAGGGTCAATTCTCTCACTCACATGGTGGTGGTAGTTGATGAATCTAGTGGAGATCACGCAACTGCTGCTTTGGAAGAACAACTTAAGGTATTGGGAGATCGATGGGCAAACATCTGTAGATGGACAGAAGACCGCTGGGTTCTTTTACAAGACATCCTTCTCAAATGGCAACGTCTTACTGAAGAACAGTGCCTTTTTAGTGCATGGCTTTCAGAAAAAGAAGATGCAGTGAACAAGATTCACACAACTGGCTTTAAAGATCAAAATGAAATGTTATCAAGTCTTCAAAAACTGGCCGTTTTAAAAGCGGATCTAGAAAAGAAAAAGCAATCCATGGGCAAACTGTATTCACTCAAACAAGATCTTCTTTCAACACTGAAGAATAAGTCAGTGACCCAGAAGACGGAAGCATGGCTGGATAACTTTGCCCGGTGTTGGGATAATTTAGTCCAAAAACTTGAAAAGAGTACAGCACAGATTTCACAGGCTGTCACCACCACTCAGCCATCACTAACACAGACAACTGTAATGGAAACAGTAACTACGGTGACCACAAGGGAACAGATCCTGGTAAAGCATGCTCAAGAGGAACTTCCACCACCACCTCCCCAAAAGAAGAGGCAGATTACTGTGGATTCTGAAATTAGGAAAAGGTTGGATGTTGATATAACTGAACTTCACAGCTGGATTACTCGCTCAGAAGCTGTGTTGCAGAGTCCTGAATTTGCAATCTTTCGGAAGGAAGGCAACTTCTCAGACTTAAAAGAAAAAGTCAATGCCATAGAGCGAGAAAAAGCTGAGAAGTTCAGAAAACTGCAAGATGCCAGCAGATCAGCTCAGGCCCTGGTGGAACAGATGGTGAATGAGGGTGTTAATGCAGATAGCATCAAACAAGCCTCAGAACAACTGAACAGCCGGTGGATCGAATTCTGCCAGTTGCTAAGTGAGAGACTTAACTGGCTGGAGTATCAGAACAACATCATCGCTTTCTATAATCAGCTACAACAATTGGAGCAGATGACAACTACTGCTGAAAACTGGTTGAAAATCCAACCCACCACCCCATCAGAGCCAACAGCAATTAAAAGTCAGTTAAAAATTTGTAAGGATGAAGTCAACCGGCTATCAGGTCTTCAACCTCAAATTGAACGATTAAAAATTCAAAGCATAGCCCTGAAAGAGAAAGGACAAGGACCCATGTTCCTGGATGCAGACTTTGTGGCCTTTACAAATCATTTTAAGCAAGTCTTTTCTGATGTGCAGGCCAGAGAGAAAGAGCTACAGACAATTTTTGACACTTTGCCACCAATGCGCTATCAGGAGACCATGAGTGCCATCAGGACATGGGTCCAGCAGTCAGAAACCAAACTCTCCATACCTCAACTTAGTGTCACCGACTATGAAATCATGGAGCAGAGACTCGGGGAATTGCAGGCTTTACAAAGTTCTCTGCAAGAGCAACAAAGTGGCCTATACTATCTCAGCACCACTGTGAAAGAGATGTCGAAGAAAGCGCCCTCTGAAATTAGCCGGAAATATCAATCAGAATTTGAAGAAATTGAGGGACGCTGGAAGAAGCTCTCCTCCCAGCTGGTTGAGCATTGTCAAAAGCTAGAGGAGCAAATGAATAAACTCCGAAAAATTCAGAATCACATACAAACCCTGAAGAAATGGATGGCTGAAGTTGATGTTTTTCTGAAGGAGGAATGGCCTGCCCTTGGGGATTCAGAAATTCTAAAAAAGCAGCTGAAACAGTGCAGACTTTTAGTCAGTGATATTCAGACAATTCAGCCCAGTCTAAACAGTGTCAATGAAGGTGGGCAGAAGATAAAGAATGAAGCAGAGCCAGAGTTTGCTTCGAGACTTGAGACAGAACTCAAAGAACTTAACACTCAGTGGGATCACATGTGCCAACAGGTCTATGCCAGAAAGGAGGCCTTGAAGGGAGGTTTGGAGAAAACTGTAAGCCTCCAGAAAGATCTATCAGAGATGCACGAATGGATGACACAAGCTGAAGAAGAGTATCTTGAGAGAGATTTTGAATATAAAACTCCAGATGAATTACAGAAAGCAGTTGAAGAGATGAAGAGAGCTAAAGAAGAGGCCCAACAAAAAGAAGCGAAAGTGAAACTCCTTACTGAGTCTGTAAATAGTGTCATAGCTCAAGCTCCACCTGTAGCACAAGAGGCCTTAAAAAAGGAACTTGAAACTCTAACCACCAACTACCAGTGGCTCTGCACTAGGCTGAATGGGAAATGCAAGACTTTGGAAGAAGTTTGGGCATGTTGGCATGAGTTATTGTCATACTTGGAGAAAGCAAACAAGTGGCTAAATGAAGTAGAATTTAAACTTAAAACCACTGAAAACATTCCTGGCGGAGCTGAGGAAATCTCTGAGGTGCTAGATTCACTTGAAAATTTGATGCGACATTCAGAGGATAACCCAAATCAGATTCGCATATTGGCACAGACCCTAACAGATGGCGGAGTCATGGATGAGCTAATCAATGAGGAACTTGAGACATTTAATTCTCGTTGGAGGGAACTACATGAAGAGGCTGTAAGGAGGCAAAAGTTGCTTGAACAGAGCATCCAGTCTGCCCAGGAGACTGAAAAATCCTTACACTTAATCCAGGAGTCCCTCACATTCATTGACAAGCAGTTGGCAGCTTATATTGCAGACAAGGTGGACGCAGCTCAAATGCCTCAGGAAGCCCAGAAAATCCAATCTGATTTGACAAGTCATGAGATCAGTTTAGAAGAAATGAAGAAACATAATCAGGGGAAGGAGGCTGCCCAAAGAGTCCTGTCTCAGATTGATGTTGCACAGAAAAAATTACAAGATGTCTCCATGAAGTTTCGATTATTCCAGAAACCAGCCAATTTTGAGCAGCGTCTACAAGAAAGTAAGATGATTTTAGATGAAGTGAAGATGCACTTGCCTGCATTGGAAACAAAGAGTGTGGAACAGGAAGTAGTACAGTCACAGCTAAATCATTGTGTGAACTTGTATAAAAGTCTGAGTGAAGTGAAGTCTGAAGTGGAAATGGTGATAAAGACTGGACGTCAGATTGTACAGAAAAAGCAGACGGAAAATCCCAAAGAACTTGATGAAAGAGTAACAGCTTTGAAATTGCATTATAATGAGCTGGGAGCAAAGGTAACAGAAAGAAAGCAACAGTTGGAGAAATGCTTGAAATTGTCCCGTAAGATGCGAAAGGAAATGAATGTCTTGACAGAATGGCTGGCAGCTACAGATATGGAATTGACAAAGAGATCAGCAGTTGAAGGAATGCCTAGTAATTTGGATTCTGAAGTTGCCTGGGGAAAGGCTACTCAAAAAGAGATTGAGAAACAGAAGGTGCACCTGAAGAGTATCACAGAGGTAGGAGAGGCCTTGAAAACAGTTTTGGGCAAGAAGGAGACGTTGGTGGAAGATAAACTCAGTCTTCTGAATAGTAACTGGATAGCTGTCACCTCCCGAGCAGAAGAGTGGTTAAATCTTTTGTTGGAATACCAGAAACACATGGAAACTTTTGACCAGAATGTGGACCACATCACAAAGTGGATCATTCAGGCTGACACACTTTTGGATGAATCAGAGAAAAAGAAACCCCAGCAAAAAGAAGACGTGCTTAAGCGTTTAAAGGCAGAACTGAATGACATACGCCCAAAGGTGGACTCTACACGTGACCAAGCAGCAAACTTGATGGCAAACCGCGGTGACCACTGCAGGAAATTAGTAGAGCCCCAAATCTCAGAGCTCAACCATCGATTTGCAGCCATTTCACACAGAATTAAGACTGGAAAGGCCTCCATTCCTTTGAAGGAATTGGAGCAGTTTAACTCAGATATACAAAAATTGCTTGAACCACTGGAGGCTGAAATTCAGCAGGGGGTGAATCTGAAAGAGGAAGACTTCAATAAAGATATGAATGAAGACAATGAGGGTACTGTAAAAGAATTGTTGCAAAGAGGAGACAACTTACAACAAAGAATCACAGATGAGAGAAAGCGAGAGGAAATAAAGATAAAACAGCAGCTGTTACAGACAAAACATAATGCTCTCAAGGATTTGAGGTCTCAAAGAAGAAAAAAGGCTCTAGAAATTTCTCATCAGTGGTATCAGTACAAGAGGCAGGCTGATGATCTCCTGAAATGCTTGGATGACATTGAAAAAAAATTAGCCAGCCTACCTGAGCCCAGAGATGAAAGGAAAATAAAGGAAATTGATCGGGAATTGCAGAAGAAGAAAGAGGAGCTGAATGCAGTGCGTAGGCAAGCTGAGGGCTTGTCTGAGGATGGGGCCGCAATGGCAGTGGAGCCAACTCAGATCCAGCTCAGCAAGCGCTGGCGGGAAATTGAGAGCAAATTTGCTCAGTTTCGAAGACTCAACTTTGCACAAATTCACACTGTCCGTGAAGAAACGATGATGGTGATGACTGAAGACATGCCTTTGGAAATTTCTTATGTGCCTTCTACTTATTTGACTGAAATCACTCATGTCTCACAAGCCCTATTAGAAGTGGAACAACTTCTCAATGCTCCTGACCTCTGTGCTAAGGACTTTGAAGATCTCTTTAAGCAAGAGGAGTCTCTGAAGAATATAAAAGATAGTCTACAACAAAGCTCAGGTCGGATTGACATTATTCATAGCAAGAAGACAGCAGCATTGCAAAGTGCAACGCCTGTGGAAAGGGTGAAGCTACAGGAAGCTCTCTCCCAGCTTGATTTCCAATGGGAAAAAGTTAACAAAATGTACAAGGACCGACAAGGGCGATTTGACAGATCTGTTGAGAAATGGCGGCGTTTTCATTATGATATAAAGATATTTAATCAGTGGCTAACAGAAGCTGAACAGTTTCTCAGAAAGACACAAATTCCTGAGAATTGGGAACATGCTAAATACAAATGGTATCTTAAGGAACTCCAGGATGGCATTGGGCAGCGGCAAACTGTTGTCAGAACATTGAATGCAACTGGGGAAGAAATAATTCAGCAATCCTCAAAAACAGATGCCAGTATTCTACAGGAAAAATTGGGAAGCCTGAATCTGCGGTGGCAGGAGGTCTGCAAACAGCTGTCAGACAGAAAAAAGAGGCTAGAAGAACAAAAGAATATCTTGTCAGAATTTCAAAGAGATTTAAATGAATTTGTTTTATGGTTGGAGGAAGCAGATAACATTGCTAGTATCCCACTTGAACCTGGAAAAGAGCAGCAACTAAAAGAAAAGCTTGAGCAAGTCAAGTTACTGGTGGAAGAGTTGCCCCTGCGCCAGGGAATTCTCAAACAATTAAATGAAACTGGAGGACCCGTGCTTGTAAGTGCTCCCATAAGCCCAGAAGAGCAAGATAAACTTGAAAATAAGCTCAAGCAGACAAATCTCCAGTGGATAAAGGTTTCCAGAGCTTTACCTGAGAAACAAGGAGAAATTGAAGCTCAAATAAAAGACCTTGGGCAGCTTGAAAAAAAGCTTGAAGACCTTGAAGAGCAGTTAAATCATCTGCTGCTGTGGTTATCTCCTATTAGGAATCAGTTGGAAATTTATAACCAACCAAACCAAGAAGGACCATTTGACGTTCAGGAAACTGAAATAGCAGTTCAAGCTAAACAACCGGATGTGGAAGAGATTTTGTCTAAAGGGCAGCATTTGTACAAGGAAAAACCAGCCACTCAGCCAGTGAAGAGGAAGTTAGAAGATCTGAGCTCTGAGTGGAAGGCGGTAAACCGTTTACTTCAAGAGCTGAGGGCAAAGCAGCCTGACCTAGCTCCTGGACTGACCACTATTGGAGCCTCTCCTACTCAGACTGTTACTCTGGTGACACAACCTGTGGTTACTAAGGAAACTGCCATCTCCAAACTAGAAATGCCATCTTCCTTGATGTTGGAGGTACCTGCTCTGGCAGATTTCAACCGGGCTTGGACAGAACTTACCGACTGGCTTTCTCTGCTTGATCAAGTTATAAAATCACAGAGGGTGATGGTGGGTGACCTTGAGGATATCAACGAGATGATCATCAAGCAGAAGGCAACAATGCAGGATTTGGAACAGAGGCGTCCCCAGTTGGAAGAACTCATTACCGCTGCCCAAAATTTGAAAAACAAGACCAGCAATCAAGAGGCTAGAACAATCATTACGGATCGAATTGAAAGAATTCAGAATCAGTGGGATGAAGTACAAGAACACCTTCAGAACCGGAGGCAACAGTTGAATGAAATGTTAAAGGATTCAACACAATGGCTGGAAGCTAAGGAAGAAGCTGAGCAGGTCTTAGGACAGGCCAGAGCCAAGCTTGAGTCATGGAAGGAGGGTCCCTATACAGTAGATGCAATCCAAAAGAAAATCACAGAAACCAAGCAGTTGGCCAAAGACCTCCGCCAGTGGCAGACAAATGTAGATGTGGCAAATGACTTGGCCCTGAAACTTCTCCGGGATTATTCTGCAGATGATACCAGAAAAGTCCACATGATAACAGAGAATATCAATGCCTCTTGGAGAAGCATTCATAAAAGGGTGAGTGAGCGAGAGGCTGCTTTGGAAGAAACTCATAGATTACTGCAACAGTTCCCCCTGGACCTGGAAAAGTTTCTTGCCTGGCTTACAGAAGCTGAAACAACTGCCAATGTCCTACAGGATGCTACCCGTAAGGAAAGGCTCCTAGAAGACTCCAAGGGAGTAAAAGAGCTGATGAAACAATGGCAAGACCTCCAAGGTGAAATTGAAGCTCACACAGATGTTTATCACAACCTGGATGAAAACAGCCAAAAAATCCTGAGATCCCTGGAAGGTTCCGATGATGCAGTCCTGTTACAAAGACGTTTGGATAACATGAACTTCAAGTGGAGTGAACTTCGGAAAAAGTCTCTCAACATTAGGTCCCATTTGGAAGCCAGTTCTGACCAGTGGAAGCGTCTGCACCTTTCTCTGCAGGAACTTCTGGTGTGGCTACAGCTGAAAGATGATGAATTAAGCCGGCAGGCACCTATTGGAGGCGACTTTCCAGCAGTTCAGAAGCAGAACGATGTACATAGGGCCTTCAAGAGGGAATTGAAAACTAAAGAACCTGTAATCATGAGTACTCTTGAGACTGTACGAATATTTCTGACAGAGCAGCCTTTGGAAGGACTAGAGAAACTCTACCAGGAGCCCAGAGAGCTGCCTCCTGAGGAGAGAGCCCAGAATGTCACTCGGCTTCTACGAAAGCAGGCTGAGGAGGTCAATACTGAGTGGGAAAAATTGAACCTGCACTCCGCTGACTGGCAGAGAAAAATAGATGAGACCCTTGAAAGACTCCAGGAACTTCAAGAGGCCACGGATGAGCTGGACCTCAAGCTGCGCCAAGCTGAGGTGATCAAGGGATCCTGGCAGCCCGTGGGCGATCTCCTCATTGACTCTCTCCAAGATCACCTCGAGAAAGTCAAGGCACTTCGAGGAGAAATTGCGCCTCTGAAAGAGAACGTGAGCCACGTCAATGACCTTGCTCGCCAGCTTACCACTTTGGGCATTCAGCTCTCACCGTATAACCTCAGCACTCTGGAAGACCTGAACACCAGATGGAAGCTTCTGCAGGTGGCCGTCGAGGACCGAGTCAGGCAGCTGCATGAAGCCCACAGGGACTTTGGTCCAGCATCTCAGCACTTTCTTTCCACGTCTGTCCAGGGTCCCTGGGAGAGAGCCATCTCGCCAAACAAAGTGCCCTACTATATCAACCACGAGACTCAAACAACTTGCTGGGACCATCCCAAAATGACAGAGCTCTACCAGTCTTTAGCTGACCTGAATAATGTCAGATTCTCAGCTTATAGGACTGCCATGAAACTCCGAAGACTGCAGAAGGCCCTTTGCTTGGATCTCTTGAGCCTGTCAGCTGCATGTGATGCCTTGGACCAGCACAACCTCAAGCAAAATGACCAGCCCATGGATATCCTGCAGATTATTAATTGTTTGACCACTATTTATGACCGCCTGGAGCAAGAGCACAACAATTTGGTCAACGTCCCTCTCTGCGTGGATATGTGTCTGAACTGGCTGCTGAATGTTTATGATACGGGACGAACAGGGAGGATCCGTGTCCTGTCTTTTAAAACTGGCATCATTTCCCTGTGTAAAGCACATTTGGAAGACAAGTACAGATACCTTTTCAAGCAAGTGGCAAGTTCAACAGGATTTTGTGACCAGCGCAGGCTGGGCCTCCTTCTGCATGATTCTATCCAAATTCCAAGACAGTTGGGTGAAGTTGCATCCTTTGGGGGCAGTAACATTGAGCCAAGTGTCCGGAGCTGCTTCCAATTTGCTAATAATAAGCCAGAGATCGAAGCGGCCCTCTTCCTAGACTGGATGAGACTGGAACCCCAGTCCATGGTGTGGCTGCCCGTCCTGCACAGAGTGGCTGCTGCAGAAACTGCCAAGCATCAGGCCAAATGTAACATCTGCAAAGAGTGTCCAATCATTGGATTCAGGTACAGGAGTCTAAAGCACTTTAATTATGACATCTGCCAAAGCTGCTTTTTTTCTGGTCGAGTTGCAAAAGGCCATAAAATGCACTATCCCATGGTGGAATATTGCACTCCGACTACATCAGGAGAAGATGTTCGAGACTTTGCCAAGGTACTAAAAAACAAATTTCGAACCAAAAGGTATTTTGCGAAGCATCCCCGAATGGGCTACCTGCCAGTGCAGACTGTCTTAGAGGGGGACAACATGGAAACTCCCGTTACTCTGATCAACTTCTGGCCAGTAGATTCTGCGCCTGCCTCGTCCCCTCAGCTTTCACACGATGATACTCATTCACGCATTGAACATTATGCTAGCAGGCTAGCAGAAATGGAAAACAGCAATGGATCTTATCTAAATGATAGCATCTCTCCTAATGAGAGCATAGATGATGAACATTTGTTAATCCAGCATTACTGCCAAAGTTTGAACCAGGACTCCCCCCTGAGCCAGCCTCGTAGTCCTGCCCAGATCTTGATTTCCTTAGAGAGTGAGGAAAGAGGGGAGCTAGAGAGAATCCTAGCAGATCTTGAGGAAGAAAACAGGAATCTGCAAGCAGAATATGACCGTCTAAAGCAGCAGCACGAACATAAAGGCCTGTCCCCACTGCCGTCCCCTCCTGAAATGATGCCCACCTCTCCCCAGAGTCCCCGGGATGCTGAGCTCATTGCTGAGGCCAAGCTACTGCGTCAACACAAAGGCCGCCTGGAAGCCAGGATGCAAATCCTGGAAGACCACAATAAACAGCTGGAGTCACAGTTACACAGGCTAAGGCAGCTGCTGGAGCAACCCCAGGCAGAGGCCAAAGTGAATGGCACAACGGTGTCCTCTCCTTCTACCTCTCTACAGAGGTCCGACAGCAGTCAGCCTATGCTGCTCCGAGTGGTTGGCAGTCAAACTTCGGACTCCATGGGTGAGGAAGATCTTCTCAGTCCTCCCCAGGACACAAGCACAGGGTTAGAGGAGGTGATGGAGCAACTCAACAACTCCTTCCCTAGTTCAAGAGGAAGAAATACCCCTGGAAAGCCAATGAGAGAGGACACAATGTAGGAAGTCTTTTCCACATGGCAGATGATTTGGGCAGAGCGATGGAGTCCTTAGTATCAGTCATGACAGATGAAGAAGGAGCAGAATAAATGTTTTACAACTCCTGATTCCCGCATGGTTTTTATAATATTCATACAACAAAGAGGATTAGACAGTAAGAGTTTACAAGAAATAAATCTATATTTTTGTGAAGGGTAGTGGTATTATACTGTAGATTTCAGTAGTTTCTAAGTCTGTTATTGTTTTGTTAACAATGGCAGGTTTTACACGTCTATGCAATTGTACAAAAAAGTTATAAGAAAACTACATGTAAAATCTTGATAGCTAAATAACTTGCCATTTCTTTATATGGAACGCATTTTGGGTTGTTTAAAAATTTATAACAGTTATAAAGAAAGATTGTAAACTAAAGTGTGCTTTATAAAAAAAAGTTGTTTATAAAAACCCCTAAAAACAAAACAAACACACACACACACACATACACACACACACACAAAACTTTGAGGCAGCGCATTGTTTTGCATCCTTTTGGCGTGATATCCATATGAAATTCATGGCTTTTTCTTTTTTTGCATATTAAAGATAAGACTTCCTCTACCACCACACCAAATGACTACTACACACTGCTCATTTGAGAACTGTCAGCTGAGTGGGGCAGGCTTGAGTTTTCATTTCATATATCTATATGTCTATAAGTATATAAATACTATAGTTATATAGATAAAGAGATACGAATTTCTATAGACTGACTTTTTCCATTTTTTAAATGTTCATGTCACATCCTAATAGAAAGAAATTACTTCTAGTCAGTCATCCAGGCTTACCTGCTTGGTCTAGAATGGATTTTTCCCGGAGCCGGAAGCCAGGAGGAAACTACACCACACTAAAACATTGTCTACAGCTCCAGATGTTTCTCATTTTAAACAACTTTCCACTGACAACGAAAGTAAAGTAAAGTATTGGATTTTTTTAAAGGGAACATGTGAATGAATACACAGGACTTATTATATCAGAGTGAGTAATCGGTTGGTTGGTTGATTGATTGATTGATTGATACATTCAGCTTCCTGCTGCTAGCAATGCCACGATTTAGATTTAATGATGCTTCAGTGGAAATCAATCAGAAGGTATTCTGACCTTGTGAACATCAGAAGGTATTTTTTAACTCCCAAGCAGTAGCAGGACGATGATAGGGCTGGAGGGCTATGGATTCCCAGCCCATCCCTGTGAAGGAGTAGGCCACTCTTTAAGTGAAGGATTGGATGATTGTTCATAATACATAAAGTTCTCTGTAATTACAACTAAATTATTATGCCCTCTTCTCACAGTCAAAAGGAACTGGGTGGTTTGGTTTTTGTTGCTTTTTTAGATTTATTGTCCCATGTGGGATGAGTTTTTAAATGCCACAAGACATAATTTAAAATAAATAAACTTTGGGAAAAGGTGTAAAACAGTAGCCCCATCACATTTGTGATACTGACAGGTATCAACCCAGAAGCCCATGAACTGTGTTTCCATCCTTTGCATTTCTCTGCGAGTAGTTCCACACAGGTTTGTAAGTAAGTAAGAAAGAAGGCAAATTGATTCAAATGTTACAAAAAAACCCTTCTTGGTGGATTAGACAGGTTAAATATATAAACAAACAAACAAAAATTGCTCAAAAAAGAGGAGAAAAGCTCAAGAGGAAAAGCTAAGGACTGGTAGGAAAAAGCTTTACTCTTTCATGCCATTTTATTTCTTTTTGATTTTTAAATCATTCATTCAATAGATACCACCGTGTGACCTATAATTTTGCAAATCTGTTACCTCTGACATCAAGTGTAATTAGCTTTTGGAGAGTGGGCTGACATCAAGTGTAATTAGCTTTTGGAGAGTGGGTTTTGTCCATTATTAATAATTAATTAATTAACATCAAACACGGCTTCTCATGCTATTTCTACCTCACTTTGGTTTTGGGGTGTTCCTGATAATTGTGCACACCTGAGTTCACAGCTTCACCACTTGTCCATTGCGTTATTTTCTTTTTCCTTTATAATTCTTTCTTTTTCCTTCATAATTTTCAAAAGAAAACCCAAAGCTCTAAGGTAACAAATTACCAAATTACATGAAGATTTGGTTTTTGTCTTGCATTTTTTTCCTTTATGTGACGCTGGACCTTTTCTTTACCCAAGGATTTTTAAAACTCAGATTTAAAACAAGGGGTTACTTTACATCCTACTAAGAAGTTTAAGTAAGTAAGTTTCATTCTAAAATCAGAGGTAAATAGAGTGCATAAATAATTTTGTTTTAATCTTTTTGTTTTTCTTTTAGACACATTAGCTCTGGAGTGAGTCTGTCATAATATTTGAACAAAAATTGAGAGCTTTATTGCTGCATTTTAAGCATAATTAATTTGGACATTATTTCGTGTTGTGTTCTTTATAACCACCAAGTATTAAACTGTAAATCATAATGTAACTGAAGCATAAACATCACATGGCATGTTTTGTCATTGTTTTCAGGTACTGAGTTCTTACTTGAGTATCATAATATATTGTGTTTTAACACCAACACTGTAACATTTACGAATTATTTTTTTAAACTTCAGTTTTACTGCATTTTCACAACATATCAGACTTCACCAAATATATGCCTTACTATTGTATTATAGTACTGCTTTACTGTGTATCTCAATAAAGCACGCAGTTATGTTAC

SEQ ID NO: 131 - protein, 109 aa, Homo sapiens
AGGAAGTTAGAAGATCTGAGCTCTGAGTGGAAGGCGGTAAACCGTTTACTTCAAGAGCTGAGGGCAAAGCAGCCTGACCTAGCTCCTGGACTGACCACTATTGGAGCCT

SEQ ID NO: 132 - protein, 446 aa, synthetic construct
  REGION 1..446: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, Homo sapiens
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 226 aa, synthetic construct
  REGION 1..226: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 137 - protein, 226 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 138 - protein, 8 aa, synthetic construct
  REGION 1..8: note = Synthetic
GYSFTSYW

SEQ ID NO: 139 - protein, 8 aa, synthetic construct
  REGION 1..8: note = Synthetic
IYPGDSDT

SEQ ID NO: 140 - protein, 15 aa, synthetic construct
  REGION 1..15: note = Synthetic
ARFPYDSSGYYSFDY

SEQ ID NO: 141 - protein, 6 aa, synthetic construct
  REGION 1..6: note = Synthetic
QSISSY

SEQ ID NO: 142 - skipped

SEQ ID NO: 143 - protein, 9 aa, synthetic construct
  REGION 1..9: note = Synthetic
QQSYSTPLT

SEQ ID NO: 144 - protein, 5 aa, synthetic construct
  REGION 1..5: note = Synthetic
SYWIG

SEQ ID NO: 145 - protein, 18 aa, synthetic construct
  REGION 1..18: note = Synthetic
IIYPGDSDTRYSPSFQGQ

SEQ ID NO: 146 - protein, 13 aa, synthetic construct
  REGION 1..13: note = Synthetic
FPYDSSGYYSFDY

SEQ ID NO: 147 - protein, 11 aa, synthetic construct
  REGION 1..11: note = Synthetic
RASQSISSYLN

SEQ ID NO: 148 - protein, 7 aa, synthetic construct
  REGION 1..7: note = Synthetic
AASSLQS

SEQ ID NO: 149 - protein, 7 aa, synthetic construct
  REGION 1..7: note = Synthetic
GYSFTSY

SEQ ID NO: 150 - skipped

SEQ ID NO: 151 - protein, 11 aa, synthetic construct
  REGION 1..11: note = Synthetic
PYDSSGYYSFD

SEQ ID NO: 152 - protein, 7 aa, synthetic construct
  REGION 1..7: note = Synthetic
SQSISSY

SEQ ID NO: 153 - protein, 6 aa, synthetic construct
  REGION 1..6: note = Synthetic
SYSTPL

SEQ ID NO: 154 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 155 - protein, 107 aa, synthetic construct [sequence omitted]

SEQ ID NO: 156 - protein, 452 aa, synthetic construct
  REGION 1..452: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 232 aa, synthetic construct
  REGION 1..232: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 159 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 160 - RNA, 22 nt, synthetic construct
  misc_feature 1..22: note = Synthetic
  source 1..22: mol_type = other RNA
gtaagtatactggatcccattc

SEQ ID NO: 161 - RNA, 23 nt, synthetic construct
  misc_feature 1..23: note = Synthetic
  source 1..23: mol_type = other RNA
gtaagtatactggatcccattct

SEQ ID NO: 162 - RNA, 24 nt, synthetic construct
  misc_feature 1..24: note = Synthetic
  source 1..24: mol_type = other RNA
gtaagtatactggatcccattctc

SEQ ID NO: 163 - RNA, 25 nt, synthetic construct
  misc_feature 1..25: note = Synthetic
  source 1..25: mol_type = other RNA
gtaagtatactggatcccattctct

SEQ ID NO: 164 - RNA, 23 nt, synthetic construct
  misc_feature 1..23: note = Synthetic
  source 1..23: mol_type = other RNA
taagtatactggatcccattctc

SEQ ID NO: 165 - RNA, 22 nt, synthetic construct
  misc_feature 1..22: note = Synthetic
  source 1..22: mol_type = other RNA
aagtatactggatcccattctc

SEQ ID NO: 166 - RNA, 21 nt, synthetic construct
  misc_feature 1..21: note = Synthetic
  source 1..21: mol_type = other RNA
agtatactggatcccattctc

SEQ ID NO: 167 - RNA, 25 nt, synthetic construct
  misc_feature 1..25: note = Synthetic
  source 1..25: mol_type = other RNA
gtatactggatcccattctctttgg

SEQ ID NO: 168 - RNA, 25 nt, synthetic construct
  misc_feature 1..25: note = Synthetic
  source 1..25: mol_type = other RNA
tactggatcccattctctttggctc

SEQ ID NO: 169 - RNA, 24 nt, synthetic construct
  misc_feature 1..24: note = Synthetic
  source 1..24: mol_type = other RNA
actggatcccattctctttggctc

SEQ ID NO: 170 - RNA, 24 nt, synthetic construct
  misc_feature 1..24: note = Synthetic
  source 1..24: mol_type = other RNA
tgtggttactaaggaaactgccat

SEQ ID NO: 171 - RNA, 25 nt, synthetic construct
  misc_feature 1..25: note = Synthetic
  source 1..25: mol_type = other RNA
tgtggttactaaggaaactgccatc

SEQ ID NO: 172 - RNA, 23 nt, synthetic construct
  misc_feature 1..23: note = Synthetic
  source 1..23: mol_type = other RNA
gtggttactaaggaaactgccat

SEQ ID NO: 173 - RNA, 24 nt, synthetic construct
  misc_feature 1..24: note = Synthetic
  source 1..24: mol_type = other RNA
gtggttactaaggaaactgccatc

SEQ ID NO: 174 - RNA, 25 nt, synthetic construct
  misc_feature 1..25: note = Synthetic
  source 1..25: mol_type = other RNA
gtggttactaaggaaactgccatct

SEQ ID NO: 175 - RNA, 23 nt, synthetic construct
  misc_feature 1..23: note = Synthetic
  source 1..23: mol_type = other RNA
tggttactaaggaaactgccatc

SEQ ID NO: 176 - RNA, 22 nt, synthetic construct
  misc_feature 1..22: note = Synthetic
  source 1..22: mol_type = other RNA
ggttactaaggaaactgccatc

SEQ ID NO: 177 - RNA, 24 nt, synthetic construct
  misc_feature 1..24: note = Synthetic
  source 1..24: mol_type = other RNA
gaaactgccatctccaaactagaa

SEQ ID NO: 178 - RNA, 21 nt, synthetic construct
  misc_feature 1..21: note = Synthetic
  source 1..21: mol_type = other RNA
aaactgccatctccaaactag

SEQ ID NO: 179 - RNA, 22 nt, synthetic construct
  misc_feature 1..22: note = Synthetic
  source 1..22: mol_type = other RNA
aaactgccatctccaaactaga

SEQ ID NO: 180 - RNA, 23 nt, synthetic construct
  misc_feature 1..23: note = Synthetic
  source 1..23: mol_type = other RNA
aaactgccatctccaaactagaa

SEQ ID NO: 181 - RNA, 20 nt, synthetic construct
  misc_feature 1..20: note = Synthetic
  source 1..20: mol_type = other RNA
aactgccatctccaaactag

SEQ ID NO: 182 - RNA, 21 nt, synthetic construct
  misc_feature 1..21: note = Synthetic
  source 1..21: mol_type = other RNA
aactgccatctccaaactaga

SEQ ID NO: 183 - RNA, 22 nt, synthetic construct
  misc_feature 1..22: note = Synthetic
  source 1..22: mol_type = other RNA
aactgccatctccaaactagaa

SEQ ID NO: 184 - RNA, 20 nt, synthetic construct
  misc_feature 1..20: note = Synthetic
  source 1..20: mol_type = other RNA
actgccatctccaaactaga

SEQ ID NO: 185 - RNA, 21 nt, synthetic construct
  misc_feature 1..21: note = Synthetic
  source 1..21: mol_type = other RNA
actgccatctccaaactagaa

SEQ ID NO: 186 - RNA, 22 nt, synthetic construct
  misc_feature 1..22: note = Synthetic
  source 1..22: mol_type = other RNA
tctccaaactagaaatgccatc

SEQ ID NO: 187 - RNA, 21 nt, synthetic construct
  misc_feature 1..21: note = Synthetic
  source 1..21: mol_type = other RNA
ctccaaactagaaatgccatc

SEQ ID NO: 188 - RNA, 20 nt, synthetic construct
  misc_feature 1..20: note = Synthetic
  source 1..20: mol_type = other RNA
tccaaactagaaatgccatc

SEQ ID NO: 189 - RNA, 23 nt, synthetic construct
  misc_feature 1..23: note = Synthetic
  source 1..23: mol_type = other RNA
gatttcaaccgggcttggacaga

SEQ ID NO: 190 - RNA, 24 nt, synthetic construct
  misc_feature 1..24: note = Synthetic
  source 1..24: mol_type = other RNA
gatttcaaccgggcttggacagaa

SEQ ID NO: 191 - RNA, 22 nt, synthetic construct
  misc_feature 1..22: note = Synthetic
  source 1..22: mol_type = other RNA
atttcaaccgggcttggacaga

SEQ ID NO: 192 - RNA, 25 nt, synthetic construct
  misc_feature 1..25: note = Synthetic
  source 1..25: mol_type = other RNA
atttcaaccgggcttggacagaact

SEQ ID NO: 193 - RNA, 23 nt, synthetic construct
  misc_feature 1..23: note = Synthetic
  source 1..23: mol_type = other RNA
ttcaaccgggcttggacagaact

SEQ ID NO: 194 - RNA, 20 nt, synthetic construct
  misc_feature 1..20: note = Synthetic
  source 1..20: mol_type = other RNA
tcaaccgggcttggacagaa

SEQ ID NO: 195 - RNA, 22 nt, synthetic construct
  misc_feature 1..22: note = Synthetic
  source 1..22: mol_type = other RNA
tcaaccgggcttggacagaact

SEQ ID NO: 196 - RNA, 25 nt, synthetic construct
  misc_feature 1..25: note = Synthetic
  source 1..25: mol_type = other RNA
tcaaccgggcttggacagaacttac

SEQ ID NO: 197 - RNA, 21 nt, synthetic construct
  misc_feature 1..21: note = Synthetic
  source 1..21: mol_type = other RNA
caaccgggcttggacagaact

SEQ ID NO: 198 - RNA, 24 nt, synthetic construct
  misc_feature 1..24: note = Synthetic
  source 1..24: mol_type = other RNA
caaccgggcttggacagaacttac

SEQ ID NO: 199 - RNA, 25 nt, synthetic construct
  misc_feature 1..25: note = Synthetic
  source 1..25: mol_type = other RNA
caaccgggcttggacagaacttacc

SEQ ID NO: 200 - RNA, 21 nt, synthetic construct
  misc_feature 1..21: note = Synthetic
  source 1..21: mol_type = other RNA
atgatcatcaagcagaaggta

SEQ ID NO: 201 - RNA, 23 nt, synthetic construct
  misc_feature 1..23: note = Synthetic
  source 1..23: mol_type = other RNA
atgatcatcaagcagaaggtatg

SEQ ID NO: 202 - RNA, 24 nt, synthetic construct
  misc_feature 1..24: note = Synthetic
  source 1..24: mol_type = other RNA
atgatcatcaagcagaaggtatga

SEQ ID NO: 203 - RNA, 25 nt, synthetic construct
  misc_feature 1..25: note = Synthetic
  source 1..25: mol_type = other RNA
atgatcatcaagcagaaggtatgag

SEQ ID NO: 204 - RNA, 20 nt, synthetic construct
  misc_feature 1..20: note = Synthetic
  source 1..20: mol_type = other RNA
tgatcatcaagcagaaggta

SEQ ID NO: 205 - RNA, 22 nt, synthetic construct
  misc_feature 1..22: note = Synthetic
  source 1..22: mol_type = other RNA
tgatcatcaagcagaaggtatg

SEQ ID NO: 206 - RNA, 23 nt, synthetic construct
  misc_feature 1..23: note = Synthetic
  source 1..23: mol_type = other RNA
tgatcatcaagcagaaggtatga

SEQ ID NO: 207 - RNA, 24 nt, synthetic construct
  misc_feature 1..24: note = Synthetic
  source 1..24: mol_type = other RNA
tgatcatcaagcagaaggtatgag

SEQ ID NO: 208 - RNA, 25 nt, synthetic construct
  misc_feature 1..25: note = Synthetic
  source 1..25: mol_type = other RNA
tgatcatcaagcagaaggtatgaga

SEQ ID NO: 209 - RNA, 21 nt, synthetic construct
  misc_feature 1..21: note = Synthetic
  source 1..21: mol_type = other RNA
gatcatcaagcagaaggtatg

SEQ ID NO: 210 - RNA, 22 nt, synthetic construct
  misc_feature 1..22: note = Synthetic
  source 1..22: mol_type = other RNA
gatcatcaagcagaaggtatga

SEQ ID NO: 211 - RNA, 23 nt, synthetic construct
  misc_feature 1..23: note = Synthetic
  source 1..23: mol_type = other RNA
gatcatcaagcagaaggtatgag

SEQ ID NO: 212 - RNA, 24 nt, synthetic construct
  misc_feature 1..24: note = Synthetic
  source 1..24: mol_type = other RNA
gatcatcaagcagaaggtatgaga

SEQ ID NO: 213 - RNA, 25 nt, synthetic construct
  misc_feature 1..25: note = Synthetic
  source 1..25: mol_type = other RNA
gatcatcaagcagaaggtatgagaa

SEQ ID NO: 214 - RNA, 20 nt, synthetic construct
  misc_feature 1..20: note = Synthetic
  source 1..20: mol_type = other RNA
atcatcaagcagaaggtatg

SEQ ID NO: 215 - RNA, 21 nt, synthetic construct
  misc_feature 1..21: note = Synthetic
  source 1..21: mol_type = other RNA
atcatcaagcagaaggtatga

SEQ ID NO: 216 - RNA, 22 nt, synthetic construct
  misc_feature 1..22: note = Synthetic
  source 1..22: mol_type = other RNA
atcatcaagcagaaggtatgag

SEQ ID NO: 217 - RNA, 23 nt, synthetic construct
  misc_feature 1..23: note = Synthetic
  source 1..23: mol_type = other RNA
atcatcaagcagaaggtatgaga

SEQ ID NO: 218 - RNA, 24 nt, synthetic construct
  misc_feature 1..24: note = Synthetic
  source 1..24: mol_type = other RNA
atcatcaagcagaaggtatgagaa

SEQ ID NO: 219 - RNA, 25 nt, synthetic construct
  misc_feature 1..25: note = Synthetic
  source 1..25: mol_type = other RNA
atcatcaagcagaaggtatgagaaa

SEQ ID NO: 220 - RNA, 20 nt, synthetic construct
  misc_feature 1..20: note = Synthetic
  source 1..20: mol_type = other RNA
tcatcaagcagaaggtatga

SEQ ID NO: 221 - RNA, 21 nt, synthetic construct
  misc_feature 1..21: note = Synthetic
  source 1..21: mol_type = other RNA
tcatcaagcagaaggtatgag

SEQ ID NO: 222 - RNA, 22 nt, synthetic construct
  misc_feature 1..22: note = Synthetic
  source 1..22: mol_type = other RNA
tcatcaagcagaaggtatgaga

SEQ ID NO: 223 - RNA, 23 nt, synthetic construct
  misc_feature 1..23: note = Synthetic
  source 1..23: mol_type = other RNA
tcatcaagcagaaggtatgagaa

SEQ ID NO: 224 - RNA, 24 nt, synthetic construct
  misc_feature 1..24: note = Synthetic
  source 1..24: mol_type = other RNA
tcatcaagcagaaggtatgagaaa

SEQ ID NO: 225 - RNA, 25 nt, synthetic construct
  misc_feature 1..25: note = Synthetic
  source 1..25: mol_type = other RNA
tcatcaagcagaaggtatgagaaaa

SEQ ID NO: 226 - RNA, 20 nt, synthetic construct
  misc_feature 1..20: note = Synthetic
  source 1..20: mol_type = other RNA
catcaagcagaaggtatgag

SEQ ID NO: 227 - RNA, 21 nt, synthetic construct
  misc_feature 1..21: note = Synthetic
  source 1..21: mol_type = other RNA
catcaagcagaaggtatgaga

SEQ ID NO: 228 - RNA, 22 nt, synthetic construct
  misc_feature 1..22: note = Synthetic
  source 1..22: mol_type = other RNA
catcaagcagaaggtatgagaa

SEQ ID NO: 229 - RNA, 23 nt, synthetic construct
  misc_feature 1..23: note = Synthetic
  source 1..23: mol_type = other RNA
catcaagcagaaggtatgagaaa

SEQ ID NO: 230 - RNA, 24 nt, synthetic construct
  misc_feature 1..24: note = Synthetic
  source 1..24: mol_type = other RNA
catcaagcagaaggtatgagaaaa

SEQ ID NO: 231 - RNA, 25 nt, synthetic construct
  misc_feature 1..25: note = Synthetic
  source 1..25: mol_type = other RNA
catcaagcagaaggtatgagaaaaa

SEQ ID NO: 232 - RNA, 20 nt, synthetic construct
  misc_feature 1..20: note = Synthetic
  source 1..20: mol_type = other RNA
atcaagcagaaggtatgaga

SEQ ID NO: 233 - RNA, 21 nt, synthetic construct
  misc_feature 1..21: note = Synthetic
  source 1..21: mol_type = other RNA
atcaagcagaaggtatgagaa

SEQ ID NO: 234 - RNA, 22 nt, synthetic construct
  misc_feature 1..22: note = Synthetic
  source 1..22: mol_type = other RNA
atcaagcagaaggtatgagaaa

SEQ ID NO: 235 - RNA, 23 nt, synthetic construct
  misc_feature 1..23: note = Synthetic
  source 1..23: mol_type = other RNA
atcaagcagaaggtatgagaaaa

SEQ ID NO: 236 - RNA, 24 nt, synthetic construct
  misc_feature 1..24: note = Synthetic
  source 1..24: mol_type = other RNA
atcaagcagaaggtatgagaaaaa

SEQ ID NO: 237 - RNA, 25 nt, synthetic construct
  misc_feature 1..25: note = Synthetic
  source 1..25: mol_type = other RNA
atcaagcagaaggtatgagaaaaaa

SEQ ID NO: 238 - RNA, 20 nt, synthetic construct
  misc_feature 1..20: note = Synthetic
  source 1..20: mol_type = other RNA
tcaagcagaaggtatgagaa

SEQ ID NO: 239 - RNA, 21 nt, synthetic construct
  misc_feature 1..21: note = Synthetic
  source 1..21: mol_type = other RNA
tcaagcagaaggtatgagaaa

SEQ ID NO: 240 - RNA, 22 nt, synthetic construct
  misc_feature 1..22: note = Synthetic
  source 1..22: mol_type = other RNA
tcaagcagaaggtatgagaaaa

SEQ ID NO: 241 - RNA, 23 nt, synthetic construct
  misc_feature 1..23: note = Synthetic
  source 1..23: mol_type = other RNA
tcaagcagaaggtatgagaaaaa

SEQ ID NO: 242 - RNA, 24 nt, synthetic construct
  misc_feature 1..24: note = Synthetic
  source 1..24: mol_type = other RNA
tcaagcagaaggtatgagaaaaaa

SEQ ID NO: 243 - RNA, 25 nt, synthetic construct
  misc_feature 1..25: note = Synthetic
  source 1..25: mol_type = other RNA
tcaagcagaaggtatgagaaaaaat

SEQ ID NO: 244 - RNA, 20 nt, synthetic construct
  misc_feature 1..20: note = Synthetic
  source 1..20: mol_type = other RNA
caagcagaaggtatgagaaa

SEQ ID NO: 245 - RNA, 21 nt, synthetic construct
  misc_feature 1..21: note = Synthetic
  source 1..21: mol_type = other RNA
caagcagaaggtatgagaaaa

SEQ ID NO: 246 - RNA, 22 nt, synthetic construct
  misc_feature 1..22: note = Synthetic
  source 1..22: mol_type = other RNA
caagcagaaggtatgagaaaaa

SEQ ID NO: 247 - RNA, 23 nt, synthetic construct
  misc_feature 1..23: note = Synthetic
  source 1..23: mol_type = other RNA
caagcagaaggtatgagaaaaaa

SEQ ID NO: 248 - RNA, 24 nt, synthetic construct
  misc_feature 1..24: note = Synthetic
  source 1..24: mol_type = other RNA
caagcagaaggtatgagaaaaaat

SEQ ID NO: 249 - RNA, 20 nt, synthetic construct
  misc_feature 1..20: note = Synthetic
  source 1..20: mol_type = other RNA
aagcagaaggtatgagaaaa

SEQ ID NO: 250 - RNA, 21 nt, synthetic construct
  misc_feature 1..21: note = Synthetic
  source 1..21: mol_type = other RNA
aagcagaaggtatgagaaaaa

SEQ ID NO: 251 - RNA, 22 nt, synthetic construct
  misc_feature 1..22: note = Synthetic
  source 1..22: mol_type = other RNA
aagcagaaggtatgagaaaaaa

SEQ ID NO: 252 - RNA, 23 nt, synthetic construct
  misc_feature 1..23: note = Synthetic
  source 1..23: mol_type = other RNA
aagcagaaggtatgagaaaaaat

SEQ ID NO: 253 - RNA, 20 nt, synthetic construct
  misc_feature 1..20: note = Synthetic
  source 1..20: mol_type = other RNA
agcagaaggtatgagaaaaa

SEQ ID NO: 254 - RNA, 21 nt, synthetic construct
  misc_feature 1..21: note = Synthetic
  source 1..21: mol_type = other RNA
agcagaaggtatgagaaaaaa

SEQ ID NO: 255 - RNA, 22 nt, synthetic construct
  misc_feature 1..22: note = Synthetic
  source 1..22: mol_type = other RNA
agcagaaggtatgagaaaaaat

SEQ ID NO: 256 - RNA, 20 nt, synthetic construct
  misc_feature 1..20: note = Synthetic
  source 1..20: mol_type = other RNA
gcagaaggtatgagaaaaaa

SEQ ID NO: 257 - RNA, 21 nt, synthetic construct
  misc_feature 1..21: note = Synthetic
  source 1..21: mol_type = other RNA
gcagaaggtatgagaaaaaat

SEQ ID NO: 258 - RNA, 20 nt, synthetic construct
  misc_feature 1..20: note = Synthetic
  source 1..20: mol_type = other RNA
cagaaggtatgagaaaaaat

SEQ ID NO: 259 - RNA, 23 nt, synthetic construct
  misc_feature 1..23: note = Synthetic
  source 1..23: mol_type = other RNA
aaatgataaaagttggcagaagt

SEQ ID NO: 260 - RNA, 22 nt, synthetic construct
  misc_feature 1..22: note = Synthetic
  source 1..22: mol_type = other RNA
tcactttactctcctagaccat

SEQ ID NO: 261 - RNA, 23 nt, synthetic construct
  misc_feature 1..23: note = Synthetic
  source 1..23: mol_type = other RNA
tcactttactctcctagaccatt

SEQ ID NO: 262 - RNA, 24 nt, synthetic construct
  misc_feature 1..24: note = Synthetic
  source 1..24: mol_type = other RNA
tcactttactctcctagaccattt

SEQ ID NO: 263 - RNA, 25 nt, synthetic construct
  misc_feature 1..25: note = Synthetic
  source 1..25: mol_type = other RNA
cactttactctcctagaccatttcc

SEQ ID NO: 264 - RNA, 24 nt, synthetic construct
  misc_feature 1..24: note = Synthetic
  source 1..24: mol_type = other RNA
actttactctcctagaccatttcc

SEQ ID NO: 265 - RNA, 25 nt, synthetic construct
  misc_feature 1..25: note = Synthetic
  source 1..25: mol_type = other RNA
ctttactctcctagaccatttccca

SEQ ID NO: 266 - RNA, 23 nt, synthetic construct
  misc_feature 1..23: note = Synthetic
  source 1..23: mol_type = other RNA
ttactctcctagaccatttccca

SEQ ID NO: 267 - RNA, 24 nt, synthetic construct
  misc_feature 1..24: note = Synthetic
  source 1..24: mol_type = other RNA
ttactctcctagaccatttcccac

SEQ ID NO: 268 - RNA, 25 nt, synthetic construct
  misc_feature 1..25: note = Synthetic
  source 1..25: mol_type = other RNA
ttactctcctagaccatttcccacc

SEQ ID NO: 269 - RNA, 22 nt, synthetic construct
  misc_feature 1..22: note = Synthetic
  source 1..22: mol_type = other RNA
tactctcctagaccatttccca

SEQ ID NO: 270 - RNA, 23 nt, synthetic construct
  misc_feature 1..23: note = Synthetic
  source 1..23: mol_type = other RNA
tactctcctagaccatttcccac

SEQ ID NO: 271 - RNA, 24 nt, synthetic construct
  misc_feature 1..24: note = Synthetic
  source 1..24: mol_type = other RNA
tactctcctagaccatttcccacc

SEQ ID NO: 272 - RNA, 25 nt, synthetic construct
  misc_feature 1..25: note = Synthetic
  source 1..25: mol_type = other RNA
tactctcctagaccatttcccacca

SEQ ID NO: 273 - RNA, 21 nt, synthetic construct
  misc_feature 1..21: note = Synthetic
  source 1..21: mol_type = other RNA
actctcctagaccatttccca

SEQ ID NO: 274 - RNA, 22 nt, synthetic construct
  misc_feature 1..22: note = Synthetic
  source 1..22: mol_type = other RNA
actctcctagaccatttcccac

SEQ ID NO: 275 - RNA, 23 nt, synthetic construct
  misc_feature 1..23: note = Synthetic
  source 1..23: mol_type = other RNA
actctcctagaccatttcccacc

SEQ ID NO: 276 - RNA, 24 nt, synthetic construct
  misc_feature 1..24: note = Synthetic
  source 1..24: mol_type = other RNA
actctcctagaccatttcccacca

SEQ ID NO: 277 - RNA, 25 nt, synthetic construct
  misc_feature 1..25: note = Synthetic
  source 1..25: mol_type = other RNA
actctcctagaccatttcccaccag

SEQ ID NO: 278 - RNA, 20 nt, synthetic construct
  misc_feature 1..20: note = Synthetic
  source 1..20: mol_type = other RNA
ctctcctagaccatttccca

SEQ ID NO: 279 - RNA, 21 nt, synthetic construct
  misc_feature 1..21: note = Synthetic
  source 1..21: mol_type = other RNA
ctctcctagaccatttcccac

SEQ ID NO: 280 - RNA, 22 nt, synthetic construct
  misc_feature 1..22: note = Synthetic
  source 1..22: mol_type = other RNA
ctctcctagaccatttcccacc

SEQ ID NO: 281 - RNA, 23 nt, synthetic construct
  misc_feature 1..23: note = Synthetic
  source 1..23: mol_type = other RNA
ctctcctagaccatttcccacca

SEQ ID NO: 282 - RNA, 24 nt, synthetic construct
  misc_feature 1..24: note = Synthetic
  source 1..24: mol_type = other RNA
ctctcctagaccatttcccaccag

SEQ ID NO: 283 - RNA, 20 nt, synthetic construct
  misc_feature 1..20: note = Synthetic
  source 1..20: mol_type = other RNA
tctcctagaccatttcccac

SEQ ID NO: 284 - RNA, 21 nt, synthetic construct
  misc_feature 1..21: note = Synthetic
  source 1..21: mol_type = other RNA
tctcctagaccatttcccacc

SEQ ID NO: 285 - RNA, 22 nt, synthetic construct
  misc_feature 1..22: note = Synthetic
  source 1..22: mol_type = other RNA
tctcctagaccatttcccacca

SEQ ID NO: 286 - RNA, 23 nt, synthetic construct
  misc_feature 1..23: note = Synthetic
  source 1..23: mol_type = other RNA
tctcctagaccatttcccaccag

SEQ ID NO: 287 - RNA, 25 nt, synthetic construct
  misc_feature 1..25: note = Synthetic
  source 1..25: mol_type = other RNA
tctcctagaccatttcccaccagtt

SEQ ID NO: 288 - RNA, 20 nt, synthetic construct
  misc_feature 1..20: note = Synthetic
  source 1..20: mol_type = other RNA
ctcctagaccatttcccacc

SEQ ID NO: 289 - RNA, 21 nt, synthetic construct
  misc_feature 1..21: note = Synthetic
  source 1..21: mol_type = other RNA
ctcctagaccatttcccacca

SEQ ID NO: 290 - RNA, 22 nt, synthetic construct
  misc_feature 1..22: note = Synthetic
  source 1..22: mol_type = other RNA
ctcctagaccatttcccaccag

SEQ ID NO: 291 - RNA, 24 nt, synthetic construct
  misc_feature 1..24: note = Synthetic
  source 1..24: mol_type = other RNA
ctcctagaccatttcccaccagtt

SEQ ID NO: 292 - RNA, 20 nt, synthetic construct
  misc_feature 1..20: note = Synthetic
  source 1..20: mol_type = other RNA
tcctagaccatttcccacca

SEQ ID NO: 293 - RNA, 21 nt, synthetic construct
  misc_feature 1..21: note = Synthetic
  source 1..21: mol_type = other RNA
tcctagaccatttcccaccag

SEQ ID NO: 294 - RNA, 23 nt, synthetic construct
  misc_feature 1..23: note = Synthetic
  source 1..23: mol_type = other RNA
tcctagaccatttcccaccagtt

SEQ ID NO: 295 - RNA, 25 nt, synthetic construct
  misc_feature 1..25: note = Synthetic
  source 1..25: mol_type = other RNA
tcctagaccatttcccaccagttct

SEQ ID NO: 296 - RNA, 20 nt, synthetic construct
  misc_feature 1..20: note = Synthetic
  source 1..20: mol_type = other RNA
cctagaccatttcccaccag

SEQ ID NO: 297 - RNA, 22 nt, synthetic construct
  misc_feature 1..22: note = Synthetic
  source 1..22: mol_type = other RNA
cctagaccatttcccaccagtt

SEQ ID NO: 298 - RNA, 24 nt, synthetic construct
  misc_feature 1..24: note = Synthetic
  source 1..24: mol_type = other RNA
cctagaccatttcccaccagttct

SEQ ID NO: 299 - RNA, 25 nt, synthetic construct
  misc_feature 1..25: note = Synthetic
  source 1..25: mol_type = other RNA
cctagaccatttcccaccagttctt

SEQ ID NO: 300 - RNA, 21 nt, synthetic construct
  misc_feature 1..21: note = Synthetic
  source 1..21: mol_type = other RNA
ctagaccatttcccaccagtt

SEQ ID NO: 301 - RNA, 23 nt, synthetic construct
  misc_feature 1..23: note = Synthetic
  source 1..23: mol_type = other RNA
ctagaccatttcccaccagttct

SEQ ID NO: 302 - RNA, 24 nt, synthetic construct
  misc_feature 1..24: note = Synthetic
  source 1..24: mol_type = other RNA
ctagaccatttcccaccagttctt

SEQ ID NO: 303 - RNA, 20 nt, synthetic construct
  misc_feature 1..20: note = Synthetic
  source 1..20: mol_type = other RNA
tagaccatttcccaccagtt

SEQ ID NO: 304 - RNA, 22 nt, synthetic construct
  misc_feature 1..22: note = Synthetic
  source 1..22: mol_type = other RNA
tagaccatttcccaccagttct

SEQ ID NO: 305 - RNA, 23 nt, synthetic construct
  misc_feature 1..23: note = Synthetic
  source 1..23: mol_type = other RNA
tagaccatttcccaccagttctt

SEQ ID NO: 306 - RNA, 25 nt, synthetic construct
  misc_feature 1..25: note = Synthetic
  source 1..25: mol_type = other RNA
tagaccatttcccaccagttcttag

SEQ ID NO: 307 - RNA, 21 nt, synthetic construct
  misc_feature 1..21: note = Synthetic
  source 1..21: mol_type = other RNA
agaccatttcccaccagttct

SEQ ID NO: 308 - RNA, 22 nt, synthetic construct
  misc_feature 1..22: note = Synthetic
  source 1..22: mol_type = other RNA
agaccatttcccaccagttctt

SEQ ID NO: 309 - RNA, 24 nt, synthetic construct
  misc_feature 1..24: note = Synthetic
  source 1..24: mol_type = other RNA
agaccatttcccaccagttcttag

SEQ ID NO: 310 - RNA, 25 nt, synthetic construct
  misc_feature 1..25: note = Synthetic
  source 1..25: mol_type = other RNA
agaccatttcccaccagttcttagg

SEQ ID NO: 311 - RNA, 20 nt, synthetic construct
  misc_feature 1..20: note = Synthetic
  source 1..20: mol_type = other RNA
gaccatttcccaccagttct

SEQ ID NO: 312 - RNA, 21 nt, synthetic construct
  misc_feature 1..21: note = Synthetic
  source 1..21: mol_type = other RNA
gaccatttcccaccagttctt

SEQ ID NO: 313 - RNA, 23 nt, synthetic construct
  misc_feature 1..23: note = Synthetic
  source 1..23: mol_type = other RNA
gaccatttcccaccagttcttag

SEQ ID NO: 314 - RNA, 24 nt, synthetic construct
  misc_feature 1..24: note = Synthetic
  source 1..24: mol_type = other RNA
gaccatttcccaccagttcttagg

SEQ ID NO: 315 - RNA, 25 nt, synthetic construct
  misc_feature 1..25: note = Synthetic
  source 1..25: mol_type = other RNA
gaccatttcccaccagttcttaggc

SEQ ID NO: 316 - RNA, 24 nt, synthetic construct
  misc_feature 1..24: note = Synthetic
  source 1..24: mol_type = other RNA
accatttcccaccagttcttaggc

SEQ ID NO: 317 - RNA, 25 nt, synthetic construct
  misc_feature 1..25: note = Synthetic
  source 1..25: mol_type = other RNA
accatttcccaccagttcttaggca

SEQ ID NO: 318 - RNA, 23 nt, synthetic construct
  misc_feature 1..23: note = Synthetic
  source 1..23: mol_type = other RNA
ccatttcccaccagttcttaggc

SEQ ID NO: 319 - RNA, 24 nt, synthetic construct
  misc_feature 1..24: note = Synthetic
  source 1..24: mol_type = other RNA
ccatttcccaccagttcttaggca

SEQ ID NO: 320 - RNA, 25 nt, synthetic construct
  misc_feature 1..25: note = Synthetic
  source 1..25: mol_type = other RNA
ccatttcccaccagttcttaggcaa

SEQ ID NO: 321 - RNA, 23 nt, synthetic construct
  misc_feature 1..23: note = Synthetic
  source 1..23: mol_type = other RNA
catttcccaccagttcttaggca

SEQ ID NO: 322 - RNA, 24 nt, synthetic construct
  misc_feature 1..24: note = Synthetic
  source 1..24: mol_type = other RNA
catttcccaccagttcttaggcaa

SEQ ID NO: 323 - RNA, 20 nt, synthetic construct
  misc_feature 1..20: note = Synthetic
  source 1..20: mol_type = other RNA
agtgttttggctggtctcac

SEQ ID NO: 324 - RNA, 21 nt, synthetic construct
  misc_feature 1..21: note = Synthetic
  source 1..21: mol_type = other RNA
agtgttttggctggtctcaca

SEQ ID NO: 325 - RNA, 22 nt, synthetic construct
  misc_feature 1..22: note = Synthetic
  source 1..22: mol_type = other RNA
agtgttttggctggtctcacaa

SEQ ID NO: 326 - RNA, 23 nt, synthetic construct
  misc_feature 1..23: note = Synthetic
  source 1..23: mol_type = other RNA
agtgttttggctggtctcacaat

SEQ ID NO: 327 - RNA, 22 nt, synthetic construct
  misc_feature 1..22: note = Synthetic
  source 1..22: mol_type = other RNA
gtgttttggctggtctcacaat

SEQ ID NO: 328 - RNA, 25 nt, synthetic construct
  misc_feature 1..25: note = Synthetic
  source 1..25: mol_type = other RNA
gttttggctggtctcacaattgtac

SEQ ID NO: 329 - RNA, 23 nt, synthetic construct
  misc_feature 1..23: note = Synthetic
  source 1..23: mol_type = other RNA
tttggctggtctcacaattgtac

SEQ ID NO: 330 - RNA, 22 nt, synthetic construct
  misc_feature 1..22: note = Synthetic
  source 1..22: mol_type = other RNA
ttggctggtctcacaattgtac

SEQ ID NO: 331 - RNA, 23 nt, synthetic construct
  misc_feature 1..23: note = Synthetic
  source 1..23: mol_type = other RNA
ttggctggtctcacaattgtact

SEQ ID NO: 332 - RNA, 21 nt, synthetic construct
  misc_feature 1..21: note = Synthetic
  source 1..21: mol_type = other RNA
tggctggtctcacaattgtac

SEQ ID NO: 333 - RNA, 22 nt, synthetic construct
  misc_feature 1..22: note = Synthetic
  source 1..22: mol_type = other RNA
tggctggtctcacaattgtact

SEQ ID NO: 334 - RNA, 23 nt, synthetic construct
  misc_feature 1..23: note = Synthetic
  source 1..23: mol_type = other RNA
tggctggtctcacaattgtactt

SEQ ID NO: 335 - RNA, 24 nt, synthetic construct
  misc_feature 1..24: note = Synthetic
  source 1..24: mol_type = other RNA
tggctggtctcacaattgtacttt

SEQ ID NO: 336 - RNA, 20 nt, synthetic construct
  misc_feature 1..20: note = Synthetic
  source 1..20: mol_type = other RNA
ggctggtctcacaattgtac

SEQ ID NO: 337 - RNA, 21 nt, synthetic construct
  misc_feature 1..21: note = Synthetic
  source 1..21: mol_type = other RNA
ggctggtctcacaattgtact

SEQ ID NO: 338 - RNA, 22 nt, synthetic construct
  misc_feature 1..22: note = Synthetic
  source 1..22: mol_type = other RNA
ggctggtctcacaattgtactt

SEQ ID NO: 339 - RNA, 23 nt, synthetic construct
  misc_feature 1..23: note = Synthetic
  source 1..23: mol_type = other RNA
ggctggtctcacaattgtacttt

SEQ ID NO: 340 - RNA, 25 nt, synthetic construct
  misc_feature 1..25: note = Synthetic
  source 1..25: mol_type = other RNA
ggctggtctcacaattgtactttac

SEQ ID NO: 341 - RNA, 24 nt, synthetic construct
  misc_feature 1..24: note = Synthetic
  source 1..24: mol_type = other RNA
gctggtctcacaattgtactttac

SEQ ID NO: 342 - RNA, 25 nt, synthetic construct
  misc_feature 1..25: note = Synthetic
  source 1..25: mol_type = other RNA
gctggtctcacaattgtactttact

SEQ ID NO: 343 - RNA, 24 nt, synthetic construct
  misc_feature 1..24: note = Synthetic
  source 1..24: mol_type = other RNA
tgtaaaaggaatacacaacgctga

SEQ ID NO: 344 - RNA, 25 nt, synthetic construct
  misc_feature 1..25: note = Synthetic
  source 1..25: mol_type = other RNA
tgtaaaaggaatacacaacgctgaa

SEQ ID NO: 345 - RNA, 23 nt, synthetic construct
  misc_feature 1..23: note = Synthetic
  source 1..23: mol_type = other RNA
gtaaaaggaatacacaacgctga

SEQ ID NO: 346 - RNA, 24 nt, synthetic construct
  misc_feature 1..24: note = Synthetic
  source 1..24: mol_type = other RNA
gtaaaaggaatacacaacgctgaa

SEQ ID NO: 347 - RNA, 25 nt, synthetic construct
  misc_feature 1..25: note = Synthetic
  source 1..25: mol_type = other RNA
gtaaaaggaatacacaacgctgaag

SEQ ID NO: 348 - RNA, 22 nt, synthetic construct
  misc_feature 1..22: note = Synthetic
  source 1..22: mol_type = other RNA
taaaaggaatacacaacgctga

SEQ ID NO: 349 - RNA, 23 nt, synthetic construct
  misc_feature 1..23: note = Synthetic
  source 1..23: mol_type = other RNA
taaaaggaatacacaacgctgaa

SEQ ID NO: 350 - RNA, 24 nt, synthetic construct
  misc_feature 1..24: note = Synthetic
  source 1..24: mol_type = other RNA
taaaaggaatacacaacgctgaag

SEQ ID NO: 351 - RNA, 25 nt, synthetic construct
  misc_feature 1..25: note = Synthetic
  source 1..25: mol_type = other RNA
taaaaggaatacacaacgctgaaga

SEQ ID NO: 352 - RNA, 21 nt, synthetic construct
  misc_feature 1..21: note = Synthetic
  source 1..21: mol_type = other RNA
aaaaggaatacacaacgctga

SEQ ID NO: 353 - RNA, 22 nt, synthetic construct
  misc_feature 1..22: note = Synthetic
  source 1..22: mol_type = other RNA
aaaaggaatacacaacgctgaa

SEQ ID NO: 354 - RNA, 23 nt, synthetic construct
  misc_feature 1..23: note = Synthetic
  source 1..23: mol_type = other RNA
aaaaggaatacacaacgctgaag

SEQ ID NO: 355 - RNA, 24 nt, synthetic construct
  misc_feature 1..24: note = Synthetic
  source 1..24: mol_type = other RNA
aaaaggaatacacaacgctgaaga

SEQ ID NO: 356 - RNA, 25 nt, synthetic construct
  misc_feature 1..25: note = Synthetic
  source 1..25: mol_type = other RNA
aaaaggaatacacaacgctgaagaa

SEQ ID NO: 357 - RNA, 20 nt, synthetic construct
  misc_feature 1..20: note = Synthetic
  source 1..20: mol_type = other RNA
aaaggaatacacaacgctga

SEQ ID NO: 358 - RNA, 21 nt, synthetic construct
  misc_feature 1..21: note = Synthetic
  source 1..21: mol_type = other RNA
aaaggaatacacaacgctgaa

SEQ ID NO: 359 - RNA, 22 nt, synthetic construct
  misc_feature 1..22: note = Synthetic
  source 1..22: mol_type = other RNA
aaaggaatacacaacgctgaag

SEQ ID NO: 360 - RNA, 23 nt, synthetic construct
  misc_feature 1..23: note = Synthetic
  source 1..23: mol_type = other RNA
aaaggaatacacaacgctgaaga

SEQ ID NO: 361 - RNA, 24 nt, synthetic construct
  misc_feature 1..24: note = Synthetic
  source 1..24: mol_type = other RNA
aaaggaatacacaacgctgaagaa

SEQ ID NO: 362 - RNA, 25 nt, synthetic construct
  misc_feature 1..25: note = Synthetic
  source 1..25: mol_type = other RNA
aaaggaatacacaacgctgaagaac

SEQ ID NO: 363 - RNA, 20 nt, synthetic construct
  misc_feature 1..20: note = Synthetic
  source 1..20: mol_type = other RNA
aaggaatacacaacgctgaa

SEQ ID NO: 364 - RNA, 21 nt, synthetic construct
  misc_feature 1..21: note = Synthetic
  source 1..21: mol_type = other RNA
aaggaatacacaacgctgaag

SEQ ID NO: 365 - RNA, 22 nt, synthetic construct
  misc_feature 1..22: note = Synthetic
  source 1..22: mol_type = other RNA
aaggaatacacaacgctgaaga

SEQ ID NO: 366 - RNA, 23 nt, synthetic construct
  misc_feature 1..23: note = Synthetic
  source 1..23: mol_type = other RNA
aaggaatacacaacgctgaagaa

SEQ ID NO: 367 - RNA, 24 nt, synthetic construct
  misc_feature 1..24: note = Synthetic
  source 1..24: mol_type = other RNA
aaggaatacacaacgctgaagaac

SEQ ID NO: 368 - RNA, 20 nt, synthetic construct
  misc_feature 1..20: note = Synthetic
  source 1..20: mol_type = other RNA
aggaatacacaacgctgaag

SEQ ID NO: 369 - RNA, 21 nt, synthetic construct
  misc_feature 1..21: note = Synthetic
  source 1..21: mol_type = other RNA
aggaatacacaacgctgaaga

SEQ ID NO: 370 - RNA, 22 nt, synthetic construct
  misc_feature 1..22: note = Synthetic
  source 1..22: mol_type = other RNA
aggaatacacaacgctgaagaa

SEQ ID NO: 371 - RNA, 23 nt, synthetic construct
  misc_feature 1..23: note = Synthetic
  source 1..23: mol_type = other RNA
aggaatacacaacgctgaagaac

SEQ ID NO: 372 - RNA, 20 nt, synthetic construct
  misc_feature 1..20: note = Synthetic
  source 1..20: mol_type = other RNA
ggaatacacaacgctgaaga

SEQ ID NO: 373 - RNA, 21 nt, synthetic construct
  misc_feature 1..21: note = Synthetic
  source 1..21: mol_type = other RNA
ggaatacacaacgctgaagaa

SEQ ID NO: 374 - RNA, 22 nt, synthetic construct
  misc_feature 1..22: note = Synthetic
  source 1..22: mol_type = other RNA
ggaatacacaacgctgaagaac

SEQ ID NO: 375 - RNA, 24 nt, synthetic construct
  misc_feature 1..24: note = Synthetic
  source 1..24: mol_type = other RNA
ggaatacacaacgctgaagaaccc

SEQ ID NO: 376 - RNA, 25 nt, synthetic construct
  misc_feature 1..25: note = Synthetic
  source 1..25: mol_type = other RNA
ggaatacacaacgctgaagaaccct

SEQ ID NO: 377 - RNA, 21 nt, synthetic construct
  misc_feature 1..21: note = Synthetic
  source 1..21: mol_type = other RNA
gaatacacaacgctgaagaac

SEQ ID NO: 378 - RNA, 23 nt, synthetic construct
  misc_feature 1..23: note = Synthetic
  source 1..23: mol_type = other RNA
gaatacacaacgctgaagaaccc

SEQ ID NO: 379 - RNA, 24 nt, synthetic construct
  misc_feature 1..24: note = Synthetic
  source 1..24: mol_type = other RNA
gaatacacaacgctgaagaaccct

SEQ ID NO: 380 - RNA, 22 nt, synthetic construct
  misc_feature 1..22: note = Synthetic
  source 1..22: mol_type = other RNA
aatacacaacgctgaagaaccc

SEQ ID NO: 381 - RNA, 21 nt, synthetic construct
  misc_feature 1..21: note = Synthetic
  source 1..21: mol_type = other RNA
atacacaacgctgaagaaccc

SEQ ID NO: 382 - RNA, 20 nt, synthetic construct
  misc_feature 1..20: note = Synthetic
  source 1..20: mol_type = other RNA
tacacaacgctgaagaaccc

SEQ ID NO: 383 - RNA, 23 nt, synthetic construct
  misc_feature 1..23: note = Synthetic
  source 1..23: mol_type = other RNA
acacaacgctgaagaaccctgat

SEQ ID NO: 384 - RNA, 22 nt, synthetic construct
  misc_feature 1..22: note = Synthetic
  source 1..22: mol_type = other RNA
gaatgggatccagtatacttac

SEQ ID NO: 385 - RNA, 23 nt, synthetic construct
  misc_feature 1..23: note = Synthetic
  source 1..23: mol_type = other RNA
agaatgggatccagtatacttac

SEQ ID NO: 386 - RNA, 24 nt, synthetic construct
  misc_feature 1..24: note = Synthetic
  source 1..24: mol_type = other RNA
gagaatgggatccagtatacttac

SEQ ID NO: 387 - RNA, 25 nt, synthetic construct
  misc_feature 1..25: note = Synthetic
  source 1..25: mol_type = other RNA
agagaatgggatccagtatacttac

SEQ ID NO: 388 - RNA, 23 nt, synthetic construct
  misc_feature 1..23: note = Synthetic
  source 1..23: mol_type = other RNA
gagaatgggatccagtatactta

SEQ ID NO: 389 - RNA, 22 nt, synthetic construct
  misc_feature 1..22: note = Synthetic
  source 1..22: mol_type = other RNA
gagaatgggatccagtatactt

SEQ ID NO: 390 - RNA, 21 nt, synthetic construct
  misc_feature 1..21: note = Synthetic
  source 1..21: mol_type = other RNA
gagaatgggatccagtatact

SEQ ID NO: 391 - RNA, 25 nt, synthetic construct
  misc_feature 1..25: note = Synthetic
  source 1..25: mol_type = other RNA
ccaaagagaatgggatccagtatac

SEQ ID NO: 392 - RNA, 25 nt, synthetic construct
  misc_feature 1..25: note = Synthetic
  source 1..25: mol_type = other RNA
gagccaaagagaatgggatccagta

SEQ ID NO: 393 - RNA, 24 nt, synthetic construct
  misc_feature 1..24: note = Synthetic
  source 1..24: mol_type = other RNA
gagccaaagagaatgggatccagt

SEQ ID NO: 394 - RNA, 24 nt, synthetic construct
  misc_feature 1..24: note = Synthetic
  source 1..24: mol_type = other RNA
atggcagtttccttagtaaccaca

SEQ ID NO: 395 - RNA, 25 nt, synthetic construct
  misc_feature 1..25: note = Synthetic
  source 1..25: mol_type = other RNA
gatggcagtttccttagtaaccaca

SEQ ID NO: 396 - RNA, 23 nt, synthetic construct
  misc_feature 1..23: note = Synthetic
  source 1..23: mol_type = other RNA
atggcagtttccttagtaaccac

SEQ ID NO: 397 - RNA, 24 nt, synthetic construct
  misc_feature 1..24: note = Synthetic
  source 1..24: mol_type = other RNA
gatggcagtttccttagtaaccac

SEQ ID NO: 398 - RNA, 25 nt, synthetic construct
  misc_feature 1..25: note = Synthetic
  source 1..25: mol_type = other RNA
agatggcagtttccttagtaaccac

SEQ ID NO: 399 - RNA, 23 nt, synthetic construct
  misc_feature 1..23: note = Synthetic
  source 1..23: mol_type = other RNA
gatggcagtttccttagtaacca

SEQ ID NO: 400 - RNA, 22 nt, synthetic construct
  misc_feature 1..22: note = Synthetic
  source 1..22: mol_type = other RNA
gatggcagtttccttagtaacc

SEQ ID NO: 401 - RNA, 24 nt, synthetic construct
  misc_feature 1..24: note = Synthetic
  source 1..24: mol_type = other RNA
ttctagtttggagatggcagtttc

SEQ ID NO: 402 - RNA, 21 nt, synthetic construct
  misc_feature 1..21: note = Synthetic
  source 1..21: mol_type = other RNA
ctagtttggagatggcagttt

SEQ ID NO: 403 - RNA, 22 nt, synthetic construct
  misc_feature 1..22: note = Synthetic
  source 1..22: mol_type = other RNA
tctagtttggagatggcagttt

SEQ ID NO: 404 - RNA, 23 nt, synthetic construct
  misc_feature 1..23: note = Synthetic
  source 1..23: mol_type = other RNA
ttctagtttggagatggcagttt

SEQ ID NO: 405 - RNA, 20 nt, synthetic construct
  misc_feature 1..20: note = Synthetic
  source 1..20: mol_type = other RNA
ctagtttggagatggcagtt

SEQ ID NO: 406 - RNA, 21 nt, synthetic construct
  misc_feature 1..21: note = Synthetic
  source 1..21: mol_type = other RNA
tctagtttggagatggcagtt

SEQ ID NO: 407 - RNA, 22 nt, synthetic construct
  misc_feature 1..22: note = Synthetic
  source 1..22: mol_type = other RNA
ttctagtttggagatggcagtt

SEQ ID NO: 408 - RNA, 20 nt, synthetic construct
  misc_feature 1..20: note = Synthetic
  source 1..20: mol_type = other RNA
tctagtttggagatggcagt

SEQ ID NO: 409 - RNA, 21 nt, synthetic construct
  misc_feature 1..21: note = Synthetic
  source 1..21: mol_type = other RNA
ttctagtttggagatggcagt

SEQ ID NO: 410 - RNA, 22 nt, synthetic construct
  misc_feature 1..22: note = Synthetic
  source 1..22: mol_type = other RNA
gatggcatttctagtttggaga

SEQ ID NO: 411 - RNA, 21 nt, synthetic construct
  misc_feature 1..21: note = Synthetic
  source 1..21: mol_type = other RNA
gatggcatttctagtttggag

SEQ ID NO: 412 - RNA, 20 nt, synthetic construct
  misc_feature 1..20: note = Synthetic
  source 1..20: mol_type = other RNA
gatggcatttctagtttgga

SEQ ID NO: 413 - RNA, 23 nt, synthetic construct
  misc_feature 1..23: note = Synthetic
  source 1..23: mol_type = other RNA
tctgtccaagcccggttgaaatc

SEQ ID NO: 414 - RNA, 24 nt, synthetic construct
  misc_feature 1..24: note = Synthetic
  source 1..24: mol_type = other RNA
ttctgtccaagcccggttgaaatc

SEQ ID NO: 415 - RNA, 22 nt, synthetic construct
  misc_feature 1..22: note = Synthetic
  source 1..22: mol_type = other RNA
tctgtccaagcccggttgaaat

SEQ ID NO: 416 - RNA, 25 nt, synthetic construct
  misc_feature 1..25: note = Synthetic
  source 1..25: mol_type = other RNA
agttctgtccaagcccggttgaaat

SEQ ID NO: 417 - RNA, 23 nt, synthetic construct
  misc_feature 1..23: note = Synthetic
  source 1..23: mol_type = other RNA
agttctgtccaagcccggttgaa

SEQ ID NO: 418 - RNA, 20 nt, synthetic construct
  misc_feature 1..20: note = Synthetic
  source 1..20: mol_type = other RNA
ttctgtccaagcccggttga

SEQ ID NO: 419 - RNA, 22 nt, synthetic construct
  misc_feature 1..22: note = Synthetic
  source 1..22: mol_type = other RNA
agttctgtccaagcccggttga

SEQ ID NO: 420 - RNA, 25 nt, synthetic construct
  misc_feature 1..25: note = Synthetic
  source 1..25: mol_type = other RNA
gtaagttctgtccaagcccggttga

SEQ ID NO: 421 - RNA, 21 nt, synthetic construct
  misc_feature 1..21: note = Synthetic
  source 1..21: mol_type = other RNA
agttctgtccaagcccggttg

SEQ ID NO: 422 - RNA, 24 nt, synthetic construct
  misc_feature 1..24: note = Synthetic
  source 1..24: mol_type = other RNA
gtaagttctgtccaagcccggttg

SEQ ID NO: 423 - RNA, 25 nt, synthetic construct
  misc_feature 1..25: note = Synthetic
  source 1..25: mol_type = other RNA
ggtaagttctgtccaagcccggttg

SEQ ID NO: 424 - RNA, 21 nt, synthetic construct
  misc_feature 1..21: note = Synthetic
  source 1..21: mol_type = other RNA
taccttctgcttgatgatcat

SEQ ID NO: 425 - RNA, 23 nt, synthetic construct
  misc_feature 1..23: note = Synthetic
  source 1..23: mol_type = other RNA
cataccttctgcttgatgatcat

SEQ ID NO: 426 - RNA, 24 nt, synthetic construct
  misc_feature 1..24: note = Synthetic
  source 1..24: mol_type = other RNA
tcataccttctgcttgatgatcat

SEQ ID NO: 427 - RNA, 25 nt, synthetic construct
  misc_feature 1..25: note = Synthetic
  source 1..25: mol_type = other RNA
ctcataccttctgcttgatgatcat

SEQ ID NO: 428 - RNA, 20 nt, synthetic construct
  misc_feature 1..20: note = Synthetic
  source 1..20: mol_type = other RNA
taccttctgcttgatgatca

SEQ ID NO: 429 - RNA, 22 nt, synthetic construct
  misc_feature 1..22: note = Synthetic
  source 1..22: mol_type = other RNA
cataccttctgcttgatgatca

SEQ ID NO: 430 - RNA, 23 nt, synthetic construct
  misc_feature 1..23: note = Synthetic
  source 1..23: mol_type = other RNA
tcataccttctgcttgatgatca

SEQ ID NO: 431 - RNA, 24 nt, synthetic construct
  misc_feature 1..24: note = Synthetic
  source 1..24: mol_type = other RNA
ctcataccttctgcttgatgatca

SEQ ID NO: 432 - RNA, 25 nt, synthetic construct
  misc_feature 1..25: note = Synthetic
  source 1..25: mol_type = other RNA
tctcataccttctgcttgatgatca

SEQ ID NO: 433 - RNA, 21 nt, synthetic construct
  misc_feature 1..21: note = Synthetic
  source 1..21: mol_type = other RNA
cataccttctgcttgatgatc

SEQ ID NO: 434 - RNA, 22 nt, synthetic construct
  misc_feature 1..22: note = Synthetic
  source 1..22: mol_type = other RNA
tcataccttctgcttgatgatc

SEQ ID NO: 435 - RNA, 23 nt, synthetic construct
  misc_feature 1..23: note = Synthetic
  source 1..23: mol_type = other RNA
ctcataccttctgcttgatgatc

SEQ ID NO: 436 - RNA, 24 nt, synthetic construct
  misc_feature 1..24: note = Synthetic
  source 1..24: mol_type = other RNA
tctcataccttctgcttgatgatc

SEQ ID NO: 437 - RNA, 25 nt, synthetic construct
  misc_feature 1..25: note = Synthetic
  source 1..25: mol_type = other RNA
ttctcataccttctgcttgatgatc

SEQ ID NO: 438 - RNA, 20 nt, synthetic construct
  misc_feature 1..20: note = Synthetic
  source 1..20: mol_type = other RNA
cataccttctgcttgatgat

SEQ ID NO: 439 - RNA, 21 nt, synthetic construct
  misc_feature 1..21: note = Synthetic
  source 1..21: mol_type = other RNA
tcataccttctgcttgatgat

SEQ ID NO: 440 - RNA, 22 nt, synthetic construct
  misc_feature 1..22: note = Synthetic
  source 1..22: mol_type = other RNA
ctcataccttctgcttgatgat

SEQ ID NO: 441 - RNA, 23 nt, synthetic construct
  misc_feature 1..23: note = Synthetic
  source 1..23: mol_type = other RNA
tctcataccttctgcttgatgat

SEQ ID NO: 442 - RNA, 24 nt, synthetic construct
  misc_feature 1..24: note = Synthetic
  source 1..24: mol_type = other RNA
ttctcataccttctgcttgatgat

SEQ ID NO: 443 - RNA, 25 nt, synthetic construct
  misc_feature 1..25: note = Synthetic
  source 1..25: mol_type = other RNA
tttctcataccttctgcttgatgat

SEQ ID NO: 444 - RNA, 20 nt, synthetic construct
  misc_feature 1..20: note = Synthetic
  source 1..20: mol_type = other RNA
tcataccttctgcttgatga

SEQ ID NO: 445 - RNA, 21 nt, synthetic construct
  misc_feature 1..21: note = Synthetic
  source 1..21: mol_type = other RNA
ctcataccttctgcttgatga

SEQ ID NO: 446 - RNA, 22 nt, synthetic construct
  misc_feature 1..22: note = Synthetic
  source 1..22: mol_type = other RNA
tctcataccttctgcttgatga

SEQ ID NO: 447 - RNA, 23 nt, synthetic construct
  misc_feature 1..23: note = Synthetic
  source 1..23: mol_type = other RNA
ttctcataccttctgcttgatga

SEQ ID NO: 448 - RNA, 24 nt, synthetic construct
  misc_feature 1..24: note = Synthetic
  source 1..24: mol_type = other RNA
tttctcataccttctgcttgatga

SEQ ID NO: 449 - RNA, 25 nt, synthetic construct
  misc_feature 1..25: note = Synthetic
  source 1..25: mol_type = other RNA
ttttctcataccttctgcttgatga

SEQ ID NO: 450 - RNA, 20 nt, synthetic construct
  misc_feature 1..20: note = Synthetic
  source 1..20: mol_type = other RNA
ctcataccttctgcttgatg

SEQ ID NO: 451 - RNA, 21 nt, synthetic construct
  misc_feature 1..21: note = Synthetic
  source 1..21: mol_type = other RNA
tctcataccttctgcttgatg

SEQ ID NO: 452 - RNA, 22 nt, synthetic construct
  misc_feature 1..22: note = Synthetic
  source 1..22: mol_type = other RNA
ttctcataccttctgcttgatg

SEQ ID NO: 453 - RNA, 23 nt, synthetic construct
  misc_feature 1..23: note = Synthetic
  source 1..23: mol_type = other RNA
tttctcataccttctgcttgatg

SEQ ID NO: 454 - RNA, 24 nt, synthetic construct
  misc_feature 1..24: note = Synthetic
  source 1..24: mol_type = other RNA
ttttctcataccttctgcttgatg

SEQ ID NO: 455 - RNA, 25 nt, synthetic construct
  misc_feature 1..25: note = Synthetic
  source 1..25: mol_type = other RNA
tttttctcataccttctgcttgatg

SEQ ID NO: 456 - RNA, 20 nt, synthetic construct
  misc_feature 1..20: note = Synthetic
  source 1..20: mol_type = other RNA
tctcataccttctgcttgat

SEQ ID NO: 457 - RNA, 21 nt, synthetic construct
  misc_feature 1..21: note = Synthetic
  source 1..21: mol_type = other RNA
ttctcataccttctgcttgat

SEQ ID NO: 458 - RNA, 22 nt, synthetic construct
  misc_feature 1..22: note = Synthetic
  source 1..22: mol_type = other RNA
tttctcataccttctgcttgat

SEQ ID NO: 459 - RNA, 23 nt, synthetic construct
  misc_feature 1..23: note = Synthetic
  source 1..23: mol_type = other RNA
ttttctcataccttctgcttgat

SEQ ID NO: 460 - RNA, 24 nt, synthetic construct
  misc_feature 1..24: note = Synthetic
  source 1..24: mol_type = other RNA
tttttctcataccttctgcttgat

SEQ ID NO: 461 - RNA, 25 nt, synthetic construct
  misc_feature 1..25: note = Synthetic
  source 1..25: mol_type = other RNA
ttttttctcataccttctgcttgat

SEQ ID NO: 462 - RNA, 20 nt, synthetic construct
  misc_feature 1..20: note = Synthetic
  source 1..20: mol_type = other RNA
ttctcataccttctgcttga

SEQ ID NO: 463 - RNA, 21 nt, synthetic construct
  misc_feature 1..21: note = Synthetic
  source 1..21: mol_type = other RNA
tttctcataccttctgcttga

SEQ ID NO: 464 - RNA, 22 nt, synthetic construct
  misc_feature 1..22: note = Synthetic
  source 1..22: mol_type = other RNA
ttttctcataccttctgcttga

SEQ ID NO: 465 - RNA, 23 nt, synthetic construct
  misc_feature 1..23: note = Synthetic
  source 1..23: mol_type = other RNA
tttttctcataccttctgcttga

SEQ ID NO: 466 - RNA, 24 nt, synthetic construct
  misc_feature 1..24: note = Synthetic
  source 1..24: mol_type = other RNA
ttttttctcataccttctgcttga

SEQ ID NO: 467 - RNA, 25 nt, synthetic construct
  misc_feature 1..25: note = Synthetic
  source 1..25: mol_type = other RNA
attttttctcataccttctgcttga

SEQ ID NO: 468 - RNA, 20 nt, synthetic construct
  misc_feature 1..20: note = Synthetic
  source 1..20: mol_type = other RNA
tttctcataccttctgcttg

SEQ ID NO: 469 - RNA, 21 nt, synthetic construct
  misc_feature 1..21: note = Synthetic
  source 1..21: mol_type = other RNA
ttttctcataccttctgcttg

SEQ ID NO: 470 - RNA, 22 nt, synthetic construct
  misc_feature 1..22: note = Synthetic
  source 1..22: mol_type = other RNA
tttttctcataccttctgcttg

SEQ ID NO: 471 - RNA, 23 nt, synthetic construct
  misc_feature 1..23: note = Synthetic
  source 1..23: mol_type = other RNA
ttttttctcataccttctgcttg

SEQ ID NO: 472 - RNA, 24 nt, synthetic construct
  misc_feature 1..24: note = Synthetic
  source 1..24: mol_type = other RNA
attttttctcataccttctgcttg

SEQ ID NO: 473 - RNA, 20 nt, synthetic construct
  misc_feature 1..20: note = Synthetic
  source 1..20: mol_type = other RNA
ttttctcataccttctgctt

SEQ ID NO: 474 - RNA, 21 nt, synthetic construct
  misc_feature 1..21: note = Synthetic
  source 1..21: mol_type = other RNA
tttttctcataccttctgctt

SEQ ID NO: 475 - RNA, 22 nt, synthetic construct
  misc_feature 1..22: note = Synthetic
  source 1..22: mol_type = other RNA
ttttttctcataccttctgctt

SEQ ID NO: 476 - RNA, 23 nt, synthetic construct
  misc_feature 1..23: note = Synthetic
  source 1..23: mol_type = other RNA
attttttctcataccttctgctt

SEQ ID NO: 477 - RNA, 20 nt, synthetic construct
  misc_feature 1..20: note = Synthetic
  source 1..20: mol_type = other RNA
tttttctcataccttctgct

SEQ ID NO: 478 - RNA, 21 nt, synthetic construct
  misc_feature 1..21: note = Synthetic
  source 1..21: mol_type = other RNA
ttttttctcataccttctgct

SEQ ID NO: 479 - RNA, 22 nt, synthetic construct
  misc_feature 1..22: note = Synthetic
  source 1..22: mol_type = other RNA
attttttctcataccttctgct

SEQ ID NO: 480 - RNA, 20 nt, synthetic construct
  misc_feature 1..20: note = Synthetic
  source 1..20: mol_type = other RNA
ttttttctcataccttctgc

SEQ ID NO: 481 - RNA, 21 nt, synthetic construct
  misc_feature 1..21: note = Synthetic
  source 1..21: mol_type = other RNA
attttttctcataccttctgc

SEQ ID NO: 482 - RNA, 20 nt, synthetic construct
  misc_feature 1..20: note = Synthetic
  source 1..20: mol_type = other RNA
attttttctcataccttctg

SEQ ID NO: 483 - RNA, 23 nt, synthetic construct
  misc_feature 1..23: note = Synthetic
  source 1..23: mol_type = other RNA
acttctgccaacttttatcattt

SEQ ID NO: 484 - RNA, 22 nt, synthetic construct
  misc_feature 1..22: note = Synthetic
  source 1..22: mol_type = other RNA
atggtctaggagagtaaagtga

SEQ ID NO: 485 - RNA, 23 nt, synthetic construct
  misc_feature 1..23: note = Synthetic
  source 1..23: mol_type = other RNA
aatggtctaggagagtaaagtga

SEQ ID NO: 486 - RNA, 24 nt, synthetic construct
  misc_feature 1..24: note = Synthetic
  source 1..24: mol_type = other RNA
aaatggtctaggagagtaaagtga

SEQ ID NO: 487 - RNA, 25 nt, synthetic construct
  misc_feature 1..25: note = Synthetic
  source 1..25: mol_type = other RNA
ggaaatggtctaggagagtaaagtg

SEQ ID NO: 488 - RNA, 24 nt, synthetic construct
  misc_feature 1..24: note = Synthetic
  source 1..24: mol_type = other RNA
ggaaatggtctaggagagtaaagt

SEQ ID NO: 489 - RNA, 25 nt, synthetic construct
  misc_feature 1..25: note = Synthetic
  source 1..25: mol_type = other RNA
tgggaaatggtctaggagagtaaag

SEQ ID NO: 490 - RNA, 23 nt, synthetic construct
  misc_feature 1..23: note = Synthetic
  source 1..23: mol_type = other RNA
tgggaaatggtctaggagagtaa

SEQ ID NO: 491 - RNA, 24 nt, synthetic construct
  misc_feature 1..24: note = Synthetic
  source 1..24: mol_type = other RNA
gtgggaaatggtctaggagagtaa

SEQ ID NO: 492 - RNA, 25 nt, synthetic construct
  misc_feature 1..25: note = Synthetic
  source 1..25: mol_type = other RNA
ggtgggaaatggtctaggagagtaa

SEQ ID NO: 493 - RNA, 22 nt, synthetic construct
  misc_feature 1..22: note = Synthetic
  source 1..22: mol_type = other RNA
tgggaaatggtctaggagagta

SEQ ID NO: 494 - RNA, 23 nt, synthetic construct
  misc_feature 1..23: note = Synthetic
  source 1..23: mol_type = other RNA
gtgggaaatggtctaggagagta

SEQ ID NO: 495 - RNA, 24 nt, synthetic construct
  misc_feature 1..24: note = Synthetic
  source 1..24: mol_type = other RNA
ggtgggaaatggtctaggagagta

SEQ ID NO: 496 - RNA, 25 nt, synthetic construct
  misc_feature 1..25: note = Synthetic
  source 1..25: mol_type = other RNA
tggtgggaaatggtctaggagagta

SEQ ID NO: 497 - RNA, 21 nt, synthetic construct
  misc_feature 1..21: note = Synthetic
  source 1..21: mol_type = other RNA
tgggaaatggtctaggagagt

SEQ ID NO: 498 - RNA, 22 nt, synthetic construct
  misc_feature 1..22: note = Synthetic
  source 1..22: mol_type = other RNA
gtgggaaatggtctaggagagt

SEQ ID NO: 499 - RNA, 23 nt, synthetic construct
  misc_feature 1..23: note = Synthetic
  source 1..23: mol_type = other RNA
ggtgggaaatggtctaggagagt

SEQ ID NO: 500 - RNA, 24 nt, synthetic construct
  misc_feature 1..24: note = Synthetic
  source 1..24: mol_type = other RNA
tggtgggaaatggtctaggagagt

SEQ ID NO: 501 - RNA, 25 nt, synthetic construct
  misc_feature 1..25: note = Synthetic
  source 1..25: mol_type = other RNA
ctggtgggaaatggtctaggagagt

SEQ ID NO: 502 - RNA, 20 nt, synthetic construct
  misc_feature 1..20: note = Synthetic
  source 1..20: mol_type = other RNA
tgggaaatggtctaggagag

SEQ ID NO: 503 - RNA, 21 nt, synthetic construct
  misc_feature 1..21: note = Synthetic
  source 1..21: mol_type = other RNA
gtgggaaatggtctaggagag

SEQ ID NO: 504 - RNA, 22 nt, synthetic construct
  misc_feature 1..22: note = Synthetic
  source 1..22: mol_type = other RNA
ggtgggaaatggtctaggagag

SEQ ID NO: 505 - RNA, 23 nt, synthetic construct
  misc_feature 1..23: note = Synthetic
  source 1..23: mol_type = other RNA
tggtgggaaatggtctaggagag

SEQ ID NO: 506 - RNA, 24 nt, synthetic construct
  misc_feature 1..24: note = Synthetic
  source 1..24: mol_type = other RNA
ctggtgggaaatggtctaggagag

SEQ ID NO: 507 - RNA, 20 nt, synthetic construct
  misc_feature 1..20: note = Synthetic
  source 1..20: mol_type = other RNA
gtgggaaatggtctaggaga

SEQ ID NO: 508 - RNA, 21 nt, synthetic construct
  misc_feature 1..21: note = Synthetic
  source 1..21: mol_type = other RNA
ggtgggaaatggtctaggaga

SEQ ID NO: 509 - RNA, 22 nt, synthetic construct
  misc_feature 1..22: note = Synthetic
  source 1..22: mol_type = other RNA
tggtgggaaatggtctaggaga

SEQ ID NO: 510 - RNA, 23 nt, synthetic construct
  misc_feature 1..23: note = Synthetic
  source 1..23: mol_type = other RNA
ctggtgggaaatggtctaggaga

SEQ ID NO: 511 - RNA, 25 nt, synthetic construct
  misc_feature 1..25: note = Synthetic
  source 1..25: mol_type = other RNA
aactggtgggaaatggtctaggaga

SEQ ID NO: 512 - RNA, 20 nt, synthetic construct
  misc_feature 1..20: note = Synthetic
  source 1..20: mol_type = other RNA
ggtgggaaatggtctaggag

SEQ ID NO: 513 - RNA, 21 nt, synthetic construct
  misc_feature 1..21: note = Synthetic
  source 1..21: mol_type = other RNA
tggtgggaaatggtctaggag

SEQ ID NO: 514 - RNA, 22 nt, synthetic construct
  misc_feature 1..22: note = Synthetic
  source 1..22: mol_type = other RNA
ctggtgggaaatggtctaggag

SEQ ID NO: 515 - RNA, 24 nt, synthetic construct
  misc_feature 1..24: note = Synthetic
  source 1..24: mol_type = other RNA
aactggtgggaaatggtctaggag

SEQ ID NO: 516 - RNA, 20 nt, synthetic construct
  misc_feature 1..20: note = Synthetic
  source 1..20: mol_type = other RNA
tggtgggaaatggtctagga

SEQ ID NO: 517 - RNA, 21 nt, synthetic construct
  misc_feature 1..21: note = Synthetic
  source 1..21: mol_type = other RNA
ctggtgggaaatggtctagga

SEQ ID NO: 518 - RNA, 23 nt, synthetic construct
  misc_feature 1..23: note = Synthetic
  source 1..23: mol_type = other RNA
aactggtgggaaatggtctagga

SEQ ID NO: 519 - RNA, 25 nt, synthetic construct
  misc_feature 1..25: note = Synthetic
  source 1..25: mol_type = other RNA
agaactggtgggaaatggtctagga

SEQ ID NO: 520 - RNA, 20 nt, synthetic construct
  misc_feature 1..20: note = Synthetic
  source 1..20: mol_type = other RNA
ctggtgggaaatggtctagg

SEQ ID NO: 521 - RNA, 22 nt, synthetic construct
  misc_feature 1..22: note = Synthetic
  source 1..22: mol_type = other RNA
aactggtgggaaatggtctagg

SEQ ID NO: 522 - RNA, 24 nt, synthetic construct
  misc_feature 1..24: note = Synthetic
  source 1..24: mol_type = other RNA
agaactggtgggaaatggtctagg

SEQ ID NO: 523 - RNA, 25 nt, synthetic construct
  misc_feature 1..25: note = Synthetic
  source 1..25: mol_type = other RNA
aagaactggtgggaaatggtctagg

SEQ ID NO: 524 - RNA, 21 nt, synthetic construct
  misc_feature 1..21: note = Synthetic
  source 1..21: mol_type = other RNA
aactggtgggaaatggtctag

SEQ ID NO: 525 - RNA, 23 nt, synthetic construct
  misc_feature 1..23: note = Synthetic
  source 1..23: mol_type = other RNA
agaactggtgggaaatggtctag

SEQ ID NO: 526 - RNA, 24 nt, synthetic construct
  misc_feature 1..24: note = Synthetic
  source 1..24: mol_type = other RNA
aagaactggtgggaaatggtctag

SEQ ID NO: 527 - RNA, 20 nt, synthetic construct
  misc_feature 1..20: note = Synthetic
  source 1..20: mol_type = other RNA
aactggtgggaaatggtcta

SEQ ID NO: 528 - RNA, 22 nt, synthetic construct
  misc_feature 1..22: note = Synthetic
  source 1..22: mol_type = other RNA
agaactggtgggaaatggtcta

SEQ ID NO: 529 - RNA, 23 nt, synthetic construct
  misc_feature 1..23: note = Synthetic
  source 1..23: mol_type = other RNA
aagaactggtgggaaatggtcta

SEQ ID NO: 530 - RNA, 25 nt, synthetic construct
  misc_feature 1..25: note = Synthetic
  source 1..25: mol_type = other RNA
ctaagaactggtgggaaatggtcta

SEQ ID NO: 531 - RNA, 21 nt, synthetic construct
  misc_feature 1..21: note = Synthetic
  source 1..21: mol_type = other RNA
agaactggtgggaaatggtct

SEQ ID NO: 532 - RNA, 22 nt, synthetic construct
  misc_feature 1..22: note = Synthetic
  source 1..22: mol_type = other RNA
aagaactggtgggaaatggtct

SEQ ID NO: 533 - RNA, 24 nt, synthetic construct
  misc_feature 1..24: note = Synthetic
  source 1..24: mol_type = other RNA
ctaagaactggtgggaaatggtct

SEQ ID NO: 534 - RNA, 25 nt, synthetic construct
  misc_feature 1..25: note = Synthetic
  source 1..25: mol_type = other RNA
cctaagaactggtgggaaatggtct

SEQ ID NO: 535 - RNA, 20 nt, synthetic construct
  misc_feature 1..20: note = Synthetic
  source 1..20: mol_type = other RNA
agaactggtgggaaatggtc

SEQ ID NO: 536 - RNA, 21 nt, synthetic construct
  misc_feature 1..21: note = Synthetic
  source 1..21: mol_type = other RNA
aagaactggtgggaaatggtc

SEQ ID NO: 537 - RNA, 23 nt, synthetic construct
  misc_feature 1..23: note = Synthetic
  source 1..23: mol_type = other RNA
ctaagaactggtgggaaatggtc

SEQ ID NO: 538 - RNA, 24 nt, synthetic construct
  misc_feature 1..24: note = Synthetic
  source 1..24: mol_type = other RNA
cctaagaactggtgggaaatggtc

SEQ ID NO: 539 - RNA, 25 nt, synthetic construct
  misc_feature 1..25: note = Synthetic
  source 1..25: mol_type = other RNA
gcctaagaactggtgggaaatggtc

SEQ ID NO: 540 - RNA, 24 nt, synthetic construct
  misc_feature 1..24: note = Synthetic
  source 1..24: mol_type = other RNA
gcctaagaactggtgggaaatggt

SEQ ID NO: 541 - RNA, 25 nt, synthetic construct
  misc_feature 1..25: note = Synthetic
  source 1..25: mol_type = other RNA
tgcctaagaactggtgggaaatggt

SEQ ID NO: 542 - RNA, 23 nt, synthetic construct
  misc_feature 1..23: note = Synthetic
  source 1..23: mol_type = other RNA
gcctaagaactggtgggaaatgg

SEQ ID NO: 543 - RNA, 24 nt, synthetic construct
  misc_feature 1..24: note = Synthetic
  source 1..24: mol_type = other RNA
tgcctaagaactggtgggaaatgg

SEQ ID NO: 544 - RNA, 25 nt, synthetic construct
  misc_feature 1..25: note = Synthetic
  source 1..25: mol_type = other RNA
ttgcctaagaactggtgggaaatgg

SEQ ID NO: 545 - RNA, 23 nt, synthetic construct
  misc_feature 1..23: note = Synthetic
  source 1..23: mol_type = other RNA
tgcctaagaactggtgggaaatg

SEQ ID NO: 546 - RNA, 24 nt, synthetic construct
  misc_feature 1..24: note = Synthetic
  source 1..24: mol_type = other RNA
ttgcctaagaactggtgggaaatg

SEQ ID NO: 547 - RNA, 20 nt, synthetic construct
  misc_feature 1..20: note = Synthetic
  source 1..20: mol_type = other RNA
gtgagaccagccaaaacact

SEQ ID NO: 548 - RNA, 21 nt, synthetic construct
  misc_feature 1..21: note = Synthetic
  source 1..21: mol_type = other RNA
tgtgagaccagccaaaacact

SEQ ID NO: 549 - RNA, 22 nt, synthetic construct
  misc_feature 1..22: note = Synthetic
  source 1..22: mol_type = other RNA
ttgtgagaccagccaaaacact

SEQ ID NO: 550 - RNA, 23 nt, synthetic construct
  misc_feature 1..23: note = Synthetic
  source 1..23: mol_type = other RNA
attgtgagaccagccaaaacact

SEQ ID NO: 551 - RNA, 22 nt, synthetic construct
  misc_feature 1..22: note = Synthetic
  source 1..22: mol_type = other RNA
attgtgagaccagccaaaacac

SEQ ID NO: 552 - RNA, 25 nt, synthetic construct
  misc_feature 1..25: note = Synthetic
  source 1..25: mol_type = other RNA
gtacaattgtgagaccagccaaaac

SEQ ID NO: 553 - RNA, 23 nt, synthetic construct
  misc_feature 1..23: note = Synthetic
  source 1..23: mol_type = other RNA
gtacaattgtgagaccagccaaa

SEQ ID NO: 554 - RNA, 22 nt, synthetic construct
  misc_feature 1..22: note = Synthetic
  source 1..22: mol_type = other RNA
gtacaattgtgagaccagccaa

SEQ ID NO: 555 - RNA, 23 nt, synthetic construct
  misc_feature 1..23: note = Synthetic
  source 1..23: mol_type = other RNA
agtacaattgtgagaccagccaa

SEQ ID NO: 556 - RNA, 21 nt, synthetic construct
  misc_feature 1..21: note = Synthetic
  source 1..21: mol_type = other RNA
gtacaattgtgagaccagcca

SEQ ID NO: 557 - RNA, 22 nt, synthetic construct
  misc_feature 1..22: note = Synthetic
  source 1..22: mol_type = other RNA
agtacaattgtgagaccagcca

SEQ ID NO: 558 - RNA, 23 nt, synthetic construct
  misc_feature 1..23: note = Synthetic
  source 1..23: mol_type = other RNA
aagtacaattgtgagaccagcca

SEQ ID NO: 559 - RNA, 24 nt, synthetic construct
  misc_feature 1..24: note = Synthetic
  source 1..24: mol_type = other RNA
aaagtacaattgtgagaccagcca

SEQ ID NO: 560 - RNA, 20 nt, synthetic construct
  misc_feature 1..20: note = Synthetic
  source 1..20: mol_type = other RNA
gtacaattgtgagaccagcc

SEQ ID NO: 561 - RNA, 21 nt, synthetic construct
  misc_feature 1..21: note = Synthetic
  source 1..21: mol_type = other RNA
agtacaattgtgagaccagcc

SEQ ID NO: 562 - RNA, 22 nt, synthetic construct
  misc_feature 1..22: note = Synthetic
  source 1..22: mol_type = other RNA
aagtacaattgtgagaccagcc

SEQ ID NO: 563 - RNA, 23 nt, synthetic construct
  misc_feature 1..23: note = Synthetic
  source 1..23: mol_type = other RNA
aaagtacaattgtgagaccagcc

SEQ ID NO: 564 - RNA, 25 nt, synthetic construct
  misc_feature 1..25: note = Synthetic
  source 1..25: mol_type = other RNA
gtaaagtacaattgtgagaccagcc

SEQ ID NO: 565 - RNA, 24 nt, synthetic construct
  misc_feature 1..24: note = Synthetic
  source 1..24: mol_type = other RNA
gtaaagtacaattgtgagaccagc

SEQ ID NO: 566 - RNA, 25 nt, synthetic construct
  misc_feature 1..25: note = Synthetic
  source 1..25: mol_type = other RNA
agtaaagtacaattgtgagaccagc

SEQ ID NO: 567 - RNA, 24 nt, synthetic construct
  misc_feature 1..24: note = Synthetic
  source 1..24: mol_type = other RNA
tcagcgttgtgtattccttttaca

SEQ ID NO: 568 - RNA, 25 nt, synthetic construct
  misc_feature 1..25: note = Synthetic
  source 1..25: mol_type = other RNA
ttcagcgttgtgtattccttttaca

SEQ ID NO: 569 - RNA, 23 nt, synthetic construct
  misc_feature 1..23: note = Synthetic
  source 1..23: mol_type = other RNA
tcagcgttgtgtattccttttac

SEQ ID NO: 570 - RNA, 24 nt, synthetic construct
  misc_feature 1..24: note = Synthetic
  source 1..24: mol_type = other RNA
ttcagcgttgtgtattccttttac

SEQ ID NO: 571 - RNA, 25 nt, synthetic construct
  misc_feature 1..25: note = Synthetic
  source 1..25: mol_type = other RNA
cttcagcgttgtgtattccttttac

SEQ ID NO: 572 - RNA, 22 nt, synthetic construct
  misc_feature 1..22: note = Synthetic
  source 1..22: mol_type = other RNA
tcagcgttgtgtattcctttta

SEQ ID NO: 573 - RNA, 23 nt, synthetic construct
  misc_feature 1..23: note = Synthetic
  source 1..23: mol_type = other RNA
ttcagcgttgtgtattcctttta

SEQ ID NO: 574 - RNA, 24 nt, synthetic construct
  misc_feature 1..24: note = Synthetic
  source 1..24: mol_type = other RNA
cttcagcgttgtgtattcctttta

SEQ ID NO: 575 - RNA, 25 nt, synthetic construct
  misc_feature 1..25: note = Synthetic
  source 1..25: mol_type = other RNA
tcttcagcgttgtgtattcctttta

SEQ ID NO: 576 - RNA, 21 nt, synthetic construct
  misc_feature 1..21: note = Synthetic
  source 1..21: mol_type = other RNA
tcagcgttgtgtattcctttt

SEQ ID NO: 577 - RNA, 22 nt, synthetic construct
  misc_feature 1..22: note = Synthetic
  source 1..22: mol_type = other RNA
ttcagcgttgtgtattcctttt

SEQ ID NO: 578 - RNA, 23 nt, synthetic construct
  misc_feature 1..23: note = Synthetic
  source 1..23: mol_type = other RNA
cttcagcgttgtgtattcctttt

SEQ ID NO: 579 - RNA, 24 nt, synthetic construct
  misc_feature 1..24: note = Synthetic
  source 1..24: mol_type = other RNA
tcttcagcgttgtgtattcctttt

SEQ ID NO: 580 - RNA, 25 nt, synthetic construct
  misc_feature 1..25: note = Synthetic
  source 1..25: mol_type = other RNA
ttcttcagcgttgtgtattcctttt

SEQ ID NO: 581 - RNA, 20 nt, synthetic construct
  misc_feature 1..20: note = Synthetic
  source 1..20: mol_type = other RNA
tcagcgttgtgtattccttt

SEQ ID NO: 582 - RNA, 21 nt, synthetic construct
  misc_feature 1..21: note = Synthetic
  source 1..21: mol_type = other RNA
ttcagcgttgtgtattccttt

SEQ ID NO: 583 - RNA, 22 nt, synthetic construct
  misc_feature 1..22: note = Synthetic
  source 1..22: mol_type = other RNA
cttcagcgttgtgtattccttt

SEQ ID NO: 584 - RNA, 23 nt, synthetic construct
  misc_feature 1..23: note = Synthetic
  source 1..23: mol_type = other RNA
tcttcagcgttgtgtattccttt

SEQ ID NO: 585 - RNA, 24 nt, synthetic construct
  misc_feature 1..24: note = Synthetic
  source 1..24: mol_type = other RNA
ttcttcagcgttgtgtattccttt

SEQ ID NO: 586 - RNA, 25 nt, synthetic construct
  misc_feature 1..25: note = Synthetic
  source 1..25: mol_type = other RNA
gttcttcagcgttgtgtattccttt

SEQ ID NO: 587 - RNA, 20 nt, synthetic construct
  misc_feature 1..20: note = Synthetic
  source 1..20: mol_type = other RNA
ttcagcgttgtgtattcctt

SEQ ID NO: 588 - RNA, 21 nt, synthetic construct
  misc_feature 1..21: note = Synthetic
  source 1..21: mol_type = other RNA
cttcagcgttgtgtattcctt

SEQ ID NO: 589 - RNA, 22 nt, synthetic construct
  misc_feature 1..22: note = Synthetic
  source 1..22: mol_type = other RNA
tcttcagcgttgtgtattcctt

SEQ ID NO: 590 - RNA, 23 nt, synthetic construct
  misc_feature 1..23: note = Synthetic
  source 1..23: mol_type = other RNA
ttcttcagcgttgtgtattcctt

SEQ ID NO: 591 - RNA, 24 nt, synthetic construct
  misc_feature 1..24: note = Synthetic
  source 1..24: mol_type = other RNA
gttcttcagcgttgtgtattcctt

SEQ ID NO: 592 - RNA, 20 nt, synthetic construct
  misc_feature 1..20: note = Synthetic
  source 1..20: mol_type = other RNA
cttcagcgttgtgtattcct

SEQ ID NO: 593 - RNA, 21 nt, synthetic construct
  misc_feature 1..21: note = Synthetic
  source 1..21: mol_type = other RNA
tcttcagcgttgtgtattcct

SEQ ID NO: 594 - RNA, 22 nt, synthetic construct
  misc_feature 1..22: note = Synthetic
  source 1..22: mol_type = other RNA
ttcttcagcgttgtgtattcct

SEQ ID NO: 595 - RNA, 23 nt, synthetic construct
  misc_feature 1..23: note = Synthetic
  source 1..23: mol_type = other RNA
gttcttcagcgttgtgtattcct

SEQ ID NO: 596 - RNA, 20 nt, synthetic construct
  misc_feature 1..20: note = Synthetic
  source 1..20: mol_type = other RNA
tcttcagcgttgtgtattcc

SEQ ID NO: 597 - RNA, 21 nt, synthetic construct
  misc_feature 1..21: note = Synthetic
  source 1..21: mol_type = other RNA
ttcttcagcgttgtgtattcc

SEQ ID NO: 598 - RNA, 22 nt, synthetic construct
  misc_feature 1..22: note = Synthetic
  source 1..22: mol_type = other RNA
gttcttcagcgttgtgtattcc

SEQ ID NO: 599 - RNA, 24 nt, synthetic construct
  misc_feature 1..24: note = Synthetic
  source 1..24: mol_type = other RNA
gggttcttcagcgttgtgtattcc

SEQ ID NO: 600 - RNA, 25 nt, synthetic construct
  misc_feature 1..25: note = Synthetic
  source 1..25: mol_type = other RNA
agggttcttcagcgttgtgtattcc

SEQ ID NO: 601 - RNA, 21 nt, synthetic construct
  misc_feature 1..21: note = Synthetic
  source 1..21: mol_type = other RNA
gttcttcagcgttgtgtattc

SEQ ID NO: 602 - RNA, 23 nt, synthetic construct
  misc_feature 1..23: note = Synthetic
  source 1..23: mol_type = other RNA
gggttcttcagcgttgtgtattc

SEQ ID NO: 603 - RNA, 24 nt, synthetic construct
  misc_feature 1..24: note = Synthetic
  source 1..24: mol_type = other RNA
agggttcttcagcgttgtgtattc

SEQ ID NO: 604 - RNA, 22 nt, synthetic construct
  misc_feature 1..22: note = Synthetic
  source 1..22: mol_type = other RNA
gggttcttcagcgttgtgtatt

SEQ ID NO: 605 - RNA, 21 nt, synthetic construct
  misc_feature 1..21: note = Synthetic
  source 1..21: mol_type = other RNA
gggttcttcagcgttgtgtat

SEQ ID NO: 606 - RNA, 20 nt, synthetic construct
  misc_feature 1..20: note = Synthetic
  source 1..20: mol_type = other RNA
gggttcttcagcgttgtgta

SEQ ID NO: 607 - RNA, 23 nt, synthetic construct
  misc_feature 1..23: note = Synthetic
  source 1..23: mol_type = other RNA
atcagggttcttcagcgttgtgt

SEQ ID NO: 608 - DNA, 22 bp, synthetic construct
  misc_feature 1..22: note = Synthetic
  source 1..22: mol_type = other DNA
gaatgggatccagtatacttac

SEQ ID NO: 609 - DNA, 23 bp, synthetic construct
  misc_feature 1..23: note = Synthetic
  source 1..23: mol_type = other DNA
agaatgggatccagtatacttac

SEQ ID NO: 610 - DNA, 24 bp, synthetic construct
  misc_feature 1..24: note = Synthetic
  source 1..24: mol_type = other DNA
gagaatgggatccagtatacttac

SEQ ID NO: 611 - DNA, 25 bp, synthetic construct
  misc_feature 1..25: note = Synthetic
  source 1..25: mol_type = other DNA
agagaatgggatccagtatacttac

SEQ ID NO: 612 - DNA, 23 bp, synthetic construct
  misc_feature 1..23: note = Synthetic
  source 1..23: mol_type = other DNA
gagaatgggatccagtatactta

SEQ ID NO: 613 - DNA, 22 bp, synthetic construct
  misc_feature 1..22: note = Synthetic
  source 1..22: mol_type = other DNA
gagaatgggatccagtatactt

SEQ ID NO: 614 - DNA, 21 bp, synthetic construct
  misc_feature 1..21: note = Synthetic
  source 1..21: mol_type = other DNA
gagaatgggatccagtatact

SEQ ID NO: 615 - DNA, 25 bp, synthetic construct
  misc_feature 1..25: note = Synthetic
  source 1..25: mol_type = other DNA
ccaaagagaatgggatccagtatac

SEQ ID NO: 616 - DNA, 25 bp, synthetic construct
  misc_feature 1..25: note = Synthetic
  source 1..25: mol_type = other DNA
gagccaaagagaatgggatccagta

SEQ ID NO: 617 - DNA, 24 bp, synthetic construct
  misc_feature 1..24: note = Synthetic
  source 1..24: mol_type = other DNA
gagccaaagagaatgggatccagt

SEQ ID NO: 618 - DNA, 24 bp, synthetic construct
  misc_feature 1..24: note = Synthetic
  source 1..24: mol_type = other DNA
atggcagtttccttagtaaccaca

SEQ ID NO: 619 - DNA, 25 bp, synthetic construct
  misc_feature 1..25: note = Synthetic
  source 1..25: mol_type = other DNA
gatggcagtttccttagtaaccaca

SEQ ID NO: 620 - DNA, 23 bp, synthetic construct
  misc_feature 1..23: note = Synthetic
  source 1..23: mol_type = other DNA
atggcagtttccttagtaaccac

SEQ ID NO: 621 - DNA, 24 bp, synthetic construct
  misc_feature 1..24: note = Synthetic
  source 1..24: mol_type = other DNA
gatggcagtttccttagtaaccac

SEQ ID NO: 622 - DNA, 25 bp, synthetic construct
  misc_feature 1..25: note = Synthetic
  source 1..25: mol_type = other DNA
agatggcagtttccttagtaaccac

SEQ ID NO: 623 - DNA, 23 bp, synthetic construct
  misc_feature 1..23: note = Synthetic
  source 1..23: mol_type = other DNA
gatggcagtttccttagtaacca

SEQ ID NO: 624 - DNA, 22 bp, synthetic construct
  misc_feature 1..22: note = Synthetic
  source 1..22: mol_type = other DNA
gatggcagtttccttagtaacc

SEQ ID NO: 625 - DNA, 24 bp, synthetic construct
  misc_feature 1..24: note = Synthetic
  source 1..24: mol_type = other DNA
ttctagtttggagatggcagtttc

SEQ ID NO: 626 - DNA, 21 bp, synthetic construct
  misc_feature 1..21: note = Synthetic
  source 1..21: mol_type = other DNA
ctagtttggagatggcagttt

SEQ ID NO: 627 - DNA, 22 bp, synthetic construct
  misc_feature 1..22: note = Synthetic
  source 1..22: mol_type = other DNA
tctagtttggagatggcagttt

SEQ ID NO: 628 - DNA, 23 bp, synthetic construct
  misc_feature 1..23: note = Synthetic
  source 1..23: mol_type = other DNA
ttctagtttggagatggcagttt

SEQ ID NO: 629 - DNA, 20 bp, synthetic construct
  misc_feature 1..20: note = Synthetic
  source 1..20: mol_type = other DNA
ctagtttggagatggcagtt

SEQ ID NO: 630 - DNA, 21 bp, synthetic construct
  misc_feature 1..21: note = Synthetic
  source 1..21: mol_type = other DNA
tctagtttggagatggcagtt

SEQ ID NO: 631 - DNA, 22 bp, synthetic construct
  misc_feature 1..22: note = Synthetic
  source 1..22: mol_type = other DNA
ttctagtttggagatggcagtt

SEQ ID NO: 632 - DNA, 20 bp, synthetic construct
  misc_feature 1..20: note = Synthetic
  source 1..20: mol_type = other DNA
tctagtttggagatggcagt

SEQ ID NO: 633 - DNA, 21 bp, synthetic construct
  misc_feature 1..21: note = Synthetic
  source 1..21: mol_type = other DNA
ttctagtttggagatggcagt

SEQ ID NO: 634 - DNA, 22 bp, synthetic construct
  misc_feature 1..22: note = Synthetic
  source 1..22: mol_type = other DNA
gatggcatttctagtttggaga

SEQ ID NO: 635 - DNA, 21 bp, synthetic construct
  misc_feature 1..21: note = Synthetic
  source 1..21: mol_type = other DNA
gatggcatttctagtttggag

SEQ ID NO: 636 - DNA, 20 bp, synthetic construct
  misc_feature 1..20: note = Synthetic
  source 1..20: mol_type = other DNA
gatggcatttctagtttgga

SEQ ID NO: 637 - DNA, 23 bp, synthetic construct
  misc_feature 1..23: note = Synthetic
  source 1..23: mol_type = other DNA
tctgtccaagcccggttgaaatc

SEQ ID NO: 638 - DNA, 24 bp, synthetic construct
  misc_feature 1..24: note = Synthetic
  source 1..24: mol_type = other DNA
ttctgtccaagcccggttgaaatc

SEQ ID NO: 639 - DNA, 22 bp, synthetic construct
  misc_feature 1..22: note = Synthetic
  source 1..22: mol_type = other DNA
tctgtccaagcccggttgaaat

SEQ ID NO: 640 - DNA, 25 bp, synthetic construct
  misc_feature 1..25: note = Synthetic
  source 1..25: mol_type = other DNA
agttctgtccaagcccggttgaaat

SEQ ID NO: 641 - DNA, 23 bp, synthetic construct
  misc_feature 1..23: note = Synthetic
  source 1..23: mol_type = other DNA
agttctgtccaagcccggttgaa

SEQ ID NO: 642 - DNA, 20 bp, synthetic construct
  misc_feature 1..20: note = Synthetic
  source 1..20: mol_type = other DNA
ttctgtccaagcccggttga

SEQ ID NO: 643 - DNA, 22 bp, synthetic construct
  misc_feature 1..22: note = Synthetic
  source 1..22: mol_type = other DNA
agttctgtccaagcccggttga

SEQ ID NO: 644 - DNA, 25 bp, synthetic construct
  misc_feature 1..25: note = Synthetic
  source 1..25: mol_type = other DNA
gtaagttctgtccaagcccggttga

SEQ ID NO: 645 - DNA, 21 bp, synthetic construct
  misc_feature 1..21: note = Synthetic
  source 1..21: mol_type = other DNA
agttctgtccaagcccggttg

SEQ ID NO: 646 - DNA, 24 bp, synthetic construct
  misc_feature 1..24: note = Synthetic
  source 1..24: mol_type = other DNA
gtaagttctgtccaagcccggttg

SEQ ID NO: 647 - DNA, 25 bp, synthetic construct
  misc_feature 1..25: note = Synthetic
  source 1..25: mol_type = other DNA
ggtaagttctgtccaagcccggttg

SEQ ID NO: 648 - DNA, 21 bp, synthetic construct
  misc_feature 1..21: note = Synthetic
  source 1..21: mol_type = other DNA
taccttctgcttgatgatcat

SEQ ID NO: 649 - DNA, 23 bp, synthetic construct
  misc_feature 1..23: note = Synthetic
  source 1..23: mol_type = other DNA
cataccttctgcttgatgatcat

SEQ ID NO: 650 - DNA, 24 bp, synthetic construct
  misc_feature 1..24: note = Synthetic
  source 1..24: mol_type = other DNA
tcataccttctgcttgatgatcat

SEQ ID NO: 651 - DNA, 25 bp, synthetic construct
  misc_feature 1..25: note = Synthetic
  source 1..25: mol_type = other DNA
ctcataccttctgcttgatgatcat

SEQ ID NO: 652 - DNA, 20 bp, synthetic construct
  misc_feature 1..20: note = Synthetic
  source 1..20: mol_type = other DNA
taccttctgcttgatgatca

SEQ ID NO: 653 - DNA, 22 bp, synthetic construct
  misc_feature 1..22: note = Synthetic
  source 1..22: mol_type = other DNA
cataccttctgcttgatgatca

SEQ ID NO: 654 - DNA, 23 bp, synthetic construct
  misc_feature 1..23: note = Synthetic
  source 1..23: mol_type = other DNA
tcataccttctgcttgatgatca

SEQ ID NO: 655 - DNA, 24 bp, synthetic construct
  misc_feature 1..24: note = Synthetic
  source 1..24: mol_type = other DNA
ctcataccttctgcttgatgatca

SEQ ID NO: 656 - DNA, 25 bp, synthetic construct
  misc_feature 1..25: note = Synthetic
  source 1..25: mol_type = other DNA
tctcataccttctgcttgatgatca

SEQ ID NO: 657 - DNA, 21 bp, synthetic construct
  misc_feature 1..21: note = Synthetic
  source 1..21: mol_type = other DNA
cataccttctgcttgatgatc

SEQ ID NO: 658 - DNA, 22 bp, synthetic construct
  misc_feature 1..22: note = Synthetic
  source 1..22: mol_type = other DNA
tcataccttctgcttgatgatc

SEQ ID NO: 659 - DNA, 23 bp, synthetic construct
  misc_feature 1..23: note = Synthetic
  source 1..23: mol_type = other DNA
ctcataccttctgcttgatgatc

SEQ ID NO: 660 - DNA, 24 bp, synthetic construct
  misc_feature 1..24: note = Synthetic
  source 1..24: mol_type = other DNA
tctcataccttctgcttgatgatc

SEQ ID NO: 661 - DNA, 25 bp, synthetic construct
  misc_feature 1..25: note = Synthetic
  source 1..25: mol_type = other DNA
ttctcataccttctgcttgatgatc

SEQ ID NO: 662 - DNA, 20 bp, synthetic construct
  misc_feature 1..20: note = Synthetic
  source 1..20: mol_type = other DNA
cataccttctgcttgatgat

SEQ ID NO: 663 - DNA, 21 bp, synthetic construct
  misc_feature 1..21: note = Synthetic
  source 1..21: mol_type = other DNA
tcataccttctgcttgatgat

SEQ ID NO: 664 - DNA, 22 bp, synthetic construct
  misc_feature 1..22: note = Synthetic
  source 1..22: mol_type = other DNA
ctcataccttctgcttgatgat

SEQ ID NO: 665 - DNA, 23 bp, synthetic construct
  misc_feature 1..23: note = Synthetic
  source 1..23: mol_type = other DNA
tctcataccttctgcttgatgat

SEQ ID NO: 666 - DNA, 24 bp, synthetic construct
  misc_feature 1..24: note = Synthetic
  source 1..24: mol_type = other DNA
ttctcataccttctgcttgatgat

SEQ ID NO: 667 - DNA, 25 bp, synthetic construct
  misc_feature 1..25: note = Synthetic
  source 1..25: mol_type = other DNA
tttctcataccttctgcttgatgat

SEQ ID NO: 668 - DNA, 20 bp, synthetic construct
  misc_feature 1..20: note = Synthetic
  source 1..20: mol_type = other DNA
tcataccttctgcttgatga

SEQ ID NO: 669 - DNA, 21 bp, synthetic construct
  misc_feature 1..21: note = Synthetic
  source 1..21: mol_type = other DNA
ctcataccttctgcttgatga

SEQ ID NO: 670 - DNA, 22 bp, synthetic construct
  misc_feature 1..22: note = Synthetic
  source 1..22: mol_type = other DNA
tctcataccttctgcttgatga

SEQ ID NO: 671 - DNA, 23 bp, synthetic construct
  misc_feature 1..23: note = Synthetic
  source 1..23: mol_type = other DNA
ttctcataccttctgcttgatga

SEQ ID NO: 672 - DNA, 24 bp, synthetic construct
  misc_feature 1..24: note = Synthetic
  source 1..24: mol_type = other DNA
tttctcataccttctgcttgatga

SEQ ID NO: 673 - DNA, 25 bp, synthetic construct
  misc_feature 1..25: note = Synthetic
  source 1..25: mol_type = other DNA
ttttctcataccttctgcttgatga

SEQ ID NO: 674 - DNA, 20 bp, synthetic construct
  misc_feature 1..20: note = Synthetic
  source 1..20: mol_type = other DNA
ctcataccttctgcttgatg

SEQ ID NO: 675 - DNA, 21 bp, synthetic construct
  misc_feature 1..21: note = Synthetic
  source 1..21: mol_type = other DNA
tctcataccttctgcttgatg

SEQ ID NO: 676 - DNA, 22 bp, synthetic construct
  misc_feature 1..22: note = Synthetic
  source 1..22: mol_type = other DNA
ttctcataccttctgcttgatg

SEQ ID NO: 677 - DNA, 23 bp, synthetic construct
  misc_feature 1..23: note = Synthetic
  source 1..23: mol_type = other DNA
tttctcataccttctgcttgatg

SEQ ID NO: 678 - DNA, 24 bp, synthetic construct
  misc_feature 1..24: note = Synthetic
  source 1..24: mol_type = other DNA
ttttctcataccttctgcttgatg

SEQ ID NO: 679 - DNA, 25 bp, synthetic construct
  misc_feature 1..25: note = Synthetic
  source 1..25: mol_type = other DNA
tttttctcataccttctgcttgatg

SEQ ID NO: 680 - DNA, 20 bp, synthetic construct
  misc_feature 1..20: note = Synthetic
  source 1..20: mol_type = other DNA
tctcataccttctgcttgat

SEQ ID NO: 681 - DNA, 21 bp, synthetic construct
  misc_feature 1..21: note = Synthetic
  source 1..21: mol_type = other DNA
ttctcataccttctgcttgat

SEQ ID NO: 682 - DNA, 22 bp, synthetic construct
  misc_feature 1..22: note = Synthetic
  source 1..22: mol_type = other DNA
tttctcataccttctgcttgat

SEQ ID NO: 683 - DNA, 23 bp, synthetic construct
  misc_feature 1..23: note = Synthetic
  source 1..23: mol_type = other DNA
ttttctcataccttctgcttgat

SEQ ID NO: 684 - DNA, 24 bp, synthetic construct
  misc_feature 1..24: note = Synthetic
  source 1..24: mol_type = other DNA
tttttctcataccttctgcttgat

SEQ ID NO: 685 - DNA, 25 bp, synthetic construct
  misc_feature 1..25: note = Synthetic
  source 1..25: mol_type = other DNA
ttttttctcataccttctgcttgat

SEQ ID NO: 686 - DNA, 20 bp, synthetic construct
  misc_feature 1..20: note = Synthetic
  source 1..20: mol_type = other DNA
ttctcataccttctgcttga

SEQ ID NO: 687 - DNA, 21 bp, synthetic construct
  misc_feature 1..21: note = Synthetic
  source 1..21: mol_type = other DNA
tttctcataccttctgcttga

SEQ ID NO: 688 - DNA, 22 bp, synthetic construct
  misc_feature 1..22: note = Synthetic
  source 1..22: mol_type = other DNA
ttttctcataccttctgcttga

SEQ ID NO: 689 - DNA, 23 bp, synthetic construct
  misc_feature 1..23: note = Synthetic
  source 1..23: mol_type = other DNA
tttttctcataccttctgcttga

SEQ ID NO: 690 - DNA, 24 bp, synthetic construct
  misc_feature 1..24: note = Synthetic
  source 1..24: mol_type = other DNA
ttttttctcataccttctgcttga

SEQ ID NO: 691 - DNA, 25 bp, synthetic construct
  misc_feature 1..25: note = Synthetic
  source 1..25: mol_type = other DNA
attttttctcataccttctgcttga

SEQ ID NO: 692 - DNA, 20 bp, synthetic construct
  misc_feature 1..20: note = Synthetic
  source 1..20: mol_type = other DNA
tttctcataccttctgcttg

SEQ ID NO: 693 - DNA, 21 bp, synthetic construct
  misc_feature 1..21: note = Synthetic
  source 1..21: mol_type = other DNA
ttttctcataccttctgcttg

SEQ ID NO: 694 - DNA, 22 bp, synthetic construct
  misc_feature 1..22: note = Synthetic
  source 1..22: mol_type = other DNA
tttttctcataccttctgcttg

SEQ ID NO: 695 - DNA, 23 bp, synthetic construct
  misc_feature 1..23: note = Synthetic
  source 1..23: mol_type = other DNA
ttttttctcataccttctgcttg

SEQ ID NO: 696 - DNA, 24 bp, synthetic construct
  misc_feature 1..24: note = Synthetic
  source 1..24: mol_type = other DNA
attttttctcataccttctgcttg

SEQ ID NO: 697 - DNA, 20 bp, synthetic construct
  misc_feature 1..20: note = Synthetic
  source 1..20: mol_type = other DNA
ttttctcataccttctgctt

SEQ ID NO: 698 - DNA, 21 bp, synthetic construct
  misc_feature 1..21: note = Synthetic
  source 1..21: mol_type = other DNA
tttttctcataccttctgctt

SEQ ID NO: 699 - DNA, 22 bp, synthetic construct
  misc_feature 1..22: note = Synthetic
  source 1..22: mol_type = other DNA
ttttttctcataccttctgctt

SEQ ID NO: 700 - DNA, 23 bp, synthetic construct
  misc_feature 1..23: note = Synthetic
  source 1..23: mol_type = other DNA
attttttctcataccttctgctt

SEQ ID NO: 701 - DNA, 20 bp, synthetic construct
  misc_feature 1..20: note = Synthetic
  source 1..20: mol_type = other DNA
tttttctcataccttctgct

SEQ ID NO: 702 - DNA, 21 bp, synthetic construct
  misc_feature 1..21: note = Synthetic
  source 1..21: mol_type = other DNA
ttttttctcataccttctgct

SEQ ID NO: 703 - DNA, 22 bp, synthetic construct
  misc_feature 1..22: note = Synthetic
  source 1..22: mol_type = other DNA
attttttctcataccttctgct

SEQ ID NO: 704 - DNA, 20 bp, synthetic construct
  misc_feature 1..20: note = Synthetic
  source 1..20: mol_type = other DNA
ttttttctcataccttctgc

SEQ ID NO: 705 - DNA, 21 bp, synthetic construct
  misc_feature 1..21: note = Synthetic
  source 1..21: mol_type = other DNA
attttttctcataccttctgc

SEQ ID NO: 706 - DNA, 20 bp, synthetic construct
  misc_feature 1..20: note = Synthetic
  source 1..20: mol_type = other DNA
attttttctcataccttctg

SEQ ID NO: 707 - DNA, 23 bp, synthetic construct
  misc_feature 1..23: note = Synthetic
  source 1..23: mol_type = other DNA
acttctgccaacttttatcattt

SEQ ID NO: 708 - DNA, 22 bp, synthetic construct
  misc_feature 1..22: note = Synthetic
  source 1..22: mol_type = other DNA
atggtctaggagagtaaagtga

SEQ ID NO: 709 - DNA, 23 bp, synthetic construct
  misc_feature 1..23: note = Synthetic
  source 1..23: mol_type = other DNA
aatggtctaggagagtaaagtga

SEQ ID NO: 710 - DNA, 24 bp, synthetic construct
  misc_feature 1..24: note = Synthetic
  source 1..24: mol_type = other DNA
aaatggtctaggagagtaaagtga

SEQ ID NO: 711 - DNA, 25 bp, synthetic construct
  misc_feature 1..25: note = Synthetic
  source 1..25: mol_type = other DNA
ggaaatggtctaggagagtaaagtg

SEQ ID NO: 712 - DNA, 24 bp, synthetic construct
  misc_feature 1..24: note = Synthetic
  source 1..24: mol_type = other DNA
ggaaatggtctaggagagtaaagt

SEQ ID NO: 713 - DNA, 25 bp, synthetic construct
  misc_feature 1..25: note = Synthetic
  source 1..25: mol_type = other DNA
tgggaaatggtctaggagagtaaag

SEQ ID NO: 714 - DNA, 23 bp, synthetic construct
  misc_feature 1..23: note = Synthetic
  source 1..23: mol_type = other DNA
tgggaaatggtctaggagagtaa

SEQ ID NO: 715 - DNA, 24 bp, synthetic construct
  misc_feature 1..24: note = Synthetic
  source 1..24: mol_type = other DNA
gtgggaaatggtctaggagagtaa

SEQ ID NO: 716 - DNA, 25 bp, synthetic construct
  misc_feature 1..25: note = Synthetic
  source 1..25: mol_type = other DNA
ggtgggaaatggtctaggagagtaa

SEQ ID NO: 717 - DNA, 22 bp, synthetic construct
  misc_feature 1..22: note = Synthetic
  source 1..22: mol_type = other DNA
tgggaaatggtctaggagagta

SEQ ID NO: 718 - DNA, 23 bp, synthetic construct
  misc_feature 1..23: note = Synthetic
  source 1..23: mol_type = other DNA
gtgggaaatggtctaggagagta

SEQ ID NO: 719 - DNA, 24 bp, synthetic construct
  misc_feature 1..24: note = Synthetic
  source 1..24: mol_type = other DNA
ggtgggaaatggtctaggagagta

SEQ ID NO: 720 - DNA, 25 bp, synthetic construct
  misc_feature 1..25: note = Synthetic
  source 1..25: mol_type = other DNA
tggtgggaaatggtctaggagagta

SEQ ID NO: 721 - DNA, 21 bp, synthetic construct
  misc_feature 1..21: note = Synthetic
  source 1..21: mol_type = other DNA
tgggaaatggtctaggagagt

SEQ ID NO: 722 - DNA, 22 bp, synthetic construct
  misc_feature 1..22: note = Synthetic
  source 1..22: mol_type = other DNA
gtgggaaatggtctaggagagt

SEQ ID NO: 723 - DNA, 23 bp, synthetic construct
  misc_feature 1..23: note = Synthetic
  source 1..23: mol_type = other DNA
ggtgggaaatggtctaggagagt

SEQ ID NO: 724 - DNA, 24 bp, synthetic construct
  misc_feature 1..24: note = Synthetic
  source 1..24: mol_type = other DNA
tggtgggaaatggtctaggagagt

SEQ ID NO: 725 - DNA, 25 bp, synthetic construct
  misc_feature 1..25: note = Synthetic
  source 1..25: mol_type = other DNA
ctggtgggaaatggtctaggagagt

SEQ ID NO: 726 - DNA, 20 bp, synthetic construct
  misc_feature 1..20: note = Synthetic
  source 1..20: mol_type = other DNA
tgggaaatggtctaggagag

SEQ ID NO: 727 - DNA, 21 bp, synthetic construct
  misc_feature 1..21: note = Synthetic
  source 1..21: mol_type = other DNA
gtgggaaatggtctaggagag

SEQ ID NO: 728 - DNA, 22 bp, synthetic construct
  misc_feature 1..22: note = Synthetic
  source 1..22: mol_type = other DNA
ggtgggaaatggtctaggagag

SEQ ID NO: 729 - DNA, 23 bp, synthetic construct
  misc_feature 1..23: note = Synthetic
  source 1..23: mol_type = other DNA
tggtgggaaatggtctaggagag

SEQ ID NO: 730 - DNA, 24 bp, synthetic construct
  misc_feature 1..24: note = Synthetic
  source 1..24: mol_type = other DNA
ctggtgggaaatggtctaggagag

SEQ ID NO: 731 - DNA, 20 bp, synthetic construct
  misc_feature 1..20: note = Synthetic
  source 1..20: mol_type = other DNA
gtgggaaatggtctaggaga

SEQ ID NO: 732 - DNA, 21 bp, synthetic construct
  misc_feature 1..21: note = Synthetic
  source 1..21: mol_type = other DNA
ggtgggaaatggtctaggaga

SEQ ID NO: 733 - DNA, 22 bp, synthetic construct
  misc_feature 1..22: note = Synthetic
  source 1..22: mol_type = other DNA
tggtgggaaatggtctaggaga

SEQ ID NO: 734 - DNA, 23 bp, synthetic construct
  misc_feature 1..23: note = Synthetic
  source 1..23: mol_type = other DNA
ctggtgggaaatggtctaggaga

SEQ ID NO: 735 - DNA, 25 bp, synthetic construct
  misc_feature 1..25: note = Synthetic
  source 1..25: mol_type = other DNA
aactggtgggaaatggtctaggaga

SEQ ID NO: 736 - DNA, 20 bp, synthetic construct
  misc_feature 1..20: note = Synthetic
  source 1..20: mol_type = other DNA
ggtgggaaatggtctaggag

SEQ ID NO: 737 - DNA, 21 bp, synthetic construct
  misc_feature 1..21: note = Synthetic
  source 1..21: mol_type = other DNA
tggtgggaaatggtctaggag

SEQ ID NO: 738 - DNA, 22 bp, synthetic construct
  misc_feature 1..22: note = Synthetic
  source 1..22: mol_type = other DNA
ctggtgggaaatggtctaggag

SEQ ID NO: 739 - DNA, 24 bp, synthetic construct
  misc_feature 1..24: note = Synthetic
  source 1..24: mol_type = other DNA
aactggtgggaaatggtctaggag

SEQ ID NO: 740 - DNA, 20 bp, synthetic construct
  misc_feature 1..20: note = Synthetic
  source 1..20: mol_type = other DNA
tggtgggaaatggtctagga

SEQ ID NO: 741 - DNA, 21 bp, synthetic construct
  misc_feature 1..21: note = Synthetic
  source 1..21: mol_type = other DNA
ctggtgggaaatggtctagga

SEQ ID NO: 742 - DNA, 23 bp, synthetic construct
  misc_feature 1..23: note = Synthetic
  source 1..23: mol_type = other DNA
aactggtgggaaatggtctagga

SEQ ID NO: 743 - DNA, 25 bp, synthetic construct
  misc_feature 1..25: note = Synthetic
  source 1..25: mol_type = other DNA
agaactggtgggaaatggtctagga

SEQ ID NO: 744 - DNA, 20 bp, synthetic construct
  misc_feature 1..20: note = Synthetic
  source 1..20: mol_type = other DNA
ctggtgggaaatggtctagg

SEQ ID NO: 745 - DNA, 22 bp, synthetic construct
  misc_feature 1..22: note = Synthetic
  source 1..22: mol_type = other DNA
aactggtgggaaatggtctagg

SEQ ID NO: 746 - DNA, 24 bp, synthetic construct
  misc_feature 1..24: note = Synthetic
  source 1..24: mol_type = other DNA
agaactggtgggaaatggtctagg

SEQ ID NO: 747 - DNA, 25 bp, synthetic construct
  misc_feature 1..25: note = Synthetic
  source 1..25: mol_type = other DNA
aagaactggtgggaaatggtctagg

SEQ ID NO: 748 - DNA, 21 bp, synthetic construct
  misc_feature 1..21: note = Synthetic
  source 1..21: mol_type = other DNA
aactggtgggaaatggtctag

SEQ ID NO: 749 - DNA, 23 bp, synthetic construct
  misc_feature 1..23: note = Synthetic
  source 1..23: mol_type = other DNA
agaactggtgggaaatggtctag

SEQ ID NO: 750 - DNA, 24 bp, synthetic construct
  misc_feature 1..24: note = Synthetic
  source 1..24: mol_type = other DNA
aagaactggtgggaaatggtctag

SEQ ID NO: 751 - DNA, 20 bp, synthetic construct
  misc_feature 1..20: note = Synthetic
  source 1..20: mol_type = other DNA
aactggtgggaaatggtcta

SEQ ID NO: 752 - DNA, 22 bp, synthetic construct
  misc_feature 1..22: note = Synthetic
  source 1..22: mol_type = other DNA
agaactggtgggaaatggtcta

SEQ ID NO: 753 - DNA, 23 bp, synthetic construct
  misc_feature 1..23: note = Synthetic
  source 1..23: mol_type = other DNA
aagaactggtgggaaatggtcta

SEQ ID NO: 754 - DNA, 25 bp, synthetic construct
  misc_feature 1..25: note = Synthetic
  source 1..25: mol_type = other DNA
ctaagaactggtgggaaatggtcta

SEQ ID NO: 755 - DNA, 21 bp, synthetic construct
  misc_feature 1..21: note = Synthetic
  source 1..21: mol_type = other DNA
agaactggtgggaaatggtct

SEQ ID NO: 756 - DNA, 22 bp, synthetic construct
  misc_feature 1..22: note = Synthetic
  source 1..22: mol_type = other DNA
aagaactggtgggaaatggtct

SEQ ID NO: 757 - DNA, 24 bp, synthetic construct
  misc_feature 1..24: note = Synthetic
  source 1..24: mol_type = other DNA
ctaagaactggtgggaaatggtct

SEQ ID NO: 758 - DNA, 25 bp, synthetic construct
  misc_feature 1..25: note = Synthetic
  source 1..25: mol_type = other DNA
cctaagaactggtgggaaatggtct

SEQ ID NO: 759 - DNA, 20 bp, synthetic construct
  misc_feature 1..20: note = Synthetic
  source 1..20: mol_type = other DNA
agaactggtgggaaatggtc

SEQ ID NO: 760 - DNA, 21 bp, synthetic construct
  misc_feature 1..21: note = Synthetic
  source 1..21: mol_type = other DNA
aagaactggtgggaaatggtc

SEQ ID NO: 761 - DNA, 23 bp, synthetic construct
  misc_feature 1..23: note = Synthetic
  source 1..23: mol_type = other DNA
ctaagaactggtgggaaatggtc

SEQ ID NO: 762 - DNA, 24 bp, synthetic construct
  misc_feature 1..24: note = Synthetic
  source 1..24: mol_type = other DNA
cctaagaactggtgggaaatggtc

SEQ ID NO: 763 - DNA, 25 bp, synthetic construct
  misc_feature 1..25: note = Synthetic
  source 1..25: mol_type = other DNA
gcctaagaactggtgggaaatggtc

SEQ ID NO: 764 - DNA, 24 bp, synthetic construct
  misc_feature 1..24: note = Synthetic
  source 1..24: mol_type = other DNA
gcctaagaactggtgggaaatggt

SEQ ID NO: 765 - DNA, 25 bp, synthetic construct
  misc_feature 1..25: note = Synthetic
  source 1..25: mol_type = other DNA
tgcctaagaactggtgggaaatggt

SEQ ID NO: 766 - DNA, 23 bp, synthetic construct
  misc_feature 1..23: note = Synthetic
  source 1..23: mol_type = other DNA
gcctaagaactggtgggaaatgg

SEQ ID NO: 767 - DNA, 24 bp, synthetic construct
  misc_feature 1..24: note = Synthetic
  source 1..24: mol_type = other DNA
tgcctaagaactggtgggaaatgg

SEQ ID NO: 768 - DNA, 25 bp, synthetic construct
  misc_feature 1..25: note = Synthetic
  source 1..25: mol_type = other DNA
ttgcctaagaactggtgggaaatgg

SEQ ID NO: 769 - DNA, 23 bp, synthetic construct
  misc_feature 1..23: note = Synthetic
  source 1..23: mol_type = other DNA
tgcctaagaactggtgggaaatg

SEQ ID NO: 770 - DNA, 24 bp, synthetic construct
  misc_feature 1..24: note = Synthetic
  source 1..24: mol_type = other DNA
ttgcctaagaactggtgggaaatg

SEQ ID NO: 771 - DNA, 20 bp, synthetic construct
  misc_feature 1..20: note = Synthetic
  source 1..20: mol_type = other DNA
gtgagaccagccaaaacact

SEQ ID NO: 772 - DNA, 21 bp, synthetic construct
  misc_feature 1..21: note = Synthetic
  source 1..21: mol_type = other DNA
tgtgagaccagccaaaacact

SEQ ID NO: 773 - DNA, 22 bp, synthetic construct
  misc_feature 1..22: note = Synthetic
  source 1..22: mol_type = other DNA
ttgtgagaccagccaaaacact

SEQ ID NO: 774 - DNA, 23 bp, synthetic construct
  misc_feature 1..23: note = Synthetic
  source 1..23: mol_type = other DNA
attgtgagaccagccaaaacact

SEQ ID NO: 775 - DNA, 22 bp, synthetic construct
  misc_feature 1..22: note = Synthetic
  source 1..22: mol_type = other DNA
attgtgagaccagccaaaacac

SEQ ID NO: 776 - DNA, 25 bp, synthetic construct
  misc_feature 1..25: note = Synthetic
  source 1..25: mol_type = other DNA
gtacaattgtgagaccagccaaaac

SEQ ID NO: 777 - DNA, 23 bp, synthetic construct
  misc_feature 1..23: note = Synthetic
  source 1..23: mol_type = other DNA
gtacaattgtgagaccagccaaa

SEQ ID NO: 778 - DNA, 22 bp, synthetic construct
  misc_feature 1..22: note = Synthetic
  source 1..22: mol_type = other DNA
gtacaattgtgagaccagccaa

SEQ ID NO: 779 - DNA, 23 bp, synthetic construct
  misc_feature 1..23: note = Synthetic
  source 1..23: mol_type = other DNA
agtacaattgtgagaccagccaa

SEQ ID NO: 780 - DNA, 21 bp, synthetic construct
  misc_feature 1..21: note = Synthetic
  source 1..21: mol_type = other DNA
gtacaattgtgagaccagcca

SEQ ID NO: 781 - DNA, 22 bp, synthetic construct
  misc_feature 1..22: note = Synthetic
  source 1..22: mol_type = other DNA
agtacaattgtgagaccagcca

SEQ ID NO: 782 - DNA, 23 bp, synthetic construct
  misc_feature 1..23: note = Synthetic
  source 1..23: mol_type = other DNA
aagtacaattgtgagaccagcca

SEQ ID NO: 783 - DNA, 24 bp, synthetic construct
  misc_feature 1..24: note = Synthetic
  source 1..24: mol_type = other DNA
aaagtacaattgtgagaccagcca

SEQ ID NO: 784 - DNA, 20 bp, synthetic construct
  misc_feature 1..20: note = Synthetic
  source 1..20: mol_type = other DNA
gtacaattgtgagaccagcc

SEQ ID NO: 785 - DNA, 21 bp, synthetic construct
  misc_feature 1..21: note = Synthetic
  source 1..21: mol_type = other DNA
agtacaattgtgagaccagcc

SEQ ID NO: 786 - DNA, 22 bp, synthetic construct
  misc_feature 1..22: note = Synthetic
  source 1..22: mol_type = other DNA
aagtacaattgtgagaccagcc

SEQ ID NO: 787 - DNA, 23 bp, synthetic construct
  misc_feature 1..23: note = Synthetic
  source 1..23: mol_type = other DNA
aaagtacaattgtgagaccagcc

SEQ ID NO: 788 - DNA, 25 bp, synthetic construct
  misc_feature 1..25: note = Synthetic
  source 1..25: mol_type = other DNA
gtaaagtacaattgtgagaccagcc

SEQ ID NO: 789 - DNA, 24 bp, synthetic construct
  misc_feature 1..24: note = Synthetic
  source 1..24: mol_type = other DNA
gtaaagtacaattgtgagaccagc

SEQ ID NO: 790 - DNA, 25 bp, synthetic construct
  misc_feature 1..25: note = Synthetic
  source 1..25: mol_type = other DNA
agtaaagtacaattgtgagaccagc

SEQ ID NO: 791 - DNA, 24 bp, synthetic construct
  misc_feature 1..24: note = Synthetic
  source 1..24: mol_type = other DNA
tcagcgttgtgtattccttttaca

SEQ ID NO: 792 - DNA, 25 bp, synthetic construct
  misc_feature 1..25: note = Synthetic
  source 1..25: mol_type = other DNA
ttcagcgttgtgtattccttttaca

SEQ ID NO: 793 - DNA, 23 bp, synthetic construct
  misc_feature 1..23: note = Synthetic
  source 1..23: mol_type = other DNA
tcagcgttgtgtattccttttac

SEQ ID NO: 794 - DNA, 24 bp, synthetic construct
  misc_feature 1..24: note = Synthetic
  source 1..24: mol_type = other DNA
ttcagcgttgtgtattccttttac

SEQ ID NO: 795 - DNA, 25 bp, synthetic construct
  misc_feature 1..25: note = Synthetic
  source 1..25: mol_type = other DNA
cttcagcgttgtgtattccttttac

SEQ ID NO: 796 - DNA, 22 bp, synthetic construct
  misc_feature 1..22: note = Synthetic
  source 1..22: mol_type = other DNA
tcagcgttgtgtattcctttta

SEQ ID NO: 797 - DNA, 23 bp, synthetic construct
  misc_feature 1..23: note = Synthetic
  source 1..23: mol_type = other DNA
ttcagcgttgtgtattcctttta

SEQ ID NO: 798 - DNA, 24 bp, synthetic construct
  misc_feature 1..24: note = Synthetic
  source 1..24: mol_type = other DNA
cttcagcgttgtgtattcctttta

SEQ ID NO: 799 - DNA, 25 bp, synthetic construct
  misc_feature 1..25: note = Synthetic
  source 1..25: mol_type = other DNA
tcttcagcgttgtgtattcctttta

SEQ ID NO: 800 - DNA, 21 bp, synthetic construct
  misc_feature 1..21: note = Synthetic
  source 1..21: mol_type = other DNA
tcagcgttgtgtattcctttt

SEQ ID NO: 801 - DNA, 22 bp, synthetic construct
  misc_feature 1..22: note = Synthetic
  source 1..22: mol_type = other DNA
ttcagcgttgtgtattcctttt

SEQ ID NO: 802 - DNA, 23 bp, synthetic construct
  misc_feature 1..23: note = Synthetic
  source 1..23: mol_type = other DNA
cttcagcgttgtgtattcctttt

SEQ ID NO: 803 - DNA, 24 bp, synthetic construct
  misc_feature 1..24: note = Synthetic
  source 1..24: mol_type = other DNA
tcttcagcgttgtgtattcctttt

SEQ ID NO: 804 - DNA, 25 bp, synthetic construct
  misc_feature 1..25: note = Synthetic
  source 1..25: mol_type = other DNA
ttcttcagcgttgtgtattcctttt

SEQ ID NO: 805 - DNA, 20 bp, synthetic construct
  misc_feature 1..20: note = Synthetic
  source 1..20: mol_type = other DNA
tcagcgttgtgtattccttt

SEQ ID NO: 806 - DNA, 21 bp, synthetic construct
  misc_feature 1..21: note = Synthetic
  source 1..21: mol_type = other DNA
ttcagcgttgtgtattccttt

SEQ ID NO: 807 - DNA, 22 bp, synthetic construct
  misc_feature 1..22: note = Synthetic
  source 1..22: mol_type = other DNA
cttcagcgttgtgtattccttt

SEQ ID NO: 808 - DNA, 23 bp, synthetic construct
  misc_feature 1..23: note = Synthetic
  source 1..23: mol_type = other DNA
tcttcagcgttgtgtattccttt

SEQ ID NO: 809 - DNA, 24 bp, synthetic construct
  misc_feature 1..24: note = Synthetic
  source 1..24: mol_type = other DNA
ttcttcagcgttgtgtattccttt

SEQ ID NO: 810 - DNA, 25 bp, synthetic construct
  misc_feature 1..25: note = Synthetic
  source 1..25: mol_type = other DNA
gttcttcagcgttgtgtattccttt

SEQ ID NO: 811 - DNA, 20 bp, synthetic construct
  misc_feature 1..20: note = Synthetic
  source 1..20: mol_type = other DNA
ttcagcgttgtgtattcctt

SEQ ID NO: 812 - DNA, 21 bp, synthetic construct
  misc_feature 1..21: note = Synthetic
  source 1..21: mol_type = other DNA
cttcagcgttgtgtattcctt

SEQ ID NO: 813 - DNA, 22 bp, synthetic construct
  misc_feature 1..22: note = Synthetic
  source 1..22: mol_type = other DNA
tcttcagcgttgtgtattcctt

SEQ ID NO: 814 - DNA, 23 bp, synthetic construct
  misc_feature 1..23: note = Synthetic
  source 1..23: mol_type = other DNA
ttcttcagcgttgtgtattcctt

SEQ ID NO: 815 - DNA, 24 bp, synthetic construct
  misc_feature 1..24: note = Synthetic
  source 1..24: mol_type = other DNA
gttcttcagcgttgtgtattcctt

SEQ ID NO: 816 - DNA, 20 bp, synthetic construct
  misc_feature 1..20: note = Synthetic
  source 1..20: mol_type = other DNA
cttcagcgttgtgtattcct

SEQ ID NO: 817 - DNA, 21 bp, synthetic construct
  misc_feature 1..21: note = Synthetic
  source 1..21: mol_type = other DNA
tcttcagcgttgtgtattcct

SEQ ID NO: 818 - DNA, 22 bp, synthetic construct
  misc_feature 1..22: note = Synthetic
  source 1..22: mol_type = other DNA
ttcttcagcgttgtgtattcct

SEQ ID NO: 819 - DNA, 23 bp, synthetic construct
  misc_feature 1..23: note = Synthetic
  source 1..23: mol_type = other DNA
gttcttcagcgttgtgtattcct

SEQ ID NO: 820 - DNA, 20 bp, synthetic construct
  misc_feature 1..20: note = Synthetic
  source 1..20: mol_type = other DNA
tcttcagcgttgtgtattcc

SEQ ID NO: 821 - DNA, 21 bp, synthetic construct
  misc_feature 1..21: note = Synthetic
  source 1..21: mol_type = other DNA
ttcttcagcgttgtgtattcc

SEQ ID NO: 822 - DNA, 22 bp, synthetic construct
  misc_feature 1..22: note = Synthetic
  source 1..22: mol_type = other DNA
gttcttcagcgttgtgtattcc

SEQ ID NO: 823 - DNA, 24 bp, synthetic construct
  misc_feature 1..24: note = Synthetic
  source 1..24: mol_type = other DNA
gggttcttcagcgttgtgtattcc

SEQ ID NO: 824 - DNA, 25 bp, synthetic construct
  misc_feature 1..25: note = Synthetic
  source 1..25: mol_type = other DNA
agggttcttcagcgttgtgtattcc

SEQ ID NO: 825 - DNA, 21 bp, synthetic construct
  misc_feature 1..21: note = Synthetic
  source 1..21: mol_type = other DNA
gttcttcagcgttgtgtattc

SEQ ID NO: 826 - DNA, 23 bp, synthetic construct
  misc_feature 1..23: note = Synthetic
  source 1..23: mol_type = other DNA
gggttcttcagcgttgtgtattc

SEQ ID NO: 827 - DNA, 24 bp, synthetic construct
  misc_feature 1..24: note = Synthetic
  source 1..24: mol_type = other DNA
agggttcttcagcgttgtgtattc

SEQ ID NO: 828 - DNA, 22 bp, synthetic construct
  misc_feature 1..22: note = Synthetic
  source 1..22: mol_type = other DNA
gggttcttcagcgttgtgtatt

SEQ ID NO: 829 - DNA, 21 bp, synthetic construct
  misc_feature 1..21: note = Synthetic
  source 1..21: mol_type = other DNA
gggttcttcagcgttgtgtat

SEQ ID NO: 830 - DNA, 20 bp, synthetic construct
  misc_feature 1..20: note = Synthetic
  source 1..20: mol_type = other DNA
gggttcttcagcgttgtgta

SEQ ID NO: 831 - DNA, 23 bp, synthetic construct
  misc_feature 1..23: note = Synthetic
  source 1..23: mol_type = other DNA
atcagggttcttcagcgttgtgt

SEQ ID NO: 832 - DNA, 60 bp, Homo sapiens
  source 1..60: mol_type = genomic DNA
tagctcctggactgaccactattggagcctgtaagtatactggatcccattctctttggc

SEQ ID NO: 833 - DNA, 52 bp, Homo sapiens
  source 1..52: mol_type = genomic DNA
attggagcctgtaagtatactggatcccattctctttggctctagctatttg

SEQ ID NO: 834 - DNA, 32767 bp, Homo sapiens
  source 1..32767: mol_type = genomic DNA
gtaagtatactggatcccattctctttggctctagctatttgttcaaaagtgcaactatgaagtgatgactgggtgagagagaaaatttgtttcaattctaaagatagagataaacctttgtgttattgactgtgcaaaaagtcttagagtacattccttggaaattgactctgattcaaagtgttgcatgacaacgggatatggggagtgttctctggagatacacccacaaggaagagaagagcacaagggagattgtgggagagtctgaaatgtgatttgtctgcagcagaggcctaagccagtctcgcaggagccctacatctgggctggctgtgcagagctgtcctgaattgcaggcagtgggcctggcccttgtattcctgatccagccagccattggccaggggctggctgctgcctgagagtggaaggacaacttggacaagttttctgaggccgaaggcaattcttagtaaggaacaccattaacaaccaatattcctagcatccagggatgtgtgcattgttcctgaagagggacaagtatgtctacaaaaatcacagaaaccacagaaacacacacagtcctactagcacctctccctgtcccatttgcaaacaatttaagagctctcccatttttagttcaagaaaaagaaaaatggattgggaggaccacaagctgacttgggggaggaatatttcctcatttagctgtagttttaacttttgttttcactgcatattttcagtctattttattttctttcctcttcagttgttgatagaaggtattcataaattctcatggcaatgttaatgctggctttgactctcaggggaaagaggccagaaaacttctttgctgtaccattccataattaggcagaactaaaaacatctttgggtgttgttttttgtttttgtttttttttttgccttgtctgcttttcaaagatcaaatgattgaagcattaaagcatggtgactggttcttcaggtaaagttgatttttattttatgtcaagtagaaaaatactgaactggaagaatcacagctggggtagcacaatcataattcattagaaggcataaatagtgcttggattaaaagaagccctacaatctggggacagtgcatctcatgtgccctctgggattactcggcagtcatcagagttagatttaacgactttggagacttaagcattatggttttttttttttgtcaatctgggacactgaaattgctgtatcagggttatactcaactgtgtcaggtttatttgtttttatgagctgtaatttttggttccctcagcgcatatgcatagtttgttcctatgttatcatttattggtgtctgttttctggctgtctctggtaggttcagcctcagactctgtaactccatgaagagattatgttccaatgatgttttataagtttgttaaactctgaactcatgagtttatgtcccatataagccacgttacacatggtaggaaggctccaaaaccagggcgccgaaatccatttaacgtgtaacttacctaaatgtaacaatgtttataagaaaaatacattggaagttccagttttgacttccagcaacatatataattcatccactttatttattaacttccatgtgttgagcatcatactggtgctgcgagtacagcatagaataaaagtctctcctttcataaaacatatattgtaattgaaagagaaagacaataaactaatgaagaaaatatatactgtctcaataattataagtgctgtagagtgtagtctacagattgatgtcaatgggtatttgttagacacacagaatctgaggcccctatcctagacccactgaatcacaatctgcattttaatagaatccccaggtgaatcctgtgcacactgacatttgagaaacaccattacagagaacaactaagcagggagatgggatgagggtattattgtctatagtgtggtcagggaagcttgtctgttaagagaacatcagaaaactgatgtaagtgaggaagtgagcctggtgtatttctgggaaaattattccaggcagggagagaaaagactgagcaacgatactgaagtaggaacaagatgacggaatattaaggagatcagtgagactagaggagtgggtcaggggaagtgtgatggaagccatgagagatactcatctttcatagcactgccctacttccttctccccaacatgagggtctcatcaccccccaccactcttgtcttctcctatgtcctccacattgctgccagtatggagagtctgggaatgccctcagctcaaagctgtttggtgatagctggcagagttgtggtagtagctaaaaaagaattaagggaaagaggaattttctcaaaagcaggtgcttttcatcctctttagcaaaccgaaacagatctgagcattaagtcaagatgttaaatacacaaatgttgaatgaaaaaaaaaacaaaaggtagtcatttaaattcagagctgcttttattaaaataagattttcttttttctttactgtggtagttcaaatatcagaataaagaattgtttctattcccgacttcctgacttgcaggaagttaatcagaaataaatgcaatataaaaaaagaaaatctaatttgtattatgcttcttgtatatgtttattatttcatgtactgtattacaatgtaatagaatttataattcattatagcagattgtttccattgcattcctactattaaatatgtagaagctacacatatacttgtagctttaacatatatgtctttatcctcaaaataactgcaaagaacatatagataatttttaaagattaaggagcctgaggtttagaggggagatagctagattaaggccacacagctagaaagcaagcaagcaagggtttcagtccacatgtcaagctccacagcctgtgttttgttttgtggctgtgctttacactatctctctgtccaagaacctaatggaaaattacagatacagatgcagctggccagcagttaatataatttaactcaatcttaaatttatctggagtaaaagtgatacaagtttccgtgttttttcttttctttctttcttttttctttgtgtgtgtgtgtgtgtgtgtgtgtgtgacagaatcttgctctgttgcccaggctggagtgcagtggtgcgatctcggtttactacaacctctgcctttcaggttcaagcgattctcctgcctcagcctcccaagtagctgggatacaggtgcgcaccaccatgcccagctaatttttgtagttttagtaaagacggggttgctccatgttgatcaggctggtcttaaactcctgacctcaagtgatccgcctgcctcagcctctcaaagtgctgggattacaggcatgagccaccttgactggcctgtttctgtgttttttctacttaagaagtagaaaaaattggttcactctatttgaatttcttagaaccatagaaatccaaacttggaataattattgcaattattatctagttgagtattcttcttttatagatgtagaagctgaggcctaaagttgcttgttttgttttatgaacactaaaccaaactaccttgtggctgcttacttaaattataaattataatggggtggccttgcctgagctgtataaattgttttaattatcaggacaaatcaacatactggaaaaaaaaagcaaaacttgcaattgttgttgcttagacacctgtccatatcagtttctattgttgcatagaagcaccccaaaacttaacagttcaaaacgagtgatttattgctcataattctgtgtgtctgaagtttgctctgtgattagctggatagttattctgatggtcttgcttggtgttacttatgtggatgcagttatctagcaagtgaacaggagctagatggtctaaaatgcactctctcagatatctgacagctggctgttggttgcagaaccttgattcttcataaggccactcatcatcctgtaggctagattaggcttcattacatggtattctcagggcagttttccaagagagggcgggtggaagctacaagaccttctgatgcctaggctttgaaacgtgtaggttacttctgctaagttatattggtcaaagcacctcaaaagatcagcccagattcaagagatgaggaaattacttcatctcgtgaaaggaggagatgccacatcgcatatcaaaggggtatgcatattggggatagaaggttttattgtcaccgtatttatacacaaatcactacattggggaaaaggagggaaactggagatcaaaagtgttggtcttaatccaacttaattctcaaaaattaccatgcgttagaaccacccagttcttcgcaaagtatagattactgggcttcaaccccagagtttctgatttactagctctggggtgagacctgcatctctctctctctctttttttttgagactgagtcttgctctgtcacccaggctggagtgcagtggtgcaatctcggctcacagcaacctctgcctcctgggttgaagcggttctcctgcctcagcctcctgagtagctaggattataggcacccgccaccacgcctggctaatttttgtatttttagtagagacagggtttcaccatgttggtcaggctggtctcgaactcctgacctcaggtgatccacctgcctcagcctcccatagtgctgggattacaggcgtgagccactgtgcccagccaagacttgcatctctgaaaagttcctaggttatgctgatgctggcctatgctttgagaactactaccacagacatacagtgagtggggaagaataaattcatcccttctgctgtgtgcagcaaggagtgggattccaatgagatccagtgctgtgaatgctaaagggaaatccatcttattttagcacctctactccccatctccccaccccgaggatgttatagcttagaagttcaaggagatggacaacacactaaaccaggcagtatttgccctgcagagctgttcagtgttcctggatgagacctctgagaagaaaagccataagttcctctagagactttcacaatcatttaggtagacaggactttgcatgggtctgaaggcttgcatggcagatggaggcaaagagccagcaaatctggttgtaaatgtcaatgtgaatcctttcttatccacaagctgctgggcctgagaacattaatgttctacaatacccgatttagcatttttgaaagaaattgcatatagacatgcttaatgtgaagactccaaatcaggatatttgattcaaatgtctcttggtaataactatggaatgaataacccattgtatatggacatatagaagagccagttaacagagttttctttttttttttttttttttttttttttttttttgagacggagtctcactctgtcgcccaggctggagtgcagtggcgcgatctcggctcactgcaagctccgcctcccgggttcacgccattctcctgcctcagcctcccccgtagctgggactacaggcgcccgccaccacgcccggctaatttttttgtgttttttagtagagacggggtttcactgtgttagccaggatggtctcgatctcctgacctcgtgatccgcccgcctcggccttccaaagtgctgggattacaggcgtgagccaccgcgcccggcccagagttttctagttgattaaacactggtaaaatcatctcttcctgtaattaagtttggagagagcaagtctcaagtgtaattaggaaagcacagtattggaggtcaagaagcctaaattctgatatctcagttgtgctaccaactatataagggatcaacagactgtttctgcaaaggaccgatactagatattttctgctttatgggtcatgtggtcaactctgtcattgtatactaaaagccacagacaatacctaaataaagggacatggctgtgctccagtgacactttatttacagaaacgaggcaggtaagatagatttgacccttagtgggctacccatttccccttagcactcagccttcctataaaccatgaagcgtctaactacaagtacctgtgagtcttcctgagtcctttctctgcctgtaggagtagctctcccacttgcagagcaggctggaagctggggaggagattatctctggaatagcacttaaccaatggacaaaagctgggggataaaggtgttggtcttcatagttttcatactgctatgaagaaatacccgagactggataatttatacaggaaaaagaagtttaatggactcacagttccacatggctggagaggcctcacaatcatggcagaaggcgaaggaagagcagaggcacttcttacatgacagcaggcaagagagcatgtgcagggaaactgctctttataaaaccatcagatctcatgagacttattcactatcaggagaacagcacaggaaaaccccacccccatgattcagttacctcccacatggtccctcccacaacacgtggggattattggagttacaattcaagatgagatttgggtgagaacacagccaaaccatatcaataaatatcccaccttcttctccccttgggtaggacacctccaatgcatgttccacactaaaaatctccagtgaattgggcagttgaccacaatgataagcatacttattagcatgcctcgtataggcttccttccgatgtgtaggctttcttcccattttggcagaatctggacccaaccccacatctcctgagtttgatttcagtcctcttcagtattctcatatatctcaactttgttccctgctgagatacaactgacagatatatatgtatcttcagctctgcagctctatcttctcttctaccatggctttctgggatcagctcccaattgaattatttgtatttaaatccttgtctcagggtctacttctgagcaaacctaaattaatacaagtcttagaattagtgccacagttataccttttgagtaagttatgttgcaattcccattttatagatgagataaccaagggtcagaaaggttaaataatttgtccaaggttacagagctagtcagtgacagagtgggtattcaaaaataagtctctatgattccagagcttatgctattaatcagtgcaccatgtatcaagcagtgatttagttatcttcatttgaattttttgaagtctctatttaaattgaatatcaaatcctcacatataaggttatttatacctttttatttgttttatttatttatttatttttgcttaacttttttttattatactttaagttttagggtacatgtgcacaatgtgcaggtctgttacatatatatacatgtgccatgttggtgtgctgcacccattaactcgtcattgaacattaggtatatctcctaatgctatccctcccccctccccccaccccacagcaggccccagtgtgtgatgttccccttcctgtgaccatgtgttctcattgttcaattcccacctgtgagtgagaatatgcggtgtttggttttttgtccttgcgatagtttgctgagaatgatggtttacagcttcatccatatccctacaaaggacatgaactcatcattttttatggctgcatagtattccatggtgatttatacattttaatttgaacttcactcacctctacaaagttaaataaagcattccatttccatttcaataatactttaaatatctaggcagttaaagcatgaggttacttggcacttaaatgtactcttgtcatactacttttgttggcttaaattgcaaaaaaaaaaaatggttaatttattgtgacaataccctacatttatctgcagtgatcattttttttgtgaaaatggccttcgtttatctgcagcaaaggaaaaagaggatggcaattagttcttgcattcttattcctctcttgggtcctgatccttctcattaatagaaacatggcaggggaggggtatataacccacaccctttcctgttgtggttatgtttccactgttgattctgcttcaggtgaacctttaggattaggcaaataaatttccgtgaggccaaatctttttcttcctcattaacagatgatttctctgctaaaaacacttacgacatggctatactattgccggttttatagttacaggctctaaaccttgaaaacttcctcaaagtctaatacgtcaggagcaagcttttgtacaaaaaatgtgaagacccttaatcagttccaataacaaaataaatccattttaaaccctatcccaagatactgcaaggccttggagcagctggagagactccttaactcttgacattaattaattaatttaaaaattcatatttgtatgtatcagtgaggtaagagtgcttgaaatataatgagatgtgtcacactgtagaaaggggagtgacaacagaaagccctggctggtgaggccccagcacttctcacactcatcagaaggaagtctttccatgaaggcagtagggggtccctggtcccaggccaggtccttcaacaatgctcactggttaacaggaaaggcactacagtggcatcattgttaacatccaagacagtgatagaatgtgaaacttctactgtttagtatttagtattcagcatttagatgttaattatccattttgtgatgaagttccctttcttctccctctcttaaccttttggtagttttattgcatggttaccatttccagttagggttgtgctttggggtctgaactgatgaaggaagagaaactctagttcatcatttctaggaaaaagagaaggctaacatcaattctgatgattagagattttttgattacctatgtcctgcattttaacagaaataaaatgttaattacagtaaacttactttaaccttcttatgctatttaacactcttcaagaaaggacttttgcttgaaatcactacagagtataccataatcttgactgtcatactctgaccaagaaaccagaactatttaggcatatttatgagagtaagtaccctaccgctcaaaatggaaggcttcaacatgtattcctaatatttcctaaaacttcactgaatagttttaaaattgaatataactttaccttcagagagaaacaagatttcgaaaggaaattgtagcagttttgtacttcaattgtcgattttaagattgtggtctgaaaagtatttgaacagtcattctttcttttccactatctcaggaaaggctctcattctattagaagcaatcttagaagcgtaactgccaactctcttcttaaaaagtgaagagcagatggagttctaccaatctgctatacttgacagtttttcaactacctaacctaagttccagttactgtctaaattatttttattcgaataggaaaaatacatttatgcttactgtacatttcgacagtgttcattctttgaggtctgttatttaatctccttcccacttttcttttctatcttaaatcagatgagtccccatggtctggcaacaaaacagctgtttctcaaatttttaacgtctctgtggccctcctcctacccttccaagtgttttccatactatttttctgttgtgggcttcagaattggatgctgttccataattactggtcttcccatgactctgtcacatcataaagtctatatatacccttttcctacgtttacatgccaaataccaggtgtgtctagctttaattcaatgactagcatatggtaggtattgcactaatattttttggtcaagtgaacatatattttacagatagatcaaatacatcaaccttcccatgtaataataataataccaagaagattaaatgatcgcgtgcttgaccagctgtttagtggcagaatctggacccaaccccacatctcctgagtttaatttcagtgctcttcagcattcttatatatctcaactttgttccctgctgagacacagctgacagatatatatatatatcttcacaatctcagtagtaagccctatttttttaagattacagtgctgatcaaagagggatattctatggcattgatgcaaatcttccaggccagatataacccctatctccttaaaccctcattcttgactgtgtcttgaaatcaaatttgagaacctctgctttacatgctaccttcctcttaaaattcataaggtttattccttctctcatcttcagtatttttatgaaaatatgttttactcttatttccaaagtgcttgccagcccatgctgagttcattagacaaacaatctaggtatcctaatatttaggtaaaattgctagcagcactaactataccagtacccacttaaattgcaatataaacctacaataggaaaaaaaaagtcaaaattatactactttcaattcctacttctggaataattatcacaccttcaaaaaaactcataattgttctccaatattaaaaaccaggaactaaattacatcactatatatatatagctatatagacatataactgtatatagtttatatatatattacatgtcactatatatggttatatatacagatagctatatctctatatagtttttatataggaaactatatatataaactcatatatatttatatataaactcatatatatataaactcatatatatttatatataaactcatatatatttatatataaactcatatatatataagagattttatatgtatatatgagagacaatattgaaataaaacaaactagccaatctccaatgtcccttcattttcccaggactcttctttattctcaaagaaatgtatgaatacacaatataaataaatgttctataatgttcattatggattataatagttttcttgtcatactgaagtgaatgggaattgtttcttcgattattagcatacttttcataactatgtgatttggtgataggcacttttgcttactaagttcagcatgatcaaaacagaactcctattatttttattgttgattcttatttctttaaggtaaaatatgcccttttcctttgaacctgcacactgtttaacaaataagcacataactcaggattgaattgtacacttcgatttgagcttttttttcaaggtcaggacccagtttcacaagaagttttattttttccaatacaactgacatccactcccaccagctgaaagacaagaaaaacttgtctaataaagctttcagattcaatttgctgcctgcatacagcttgaggaatctctggaggtcactcacagcatgtgttgcaaccccaacagggagaagtaatgaaaagattctagttaaaaagctgacactgccccttccaacctctttgaatgtgaatataataagccagtttacagacgcaaatctctatgattctggggatttccatcttgatctctgactccaaggaacatttgaatgcatggatttgtatccattatctgggtgaataaatgcttcatattgaaaaaaggggtgctttaacaacataagtctgatgtaaatcaggcaaaacaacattgtcacttcatgtttaactctcctggagggtctctaaggtctcacagtttggttctattccagtaatatataggcctatcatagccattttcaaaaataatacctgctttcatttcgattattccccctagcttttgcattgacccgaacatacctaatatttatcttagggctaacacgcattaatgccttgctctgtaccaggcattttgccaagtattctttgtgcatttttctgtttaatcttacagcagccttatgaaataggtactacattattattatttttcaccatgagaggaaatgaaagcctagagagaatggttatccaaaaacacccagctactaagtgggcacagcatggccttgaacctgagtctttataaagttcatgcctgtcttttacctttatgttaaacatactgaatcttggtcatgcagtctatgaatgaagactccatatactctaggaccaattctaccatattgtgcatgcttttgtacatattttccaaatgaaattaaacaacaatactctctcttcccctttcttttccgttttgcatgacatctaaatcttttattaaatctccgtgggtggaattgggccttaagtagtagtacctttgaagcttaatactataacctcagagttacggaagtggtttcaatatgaagaaatatatacgttcttttcttttcttttcttttctttttttggctcctctagaatagaaggcaatcaggagagaaaaagacattaagatgataggcttgattctcccacagtgttactacttagctttatttattctcccacatctgtaactgtcaattcaaggtcaaaaagccagtacagcagcagaattacaaaaagtagatctggaaaatctatagggtaccatagtccagcaccctgctgcaagtcaaaatcaattaaataaatatgtttaaacagcacttcatttaatcttaagggcaccacgactcccttggaaaattcttttttgtgttcagacctgattctctgaaagtattttctagtgttttctttgttcggtggctgtggtgaataagtggacccaattgcctacagcatgaaatcgtagaaatgaatatgggctggaacttcaatcaatcacacaaaccagaaacatagagttcatcacctcttctggaaagcgtccccttcggccgtcaaaagggtagagtttcctccaacatatgttcatacttccctttgcctcccatctgcaatttcaaccagtctgttgctctcgccctttttaggcttcacataccggatttcttcttaggagctcacttgaaaaaagggttgtatgctttaaaaatactaaaagtcactggactagatgataataaaatttctgtaaaataaaaagggagttaacagaggtgttaccacttatcataatggatattgttgacctttcctctagccagtgtcccacttaagcatgcttcttggctggaatttatttttctcatctggataaatctgaagcattatagtaaaacccagtgtgagcaaggccatggatgcaagtggattgagatgaatgagtggatagacctgcgtgggcagaaataatgaggtcagcaataagccataccaagggatgcacactcaaggaataaattggaggtagcagaataactgaacaaatgatacatttgatcagactgtcacatgaatatcaactgaaacaagttatgactgtagttagtgaaccctagagtgaagaaaacaaaatattgaaatccatctgatcaaaaaataataaaattgtttacatgtatatattatctaaaatttcccttaagaatgaagattataaattcccgcagttgaggaaactgaataagaggagaatcaggacagaatcttttctcttcgtattcctagtccatttttcttctaactacagtagtgagaacaagaatttattcaccaaatcataatacattgcaattaggggatgccatttgaatcttggaagacacgatttgaggcaaataaaaaatccatatttataaagtcaattattggtttatataatttactgtttcagcgggaggtataggttagtaatgtaagtcacttcaagaaggttttggtcgaagttttgccaattttttctgtaaaaagccagatagtaaatattttgggttttacaagccagaagatctttgttgaagttactcaactctgctaatgaggtgcaaagcagccataggcagtttgtaaatgcataagtgtggctgtgttccaataaaagtgtgtttgcaagaacaggcagttggttggatttggcccacaagccgtagttcactgacccctgttttagataaatactagtcaatagattccttgtagctaataaagggaatatagattgtttaatgtgtttccctaatcttcttaaggcttcctccaggggaaaaaatatttcttatcatcagcagtgttattaatgcttactcagaacaaagaataccacttacttagcatagaatggacatttataaagccactcatggaaagaataaagagatgtgaaatacctggtacaactcttaagagtgttccactatcatttgaaaaaatgatgagtagtagttatataaaaggactcgagcctaaaacatttgttctcactttggataacgttttcctatctcagcatcaaaaaatacaaagaaaggggataactaggtcttagattttctaaattctcatgactataagtcccatatatttagatattgaagtacccttaaatatacctaagtagaagtaataatttcgatattatgggaaatctaattactgctcatagttctcgagattaaaatgttatacaccacatctcagctttctttgaaacttgaaatggcatacttgtacaaaggtagaatggtctcccaaagcagcgtcatagattagcagaaaagagctatgataatgccacttcaactgactatcatttaaataatgaattagttaggaagagttaaattatatagttacaggcagcaggagatgtcaccatcagggggataagaagccattttccagtacactatttgacttaactggtgcagttccttctttaactgtaggttactcatttgtatccttaatttatcaatatactaatctgacaaataatttattaaggattactgtgtgccctcctgtgtgttagattctagggatacaatatggatcaagataagcgtgattcctagactcctagaaagtaagaaaactaaataactgtacgtgacaaatactaggatgtagaatactagggaagaatcaggaagaatcacctcacccagtctggagtattgcaacagagcgatgtcagaggaaaaattctggaaaaaaaaaaaaaaaaggaaatttgaagatgagacttgaaggataagtaggtgtacctaggcaaacaagtaggggagagtgttccaggcaagagaaacaagtaacagttggaggcaaaatggagcctagcacgttttaaaaatatcagtatggctgaagcactgagttggggttgggaatgggtgaggagcgatgtgtcagggaggacattccagatgatgctggaaatgcatgcatgttcgcaaactgcattttaagacatgttagagaaatcaaacacattttatttgaagggtaattgaaagccactgaagcattgtagagagactagtgacatgagcatatttgggttttagagggattactctggctgaagaataggtaactgattggataagagcaactttggaggctattccagtggttcaggtgatagataataatgacctaaactaaagaagtaataaagggaatggagacaaagtagacagattcaagtgatacttggcacgtggaaacaacaggccctggtaactgattggacataaaatggagagatacagagaacgcgaatagaacacccaggcatttggcttgataaaccgagtagaccgtggtagaaattactaaactatggagtattttagggttcaggggaactgtgagttcaattttagacatctagttttgcaaggccactgaggtaactgagcatatgagtcaggagagaagtttaggctaaaaatatggatgccagagttaaccagcgttatgataaaacaaagctgaagtctcgagggtaagaacacctaaacatctaggctggagaaggcagcaggtaaaaagtaaaaggagaggccaggcacggtggctcacgcctgtaatcccaacactttggggggccgaggtgggtggatcacctgaggtcgggagtttgagaccagcctgaccaacatggagaaaccgtgtctctactaaaaatacaaaatcagccaggcatggtggcgcatgcctgtaatcccagctactcgggaggctgaggcaggagaatcgcttgaacccaggaggtggaggttgcggtgagccaagattgtgccattgcgctccagcctgggcaacaagagtgaaactccgtctcaaaaaataaaaagaaagagagagagagagagagagagagaaacagagaaagagagagaggaagacaaagaaaggaaggaagggagggagggagggagggagggagggaaagaaaagaaaaggagaaaaattcaggagaatggttacttccagggagatggaggcgattgtgctgtggggaacacaagggtggggtcaaggtattagcagtattctatttcttgattggggttgtatttacataaagtgttgctttataattattcttcacactttatgtgtacgttctatgtaatcatctatagataagacagatttcactgtaaaagaaaataaaagcttccaaaagattatcatcacaattgtaacagattcccctggtgcctggagtcacacgccattttcctgcactgcagttgcagctgcagtggacagccctgtgtgagttcagacttgcctttagctgacagcatcccatgtcaagggaatggctcccatttttctactttctatctaagggacttctctgacatcccaggagcccacagattttgtgagctttctcacccttgaagttttagtgagtgagcaaccttcaaccaatggagatgggagcccatggatatatttttaaccactattccttccgggggcaaggggaattctctgtgattctcaggaacatacaaaagttctgtcaaaatacagtccccatggtccataagcattaccttgatgataatacatttgattggcatttcctccccctctgtctcactcttttggtttttcattcttgcttcctagggatcagcttccaaatgagctatctgtacccaagtcctcatccaggccctgttttcaggggacccaaagacaacaataagaaaaatggaactgaaagaggagaagactttaaggcatgaaaaaagttcctctgtattccatactgcatatttaactgcctactcaacagttccacttagatgtctcaaaaataatctcatgatcttttactgctatgaaatgcatgaccttcccctgatatattcctttcttcaggttttgtagcaccacttagctatccagtaacaaaatcttgggggtcattcttaaaaccttcccacctcacccctgctggacatccaccactaagttcagttgatttttttgctcctaaatatttcttggttcagctttcatttttagatttatcctgcatacccctgtacaatctcatcatctctttcctggactgttacagtagtcttatattaggatgatcatagtcttgatttgcctagagaaaacatgctttatgtcattgcctcagagtgactaatagtcccacctttcacttgtaacactatgcaggttattgggttaaataatactgtcatccaaacctaatctctcctcattcatttcttctctcctatgcatcttagctgtgtgaaaattcaaaatataaatttgatgacagacagaactctgtttaaaatgctccagtaccttaccatttatctcaaaataaaaatttcaaaaaaagaaatgatatcccacaggatcttgtatagtgtgaccctttccatttcatcatccttataccagatactgtgaactacttactctcttatctccacattgacctccctcctagttttgttttgcttatgaaagtgcttattttctgttttttaaagccattgcacatattggtctctctgattgagacactatccttttgaatttttgacctcatacctactcacttttcaggtctcagctcaaatgttatactcaggaaagacgctccttacctcccaaactaggttagtgtaaatggcactatttatactcctcttccagagcacacaccaaatcttatatttatttgtgggacaatttgaattcatgttgttccccctactcaattgtgagcttcttgaggacatactctgcctcctccttacctagctttattgaagtataattgagaaataaaaactgtatgtattcaaggtatacaacatgatgattttatatgactatattgtgagatgattaccacaatcaaattaattaatacatctagcacaagaaatagttactattgtgtgtgtgttgggggggggggatgaggacacttaagatccagtcttgtagcaaatttcaagtaaacagtaaagtattattaactatagtaaccataatgtacattagatccccagacatcttataactgaaagtttgtgccctttgaccaatgatatggtttggctgtgtccccacccaaatctcaccttgaattgtaatccctgtaattcccatgtgttgtgggagggacccagggggaagtaattgaatcatgggtttgtttccccccatgctgttgtcgtgatagtgaatgagttctcatgagatctgatggttttataagcatctggcatttcccttgctggcactcattctctctcctatcaccctgtgaaaaggtgccttcctccatgattgtaagtttccggaggcctctgaagccatgcggaactgtgagtctattaaacctcttttctttataaattacccggtcttgggtatttcttcatagcagcatgagaacggactaatacagtaaattggtactgcagagagtggggtattgctgtaaatatacctgaaaatgtggaagtgacttggaactgggtaagaggcacaggttggaacagtttggagggctagaagatgacaggaaaatgtgggaaaatttgaaacttcctagagacttgttgaatggttttgaccaaaatgccgatggtgatgtggacgatgaagtccaggctgaggtggtctcagatggagattaggaacttcttgggaactggagcaaaggacactgttgctaagctttagcaaagagactggcagcattttcccctggcctagagatctgtggaaatttgaacttgagagagatgatctgaaattggaactttgttttaaagggaagcagagcatcaaagtttggaaaatctgcagcctaacaatgtgatagaaaagaaaaacccattttctgagaaatacaagctggctgcagaactttgcttaagtaaaggagccaaatgttaagcgccaagacaatggggaagatgtctccagggcatgtcagaggtcttaatggcagcccctcccatcacaagcccagaggcctaaaaggaaaacatggtttcacgggccaggcacagggccgtgctgctttgtggagtctcaggacttcgtgccctgcataccagctgtggctcaaagaagccaaagtacagctcaggctgttgcttcagagggtgcaggccttagtggcttacatgtggtgttgggcctggggttggacagaagtcaagaattgaggtttgcaacctctgcctagaattcagaggatgtatggaaaagcctaggtgtccaggcagaagtttgctgcaggggcagggccctcatggaaaacctctctgctgggacagtgcagaaggaaaatgtggggtcggagctccacacagagtccccactggggcactgcctagtggagctgtgagaagaggaccaccatcatccaaaccccagaatagtcagaaatgctgatagtttgaaccatgcatctagaaaagctgcagatactcagtgctagccatgatagcagctgggagggggctgtaccctgcaaagccacaagggcagagctgcccaaggccatgggagcccacctcttgcatcagcatgacctggatgtgagacatgtagtcaaaggagatcatttgggcagttcaaagtgtaatgactgccctattggatttaagacttgcatgtggcctgtaacccctttattttggccaatttctcccattcggaacaggtgtatttacccaatgcctgtaccccccattgtatcctggaagtaactaacttgttttggattttcaggctcataggcggatgggacttgctttgccttagatgaaactttggacttggacttttgggttaatgttggaatgagttaagactttgggtgactgttgggaaggcattattgtgttttgaaatgtgatgacatgagatttgggaggggccaggagcagaatgatatgttttggctgtgtccccacccaaatctcaccttgaattgtaatctccaaaatccccaggtgtcatgggagggacccagtgagaggtaattgaatcatggaggcagtttcccccatgctattctcatgatagtgggtgatttcacatgagatctgatggttttataagtgtctggcgtttcccctgctggcactcattctccctcctgccaccctgtgaagaggtgccttctgccatgattgtaagtttcctgaggcctccccagccatgcagaatggtgaatcaattaaaactgttttcttcataaattacccagtctcgggtatttcttcatagcagcatgagaacagactaatacaaccaacatcccctcttgtcgattttcattgaatccccattacctgggaatgaatgaggaaggcagaaatagaaatcagcgtattgttcaatatagaaatcctgaagttacacaagataattacgagcaagactcagtacagagaaggaacccttgagctggggatcagagtcttctggaaacaaggagtaccaaaggggagtgaaaaagagaagtggagaatgagtagtagcagtaaaagaggaagtatgtaaagaaatggaaataaagatgaaataaactgtcatcttgttccagagacaaagctagctaggggagctaaggaaatttagagggataaaaagtctcatccaaaccttcataatgagctaagggctagtttttcatagaccctagcccaacttgcaaccctctcactctcttacttttttcctggctctgttttgctttactgcctttcatataatctaaaatgatgttaaatgtatatttcctgtctcttttctatatcacaatataaattccatgaggccattgcctgtgccacatttaccactgtactcccagtgcctagaatggtgcccagatcaagtagactcccagagtgtctgttgtcaaatgattctcagtccagtgatgtttgcactacatggtgctgccacttttttttttttttttacttcattccatttggaatttggatcattttgccaactttagggtttattcctctttatttcccctgtgtttgctgaatatctgactatctatttttctcagctctcttctcctcacactttgcctccaacttcctctctgctacaaattatacttagcacaagactggagccacttttcattgattttgatgtcagtgcttgagtgacaactccatgtggagaactttcaacctcctgctgtctcgttgtttgctttgaaaaaaaaaaatactcatcttcttcctgaacccactgggtaaatttatgctcacaaaaaagacaaaacttctagaacaaaaactgtatgagcttcaaaagtattagttttcccttgattttgagactcaaaagtaccatagaaattttaaaagggggatgaagtaattttgtcattcaacccgatatattttacaatttgaatgtcagaaccatgaaatgtcttttcagtgtactatcatcctcactatttccacctataaaagaaaagcaaaaatattgtttgagaacaaaaaaatattgcagtagcggaaaaattcactttaagtctttttttggaggttgccacatcacgcctgtcttcagctcctctcactgaagattctttgttttcttgaaaccagttggtcacttccaattcttaccacctataaatcttcctggttctttatatttcactttatatgtcccttatctcctctgatttttgttccagtctttcattttttacattctttgtcatcctatatgtcagaaagaatctgccactccaaattccttaatatatttatctaccagtgtttatttgcctgtcacatgtactcttcagtaatccattgacttgaaaactttctactctgtctttcttactatagtttgcttcttgagttcaaattattaatatattaggtcaagtaattctatttttatgcacattttacctaacattcttggtaaatataattgttttttaaattgcatcacatggtgagtattaatttaagacgtaattagtattaatctactggacaagattttatttattaactgttttgccctttgctagggttctgtaatctcaccctcagtgcctttaaatagtcttacccaaggatcacctatccctcttcaatttaaattctctattgtgttagtttctgagattgcacattctctggtttggtttggtttttcagtgatctctgtggtcacttccaaccatttctacttagtttacttttttccctgttagccctcttcagtgttaatatcacctggaagttaacccattcctagtctgcattgctctatggccagcatcttggctgattggccatcacctatcctgtacttattgtcaatttttttaaattttttattgtggcaaacagcacataacggaaaatttaccatctgtctttgttcatgtatgttgctataaaggaatgccagagactgggcaatttataaagaatggaaggtttatttggctcatggttctgcaggctgtacaaaaaagcatggcaccactaccaacttctcatgagggcctcaggcttcttccattcatggtgaagggcaaaggggagctggtgtgtagagatcacatggttagagagcataaacaagagagagaggggaagtgccaggctgttatttggcaactagctcttgcagaacctaacagaatgagcagtcactcaacctgcccccaggaagggcattaagccattcaagaaggatccacccccatgaccccacacttcccgttaagccccacctccaatattgaggatcaaatttcaacatgagatttggaggggacaacatctgaactatagcattatcttaattcaaataattttactaccaattccatttattagagaatctaagattaagactactgaaactatgccaatgattatctctagttcaggcttctctcttcaattccaaactcatattttcacctgctgacaagctaccacgtagatgttccacaggtatttctaactcaggatatgtaaaattgatgttatcattattttctgaaaatctattcctcttactatattccctatttttgtgaatgacacaatctaatggactggccatgttaaaaatcaggaagtttttctggcactttctttattccttaatattaagccccacgataaattaccaatcctttcgatcttgttaagacaaataagcttgaaatttcttatctcttatttagcactgcaattttgttttctcattcattcagatagcatcttctacaaaacctcttcactagtttttctgtctctcagctcatcttgtacactgcaaatccattaaaactacactttttaacatgccaatcataatgtattagtaaaggttgtataatagttctctttctcttctaggataaaagctaaatttttcagcttggtacacaaggccgttcatggatttggtacctactagtctatctagggtatttcctacatcttccctcctctctcttgaaaaatcaagtaatactaaactatttgtaactccctaatcaccaattgctgcctttatactttaacatatattcttccttttgctagaaatacatttgtcataataggctctttggtaaacttaccattacattctcagtttataatattatttgtgtaagtaccttttttcctacttagtcctccaggggaatgcatgtgcttctctctccatattatcattttaaaatttactcctatttcagcaagtatcataatattttactgagattaattgttggcatatttatgttaccctggtgacatatttaatagttaagatgctttgggctgcaagaaagagaatgcactttcaaagtggcttcaaaaataagaataattcaactcacataactggaagttcagaggttaaagctggctccagatgaagtacaagcagagctctgtcactctttctctatgactttctttgtcctgatttttttctctgcgtcagctttgacctcctcactagttgccctccaggttccaagatgactgccagcaacaaatgggtaacataatttcttgttacagtgagagacataaacacttacctcacagattcagagatttcaccctcagataagaacaatcaaaattttaattgtgtatctactgtatcattagtatatgtattaggtattcatatatatctactattgtactaaagatgattcagaatggtttcataagctggttgatttaaacttttcttcctgaaccaagaggatgagattatcttgactggcttagaatcgaagatttaggtgaatctaaatccatctccagaactgaggatggattccgtagaaacttgaagacaattggaattctcctagaaagcagaatttcagagtgggtgtagggatatctttgaatggaagttgtttaagccaccaacaatattcactccattggggtgacactgggcccaaaggtacccagttaatatttgttcaacgaacctataggaagacaatattatctgcaagctatttataaactaaatgacaacgagaagtatttttagaaaagttaatcatatacaatttagtcattcacattgggaggctcttcctaaatttcgtttctgatccttatcttgatctttgacgcaatcatatcttggcctccatctaaaagccagtagaagcctctggccattttttaaacaattggtttaacaacattaattggaatctatatagctcaacttaaaaaaaaaaaaaagaggaactagcttatcataatacaatattgtcaaggcaagtgatacttccatgcagctattcagcatttggtgtattcttggtttctgcagacctaataaaaagagattatctcagcgtcctctaggtttaattttaaaaaacaagaaaccatgaacaatgaaaatgatttcaaaacatcttcactcacatctaaaatatgaaatagctagatcagaggttcttaataatttaatcggtgaagcataactgaaggatttagaaaaagtgcctaaactattaaaagattcttcagctgctgcttaatgttgatatcaagttattttttacactggcaaacagctaagccattccctggtcacatccacttttcagcatttagcgctcctctcaatcatcccgatcacagccccaggaatgtttcatggcatctccgcaataatagtatattactattgggtcctgaattttgaggtagtttatctttcagaaaggatatgaatagagcaataagtccaagtagagtgggctttattaatgatgcccaagattttagacgtatactataatcttcaatcaattttaacaccacttcccttaccctagttttagtctaaaccagtccaactgtgctgtagtctcagataatacaatgttagattttttttttcaagatatagaattgtcgggaaaacttgtatgtaacctataccaatctaaatttcttagcatttaactctaaagagtaaagcttttagcacttcctgtgtaatacaagggcaagtgctgatttctctgaatataattttcctttgttagcacatatgttcactgtttaataaaaataagaatgcttcaattatctccttttgtcaagggcctgagaaataaagaaatatacagagggtcccaatctaaaaatgttttgatttacaatggttcggcttaggatatttttattttacaatggtgtgaaagtaatatgcactcagtagaaatcatacttcaagtactcatacaactgttctgcttttcatgttcaatacagtattcaataaattacatgagatgttcaacacctttaataaaataggctttgtgttagatgattttgcccaactgtaggctaatgtaagtgttctgaccatgttgaaggtaggctaggctaagcaatgatgttcagtaggttaggtttattaaatgcatttttcaatttatgatgttttcaacttatgatgggtttttcaatttatgatgttttcaacgtatgatgggtttatcaggaaataatcccattgtacgttgaggagcacctgtatcaatataggcatttacacaactctcgtactgatagcggcaggaggcagagaagctctaggcagaaaagggatggtccccagcgaaaaccccaccctcaagccaaaaagcctgaaaccgcagctcaaagtgggaacttatatcccagttttcctgctcgaatgttgcctttttctaaaccacccatggccccaccccaccccatcctgtgcctataaaaaccccagactcagctggtagacaggactacagctggacatcagagagaagcagcttgacttcagagggacaacttgatggcataactttagagaagaatccggctggacttcaggggaagattacttgccacccccatccccttttcagctccccttcccactgagagccactttcatcggcagtacaatccctcacatttacaatccttcaatttgttcatgtgacctcattttccctagatgctggacaagagctcaggagccacaagtgtgaatacaaaaggccctttgcccttgctggtggagggcagctgcctcctgtgaaaagacaaatggcccactgagctgttaacgcctaagctgtccgtggatggcagagctaacagagcactgtaacacaccctctggggcttcaggggtcgcagacgcctccacctagatgctgctccagtgctcatgcactccagttcccacctcgtttgcttgcacactccctccagtgaggagttgagagcagtgggctaagtaaataaggcacccctgttgcgagttccacaaaggggtcagggaaatatcctgcttcgttactaagtaggtataaaccctttactaagttagtaattattcagtaataatacttaaatgaaagctattctgttagactaaattcagattatcaaagacacgacagaaaacagctttctcattcatgaacctattatttccttttgtaaaatatgttaaatgaaaatacagccgtcatgctaatttctaatggtagaacatattttgaaaactcctttatgtttggaagattttgctttagtgcagataatcagaatgatgtgatatactaagaaataatttttaaaatgagatgtgacatttctcataatctaaataagaaatggcaaaacattgtcctaagctaaataactcatatgaagtgataaaatattgctttctaaaggtccatgatatgtagtgatttctaatgtgtttaatagcattgatcctattgggtaattgggtggttctaacatgtaaggaaggcctccagtactaatttcatgtactgggaaactactgggcagggatgaatcccttaactcctaagtaagactatcagatcatataaatctgctttttgatttgcaaagactcttaggcatacctcttgagaatattaaacatctactaaattatatgtaaagcatttcagtctaagatttacaatgctcaaaggagaaagattttaaagttcagcattggaatttccataatttccttccaattgtagaattttacaattgagatagcaaataataatataaataaatatgtaagactgaaatcaacataggagtttggaaagaagactgtatgggttaactagagttgtcaacaaagacttcacagaagattgtgatcgtagacattagtaaggaataaaatgggtgcaaaaaaaagtctaagaaacaacaaggataaatttattttgtaaagagtgaaagcatattgggtattacataaagagactggctagtttgacacaatgtgtacttttggggtaagcagtgggaaatcactttctgtaagtaaagtggaagagaattcaggtattggatatggtcatataggatttttcaacctttgttttgcccccatccctatcctaccagtatcatcacagatgttggcaatattcactcacttgcctgaagatagatattcatgtcaggggctacagatatgtaaaggtgaatgaagttaaatagcaaaattcaagcacactagttctgactgtgcaccctctctcactcagaaaagcactttggaatgcgagagtactcttatattgacaacctttagttggttctaatttataaaaaaatataaaaatatttagacaatttggatattttatatctatgccacaacccattgtcaaatgattgagatatcattgtttggcaacaatgaaactgaaatcaactctaagagtaaagagtacgacttttaaagatatttggttaagagtaatgttgcaaggcagagaccaacattatagaagttctcaacctgtgtgctggatatagctaaaatttcccaggaccattgaaagaaattctcaaatccacaaccacctcaagtgttgtctgtgaatcaaaggagtgatgcattacccacaaacatgtagtagacattttttaagtgatgtagatgctactgcaattaataaatgcaaaattatattacaatattactgaattcagaataagttgattttgtcactatttttctcaatctacgcatgctagatataaagttattatatagagacgtaacatttttaatatttcagaagcatgctcatgatattgtaaaccaggcactgttaaaagatcactggattgggaattaataaacctgggatctagtcctcatttgatctcttgttggctatggtttgggggttttgggcaaactattcaaccttttctgccttggtttaattgataaatgatggtgttttcccagataaatcccttcccaacttaatattaccagatgtagcatctatggtttagaatgtacagtataaattaaccttcctgaagatcttcacaagttactataacctatattttcatggcactgaaaactaagtttttgatagcttacgtttttataaataatttacttactcatttttatcataataataaatctgattcactaatcaccaaaatatcatttttgaaaatagatgcatgaaaggatccgaacttgttatggttttatctgttcaagtcacctaattttggtagccacaggcccccattgtcaataggggaagattatccattttagcaatacagtctctgataacttcagcccatatgcccttcaattccacttttacgtcagattgaatgggaatgtgaggcccccagtagtaaactaaccctactttctctttggaaattggtctttctacctagctctttgcctctgttcacgttcttgtattccacagaaaatatatacattaggtgttaaaatcacaatgattaacaatttttaagtagaaatagttattaagtatagcataatcatgcctttgaattagtacaaaagtaggaaacagagctttagtgacttttttcattctttccaccatttacagggcaaaaatgaagaattttaccaattcaaaactatgcacatgtatagtttccaccagtatttagtagttatgtttctcaagatgtataattcctttcctttctgttttctgtagtttgataaaccctagataggagttaatgttgtttcagttaggtttattatttcttttatgtggtttaatttcatgcaataagctagagattttgtaacataacttgataaaaattttctcccttcgtatctttttgttttaaaataatggattaatatagattgtaattttacagtagaggaaatacattttacttttagttcttatctagatatcttaggaaaaaagaaacatcatttttaaggattattatttttctactaatgaaaaaaatagcatgattttccattctggactttgtaattaactttaccttggaataattgaatcttaaatataacttcattgaaaattttattttcaagtaacattttaaaatgcaaaatttgtgtgatctctactaaaaagaactctcacatcccaatgtgcttatagctacaataattggttaaaagagacaaattataaagaagatataggttgtgacattgaaattacaaaatgtggattagggaagtaagcgtgtagagttttgtatgcaatcaaagttaagttattaccagcttaaaaaacccttacgtaagatgtaaagaaaagcaaaaacctatagtagcaaaagcaaaaacctatagtagatatacaaaaggtaaaaagtaaggaatcaaagtatactactgagcaaaacagtcaaaccataaaagaagacaacaaaaaaaggcatatagaacaaaggatctacaaaacaattagaaaacaactttttaaatggcagtagtaaattcctacctatcaatatttactttgaatgtaaatggattaaattcaccagtctaaagatagagtggccaaatggattaaaacaacaagactcaactacatgctgcccataagagactcacgtcatcttttaggacacatgttgactgaaatagaagagatagaaaaagatattttggtttccatgcaaatggaaaccaaaagagaatggggatagccatacttctattagacaaaataggctttaaaaatcaaaaactaaaaagacacaaagaaggtcattaaataatgataaaaggatcaattcatcaagaaggtataacaattgtaaatatatatgcacccaacattggagtacctaaatatataaagcaaatataaagtgataaaaagaaagagacaaactacagtacaataatagtaggggacttctaccccaaattcaacaatagacagaaaatccacattaaaaaatcaataagaatgcattggactttatgctttagatcaaatagacctagcagacatatacagtacatctcatctaacagcaggagaatatacattcttatcaagtgcacaagaaacaattcttcagggtagatcatatgttaggccacaaaatgagtcctaacaaatttaacaagattgaaagcatgtattttatgtaaaacaacgccatcatggaaaaagtactagtgtaaacaaggtttaaatatgatttactgattgtttaaaaaggaattatcttagccctgatctgatgggatttcccctttgtaagcagcaaaaataagttcataatgaagcaactgtaataatacagcttcacagatctttctgaataaacagagttggatatgtttctacttcagaaaccatttactgtgggctcacagcttttccatacactctttacactcttaattttaaacccattcatcaaaaggattaagacaatgagattcaagtccaagacaataggaagtatgtgcatcaaaactgtcatgctaatgctctgaggaacattgttatttcaatagcataatttaaaaccactgaaaccatgttttatttatgattacctttcatacgttcaaaaagaatttgagatggtttggcagggatgtcttaaagaaacaaacaccaaatttttatttgttgtgcttctaacaagcaatttttcctacgtaagtgttacctgttttctccccttgattttgatctcttttgtatggttggtagttgtcgcacttctgggcatagtatggttttctacaagttaaataaaaacataaattgattttgatgtagctagttccactatttcagttagtgtgttttccatacctgcaattcataacatgtttactgacccagaacgttaaaactcaagtttagttcaatacagtcacttcaaaccaacttaagatagcagttacatttccaaggtcattatgaagacggagttcaagacttcagcagttgaatttgtcaagctcatgggtcttttagttacaggaagtgtgcatatttcacataagaaacagcaactctgatgatacattgaaactcaaatatacccaaggagtgtaaaactactttataagcccttaaacaataaatatgccaacaatcctctgcatactttttgtcattttttagagcattcaattgaattatataacatgtgataccaataaataattaactttttatttatttatttagagaatgattcttgctttgtctcccaggctggagtgcaatggcatgatctcagctcactgcaacctccacctcttagtttcaagtgattctcctgcctcagcctcccaagtagctgggattacagccatgcaccaccacgcctggctaattttgtacttttag

SEQ ID NO: 835 - DNA, 50 bp, Homo sapiens
  source 1..50: mol_type = genomic DNA
gtaagtatactggatcccattctctttggctctagctatttgttcaaaag

SEQ ID NO: 836 - DNA, 250 bp, Homo sapiens
  source 1..250: mol_type = genomic DNA
cgttttttaaaaaattgttaaatgtatattaatgaaaaggttgaatcttttcattttctaccatgtattgctaaacaaagtatccacattgttagaaaaagatatataatgtcatgaataagagtttggctcaaattgttactcttcaattaaatttgacttattgttattgaaattggctctttagcttgtgtttctaatttttctttttcttcttttttcctttttgcaaaaacccaaaatattttag

SEQ ID NO: 837 - DNA, 60 bp, Homo sapiens
  source 1..60: mol_type = genomic DNA
tcctttttgcaaaaacccaaaatattttagctcctactcagactgttactctggtgacac

SEQ ID NO: 838 - DNA, 233 bp, Homo sapiens
  source 1..233: mol_type = genomic DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 839 - DNA, 46 bp, Homo sapiens
  source 1..46: mol_type = genomic DNA
tgacacaacctgtggttactaaggaaactgccatctccaaactaga

SEQ ID NO: 840 - DNA, 44 bp, Homo sapiens
  source 1..44: mol_type = genomic DNA
ggttactaaggaaactgccatctccaaactagaaatgccatctt

SEQ ID NO: 841 - DNA, 42 bp, Homo sapiens
  source 1..42: mol_type = genomic DNA
gaaactgccatctccaaactagaaatgccatcttccttgatg

SEQ ID NO: 842 - DNA, 65 bp, Homo sapiens
  source 1..65: mol_type = genomic DNA
tgacacaacctgtggttactaaggaaactgccatctccaaactagaaatgccatcttccttgatg

SEQ ID NO: 843 - DNA, 50 bp, Homo sapiens
  source 1..50: mol_type = genomic DNA
tgctctggcagatttcaaccgggcttggacagaacttaccgactggcttt

SEQ ID NO: 844 - DNA, 60 bp, Homo sapiens
  source 1..60: mol_type = genomic DNA
gatatcaacgagatgatcatcaagcagaaggtatgagaaaaaatgataaaagttggcaga

SEQ ID NO: 845 - DNA, 52 bp, Homo sapiens
  source 1..52: mol_type = genomic DNA
tatcaacgagatgatcatcaagcagaaggtatgagaaaaaatgataaaagtt

SEQ ID NO: 846 - DNA, 32767 bp, Homo sapiens
  source 1..32767: mol_type = genomic DNA
gtatgagaaaaaatgataaaagttggcagaagtttttctttaaaatgaagattttccaccaatcactttactctcctagaccatttcccaccagttcttaggcaactgtttctctctcagcaaacacattactctcactattcagcctaagtataatcaaggatataaattaatgcaaataacaaaagtagccatacattaaaaaggaaatatacaaaaaaaaaaaaaaaaaaaagcagaaaccttacaagaatagttgtctcagttaaatttactaaacaacctggtattttaaaaatctattttataccaaataagtcactcaactgagctatttacatttaaactgtttgttttggcactacgcagcccaacatattgcagaatcaaatataatagtctgggaattgattattatccactcttctaagttgtctgtgccaatttgccttctccaatgataaggataattgaaagagagctataacttaaaaagagaaaagtaacaaaacataagatatttaaaattaccctagatcttaaagttggcatttatgcaatgccatgttcaaatgaacatgtttttaatacaaatagtgcatttttcagcctcagtgtaatccatttggtaaaattatgacatcaactagaaacattagaatacattgatgtaaatatggtttacctagctagatcaaatatactatatatcttttatatttgtgaatgattaagaaaaataatgttggaattgttatacattaaagttttttcacttgtaacagctttcaagcctttctaaagaaatacaaagttgtgctgaaggtatttaggtattaaagtactaccttttgaaaaaacaagaagtgaggcagacagagtaaggggaatttctttgtaaaataaacttcaccaattccataggaataaaagtaatttgatagtaaacaacctgcatttaaaggccttgagcttgaatacagaagacctgaattcagtgccatttgcaaatgatgattgtggtcaagccatctctggatcttcgtttcctattctgagtacagagcatacagagtacacattcacattcacaatatagttatggatatggatgtatataaatatatgtaaatactacatatatgtacctaaaatttgttttacttctgctttaaaaaaagtaattatagccacatttttcagaaaaagtaactgaggctcatagatgtcaaattcccagtaagtagcagaacaaggattcaaatccaagtccatttgattcctaagcttgtgttattacttgctactgcagagagtatacgtagcaagtaatatatgtactgcaagcaatacatactattgctgcggtaataactgtaactgcagttactatttagtgatttgtatgtagatgtagatgtagtctatgtcagacactatgctgagcattttatggttgctatgtactgatacatacagaaacaagaggtacgttcttttacaataccatattgagttatataatactcccaggacttttatttaccaaaggaaacaatattttataatgtttaaagcccaggttttgaagttacattgtctgggttcaaagcttggctcccaagctgtgtgaccttgagtaagttattctgcctacctgagcccaagtttatctagctataaaatggggatagttgtactatctgccttgcagtttgtcatcaggattaagttggttggtacatgaaaaatgcttcccactttgccttgcttactgcttactgctagtattgaacaaatgttagtaattatatttggttccaccacgaactctagaaatctaaccaatgatggcatttgtattatgcaaactgtatatcacatcataatattatatggaaatgagagcttgtttccgcttctgtagcctagtctaccattgacatagcttcctgcagaagttaccagataatagattgggagagaaagtccacacttccttgtgacgggtttgtgagtccagcatttagggaagccattgatgtgctcagtagtctccagagttctctaaataaatgtgtccttttcagaaaggactactgatttgatgccccctcacagagatcgtctttaaatataggtcaaaaactaatgtagagggccaggtgcaatgtttcacgcctgcactcccagcgctttgggaggctgaggcaggtggatcacttgaggtcaggagtttgagaccagcctggtcaacatggccaaaccccatctctactgaaaataaaaaaattagctggtgtggtggcccatgcctataatcccagctactagggaggctgaggcaggagaatcacttgaatcctggaccagaggttccattgagctgagatcacaccattgcactccagactgagtgacagagtgagactccatctcagaaaaaaaaaaatttagggggaaaaatcaaaagccatttctgagacacaaaaatacaggatttataaattatatatggtatatataaaaatatttttaaaatagtatatatagcatattatatataatgatatgtaatgttcatatattacatatttataaaaaaatctaatctcccttctcttgcttgctgaatagggggatgctttgcctgcctcttcctcttatattaaaaaataattcttaaagacattgtcagttcttggcttttatagcctcaatcaccaaattgtcggtaaaatggccctaaataatcattaaacaaatgtgtgtgagaggggaaataagaaggataagtaagtatggggaggattttgttataatttcaggaaatcaatatcaattttatgtaaagttttaaataaagcaatcccaactttaatgtttgatgtgtgaaaaattaggcaaaattccaaaagggctttataaactgaaaaaaactttactaacacctatccatttttattattttaaccaacttctattgagctgccactaagtacctgggaaacataaagttgtacaacatagaatgtgcaggtaaaagaggttgaaggaagaaaataataacactatgatagagataaattttaggataatagctaacacatatgatatgccagtcattgatctaagtacttcacgtgaattctttaatgcttacaacattactgtgaggtagatagagaggcacagtaaggataataacctgcctgagatcgaggaagaaagacaatgatgagatgtgaactcaggcagtttggttccagagtcctctcccttaaacctcatagttttcaacttctctgatattgtgtgggtgatgctgttggggctttcttcagggaaaactaagccaggagagagaatggatgctagtgagatattcctgaagaaggaaaaacttaagccaggcattaaagaatgagttggaattacctagctagataaaacgagaagggcaatccaggcagagggaacagactgtgcttttcactgaggtggaaaaaaaacagagtatatcagaggaattgtgattccatatggctgaagttaagggtatatgatgaggaagaaattgatgaggttgaatagagaggactggggctaaataatgggaatcctttgttgccagactgaggaattttgatgatggcctacaggcagtggcaactctgaaaggattgtaaacaggaaaataaaatcatcacatatagtttagttgcctatcaattagagctctctggatgcaagcaacagaaatcattctctgattaaatcaggcagaaagtaaatgtgctgtaattagcacaaaggcattggaacaaaacttacaaaaggaaaaagaatctgagcatgcctttctgggcatgtggctagcaagaagtattccagtctgtttgtgatactctcttttctccatcctgtgtgtaactctgttcaaattttaaagtcttaaaagagagtccagttcaccttgtttgggtcacatgttaatacatgagctagaagggagcagaaaactttgatttaaatccctctcctcccaaagtctcaaaattagggaaaggcaattctcctgaatagaaactgggttctattgacaatagaagaaggaaatgattctgaccaaccactaaacaataattgtccactgaactcagtcaagaacatgtagaataagttggaggatagagcaaataaaggagatttgtaggaggtaattattatgatctaaagcaagcttgttcaactcatggcctgtgagccacatgcgtcccaggatggctttgaatgtggcccaacacaaatttgtaaactttcttaaaacatgagatactttttgtgacttttttttgctcatcagctatcattagtgttagtgtattttatgtgtggcccaggacaattcttcttccagtgtggcccagggaagccaaaagattggatacagctgatctaaagcaacaggttcatctactcaacttcacaacgtgtagacctgaaataaagaccattcatataccaatacctgaaatataaatttgtttgaccatgacacgtacagtaattggttctcaataaatgtggatagcttgatggataatgtgaatgcaatgtgataaggaaacttcatattcaacaaagactggaatgtgaggattataattccaaagcaccagaagatagataagataatgcaatgagacattttatgactcaaggcaaagttagttatgagattcagaccaaaccttagacgtgcagtaattgaaatatttgccacagaaggggtataaggacatgacattcaagtaagctaacctttcactagctttagactttgaactcagaaaacatatttggtgaaaagcttatggtcccctttagtatgtattgcttgattaaagtattattttagaaaatggtgagctgcttccattttgaaataaaaataatttttactaagtgaattatattcagtgaaaaaaatggaagctacaattacaactttaatttttttaagttttaagaatacagccatttaaaaaaattaagcaaatctgcttcattttagacagtagaaaatataccattatcttttagaagaatagagatgtgaaatatgcaaattaagcctttagaagtaaagcacacatgaagttcaaagtttaatttctagaattgtgaatcaatagcagtggatgatttgtactttatagcttagtgtcggagaaatctgattaaaaaatgctttttctgtttcatcacataaacataagtaaaattgctctgaaacaacaatatttgacaagaattagcagttttcttttttgacataatctatcaaatgaagggaaaaatatgtcctgggttttgctttgagagtgattactaaatctgacccttaaggaaaggaaggagagaacaaagaagggaggaaagaaagggaaggaaggaggaggaaagggaaaaaaagaaggaagagaggaaggaaagcaggaaaaaggggaaggagagagaaaagacaaaagaaaggtagcaaggaaagaaaaaaagacaagaaaggaatattaaagaggacaaaagaggagtgaggaaaggaggaaatggaagagggatggtgggagacaggagggagaaaggtggagggggaaatatgaagagaggttcccagcagtggagactagtgttgctatcaacaaatagaatttagatggccatatgatattatttttcataatactggtgtctgattgcctgtgctgagttaattgtagtcttttttttcaattccgtttggccaggtgttcaggataattcaccacaaaatctcaaccactgcacttgtattgaataaagaattgagttggcaaaggcattttatcctccagtaagacctttccagattggggttgagacaaattggccaatctggacaagatgataatagcattgttcaagattaatttttaaccacacattgcactgttacctgggagatttcattatctaaaaattgaatgagcagttttagtgggtatagtgtatatttaaatgggacataattacttgaatgagtttaatttttgttgttgttgttaaggtcaaagtacttaaaaattatgattttttaaaactctgtctatacacaaaaagcatttgaattagctacagaataattctgattataacttttggtgaatagattcagtcaaaatctgattactaaacaacttgtgtagtatagccctggaagaattgatgggacaatgtgtgggtaaagtggcattggctatttaaactaaaagcaatacaaaacagaatgtttcttggttttattctgttgtcacaaacccagcagaaagtggctattacaatagtttcccttattcaacaaatgagagaagttatagacaatttagttaattgatctaaagtcacttagtaaatgtaattgtcctaacataaacccagacccccagacctcttgggaatagataatgtttcttacttcttttctatttcctcagccacccccctcaacttcttacacatctcatttctccatccaaattataacaaaacaaagcaaacatggtttatttccatgggcatcaaatggatttcacgaggttgggtgacagtcatcttagggtgaggagattgattattctgtttttctctttcatcgatcaacaatccagcccttctcatctcatcatttcatttctgcacaaacttgtttaagaaataccaattaagaaattaattaagaaattaatgttgtaatctgtttggctgaagatatttacaaattttgtgctttaattatcttccaacaaatgtacatgtctctggtagacagcttgcgaccatctggatgactgatccatatttatataattttctttctttacctaatgagaccaaatccactattatcttcaacgaaggatgtaaagatatgtcagtgtcagtaatgtgacttattttatattctctggtcataacaaaaataaaccgccccttaaataaaaaggtcatagagttgcaaacacacacacacacacacacacacacacaaaatcatattttctaagtctcctaattacctttttatggaaaatgataccatatgcttttttcttaaagaaactacataaacttataaactatactaaactacacatttcaaagtctatgaatggaaatgtgtatcttattatattttaattcaattcactgtaaacttttctgtcaaaatcttatcaagcaaaactgatccaggatatttacatgaattctgatggaagtcactgtactgtgttttccataaaataccagtgggattctgataaggaagtttatgtttgccattgtgtttaaatagagaattctgggccgggcatggtagctcacgcctgtaatcccagcactttgggaggctgaggcgggtgtatcacctgaggtcaggagtttgagaccagcctgaccaacatggagaaaccccgtctctactaaaaatacaaaattagccgggcgtggtggcgcatgcctgtagccccagctactcctgaggctgaggcaggagaatcgcttgaacccgggaggcagatgttgcagtgagccgagatcacaccattgcactccagcctgggcaacaagagcgaaactccgtctcaaataaataaataattagagaattctatttacaaatttcctttcttggatctagttagggctcctttatatggagtgatttttattgttttcatagaaatacgtagaatctgggtcttctctaactttcttacaggaaagcaatgtaataaggtttttttttaattttctgaaagttatataatgttattgttccctaaagtttaggacctgccttttaggctttccatttcaccataacttttggtccttaaagtctgtaattgaagttacagtgtgttatgatgtaaatttttcttattattacctttaatgttagggtaatgttaactaatgttaatgttaggtatatggttgtttttttcattccttcgttcaacaaattgtctttgaaacccatgttacaaagcactctagagtcaggttgaagactattaagaaaggagaatagaaagagacactagagtaataatttggatttaaatttgatttccttgtgtatgatagtgaataagtgtgaataagatgaggcagtgatcacacatcactattagtaaaagtgtttctgtacctgtatccacacttttatgtatatggttacttatgttaaagtgatacatattatataaaattaacgtatacattaagtagatattttaatagtctgtaattaaatactactagtattttctttcctccttcaagtgcttacttttgatacctcgagttacagtgtcataaagattctttagaaatatattgactgtcttttaagagcttttgatacaatactgagtttacattcatctgttatttattgaacacttgctggtgaaaggcatcagtgttatctgctcttagggaacaaaaattaaaaagggataggccctaattttagagtgtatcctctataagaaaaacataaaagatagggcagtcatggccacaaaagaaaaaagtgttatggtggtttcaatcatatatgtattagaatgaataaatcaactgatcaattgtgatttcttattctaaatatgtgcctgcctttttcatatagatgaaaattaagctatgtttatctttccagggatcttgttgatttttattcaataacttgggagtgaaagttgatttttgcatatgttttaatgtttttaaatttcataaatgaattgatcagtaatttccaaggtagtaatggctgcattgtttttgaaaaaaaaaaagcaacaggatttgattgtgcttttatgatttttaaagaattcattaaaaataatgccacggtttctaaaatgatttgagtcaatttcttattcgatttataaaaataactttgaatacaattttagtaattcacaaatgctttcagttcccttacctttatattttatattctgtgtaaacaagtgacataatatttaagaattatatatctcctatgatttattcaagaaaagaatatatactgtattatttatttcaagaacagaaatgctttgatttaactgtcatcttctctcttcaattatggaagcaaaataaactgtaatgaccaatgtaacccctcccccatatcaagttaatctatgttcaactccagaattatttttgaacactcaaactagaaattaaaaaaaattaaatccatgaagacgatttttgccaaaagcatatagataaattgagttgattctatacttaagaaagtggagaggagagagtaatttggagagagtaatttactcttaatcccatattttttccctaaatgtgaaagaagtagattgtagtgagagggaaaataacctgtagcaacttcattgaggctaagctttctgtcatgttatattatacgaaagtaatgaaatgcttccacagatagaatcagaagtcccctctgagaaattctacataaaaattagcctgccactttaccacacttactcaagtttgatttttttaagttatgtaatagatgttaggcactagaagaggacatttactgggggcaaagatcagtagttggaaagaatgcaagcaggcaagaagctatatataatgagattttacagtacaattgttttctaaatgaaaatgaggacgggtccagacacaatggctcacacttgtaatatcagtgccaggatggaggatcccttgaggccagaagttcaagagcaacctgggcaacagagtgagacttcatctctacaaaaaaatgaaataaaaagttagctggctgtgatggtgtgggcttgtagccttagctactcaggaggctgaggtgacatgatctcttgggcccaggagttcgaggctgcagtgagctatgatagcgccactggattccaacctgggcaatggaacaaaactccatttctaaaaaagaataaaatataaaactaaaataataaataaataaaaatgaggatatattttattttaacatttggaaactttgtaggtgaggaccatgcaaacattcaaggtgtgagttctgaccaaatccaattattaaccataccaatgacttaaggtttcttcacactccttaaagttgattaatataatgattatatagttgactggtatgtcacagcttgaagcctttgagatttattcctgccttttctgtaaaggttgttttgttaattccagtatgtactggtcgtttttgttttgttttgtttttgtttttgttttgtttttttgagatggagtctcgcactgttgcccgggctggagtgcagtggcacgatctcggctcactgcaacctccgccacctaggttcaagcgattctcctgcttcagcctcctgagtagctgggattacaggcactcaccaccacacccggctaattttttttatttttagtagagatggggtttcactatggtggccaggctggtctcaaatacctgacctcatgatccacctgccttggcctcccaaagtgcggggattacaggcgtgagccaccgtgcccggctgccaatatgtattggtctttttcatcaatgattcagtccaaaatcattttgtcctttaactatatattttcttgtaaagctgcttctgttgtcttgaacttttcttttcaaatgtatgttgtcatttgactttttagattgttattttctggtcctcgaaataaatttaaatttcctgtaaaggaaggtgtaatattctatttgacatagccgctaaagatgtactaggtgctttataaatattgttgatttactttatcttcacagattactagttttacttagtatttggaatatgacaacattttatagagctatattcatatatatgtttatcttaactgttaaatgcaatatgattcatgtcttgttttggtcaatgatgaatgaaagtctcctgagaattaaatttactgcatcgatgcaaaaacaatcataattttagacactctaagaattttagaaattaaaggattttttttttccagtttactctgttaagattgtgtttagctatgcgtgacagcattctcactacagtggcttatccagatagtttctttttctcatagagcaagacttccagaattatgtgttccagggtcagtgcagcacctccaaaaccgtatgtcccaactttttcctccaaccccagtcatctccaacatgagactttctttttgttttgttttgttgtttttgtttttgtttttgttttgagatggagtctctgtcgccaggctggagtgcagtggcgcgatctcgactcactgcaacctctgactccctggttcaagggattctcctgcctcagcctcctgagtagctgggattacgggaacgcaccaccacgcccagctaatttttgtatttttagtagagacggggtttcaccatgttggccaggatggtctcgatctcctgacctcatgatctgcctgcctcagcctcccaaagtggtgggattacaggcgtgagccaccgtgcccagcaagactttttttcctgtggtctcaacatggctactctgcctccaggcactatgtctgtactttaaaatggaagaagggaaaatggggaaagtaaaagcatattccagctgtgtcagctcctgtttgtaaggaaaaccagtgcttttctggcagccccacacagaagagtttctacttgaacagtgcattaaccagaaatgtgtcacgtgaccattcctaacttctaaggatcttgggaggattgagtgttttaactgaataggtgtgtttctttcttcataattcaaaatgtgaaaattggtaacttagttataaaaccttgctagtctgaacagaatttggatttttttagctaagaaggaaagaaagggtatcggataggcagctggctatgctagccaagatactcttaataatgcacatttttcttctttggacataagcagttttaacttagctaaatatgatgtgattgttttcctgtcttcttagttctgtttaaatttgtttcagaaatcaaggaaataaaatggagaaaaaactctattattcatgtctatctttctgcctctgaatatttttatgttggagaaagagaaagcagtaactttcataatagcttacatagtctgacaaattctaaacatgtccgttagcatcaatatacgtggtattaggtcatcagtttttatattctgaattattaagacccaaataaacccactgagttcaagagaaagtatacactgagcaataaaaacattaccagttctagcaatgataatcaaacaagaaaacaagtaatatagtctgtttagaataacatgttttaaagatcaagtttttcttccttaccaatgttgcctttcttgtaacacttttttttccttcttgagataggctttcctatcttttgtcacaaaccccaatatttacatggccattcgtagtctattcatagcagcaccaccccatggcccaaacttgtagatattgccctccttctatggttgttctaataagaataaccactcttgtctctcataatctcagctgttttgtgccgttaaaatggaaaataatgagtattaagatactaactaggggccaggcgcggtggctcacgcctgtaatcccagcactttgggaggatgaggcgggcggatcacgaggtcagaagattgagaccatcctggctaacacggtgaaaccacgtctctactaaaaatacaaaaaattagccaggcgtggtggtgggtgcctgtggtcccagctactcgggaggctactgcactccagcctgggcgacagagcaagactccatctcaaaaaaaaaaaaaaaaaaaatactaactaggtttcagtcatatgagatgaataagtcctacaaatctgtacagcctagggcttgtagttaacaatgttttatatttaaaactttgctaagagagtaaatctttttttacctgttattatcgtacatgcaaaaataatagtaaataaggaaggtaagagaaaaattttggaggtgatgcataggtttatggcatagattctggacatgatttcacaggggtatacagtcatgcattgctcaacaacagatatacattcggagaaatgcatggttagctgattgatcttgttgtgcaaacatcatacagtgtacttacacaaacctagatggtacatcctactatacatctaggttgtatggtatagcctattgctcctaggctataaaccagtacagcatgtgactgtactgaatacttcaggcaattggaacacgatggcaagtatttgtgtatctaatcacatctaaacatagaaaagttacagaaaaatattataatcttataggaccactgtcctacatgtatacagactgtgcacatgatagtggtccattgatcaaaatgtccttatgcagcacataactgtaattatctcgaaactcatcaagctgtgttcattaaatgtgtatagctttttatgtcaataaagtggttaagaaatcaataaagtggttaaaaaatattttgactaggaaatatactatcatttctagttgataaaagatctcaacatttccaaaattgtcctacagaaaaccaggttcatcaggtgttcatacatgatcctcatgaaaaggtcaaataagctgaaaaacatgcatagacgttgcctatcctagcaatctatgatgtacatctccatagtaaggtcactgaaaagtcttttaggaatgttagtattgttagctcagtatttctcagtgatttctccatggaaaccatttgtgtagagcatcttgaggagcacagctgagagaacattatcttagttggatgtgtatgtccctctgagtcacttgatttctctacatatgcttttaccaaattaatcttttagaaatcttttctcttcgcactatgtctataatttgtgaagttgttaccaggataacatttgtgcctctcaccatgatgtacctaccagggtccaagcagccattccttctctagagcaactgtctgagggaaagaatttaacacagcattctacgaaatcattttatttataaaaatagattactgctttacatatagtaatttatatttagaatattgattaattattaaaatctgcatgagagctttaaagagtagtacataatatatagcagtttgtactcaaactgtcttctaaaaaggattcactttttgtttgtattctattgtcctattcgttgatagtgttacgtaagtaattataaaacttaaaatctggaaagagaatgtggactcagaatgccatctcttttgttatttcaaatggattagaaatgaacatacatattcattttctttcattacacatccagagaaatagaatggattttataaatatgtaaaagcaaggattttgatcactgataaaaagggaaggtttggtcactaccttatttcattccttttttcttatccttttttttttttttgtcaattatttgatgacatctctgaacatcaccttttattcatgacaagaattgggtatcatggtaaagaacactgttaatataattcagttacttcaccccctcctgaaatatagagaagctttaagactatgtgaatatttttttctggttttcttgtatttgtagaaatagcatgagctttgtttaaagtcaggcatctaaaaccttgccctgtatgttattgacaacctgcacaaattttaggatctattctattacagtttgttcaactgtaaaactaggatagcaaactctatgtcatattttcgttatcagaatttaaaaagcatgttttaagatcttagtaaataataaatctctactctgtagttgaatttgttctatattctttaagaaattccctttgatggttatgccaacctctgtattacttttcttcacactttaactttgcgctgaaatcatagtagtattttacgttatcagtcaaaataacagtcatccttaaaacaaatatgaattttagatgattaaatagatttgtatggaggttcttcttgctaatcatagcagttatccttggtgaaaaatgatagacacttgaaaaaaccaattaatcatgatggctatttttgcatcataaataaagctttcaaatttgagagggaatcaaaagggcaatggtagtatagtgtctcaaagcccctttccaattgatggtacaaatttaaaaagagagagagagagagaaacatgtttcactgtaattgttttctaagagcttccaaaaaagcgtattttcttaatagattcaaatttttcagttggattgaaagggaagtcttggagtgtagtgaggagggcaccttctgttgagaggtgttcagacgacagagtgtgcccaaggccaaagatgagatggttttgcgaaagtcagtggccacaaacaggtgtgtttgacccctgagagatatgcaggaagtctaccccactttaattcttccaaatattctttaccttaattcccaagtacttgataaaggagcaatggggagaaaatatgcacactattatggaaaagttttgacctacactttggagagttttagattaagagcattctagaaatcagtcccaaatgcctagggtttacttacttaaagataatatcatagtttgggtgactgggaagcataccctgagattgaggtgagcatgcagtatgtctatttaggagtgttcttggggtcaacgtgtaggggcagagggagaagttgagctctgacgcagtcttagtaagggcctcagctgaccgttcagggagttcttaagctggaatgacccttcagaagtgctaggaaacgaagaaaggggactggatctttataaccccgtgtcaagtcatgcactggatgtgggctactccaggaaggcaacgaactttagcaagatgattctctttagccacgggaatttccataagggggctgctatggtctgaatgtttttgtccctccaaaatgtgtatgttgaaacctaacactcaaggtgatggtattagaaggtgggggtttgggggggtgattaggtcatgcgggctctgccttcagaaacaggatcagtgcccttataaaagcggctccagaaagcttccttgccctcccaccatgtaaggacacaccgaagatgccatttaacaggagtgggccctcaccagacaatgaatctgctgatgtcttgatcttggaattcccagcctccagaactataagcaataaattctgttgtttataaattacccagtctaaggtatttagctatagcggcccagactaagacaagggctgacagctgaaggctgtctaccagcagcactcctagcagctggggaactaagtccttcatttccaaaggggaatctaggcagcatatttacagcttttcactacagataagctcattatttcaaatagggactagcaggaaaaaattaaattgcccaaaatttagtgggatgctgaaatagattgtggtgtgtaaattggagtatagtgaggagagcaccttcaaaccagtatgtactacatgatattgtttttgttgcaatatttattatatacccaaacacacatatattacttttagaaacacacaccacatatatatctatgaatattttatatacacatagggaaggattgttgatgttatttatgctattttaaagatcgatgttttcatataattatgtattggttatatattatttcttgatataaggtaaaaaaaaaaagcaaaacaaactttaagtgatcactatgaaaagaatcccaatgctgcacatttaggtttatccaactcttcccattaaaatattaaatagtagaaataattgtgaataagaaagagcagattttgaaaaatggaaagaaatgcttaaagacatagcattgttgcccaaccatcattatttaaacatacagtgtttggctttgaccaaattgccttcaaacacttccttttggcccaaaatgttaggtcatatatactaccataaaattcatgatgcttaccatgcattaatttctagtatataccaggcattgtgctatgcatatcatattcaatatttctaatcctctcaaaagtggtacaagctaactggcgtttttcttgttttgaaagggagaaactcagagaggttaagtgacttgcccaaggcaatgccattgataagtgccagattctatcacaggtttattggcaacaaaccatatgtgcgcgtgcatgcgcgtgtgtgtgtgtgtgtgtgtgtgtgtacacatacacgaataacatatatggtataaatacgtggaaacataataaactgcattgagctgcgtttataattagtatttaggacatgtttggcaaataaaaacagtggagattgaaatggatttgcttaggaaaaatgatacattaaaataggctttattatgagtcttcaactattctgtgaaaatagatacccagggaagaaataatagagaatatgaatcttgagcaggcaactgagaacttgtcgaagagccaagataaaaatgtcagagaggagaatattttggcagctcagatgagcccccagagggtgggaggcaatgatctcaccgcagtctcgtatctgaaccccaggtttttgcatctccataaagtaatttcttacacccctcaataatgatcgggcttactctcaatctctcgctctctctctgtgtctctctcacgcacacaaacatgcagaacatttcttgcacatgcataactcataagacgattatgtaaataccagcctttttatttcataactaaattacaaggcctggttattgtttggactgtgaaaaaataattatgtgaataggtgcctcaagatgaaagacaaggcaagattgtgaaattattcatatgatagtaatagtatgcaaaaaataacacaatctttaaagatctttaacgacctagttataaaaccatgctttataacaaatataaccatgaggaaataaaaagaaaaatgtaataatatactccaagaataaagtcaaatgtattgttgaatgtaaggagttggttacacttccttatagtggaggttattttaaaatttgtggcttacgtggtgttatgaattgccctagatcaacactattatgcaaggccaactattaggttatttttggtagataaccacagcaaaactttagtataataggtaaaggttagctacactcccataccctcactctcaggtgttgtcatactccgtataaaaggttcaatcaagggagacatgagaatattccagaatctagaggcaggatgcagttaaccttagagaaggcatcagacaactagaatcttcggattcaatgtggaaacaaagcatagtttaggcattaaatcttgggcaccattccaaagaatacaggttccataacttactatatttttatacctagcaagctagagatgaggaattgctctcaaatattttaaccaaagcatgtatcttaagtaacactaatctcataagtgaaaactcatttctaatattcattttgctcattagcaaggcctctagtgttgactgtgataaaaaatagttcaaatgctggtagaacccaccccaggagactggcctttctgattaaattctaactctatccccacgtgaattcctgacttaagtaactgagttcctgcacatcagaatataagtatattatagatataaaaacatatgtaattaataaatattttaagtgagacacttctttcatctttatggcttaactatatcagacatttgattatttttagcggtctaactacaaaacaaaacacaaagcccacaactaaaaatttctttgtatatattgcaaagaggcaaccatttggtgtcaattcaatcatgagtgaaatgctattatacgagtacatctccctggcttgtatgggggtaatagggcatggaatttacagattcacaataactgagatattcacaataacaaagatatcaatatgtagcttttcccataactttgtgtaatgaaatcctcagtttgtgctgtgtaaaaagcttattgtttacttctcatgaaaatcatcttagtttttatctttatttaatagtctgtaatttgggggtaatacattcgttttgttgatactatgtgaagtggcaagcagaaaattctaacaggaatagataagcaagtatcctataaatcagagtcagtgtctctctctctctcttttaatgagtcagtctgtctctctctctttttccctgcctggctatctatctgtatttttcagttttgctttgcaaataagagaattgtgtgttgtaaaccaaccaacttaccattaattttctctgaattcaaaagcaattacaagcggactcttgagtttgtgctgcctggttgtctgcatataggccagatgtctagaataggatctttatttactatttttaccctcctaatttcatggtaactccaaggtagatgatatttgtaatcgtacactacttgtcagaaatctttctaataacactgctattttataaaaataaacattaattcagtataaaattttattttaaattgttaattcaagcaaatcagtgaggtaacttttacactgccgagcgtacgtgtgtgtggattagtacagccatgccatagacttcacttgtaatcttttctttatattttttatacacctgaaatgttcatcattgtgctgtagaaaacaatctcattgtgtttttaaaagctagagtgggtattgagaaggggaagaggatcatagaaaaagttggttaacatgctacttaacacttcaaatctttactcgatgtcatcatcagcaacattttaaatttatgcttctactagtttgcagttcttttcctttgattattcttatgatacaagcctttccacacaaaatttatgtacaggaattgtgtagaatttttctttggaaaatatggtgatttattacaatttgggcaacatcatcattttaaaaattcagaatttgatttttctcagaatcatcagaaagaataaagcatatatatggttcatgtcaggagaattagaacatgagaattaatatatctctgatcttttaaaatattttcatgtttgtgaatcagcagatttttcctagtttgagatttaaaaaatctagatataattaaaatctcactgatgtttcaccatcagatgattttatatttgtattttcttccacttcataacttgtatagagaagaatagaagaaagaaaaagggaggattgataatctttctctctcagttcttatagcacttcattttttaaacttattacttccttctgcctgctttgtttgtctacatgtttgtatttcatgatttcttagaaatccatctactgccattctgaaggtcatttacctgaaaatgatagaaagcagcatatattcaaacaactgcagagtaattgtctatatcagttatcattgttcattacttttctgttttaggattgaggggctgcctcgccacctccctcacacccccagcatattatcacaaagcctactgattcattcacatccctgggctgaatttgccacccactgtgtgttcctgttgttttgtgtatggaagtgaaaagatttaatttgatgttgttgaaaagacacagaggctaactttcaattttcatatgtagttcttccctctccctctgcaccacctcctttacttgttgagaaaattgccctctccatggtaacaatagaagaagctttcagattttagtagtagttgttgcagagaaaagaattcaaaaagtagatgaagtttaaaaatgaaaaagagagaggaagacagctgggaagaaggcttaatgtttatgagtgggtgtggaggggaagaactaagttgaatgaacaaagctgagctaaggggaagatggtttttctgcatcccagaaggcaataccctagcctttcctgcagccttcactccccaaaagataagagctttatctgaaattcttataggattcattcctgaagagcagcttgtcaccaaacagaaacactgtgatttcctcagggagtcacagtttattattatttttttaatgtaacgcttttgtgaactccagtttccacctcaattcaaatggtcttttggttacagggtgaaagagacccaacaatacacctttcccacttccggaggcctttggttaaaccatgtctgccacaaggacacaggagcctggtatgactggttgttttttgtttgcttttttgcctcctgtgctttctagattgtgagatactgtaactcttgtcgatgacacatagtaccgaacccacccgaagaagtatgtcagtatgtcacattgtgacaaacagcttctcatgctaagtaaatgcagaaccattgtgaaaggtttaataatgcccactcctcccccgccaaagatgtccatatcctaatcccaggaacctgtgaatatgttaccttacatggcaaaaggctttgtattaacagatgtggttaagttaaaaatcttgacacggagagatagcctgggttaccccagtgcgcccaatgtaatcacaagagtcctcctaagagagaaggaggtgatgatacaagcagagtaaaagagagattggaagatgctacactactggcattgaagatgaaggacagggccaagagccaagaaatgcaggcaggctctaaaagctggaaaaggcatggaaaagaatcctcccctacatcccttagagggaatgcaagctctgccaacacattgtttctagcttgtgagacccattttttggactttggacctccaaaattgtaagataataaatttgggttgttttaagccattaagtctgtaatcatttgttacaacagccacaggcagctaatacagccatgaacatttagtaatgactaactttgcacaattttaatacaagcttcttattaaggtttattttttcttaattacaaggaataaaagtggggtctgggggcaatgtcatggtccactccgttttagccatatgaatttgtatttccagcattagaacaaaaggtgacaaatctgaatgtatttgtgtgaaataataataaagcagaacaaaaagggaaaagtgtccagctggaaatgaagttagagaaagatgaggagaagcaagccaattgtgtagttttcccttctgctttttaaaatcatgatttgtttaacccactgaattctattttagaaacaggactgcaaggaagtgttgatggatttggtggcatgagaaccagagtcacagaggcaggaaagtaaggaataagtgttagaataggaagcagagttgcttgggaagagaccttatgacatgtggacagggctagacttaggagtcagaaagacctgagttcaaatgctatcctttagtatagtttgaagtcaggtagcgtgatgcctccagctttgttcttttggcttaggattgacttggcgatgcgggctcttttttggttccatatgaactttaaagtagttttttccaattctgtgaagaaagtcattggtagcttgatggggatggcattgaatctgtaaattaccttgggcagtatggccatttacacgatattgattcttcctacccatgagcacggaatgttcttccatttgtttgtgtcctcttttatttccttgagcagtggtttgtagttctccttgaagaggtccttcacatcgcttgtaagttggattcctaggtattttattctctttgaagcaattgtgaatgggagttcactcatgatttggctctctgtttgtctgtcgttggtgtataagaatgcttgtgatttttgtacattgattttgtatcctgagactttgctgaagttgcttatcagcttaaggagattttgggctgagacaatggggttttctagatatacaatcatgtcgtctgcaaacagggacaatttgacttcctcttctcctaattgaataccctttatttccttctcctgcctgattgccctggccagaacttccaacactatgttgaataggagtggtgagagagggcatccctgtcttgtgccagttttcaaagggaatgcttctatagtacaaggctacagtaaccaaaacagcatggtactggtaccaaaacagacatatagatcaatggaacagaacagagccctcagaagtaacgccgcatatctaccactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagacttaaacgttagacctaaaaccataaaaaccctagaagaaaacctaggcattaccattcaggacataggcatgggcaaggacttcatgtctaaaacaccaaaagcaagggcaacaaaagccaaaattgacaaatgggatctaactaaactaaagagcttctgcacagcaaaagaaactaccatcagagtgaacaggcaacctacaacatgggagaaaattttcgcaacctgcttatctgacaaagagctaatatccagaatctacaatgaactccaacaaatttacaagaaaaaaacaaacaaccccatccaaaagtgggcgaaggacatgaacagacacttctcaaaagaagacatttatgcagccaaaagacacatgaaaaaatgctcaccatcactggccatcagagaaatgcaaatcaaaaccacaatgagataccatctcacaccagttagaatggcaatcattaaaaagtcaggaaacaacaggtgctggagaggatgtggagaaataggaacactcttacactgttggtgggactgtaaactagttcaaccattgtggaagtcagtgtggcgattcctcagggatctagaactagaaataccatttgacccagccatcccattactgggtatatacccaaaggactataaatcatgctgctataaagacacatgcacatgtatgtttattgaggcactatttacaatagcaaagacttggaaccaacccaaatgtccaacaatgatagactggattaagaaaatgtggcacatatacaccatggaatactatgcagccataaaaaaggatgagttcatgtcctttgtagggacatggatgaaattggaaatcatcattctcagtaaactattgcaagaacaaaaaaccaaacaccgcatattctcattcataggtgggaattgaacaatgagaacacatggacacaggaaggggaacatcacactctggggactgttgtggggtggggggaggggggagggatggcattgggagatatacctaatgctagatgacgggttagtgggtgcagcgtgccagcatggcacatgtatacatatgtaactaacctgcacattgtgcacatgtaccctaaaacttaaagtataataataataataataataaaatctcaaaataattaaaaaaagaaacaaacaaatgctatcctgatcctaactggctggctgtctttggggaagttggtaatcttttctgtgcttatttcctcatgtgtaaaaaaatgaatatagtacccagctaggtagagttgttgttgggattaaatgatgactataaagcatctagcccagcttcggctacattatagctgcttacgaaattgtagttacgatgtaaaagagaaaaacactggaaaaggaggatatgggccattttattccaccttcaccaccttttagcttggtgaccttgggcaaattatgcttcattccgtgcttcattttccttgtctataaaagggtgtaagtacagaaccattgaggggtggtcattattaacctacctcaaatggtgtctgtaagttaatatatattgtgcttttcctatgtacaatatctagcacataattacaaatcaaatccatcccatgtgcaatatctagcacataggaaaagcacaataactagttattactcttgttgtagtaattgctacgctgtaggagtttgaattgtaaggcagtggagagtcactgacctttacgagaaagtgtagcagaacatttgagtagatagtaatggggaatattacataaatggatagatattaggggcagatattactattaaaatattacagcatggatatttattaaggccaaactggttaattagttgcatctctcaggttcctaatgttgcttaattttttaacctcccattttgtgctgccctttgtacgaatatttaatgctcccaacacctcttcagtagcacatgtactgtgagtttgttttgttattacttgtgtgtattagcattcctttgtgaaccaaaagcatggaattagctgttgcctctaggctacctagttttgtagtttggattgaagccttcacctcagtaacacctattctgtctactatcttacagaaaacttgtaaaattaagacagatcattaatatagcagaaagagacaaagggcagagaacattgagatactggatattggaaccacccaatagtgttgatttatttatgattatcagtttttgtctctgcctagcctcatgccactaaagtctctgaggcaacaaagaataagcaattttgctcaccttatacaaataaaacacagaaaaaggaatcactagagaaatggtactgcagcctttctgcagggattactgcttatttttaaattacttaaaaggtattgaaattattgttcataatgagaaacctgcctaataaaacagaaaattaaacttaacacttccctataatgtaaacagctcggttaggaacacaacattacagaaaccacttaagaattgattgtacttgttcttggagcagaactagaagctcaccgtttagaagctgtgcacatttccctatcaaacagtacataaagtttccatattcctcagaatcggcttcatttgtgccatgtgtttgcttggaactatgccacagaaagcagttctccccctcaagctgggctcctttcatgccgcagtgcaagtgtgtgatatactggcaccatgtgctaatgtagacccatttttatatgataagaattagtacggcctagggaatagacaagtatgtctaaaatcctccccatagaatatgtcccttcctttaaaagctgtcatactgtaagttccagctgagttaaaggccactgtgctcctatagggaaatatattctattgtaatttttacgttctccaataacagtctgttctttgtttactgaagagagctttcatgtcataaaatggtgttttttgacagagaagcagaatcattgttttattatagaaatttgctcttacaacagcaaaaataaatagctcatctcttaagctcctgatcaatgtctaacacctcctacccccagcaacacttcactgcaagtatattaacactctataatagcaattccactcacctaccaagaaatgatcttcacaaatgatttacagctaaaccagagcttaaacacatagcacccaatcaagggcagatttttatctttttcccagtcatataagttctgagaagaaatagattaatgttgatctcccagacaactgctgagaaaatgtacaaaggatgttgtttattttgaagaatgagacctagttgttaagcactttttccccttatatgtacgtccaaaggtaaccattacaccattttgatgcaaatttaggatatatatttattcatacctctcttctccattcggatgttgtctgtgtgagtgctcacagacacatgcacacatacacacatgcactcctgtttcacacttatttgtaaaactcacaaggatttccaagccattaatatagcattgtttaaggtgaacacatggttgttcaccatccatatgtatcttcactttgtagcactcagaatttggcaaaatcagaaggctgaaacctcatggattaaatatattctatataacatatgtcttaattgctgttactgtaaagaaacctggactagccatatttgactaatttctacctaaggtatttgaattcttataaatagattcattgctttaatcacacaagagtggtttatatgaatgtaattatctccactttatagctgaataaactgagcctgattctatccctatatgggaaacatgaattgaacagctgtgccaattattttgataattcaaatttcacatctaccatgtgaagacagcagaagagggttagggggcttgaattattctgattaactgtgttcatgagtgtaatcgcctctagataatcactcatttcttcacttcacttcccatctacaggtagtatcagcgaatggtaacatctcttgctttgcctcagtttattgatggctgccgttagaaataaaaagcatggttttgtttcagtacttaaatgaataatatcttcaaaatgtttttaaaacgtgaaaaggttgaatgcatttttaacaaatgttttgttagctttgacttttatttttgaacagatgagcacaataccccagtactcctttcctagaaataggagtactacctgaagacttatttcccaaagaaaaatatcaggtctagtgcagcaatacgtattaagggcattgaaaagttatattcacaaaatgtgacatcataacatatgttaatacttcttatcactgataataatccttgaagttgtatttccagagagatctcaatttcttctcacactctgaaagtctctgtttatcctttagagtaggaatgtaagaatttaacaaaacattctgaatgtttaccttttttctaaactgaaactacaatccctttttacccctatatagtaaaatataatattacaagtagaatcagcaaatttgtttaataattctttgggacagtttttacaagcaatggggttgaatttatgttccttgtgtgcagtggagttattatattcttcttaaaaagatgcatgaaggtaaattagaaatgttttacatgttttcatgacaggacatttaatcaaagaggagatacaagaggcttttcttgggttactgcaatttaattttccatttctttcttggaagggacattggatgcagttgtacgaggttaatatttctaacatgccacttttattgtggcattcttctgctctcaaacctctgatgatttcccatggccttcaacatgatgtccttatttgggcatctagtgttctgagccctcacattctggccccagcttcccttctcaacttgatcacagccatcgtaattctgctaactaattacacagctgcccttcccactctttctcaaccactctgctgtattctgacttctacactttagttttaaagctactccttgtcctgaaattccttctcccattaggtcattattaattgagttcatcctctaagttacagttcatgttgctgttcctctatggcaccttccctgaacctggtcccatataaaatcccatctctcagaatcccattagggtaggatatgtggtctgtagactattacgtttgtcatttacatattgtcttctattattgggtaactgcgtgtgcgagcatgcatgggctggcctgaaggtcacctccccaactgcattgaaagttcatcacaagttcaattatttcactagagagtctctttcatgtcatccatgaggcacattccctactgtgatgtgttatgcatacaattatttcaataaatattttctagctctttgattgaccaaagcttaattacctgtcaactctagcctcttgtatctggaatttctacagtctttggatagtatctttaggatgcaaaattaggaggagtatgtaccaggcaaactattacaaataatgccctcaaatagttacatttcactattcatgtcttgtaatttatcttctgctttggtattttagtacacttatgattcaatttgctgtatagattcctctgaatagggacaagagaattcgtcttgataagtggaagttcgaaggattccaaaatgatgttattcaaggtagaacaagaaattaatactgaaaaaattgaggagtaataatccccaaatatgtacatgcgtatctcgttttattgggtttcactttattgcactttgcagatatttcactttttgtaaattgaaagtttgtggcaaggctgcattgagcaagtccgtcgggcacaatttttccaacagcatgtgctcgctttacgtctctgtgtcacgttttggtaatttgctcaatatttcaaacattattattattattatatttgttatgatctgtgatcagtgacctttgatgttactattgtaattgttttgagatgtcatgaactgcactcatatgagatggcaaactttgtggggtgcagtggctcacacctgtaatcctaacactttgggaggccaaggcaggaggatcgtgttagcccagaagtttgagaccagtctgggaaacaaagtgagaccctgtctttaaaatatatatgtagaaaaattaactgggcatggtggcacatggctgtaaggagccctgccagctgcatgggaggctgacacaggaggatcacttgagcccaggaggtcaaggcggcagtaagccatgttcactccagtgccctccagccagaatgacagagcaagaccctgtgtggaaaaaaaaaaaaagacaaacattttttcaactttgattttagattcagggagtacatgtgtaggtttattaccttgatatattacatgatgccgaggtttggagtacaaatgatactgtcacccaggtactgagcatagtaaccgatagttagtttttcaacccttgtttccctccctccccactctagtagtcctccgtttctattgttgccatcattatgtccatgacaacgcactgtttagctccacatgagaacacgtggtatttggtattgtgtttctgcattaattcacttagaataatggcctccagctgcatccatgttgctgcaaaggacatgattttgttcttttttatggctgcatagtattccatggtgtatatgcaccgtattttctttctccagtctgccactgatgggcacctaggctgacttcatacctttgctattgtgaatagtgctgaaatgaggatgagaatacatgtggttttttagtaaagcaatttgttttatttgggctatatgcccagtaatgggatcactaggttgaacgatagttgtgttttaagtcctttgagaaatcttcaaactgtttcactgtggctgaactaatttgcattcccagcaacagtgtatcagagttccctcttctctacagcgtcagcagcatctgtcatttttttgactttttaataatagccagtatgaatggtgtgagacggtatctcgttgtggttttgatttgcatttctctgatgttgagtgatgtggagcattttttcatgtttgttggccacttgtatgtcttcttttgagtagtgtctgtttatgtcttttgcccattttttttgatggggttatttgtttttgacttgttgaattgtttaagttccctatagattctgaatattagacctttgtcagatgcatagtttgcaaatatattctcttattctgtagactgtctgtttactctgttgataaattctttcactgtgaagagctctttagtttaattaagttccacttgtcaatttttggttttgttataattgcttttgaggacttagttataaattctttcccaagtctgatgtccagagtggtgtttcctaggctttcttataggattcttataatttgagatctaatgtttaaacctttattccatcttgagttaatttttgtatatggtgtaaggagggggtccagttgcattcttctgcatatggctaaccagccatcccagcatcatgtcttaaatacagagtccttttcccattacttattttcataagatggcaaacttaatcaatcaatgttttgtgtgttctggctactccactgatcagccattccctcatctctcttcctctccttgggcctccctattccctgagacacaacaatattgaaattatgccagtcagtaaccctacaatgtcctctaagtgttcatgggaaaaaaaagagtcacatgtttgtcactttaaatcaaaagtcagaaatgattaagattggtgaggaaggcatgtcaaaagccaagacaggctgaaagccagacctcttgtgccagttggccaagttgtgaatgcaaaggaaacgttcttgaagaaaattaaaagtgctactctgttgaacacaggaataagaaagtggaacggccttatttttaatatggagaatgtcttagtggtctggatagaggatcaaaacagccacaccattatcttaagcaaaaggctaatctaaagcaaaggactaattctctgcaattctgtgaatgccgagagaggtgaggaagctgcagcagaaaagttggaagctagcagaagtatgttcatgagccttaatgaataagccctctctataacataaaagtgcaaggcagagcaacaagtgctgatggagaagctgcagcaaactatccagaagatcaaactaacatctaagttaataaaggtggcaatactaaacaacacattttcaatatagacgaaacagccttctattggaaaaggatgccatctaggactttcatagctagagagaagtcaatgcctggcttcaaaagttcaaaggacaggctgactgtcttgtgaggggctaatgcaactggtgactttcagttgaagccagtgctcgtttaccattccaaaaatcctagggcccttcaggttatgctagatctagtctgcctatgctctgtaaatcaaacaacaaagactagatgactgcacatctctttacagcatggtttgatgaacattttgagccctctgttgagacctacttctcaaaaaaatgtgtctttcaaaatattactgctctttgacaatgtccctggtcacccaagagccctgaggaatatgtccaaagagaatgattttattttcatacctgctaacacaacatccattctgcagtccttggatcaagaagtcacttcaactttcaagtcttattacttaaatcatacatttcataaaggtatagctgctatattgtggtgagtccactgatggatctggataaagtaaactgaaaacggaaagtcctcaccattccagatgccatgaagaaaattcatgattgaggggaggaggtcaaaatatcaacattatcaggagtttggaagtagttaattccaacactcatcaatgactttgagggtccaa

SEQ ID NO: 847 - DNA, 50 bp, Homo sapiens
  source 1..50: mol_type = genomic DNA
gtatgagaaaaaatgataaaagttggcagaagtttttctttaaaatgaag

SEQ ID NO: 848 - DNA, 43 bp, Homo sapiens
  source 1..43: mol_type = genomic DNA
gtatgagaaaaaatgataaaagttggcagaagtttttctttaa

SEQ ID NO: 849 - DNA, 63 bp, Homo sapiens
  source 1..63: mol_type = genomic DNA
tttccaccaatcactttactctcctagaccatttcccaccagttcttaggcaactgtttctct

SEQ ID NO: 850 - DNA, 54 bp, Homo sapiens
  source 1..54: mol_type = genomic DNA
tatttctaaaagtgttttggctggtctcacaattgtactttactttgtattatg

SEQ ID NO: 851 - DNA, 55 bp, Homo sapiens
  source 1..55: mol_type = genomic DNA
ctttgtattatgtaaaaggaatacacaacgctgaagaaccctgatactaagggat

SEQ ID NO: 852 - DNA, 250 bp, Homo sapiens
  source 1..250: mol_type = genomic DNA
cggaatgtctccatttgagcctttaaatgaagaaaatctatagtcaagattttcatttgaaatatttttgatatctaagaatgaaacatatttcctgttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacag

SEQ ID NO: 853 - DNA, 60 bp, Homo sapiens
  source 1..60: mol_type = genomic DNA
cctgatactaagggatatttgttcttacaggcaacaatgcaggatttggaacagaggcgt

SEQ ID NO: 854 - DNA, 118 bp, Homo sapiens
  source 1..118: mol_type = genomic DNA
gcaacaatgcaggatttggaacagaggcgtccccagttggaagaactcattaccgctgcccaaaatttgaaaaacaagaccagcaatcaagaggctagaacaatcattacggatcgaa

SEQ ID NO: 855-942 - skipped (88 entries)

SEQ ID NO: 943 - protein, 7 aa, synthetic construct
  REGION 1..7: note = Synthetic
ASSLNIA

SEQ ID NO: 944 - protein, 12 aa, synthetic construct
  REGION 1..12: note = Synthetic
SKTFNTHPQSTP

SEQ ID NO: 945 - protein, 12 aa, synthetic construct
  REGION 1..12: note = Synthetic
TARGEHKEEELI

SEQ ID NO: 946 - protein, 9 aa, synthetic construct
  REGION 1..9: note = Synthetic
CQAQGQLVC

SEQ ID NO: 947 - protein, 9 aa, synthetic construct
  REGION 1..9: note = Synthetic
CSERSMNFC

SEQ ID NO: 948 - protein, 9 aa, synthetic construct
  REGION 1..9: note = Synthetic
CPKTRRVPC

SEQ ID NO: 949 - protein, 20 aa, synthetic construct
  REGION 1..20: note = Synthetic
WLSEAGPVVTVRALRGTGSW

SEQ ID NO: 950 - protein, 9 aa, synthetic construct
  REGION 1..9: note = Synthetic
CMQHSMRVC

SEQ ID NO: 951 - protein, 7 aa, synthetic construct
  REGION 1..7: note = Synthetic
DDTRHWG

SEQ ID NO: 952 - protein, 10 aa, synthetic construct
  REGION 1..10: note = Synthetic
GYTFTNYWMH

SEQ ID NO: 953 - protein, 17 aa, synthetic construct
  REGION 1..17: note = Synthetic
EINPINGRSNYGERFKT

SEQ ID NO: 954 - protein, 7 aa, synthetic construct
  REGION 1..7: note = Synthetic
GTRAMHY

SEQ ID NO: 955 - protein, 11 aa, synthetic construct
  REGION 1..11: note = Synthetic
RTSENIYNNLA

SEQ ID NO: 956 - protein, 7 aa, synthetic construct
  REGION 1..7: note = Synthetic
AATNLAD

SEQ ID NO: 957 - protein, 10 aa, synthetic construct
  REGION 1..10: note = Synthetic
QHFWGTPLTF

SEQ ID NO: 958 - protein, 116 aa, synthetic construct
  REGION 1..116: note = Synthetic
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 959 - protein, 107 aa, synthetic construct
  REGION 1..107: note = Synthetic
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 960 - protein, 9 aa, synthetic construct
  REGION 1..9: note = Synthetic
YTFTNYWMH

SEQ ID NO: 961 - protein, 17 aa, synthetic construct
  REGION 1..17: note = Synthetic
EINPINGRSNYAQKFQG

SEQ ID NO: 962 - protein, 17 aa, synthetic construct
  REGION 1..17: note = Synthetic
EINPINGRSNYAEKFQG

SEQ ID NO: 963 - protein, 17 aa, synthetic construct
  REGION 1..17: note = Synthetic
EINPIQGRSNYAEKFQG

SEQ ID NO: 964 - protein, 7 aa, synthetic construct
  REGION 1..7: note = Synthetic
AATNLAE

SEQ ID NO: 965 - protein, 11 aa, synthetic construct
  REGION 1..11: note = Synthetic
RTSENIYSNLA

SEQ ID NO: 966 - protein, 7 aa, synthetic construct
  REGION 1..7: note = Synthetic
AGTNLAD

SEQ ID NO: 967 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 968 - protein, 116 aa, synthetic construct
  REGION 1..116: note = Synthetic
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 969 - protein, 116 aa, synthetic construct
  REGION 1..116: note = Synthetic
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 970 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 971 - protein, 107 aa, synthetic construct
  REGION 1..107: note = Synthetic
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 972 - protein, 107 aa, synthetic construct
  REGION 1..107: note = Synthetic
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 973 - protein, 107 aa, synthetic construct
  REGION 1..107: note = Synthetic
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 974 - protein, 107 aa, synthetic construct
  REGION 1..107: note = Synthetic
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK